(19) United States
(12) Patent Application Publication
(10) Pub. No.: US 2023/0257401 A1
(43) Pub. Date: Aug. 17, 2023

(54) LIGHT EMITTING DEVICE AND NITROGEN-CONTAINING COMPOUND FOR LIGHT EMITTING DEVICE

(71) Applicant: Samsung Display Co., Ltd., Yongin-si (KR)

(72) Inventors: MINJU HAN, Seoul (KR); YOONKYOO LEE, Seoul (KR); HAN GEUL LEE, Hwaseong-si (KR); CHANGWOONG CHU, Hwaseong-si (KR)

(73) Assignee: Samsung Display Co., Ltd., Yongin-si (KR)

(21) Appl. No.: 17/994,703

(22) Filed: Nov. 28, 2022

(30) Foreign Application Priority Data
Feb. 17, 2022 (KR) 10-2022-0020820

(51) Int. Cl. C07F 7/08; C07F 15/00

(52) U.S. Cl. CPC C07F 7/0812; C07F 15/0086; H01L 51/0094; H01L 51/0087

(57) ABSTRACT

Embodiments provide a light emitting device that includes a first electrode, a second electrode facing the first electrode, and an emission layer disposed between the first electrode and the second electrode, wherein the emission layer includes a first compound represented by Formula 1-1 or Formula 1-2, which are each explained in the specification.

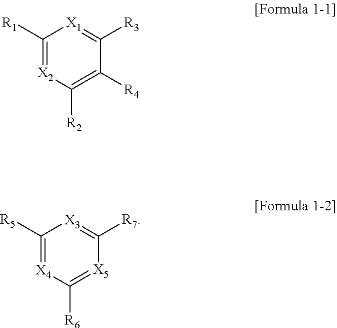

### CROSS-REFERENCE TO RELATED APPLICATION(S)

This patent application claims priority to and benefits of Korean Patent Application No. 10-2022-0020820 under 35 U.S.C. § 119, filed on Feb. 17, 2022, in the Korean Intellectual Property Office, the entire contents of which are incorporated herein by reference.

### BACKGROUND

### 1. Technical Field

The disclosure relates to a light emitting device and a nitrogen-containing compound used in the light emitting device.

### 2. Description of the Related Art

Active development continues for a luminescence display as an image display. The luminescence display is different from a liquid crystal display and is a so-called self-luminescent display in which holes and electrons respectively injected from a first electrode and a second electrode recombine in an emission layer so that a light emitting material in the emission layer emits light to achieve display.

In the application of a light emitting device to a display, there is a demand for a light emitting device having a decreased driving voltage and increased emission efficiency and service life, and continuous development is required on materials for a light emitting device which are capable of stably achieving such qualities.

It is to be understood that this background of the technology section is, in part, intended to provide useful background for understanding the technology. However, this background of the technology section may also include ideas, concepts, or recognitions that were not part of what was known or appreciated by those skilled in the pertinent art prior to a corresponding effective filing date of the subject matter disclosed herein.

### SUMMARY

The disclosure provides a light emitting device having improved emission efficiency and device life.

The disclosure also provides a nitrogen-containing compound which may improve the emission efficiency and device life of a light emitting device.

An embodiment provides a light emitting device which may include a first electrode, a second electrode facing the first electrode, and an emission layer disposed between the first electrode and the second electrode, wherein the emission layer includes a first compound represented by Formula 1-1 or Formula 1-2.

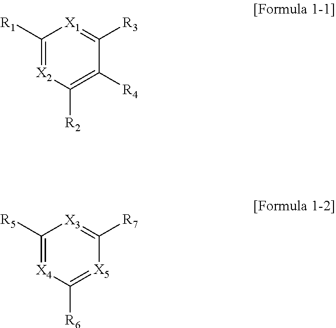

In Formula 1-1 and Formula 1-2, X₁ and X₂ may each be N; X₃ to X₅ may each independently be N or C(R₈); two of X₃ to X₅ may each be N; R₁ to R₄ and R₈ may each independently be a hydrogen atom, a deuterium atom, a halogen atom, a substituted or unsubstituted alkyl group of 1 to 20 carbon atoms, a substituted or unsubstituted aryl group of 6 to 30 ring-forming carbon atoms, or a substituted or unsubstituted heteroaryl group of 2 to 30 ring-forming carbon atoms; R₅ to R₇ may each independently be a substituted or unsubstituted o-biphenyl group, or a substituted or unsubstituted heteroaryl group of 2 to 30 ring-forming carbon atoms; at least one of R₅ to R₇ may each independently be a substituted or unsubstituted o-biphenyl group, or a substituted or unsubstituted six-member heterocyclic group including a nitrogen atom as a ring-forming atom; and at least one of R₁ to R₄ may each independently be a group represented by any one of Formula 2-1 to Formula 2-3.

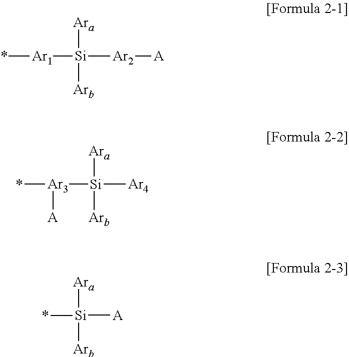

In Formula 2-1 to Formula 2-3, Arₐ, Ar_{b}, and Ar₄ may each independently be a substituted or unsubstituted aryl group of 6 to 30 ring-forming carbon atoms; Ar₁ and Ar₂ may each independently be a substituted or unsubstituted arylene group of 6 to 30 ring-forming carbon atoms; Ar₃ may be a substituted or unsubstituted trivalent aryl group of 6 to 30 ring-forming carbon atoms; and A may be a group represented by Formula 3.

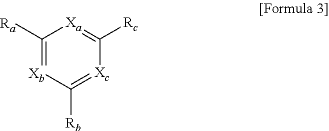

In Formula 3, Xₐ to X_{c} may each independently be N or C(R_{d}); and Rₐ to R_{d} may each independently be a hydrogen atom, a deuterium atom, a halogen atom, a substituted or unsubstituted alkyl group of 1 to 20 carbon atoms, a substituted or unsubstituted aryl group of 6 to 30 ring-forming carbon atoms, or a substituted or unsubstituted heteroaryl group of 2 to 30 ring-forming carbon atoms; except that one of Rₐ to R_{d} is a connecting part to the group represented by one of Formula 2-1 to Formula 2-3.

In an embodiment, the first compound represented by Formula 1-1 may be represented by any one of Formula 4-1 to Formula 4-7.

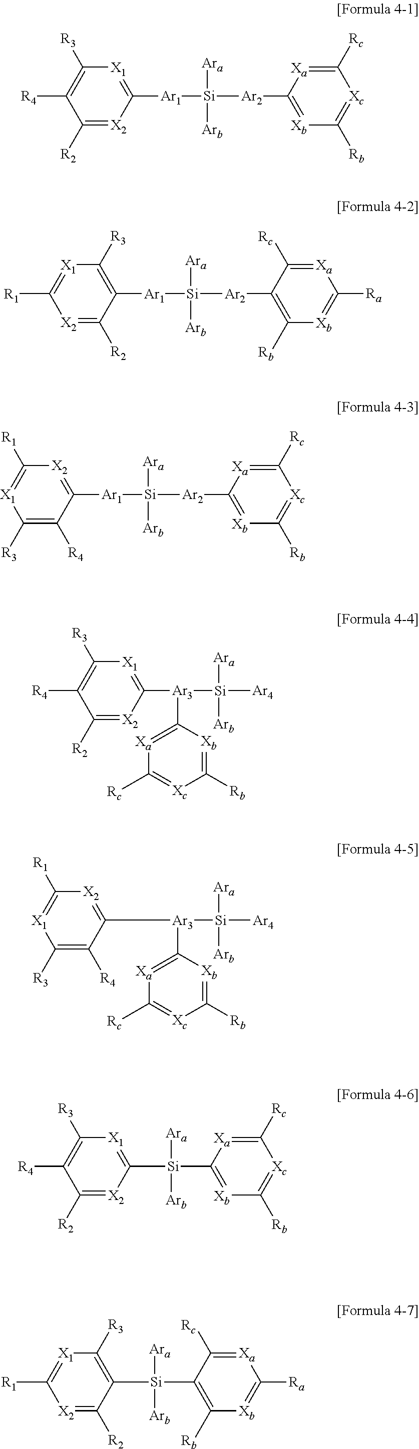

In Formula 4-1 to Formula 4-7, X₁, X₂, R₁ to R₃, Xₐ to X_{c}, Rₐ to R_{c}, Arₐ, Ar_{b}, and Ar₁ to Ar₄ are each the same as defined in Formula 1-1, Formula 2-1 to Formula 2-3, and Formula 3.

In an embodiment, at least one of R₁ to R₄ may each independently be a group represented by any one of Formula 5-1 to Formula 5-3.

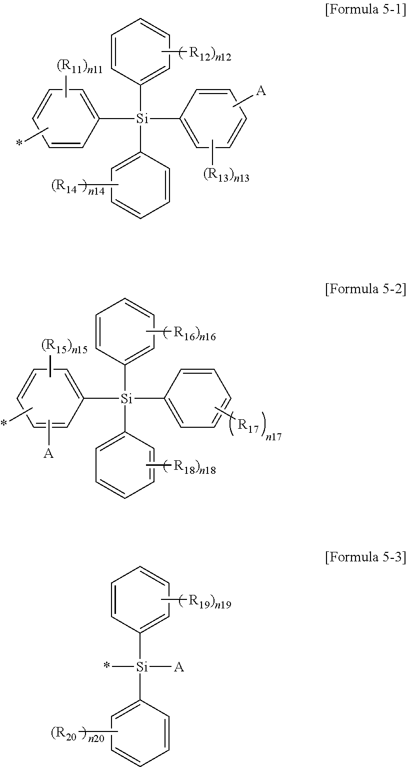

In Formula 5-1 to Formula 5-3, R₁₁ to R₂₀ may each independently be a hydrogen atom, a deuterium atom, a halogen atom, a substituted or unsubstituted alkyl group of 1 to 20 carbon atoms, a substituted or unsubstituted aryl group of 6 to 30 ring-forming carbon atoms, or a substituted or unsubstituted heteroaryl group of 2 to 30 ring-forming carbon atoms; n11 and n13 may each independently be an integer from 0 to 4; n12, n14, and n16 to n20 may each independently be an integer from 0 to 5; and n15 may be an integer from 0 to 3.

In Formula 5-1 to Formula 5-3, A is the same as defined in Formula 2-1 to Formula 2-3.

In an embodiment, at least one of R₁ to R₄ may each independently be a group represented by any one of Formula 6-1 to Formula 6-3.

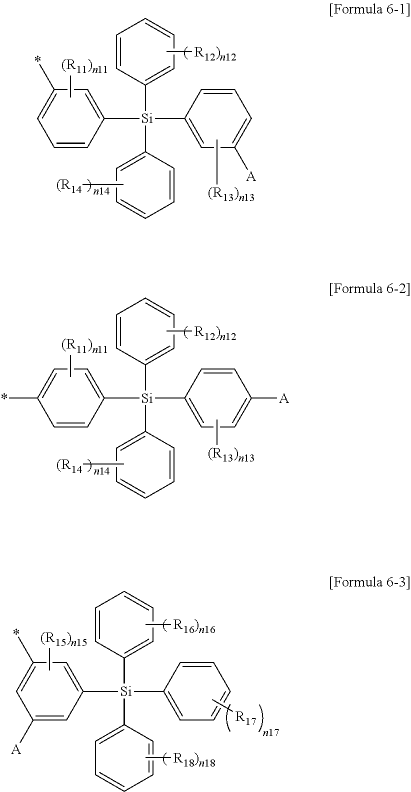

In Formula 6-1 to Formula 6-3, R₁₁ to R₁₈, n11 to n18, and A are each the same as defined in Formula 2-1 to Formula 2-3 and Formula 5-1 to Formula 5-3.

In an embodiment, at least one of R₅ to R₇ may each independently be a group represented by any one of Formula 7-1 to Formula 7-5.

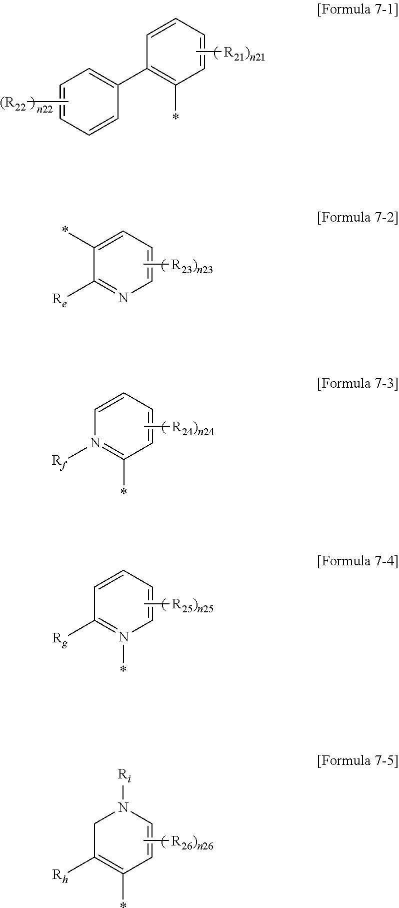

In Formula 7-1 to Formula 7-5, Rₑ to Rₕ may each independently be a substituted or unsubstituted aryl group of 6 to 30 ring-forming carbon atoms; Rᵢ and R₂₁ to R₂₆ may each independently be a hydrogen atom, a deuterium atom, a halogen atom, a substituted or unsubstituted alkyl group of 1 to 20 carbon atoms, a substituted or unsubstituted aryl group of 6 to 30 ring-forming carbon atoms, or a substituted or unsubstituted heteroaryl group of 2 to 30 ring-forming carbon atoms; n21 and n26 may each independently be an integer from 0 to 4; n22, n24, and n25 may each independently be an integer from 0 to 5; and n23 may be an integer from 0 to 3.

In an embodiment, at least one of R₅ to R₇ may each independently be a group represented by any one of Formula 8-1 to Formula 8-5.

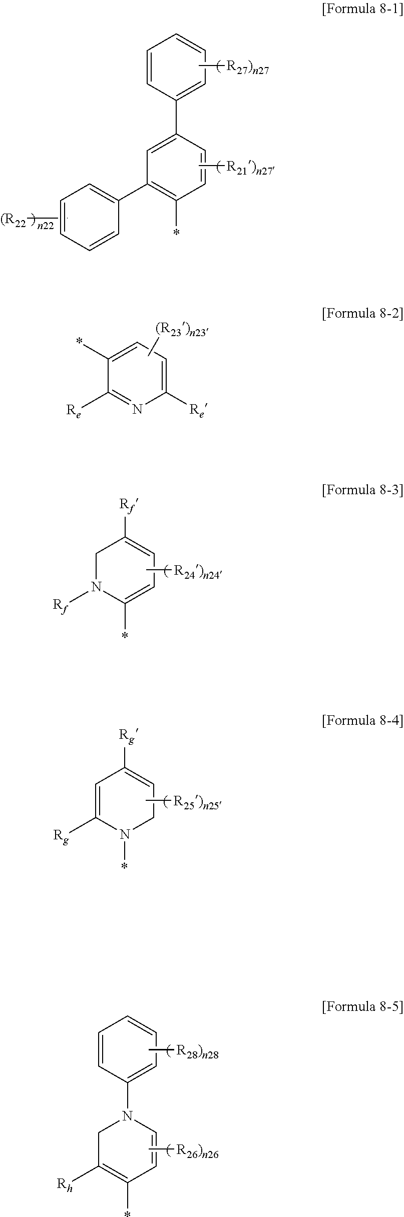

In Formula 8-1 to Formula 8-5, R₂₇ and R₂₈ may each independently be a hydrogen atom, a deuterium atom, a halogen atom, a substituted or unsubstituted alkyl group of 1 to 20 carbon atoms, a substituted or unsubstituted aryl group of 6 to 30 ring-forming carbon atoms, or a substituted or unsubstituted heteroaryl group of 2 to 30 ring-forming carbon atoms; Rₑ′ to R_{g}′ may each independently be a substituted or unsubstituted aryl group of 6 to 30 ring-forming carbon atoms; R₂₁′ and R₂₃′ to R₂₅′ may each independently be a hydrogen atom, a deuterium atom, a halogen atom, a substituted or unsubstituted alkyl group of 1 to 20 carbon atoms, a substituted or unsubstituted aryl group of 6 to 30 ring-forming carbon atoms, or a substituted or unsubstituted heteroaryl group of 2 to 30 ring-forming carbon atoms; n21′ may be an integer from 0 to 3; n23′ may be an integer from 0 to 2; n24′ and n25′ may each independently be an integer from 0 to 4; and n27 and n28 may each independently be an integer from 0 to 5.

In Formula 8-1 to Formula 8-5, R₂₂, R₂₆, n22, n26, and Rₑ to Rₕ are each the same as defined in Formula 7-1 to Formula 7-5.

In an embodiment, the first compound represented by Formula 1-2 may be represented by one of Formula 9-1 to Formula 9-5.

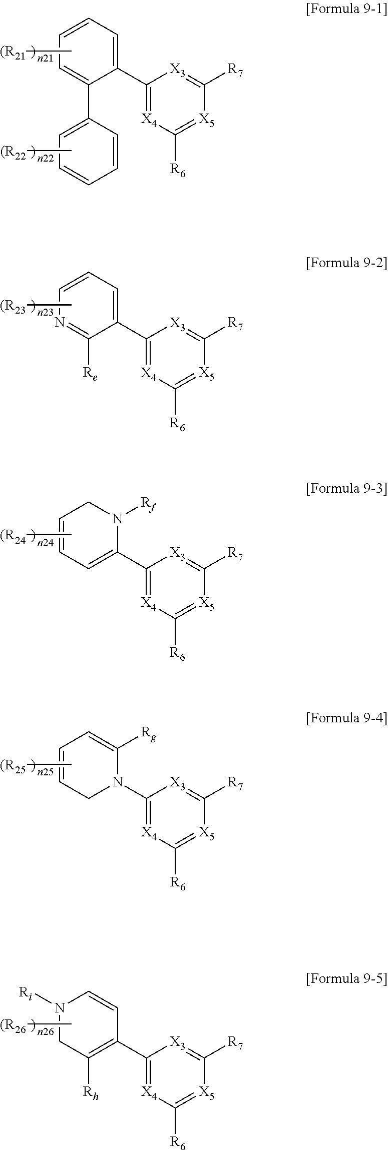

In Formula 9-1 to Formula 9-5, Rₑ to Rₕ may each independently be a substituted or unsubstituted aryl group of 6 to 30 ring-forming carbon atoms; Rᵢ and R₂₁ to R₂₆ may each independently be a hydrogen atom, a deuterium atom, a halogen atom, a substituted or unsubstituted alkyl group of 1 to 20 carbon atoms, a substituted or unsubstituted aryl group of 6 to 30 ring-forming carbon atoms, or a substituted or unsubstituted heteroaryl group of 2 to 30 ring-forming carbon atoms; n21 and n26 may each independently be an integer from 0 to 4; n22, n24, and n25 may each independently be an integer from 0 to 5; and n23 may be an integer from 0 to 3.

In Formula 9-1 to Formula 9-5, X₃ to X₅, R₆, and R₇ are each the same as defined in Formula 1-2.

In an embodiment, at least one of R₆ and R₇ may each independently be a group represented by Formula 10.

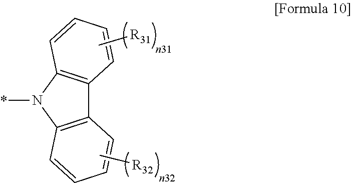

In Formula 10, R₃₁ and R₃₂ may each independently be a hydrogen atom, a deuterium atom, a halogen atom, a substituted or unsubstituted alkyl group of 1 to 20 carbon atoms, a substituted or unsubstituted aryl group of 6 to 30 ring-forming carbon atoms, or a substituted or unsubstituted heteroaryl group of 2 to 30 ring-forming carbon atoms; and n31 and n32 may each independently be an integer from 0 to 4.

In an embodiment, the first compound may include as least one compound selected from Compound Group 1, which is explained below.

In an embodiment, the emission layer may further include a second compound represented by Formula H-1.

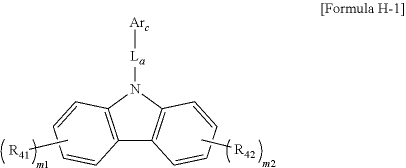

In Formula H-1, Lₐ may be a direct linkage, a substituted or unsubstituted arylene group of 6 to 30 ring-forming carbon atoms, or a substituted or unsubstituted heteroarylene group of 2 to 30 ring-forming carbon atoms; Ar_{c} may be a substituted or unsubstituted aryl group of 6 to 30 ring-forming carbon atoms, or a substituted or unsubstituted heteroaryl group of 2 to 30 ring-forming carbon atoms; R₄₁ and R₄₂ may each independently be a hydrogen atom, a deuterium atom, a halogen atom, a substituted or unsubstituted alkyl group of 1 to 20 carbon atoms, a substituted or unsubstituted aryl group of 6 to 30 ring-forming carbon atoms, or a substituted or unsubstituted heteroaryl group of 2 to 30 ring-forming carbon atoms; and m1 and m2 may each independently be an integer from 0 to 4.

In an embodiment, the second compound may include at least one compound selected from Compound Group 2, which is explained below.

In an embodiment, the emission layer may further include a third compound represented by Formula D-1.

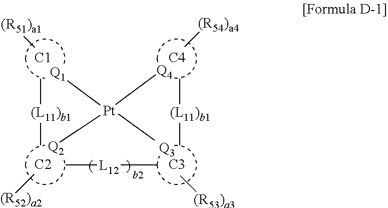

In Formula D-1, Q₁ to Q₄ may each independently be C or N; C1 to C4 may each independently be a substituted or unsubstituted hydrocarbon ring of 5 to 30 ring-forming carbon atoms, or a substituted or unsubstituted heterocycle of 2 to 30 ring-forming carbon atoms; L₁₁ to L₁₃ may each independently be a direct linkage, *—O—*, *—S—*,

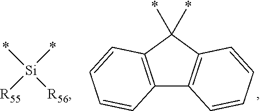

a substituted or unsubstituted divalent alkyl group of 1 to 20 carbon atoms, a substituted or unsubstituted arylene group of 6 to 30 ring-forming carbon atoms, or a substituted or unsubstituted heteroarylene group of 2 to 30 ring-forming carbon atoms; R₅₁ to R₅₆ may each independently be a hydrogen atom, a deuterium atom, a halogen atom, a cyano group, a substituted or unsubstituted amine group, a substituted or unsubstituted alkyl group of 1 to 20 carbon atoms, a substituted or unsubstituted aryl group of 6 to 30 ring-forming carbon atoms, or a substituted or unsubstituted heteroaryl group of 2 to 30 ring-forming carbon atoms, or may be combined with an adjacent group to form a ring; a1 to a4 may each independently be an integer from 0 to 4; and b1 to b3 may each independently be 0 or 1.

In an embodiment, the third compound may include at least one compound selected from Compound Group 3, which is explained below.

Another embodiment provides a nitrogen-containing compound which may be represented by Formula 1-1 or Formula 1-2, which are explained below.

In an embodiment, the nitrogen-containing compound represented by Formula 1-1 may be represented by any one of Formula 4-1 to Formula 4-7, which are explained below.

In an embodiment, at least one of R₁ to R₄ may each independently be a group represented by any one of Formula 5-1 to Formula 5-3, which are explained below.

In an embodiment, at least one of R₅ to R₇ may each independently be a group represented by any one of Formula 7-1 to Formula 7-5, which are explained below.

In an embodiment, the nitrogen-containing compound represented by Formula 1-2 may be represented by any one of Formula 9-1 to Formula 9-5, which are explained below.

In an embodiment, at least one of R₆ and R₇ may each independently be a group represented by Formula 10, which is explained below.

In an embodiment, the nitrogen-containing compound may be selected from Compound Group 1, which is explained below.

It is to be understood that the embodiments above are described in a generic and explanatory sense only and not for the purpose of limitation, and the disclosure is not limited to the embodiments described above.

### BRIEF DESCRIPTION OF THE DRAWINGS

The accompanying drawings are included to provide a further understanding of the embodiments, and are incorporated in and constitute a part of this specification. The drawings illustrate embodiments of the disclosure and principles thereof. The above and other aspects and features of the disclosure will become more apparent by describing in detail embodiments thereof with reference to the accompanying drawings, in which:

FIG. 1 is a plan view of a display apparatus according to an embodiment;

FIG. 2 is a schematic cross-sectional view of a display apparatus according to an embodiment;

FIG. 3 is a schematic cross-sectional view showing a light emitting device according to an embodiment;

FIG. 4 is a schematic cross-sectional view showing a light emitting device according to an embodiment;

FIG. 5 is a schematic cross-sectional view showing a light emitting device according to an embodiment;

FIG. 6 is a schematic cross-sectional view showing a light emitting device according to an embodiment;

FIG. 7 and FIG. 8 are each a schematic cross-sectional view of a light emitting apparatus according to embodiments;

FIG. 9 is a schematic cross-sectional view showing a display apparatus according to an embodiment;

FIG. 10 is a schematic cross-sectional view showing a display apparatus according to an embodiment; and

FIG. 11 is a graph showing emission spectra of light emitting devices of an Example and a Comparative Example.

### DETAILED DESCRIPTION OF THE EMBODIMENTS

The disclosure will now be described more fully hereinafter with reference to the accompanying drawings, in which embodiments are shown. This disclosure may, however, be embodied in different forms and should not be construed as limited to the embodiments set forth herein. Rather, these embodiments are provided so that this disclosure will be thorough and complete, and will fully convey the scope of the disclosure to those skilled in the art.

In the drawings, the sizes, thicknesses, ratios, and dimensions of the elements may be exaggerated for ease of description and for clarity. Like numbers refer to like elements throughout.

In the description, it will be understood that when an element (or region, layer, part, etc.) is referred to as being “on”, “connected to”, or “coupled to” another element, it can be directly on, connected to, or coupled to the other element, or one or more intervening elements may be present therebetween. In a similar sense, when an element (or region, layer, part, etc.) is described as “covering” another element, it can directly cover the other element, or one or more intervening elements may be present therebetween.

In the description, when an element is “directly on,” “directly connected to,” or “directly coupled to” another element, there are no intervening elements present. For example, “directly on” may mean that two layers or two elements are disposed without an additional element such as an adhesion element therebetween.

As used herein, the expressions used in the singular such as “a,” “an,” and “the,” are intended to include the plural forms as well, unless the context clearly indicates otherwise.

As used herein, the term “and/or” includes any and all combinations of one or more of the associated listed items. For example, “A and/or B” may be understood to mean “A, B, or A and B.” The terms “and” and “or” may be used in the conjunctive or disjunctive sense and may be understood to be equivalent to “and/or”.

In the specification and the claims, the term “at least one of” is intended to include the meaning of “at least one selected from the group of” for the purpose of its meaning and interpretation. For example, “at least one of A and B” may be understood to mean “A, B, or A and B.” When preceding a list of elements, the term, “at least one of” modifies the entire list of elements and does not modify the individual elements of the list.

It will be understood that, although the terms first, second, etc. may be used herein to describe various elements, these elements should not be limited by these terms. These terms are only used to distinguish one element from another element. Thus, a first element could be termed a second element without departing from the teachings of the disclosure. Similarly, a second element could be termed a first element, without departing from the scope of the disclosure.

The spatially relative terms “below”, “beneath”, “lower”, “above”, “upper”, or the like, may be used herein for ease of description to describe the relations between one element or component and another element or component as illustrated in the drawings. It will be understood that the spatially relative terms are intended to encompass different orientations of the device in use or operation, in addition to the orientation depicted in the drawings. For example, in the case where a device illustrated in the drawing is turned over, the device positioned “below” or “beneath” another device may be placed “above” another device. Accordingly, the illustrative term “below” may include both the lower and upper positions. The device may also be oriented in other directions and thus the spatially relative terms may be interpreted differently depending on the orientations.

The terms “about” or “approximately” as used herein is inclusive of the stated value and means within an acceptable range of deviation for the recited value as determined by one of ordinary skill in the art, considering the measurement in question and the error associated with measurement of the recited quantity (i.e., the limitations of the measurement system). For example, “about” may mean within one or more standard deviations, or within ±20%, ±10%, or ±5% of the stated value.

It should be understood that the terms “comprises,” “comprising,” “includes,” “including,” “have,” “having,” “contains,” “containing,” and the like are intended to specify the presence of stated features, integers, steps, operations, elements, components, or combinations thereof in the disclosure, but do not preclude the presence or addition of one or more other features, integers, steps, operations, elements, components, or combinations thereof.

Unless otherwise defined or implied herein, all terms (including technical and scientific terms) used have the same meaning as commonly understood by those skilled in the art to which this disclosure pertains. It will be further understood that terms, such as those defined in commonly used dictionaries, should be interpreted as having a meaning that is consistent with their meaning in the context of the relevant art and should not be interpreted in an ideal or excessively formal sense unless clearly defined in the specification.

In the description, the term “substituted or unsubstituted” may describe a group that is substituted or unsubstituted with at least one substituent selected from the group consisting of a deuterium atom, a halogen atom, a cyano group, a nitro group, an amino group, a silyl group, an oxy group, a thio group, a sulfinyl group, a sulfonyl group, a carbonyl group, a boron group, a phosphine oxide group, a phosphine sulfide group, an alkyl group, an alkenyl group, an alkynyl group, an alkoxy group, a hydrocarbon ring group, an aryl group, and a heterocyclic group. Each of the substituents listed above may itself be substituted or unsubstituted. For example, a biphenyl group may be interpreted as an aryl group, or it may be interpreted as a phenyl group substituted with a phenyl group.

In the description, the term “combined with an adjacent group to form a ring” may mean a group that is bonded to an adjacent group to form a substituted or unsubstituted hydrocarbon ring, or a substituted or unsubstituted heterocycle. The hydrocarbon ring may be an aliphatic hydrocarbon ring or an aromatic hydrocarbon ring. The heterocycle may be an aliphatic heterocycle or an aromatic heterocycle. The hydrocarbon ring and the heterocycle may each independently be monocyclic or polycyclic. A ring that is formed by adjacent groups being combined with each other may itself be combined with another ring to form a spiro structure.

In the description, the term “adjacent group” may mean a substituent that is substituted for an atom which is directly combined with an atom substituted with a corresponding substituent, another substituent that is substituted for an atom which is substituted with a corresponding substituent, or a substituent that is sterically positioned at the nearest position to a corresponding substituent. For example, in 1,2-dimethylbenzene, two methyl groups may be interpreted as “adjacent groups” to each other, and in 1,1-diethylcyclopentene, two ethyl groups may be interpreted as “adjacent groups” to each other. For example, in 4,5-dimethylphenanthrene, two methyl groups may be interpreted as “adjacent groups” to each other.

In the description, examples of a halogen atom may include a fluorine atom, a chlorine atom, a bromine atom, or an iodine atom.

In the description, an alkyl group may be linear, branched, or cyclic. The number of carbon atoms in an alkyl group may be 1 to 50, 1 to 30, 1 to 20, 1 to 10, or 1 to 6. Examples of the alkyl group may include methyl, ethyl, n-propyl, isopropyl, n-butyl, s-butyl, t-butyl, butyl, 2-ethylbutyl, 3,3-dimethylbutyl, n-pentyl, i-pentyl, neopentyl, t-pentyl, cyclopentyl, 1-methylpentyl, 3-methylpentyl, 2-ethylpentyl, 4-methyl-2-pentyl, n-hexyl, 1-methylhexyl, 2-ethylhexyl, 2-butylhexyl, cyclohexyl, 4-methylcyclohexyl, 4-t-butylcyclohexyl, n-heptyl, 1-methylheptyl, 2,2-dimethylheptyl, 2-ethylheptyl, 2-butylheptyl, n-octyl, t-octyl, 2-ethyloctyl, 2-butyloctyl, 2-hexyloctyl, 3,7-dimethyloctyl, cyclooctyl, n-nonyl, n-decyl, adamantyl, 2-ethyldecyl, 2-butyldecyl, 2-hexyldecyl, 2-octyldecyl, n-undecyl, n-dodecyl, 2-ethyldodecyl, 2-butyldodecyl, 2-hexyldocecyl, 2-octyldodecyl, n-tridecyl, n-tetradecyl, n-pentadecyl, n-hexadecyl, 2-ethylhexadecyl, 2-butylhexadecyl, 2-hexylhexadecyl, 2-octylhexadecyl, n-heptadecyl, n-octadecyl, n-nonadecyl, n-eicosyl, 2-ethyleicosyl, 2-butyleicosyl, 2-hexyleicosyl, 2-octyleicosyl, n-henicosyl, n-docosyl, n-tricosyl, n-tetracosyl, n-pentacosyl, n-hexacosyl, n-heptacosyl, n-octacosyl, n-nonacosyl, n-triacontyl, etc., without limitation.

In the description, a hydrocarbon ring group may be any functional group or substituent derived from an aliphatic hydrocarbon ring. For example, a hydrocarbon ring group may be a saturated hydrocarbon ring group of 5 to 20 ring-forming carbon atoms.

In the description, an aryl group may be any functional group or substituent derived from an aromatic hydrocarbon ring. The aryl group may be monocyclic or polycyclic. The number of ring-forming carbon atoms in an aryl group may be 6 to 30, 6 to 20, or 6 to 15. Examples of the aryl group may include phenyl, naphthyl, fluorenyl, anthracenyl, phenanthryl, biphenyl, terphenyl, quaterphenyl, quinquephenyl, sexiphenyl, triphenylenyl, pyrenyl, benzofluoranthenyl, chrysenyl, etc., without limitation.

In the description, a heterocyclic group may be any functional group or substituent derived from a ring including one or more of B, O, N, P, Si, and S as heteroatoms. The heterocyclic group may be an aliphatic heterocyclic group and an aromatic heterocyclic group. An aromatic heterocyclic group may be a heteroaryl group. The aliphatic heterocyclic group and the aromatic heterocyclic group may be monocyclic or polycyclic.

In the description, a heterocyclic group may include one or more of B, O, N, P, Si, and S as heteroatoms. If the heterocyclic group includes two or more heteroatoms, two or more heteroatoms may be the same as or different from each other. The heterocyclic group may be monocyclic or polycyclic. The heterocyclic group may be a heteroaryl group. The number of ring-forming carbon atoms in the heterocyclic group may be 2 to 30, 2 to 20, or 2 to 10.

In the description, a heteroaryl group may include one or more of B, O, N, P, Si, and S as heteroatoms. If the heteroaryl group includes two or more heteroatoms, the two or more heteroatoms may be the same as or different from each other. The heteroaryl group may be monocyclic or polycyclic. The number of ring-forming carbon atoms in the heteroaryl group may be 2 to 30, 2 to 20, or 2 to 10. Examples of the heteroaryl group may include thiophene, furan, pyrrole, imidazole, pyridine, bipyridine, pyrimidine, triazine, triazole, acridyl, pyridazine, pyrazinyl, quinoline, quinazoline, quinoxaline, phenoxazine, phthalazine, pyrido pyrimidine, pyrido pyrazine, pyrazino pyrazine, isoquinoline, indole, carbazole, N-arylcarbazole, N-heteroarylcarbazole, N-alkylcarbazole, benzoxazole, benzoimidazole, benzothiazole, benzocarbazole, benzothiophene, dibenzothiophene, thienothiophene, benzofuran, phenanthroline, thiazole, isooxazole, oxazole, oxadiazole, thiadiazole, phenothiazine, dibenzosilole, dibenzofuran, etc., without limitation.

In the description, the above description of the aryl group may be applied to an arylene group, except that the arylene group is a divalent group. The above description of the heteroaryl group may be applied to a heteroarylene group, except that the heteroarylene group is a divalent group.

In the description, a silyl group may be an alkyl silyl group or an aryl silyl group. Examples of the silyl group may include a trimethylsilyl group, a triethylsilyl group, a t-butyldimethylsilyl group, a vinyldimethylsilyl group, a propyldimethylsilyl group, a triphenylsilyl group, a diphenylsilyl group, a phenylsilyl group, etc., without limitation.

In the description, the number of carbon atoms in an amine group is not specifically limited, but may be 1 to 30. The amine group may be an alkyl amine group or an aryl amine group. Examples of the amine group may include a methylamine group, a dimethylamine group, a phenylamine group, a diphenylamine group, a naphthylamine group, a 9-methyl-anthracenylamine group, a triphenylamine group, etc., without limitation.

In the description, a direct linkage may be a single bond.

In the description, the symbol —* represents a binding site to a neighboring atom.

Hereinafter, embodiments will be explained with reference to the drawings.

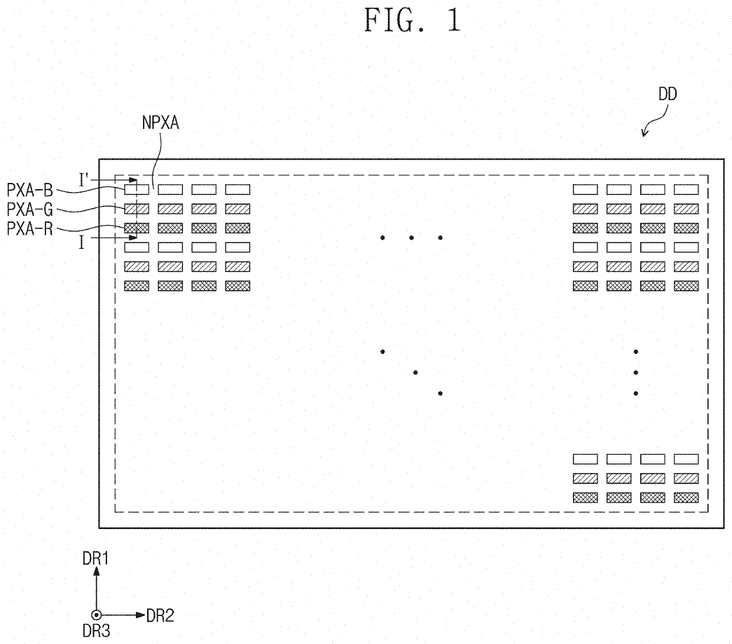

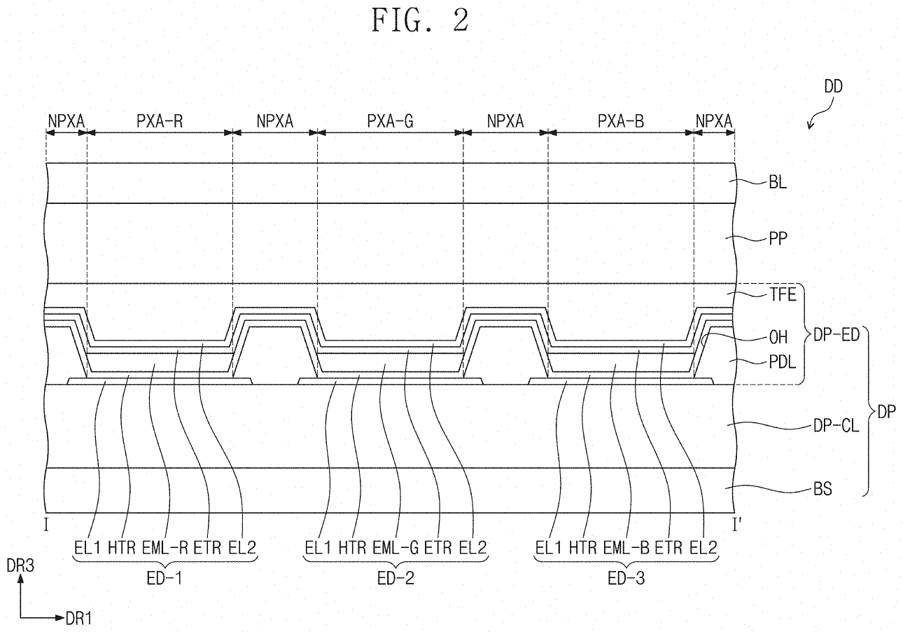

FIG. 1 is a plan view showing an embodiment of a display apparatus DD. FIG. 2 is a schematic cross-sectional view of a display apparatus DD according to an embodiment. FIG. 2 is a schematic cross-sectional view showing a part corresponding to line I-I′ of FIG. 1.

The display apparatus DD may include a display panel DP and an optical layer PP disposed on the display panel DP. The display panel DP includes light emitting devices ED-1, ED-2, and ED-3. The display apparatus DD may include multiple light emitting devices ED-1, ED-2, and ED-3. The optical layer PP may be disposed on the display panel DP and may control light reflected at the display panel DP from an external light. The optical layer PP may include, for example, a polarization layer or a color filter layer. Although not shown in the drawings, in an embodiment, the optical layer PP may be omitted from the display apparatus DD.

A base substrate BL may be disposed on the optical layer PP. The base substrate BL may provide a base surface where the optical layer PP is disposed. The base substrate BL may be a glass substrate, a metal substrate, a plastic substrate, etc. However, embodiments are not limited thereto, and the base substrate BL may include an inorganic layer, an organic layer, or a composite material layer. Although not show in the drawings, in an embodiment, the base substrate BL may be omitted.

The display apparatus DD according to an embodiment may further include a plugging layer (not shown). The plugging layer (not shown) may be disposed between a display device layer DP-ED and the base substrate BL. The plugging layer (not shown) may be an organic layer. The plugging layer (not shown) may include at least one of an acrylic resin, a silicon-based resin, and an epoxy-based resin.

The display panel DP may include a base layer BS, a circuit layer DP-CL provided on the base layer BS, and a display device layer DP-ED. The display device layer DP-ED may include a pixel definition layer PDL, light emitting devices ED-1, ED-2, and ED-3 disposed in the pixel definition layer PDL, and an encapsulating layer TFE disposed on the light emitting devices ED-1, ED-2, and ED-3.

The base layer BS may provide a base surface where the display device layer DP-ED is disposed. The base layer BS may be a glass substrate, a metal substrate, a plastic substrate, etc. However, embodiments are not limited thereto, and the base layer BS may include an inorganic layer, an organic layer, or a composite material layer.

In an embodiment, the circuit layer DP-CL is disposed on the base layer BS, and the circuit layer DP-CL may include transistors (not shown). Each of the transistors (not shown) may include a control electrode, an input electrode, and an output electrode. For example, the circuit layer DP-CL may include switching transistors and driving transistors for driving the light emitting devices ED-1, ED-2, and ED-3 of the display device layer DP-ED.

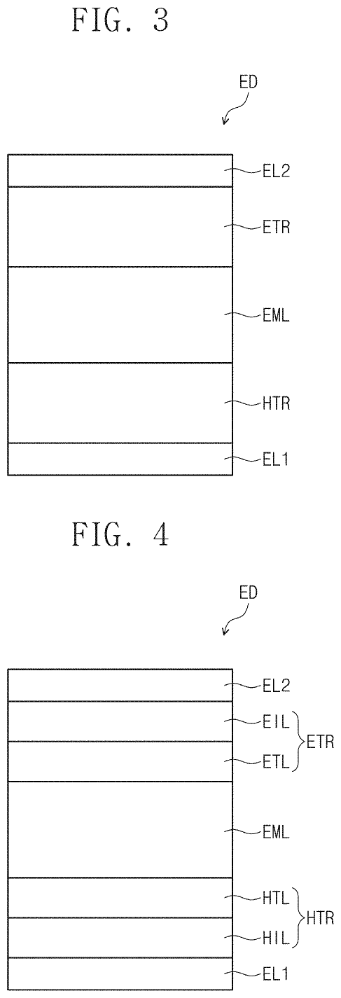

Each of the light emitting devices ED-1, ED-2, and ED-3 may have a structure of a light emitting device ED of an embodiment according to one of FIG. 3 to FIG. 6, which will be explained later. Each of the light emitting devices ED-1, ED-2, and ED-3 may include a first electrode EL1, a hole transport region HTR, emission layers EML-R, EML-G, and EML-B, an electron transport region ETR, and a second electrode EL2.

FIG. 2 shows an embodiment where the emission layers EML-R, EML-G, and EML-B of light emitting devices ED-1, ED-2, and ED-3 are disposed in openings OH defined in a pixel definition layer PDL, and a hole transport region HTR, an electron transport region ETR, and a second electrode EL2 are each provided as a common layer for all of the light emitting devices ED-1, ED-2, and ED-3. However, embodiments are not limited thereto. Although not shown in FIG. 2, in an embodiment, the hole transport region HTR and the electron transport region ETR may each be patterned and provided in the openings OH defined in the pixel definition layer PDL. For example, in an embodiment, the hole transport region HTR, the emission layers EML-R, EML-G, and EML-B, and the electron transport region ETR of the light emitting devices ED-1, ED-2, and ED-3 may each be patterned by an ink jet printing method and provided.

An encapsulating layer TFE may cover the light emitting devices ED-1, ED-2, and ED-3. The encapsulating layer TFE may encapsulate the display device layer DP-ED. The encapsulating layer TFE may be a thin film encapsulating layer. The encapsulating layer TFE may be a single layer or a stack of multiple layers. The encapsulating layer TFE may include at least one insulating layer. The encapsulating layer TFE according to an embodiment may include at least one inorganic layer (hereinafter, encapsulating inorganic layer). The encapsulating layer TFE according to an embodiment may include at least one organic layer (hereinafter, encapsulating organic layer) and at least one encapsulating inorganic layer.

The encapsulating inorganic layer protects the display device layer DP-ED from moisture and/or oxygen, and the encapsulating organic layer protects the display device layer DP-ED from foreign materials such as dust particles. The encapsulating inorganic layer may include silicon nitride, silicon oxynitride, silicon oxide, titanium oxide, or aluminum oxide, without specific limitation. The encapsulating organic layer may include an acrylic compound, an epoxy-based compound, etc. The encapsulating organic layer may include a photopolymerizable organic material, without specific limitation.

The encapsulating layer TFE may be disposed on the second electrode EL2 and may be disposed to fill the openings OH.

Referring to FIG. 1 and FIG. 2, the display apparatus DD may include a non-luminous area NPXA and luminous areas PXA-R, PXA-G, and PXA-B. The luminous areas PXA-R, PXA-G, and PXA-B may each be an area emitting light produced from the light emitting devices ED-1, ED-2, and ED-3, respectively. The luminous areas PXA-R, PXA-G, and PXA-B may be separated from each other in a plan view.

The luminous areas PXA-R, PXA-G, and PXA-B may be areas separated by the pixel definition layer PDL. The non-luminous areas NPXA may be areas between neighboring luminous areas PXA-R, PXA-G, and PXA-B and may be areas corresponding to the pixel definition layer PDL. For example, in an embodiment, the luminous areas PXA-R, PXA-G, and PXA-B may each correspond to a pixel. The pixel definition layer PDL may separate the light emitting devices ED-1, ED-2, and ED-3. The emission layers EML-R, EML-G, and EML-B of the light emitting devices ED-1, ED-2, and ED-3 may be disposed in the openings OH defined in the pixel definition layer PDL and separated from each other.

The luminous areas PXA-R, PXA-G, and PXA-B may be arranged into groups according to the color of light produced from the light emitting devices ED-1, ED-2, and ED-3. In the display apparatus DD according to an embodiment shown in FIG. 1 and FIG. 2, three luminous areas PXA-R, PXA-G, and PXA-B respectively emitting red light, green light, and blue light are illustrated as an embodiment. For example, the display apparatus DD according to an embodiment may include a red luminous area PXA-R, a green luminous area PXA-G, and a blue luminous area PXA-B, which are separated from each other.

In the display apparatus DD according to an embodiment, light emitting devices ED-1, ED-2, and ED-3 may emit light having different wavelength regions from each other. In an embodiment, the display apparatus DD may include a first light emitting device ED-1 emitting red light, a second light emitting device ED-2 emitting green light, and a third light emitting device ED-3 emitting blue light. For example, each of the red luminous area PXA-R, the green luminous area PXA-G, and the blue luminous area PXA-B of the display apparatus DD may respectively correspond to the first light emitting device ED-1, the second light emitting device ED-2, and the third light emitting device ED-3.

However, embodiments are not limited thereto, and the first to third light emitting devices ED-1, ED-2, and ED-3 may emit light in a same wavelength region, or at least one thereof may emit light in a different wavelength region. For example, the first to third light emitting devices ED-1, ED-2, and ED-3 may all emit blue light.

The luminous areas PXA-R, PXA-G, and PXA-B in the display apparatus DD according to an embodiment may be arranged in a stripe configuration. Referring to FIG. 1, the red luminous areas PXA-R, the green luminous areas PXA-G, and the blue luminous areas PXA-B may be arranged along a second directional axis DR2. In another embodiment, the red luminous areas PXA-R, the green luminous areas PXA-G, and the blue luminous areas PXA-B may be arranged by turns along a first directional axis DR1.

In FIG. 1 and FIG. 2, the luminous areas PXA-R, PXA-G, and PXA-B are shown as having similar areas to each other, but embodiments are not limited thereto. The luminous areas PXA-R, PXA-G, and PXA-B may have different areas from each other according to a wavelength region of light emitted. For example, the areas of the luminous areas PXA-R, PXA-G, and PXA-B may be areas in a plan view that are defined by the first directional axis DR1 and the second directional axis DR2.

The arrangement type of the luminous areas PXA-R, PXA-G, and PXA-B is not limited to the configuration shown in FIG. 1, and the arrangement order of the red luminous areas PXA-R, the green luminous areas PXA-G, and the blue luminous areas PXA-B may be provided in various combinations according to the display quality properties which are required for the display apparatus DD. For example, the luminous areas PXA-R, PXA-G and PXA-B may be arranged in a pentile configuration (for example, in a PENTILE™ configuration), or in a diamond configuration (for example, in a Diamond Pixel™ configuration).

In an embodiment, the areas of the luminous areas PXA-R, PXA-G, and PXA-B may be different from each other. For example, in an embodiment, an area of the green luminous area PXA-G may be smaller than an area of the blue luminous area PXA-B, but embodiments are not limited thereto.

Hereinafter, FIG. 3 to FIG. 6 are each a schematic cross-sectional view showing a light emitting device according to embodiments. The light emitting device ED according to an embodiment may include a first electrode EL1, a hole transport region HTR, an emission layer EML, an electron transport region ETR, and a second electrode EL2, stacked in order as shown in FIG. 3.

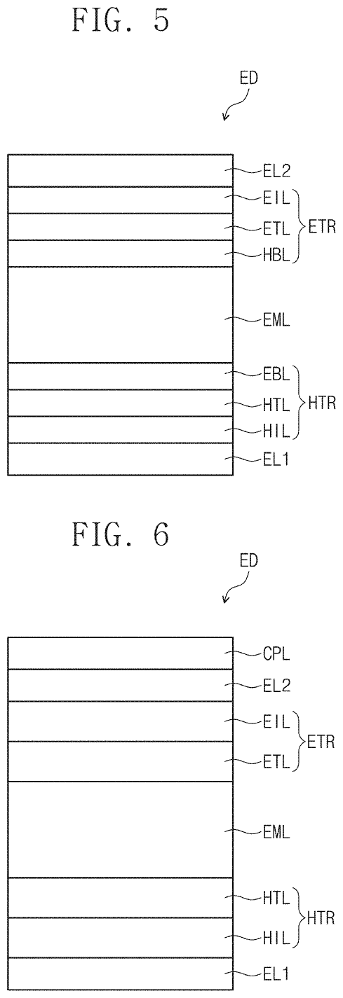

In comparison to FIG. 3, FIG. 4 shows a schematic cross-sectional view of a light emitting device ED according to an embodiment, wherein a hole transport region HTR includes a hole injection layer HIL and a hole transport layer HTL, and an electron transport region ETR includes an electron injection layer EIL and an electron transport layer ETL. In comparison to FIG. 3, FIG. 5 shows a schematic cross-sectional view of a light emitting device ED according to an embodiment, wherein a hole transport region HTR includes a hole injection layer HIL, a hole transport layer HTL, and an electron blocking layer EBL, and an electron transport region ETR includes an electron injection layer EIL, an electron transport layer ETL, and a hole blocking layer HBL. In comparison to FIG. 4, FIG. 6 shows a schematic cross-sectional view of a light emitting device ED according to an embodiment, wherein a capping layer CPL is disposed on the second electrode EL2.

The first electrode EL1 has conductivity. The first electrode EL1 may be formed of a metal material, a metal alloy, or a conductive compound. The first electrode EL1 may be an anode or a cathode. However, embodiments are not limited thereto. For example, the first electrode EL1 may be a pixel electrode. The first electrode EL1 may be a transmissive electrode, a transflective electrode, or a reflective electrode. The first electrode EL1 may include Ag, Mg, Cu, Al, Pt, Pd, Au, Ni, Nd, Ir, Cr, Li, Ca, LiF, Mo, Ti, W, In, Sn, Zn, an oxide thereof, a compound thereof, or a mixture thereof.

If the first electrode EL1 is a transmissive electrode, the first electrode EL1 may include a transparent metal oxide such as indium tin oxide (ITO), indium zinc oxide (IZO), zinc oxide (ZnO), and indium tin zinc oxide (ITZO). If the first electrode EL1 is a transflective electrode or a reflective electrode, the first electrode EL1 may include Ag, Mg, Cu, Al, Pt, Pd, Au, Ni, Nd, Ir, Cr, Li, Ca, LiF/Ca (stacked structure of Li and F), LiF/Al (a stacked structure of Li and Al), Mo, Ti, W, a compound thereof, or a mixture thereof (for example, a mixture of Ag and Mg). In another embodiment, the first electrode EL1 may have a structure including multiple layers including a reflective layer or a transflective layer formed of the above materials, and a transmissive conductive layer formed of ITO, IZO, ZnO, or ITZO. For example, the first electrode EL1 may have a three-layer structure of ITO/Ag/ITO. However, embodiments are not limited thereto. The first electrode EL1 may include the above-described metal materials, combinations of two or more metal materials selected from the above-described metal materials, or oxides of the above-described metal materials. A thickness of the first electrode EL1 may be in a range of about 700 Å to about 10,000 Å. For example, the thickness of the first electrode EL1 may be in a range of about 1,000 Å to about 3,000 Å.

The hole transport region HTR is provided on the first electrode EL1. The hole transport region HTR may include at least one of a hole injection layer HIL, a hole transport layer HTL, a buffer layer (not shown), an emission auxiliary layer (not shown), or an electron blocking layer EBL. A thickness of the hole transport region HTR may be, for example, in a range of about 50 Å to about 15,000 Å.

The hole transport region HTR may be a layer formed of a single material, a layer formed of different materials, or a structure including multiple layers formed of different materials.

For example, the hole transport region HTR may have a structure of a single layer of a hole injection layer HIL or a hole transport layer HTL, or the hole transport region HTR may have a structure of a single layer formed of a hole injection material and a hole transport material. In an embodiment, the hole transport region HTR may be a single layer formed of multiple different materials, or the hole transport region HTR may be a structure in which a hole injection layer HIL/hole transport layer HTL, a hole injection layer HIL/hole transport layer HTL/buffer layer (not shown), a hole injection layer HIL/buffer layer (not shown), a hole transport layer HTL/buffer layer (not shown), or a hole injection layer HIL/hole transport layer HTL/electron blocking layer EBL, are stacked in its respective stated order from the first electrode EL1, but embodiments are not limited thereto.

The hole transport region HTR may be formed using various methods such as a vacuum deposition method, a spin coating method, a cast method, a Langmuir-Blodgett (LB) method, an inkjet printing method, a laser printing method, and a laser induced thermal imaging (LITI) method.

The hole transport region HTR may include a compound represented by Formula H-2.

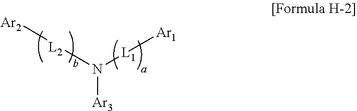

In Formula H-2, L₁ and L₂ may each independently be a direct linkage, a substituted or unsubstituted arylene group of 6 to 30 ring-forming carbon atoms, or a substituted or unsubstituted heteroarylene group of 2 to 30 ring-forming carbon atoms. In Formula H-2, a and b may each independently be an integer from 0 to 10. If a or b is 2 or more, multiple Li groups or multiple L₂ groups may each independently be a substituted or unsubstituted arylene group of 6 to 30 ring-forming carbon atoms, or a substituted or unsubstituted heteroarylene group of 2 to 30 ring-forming carbon atoms.

In Formula H-2, Ar₁ and Ar₂ may each independently be a substituted or unsubstituted aryl group of 6 to 30 ring-forming carbon atoms, or a substituted or unsubstituted heteroaryl group of 2 to 30 ring-forming carbon atoms. In Formula H-2, Ar₃ may be a substituted or unsubstituted aryl group of 6 to 30 ring-forming carbon atoms.

In an embodiment, the compound represented by Formula H-2 may be a monoamine compound. In another embodiment, the compound represented by Formula H-2 may be a diamine compound in which at least one of Ar₁ to Ar₃ includes an amine group as a substituent. In still another embodiment, the compound represented by Formula H-2 may be a carbazole-based compound in which at least one of Ar₁ and Ar₂ includes a substituted or unsubstituted carbazole group, or a fluorene-based compound in which at least one of Ar₁ and Ar₂ includes a substituted or unsubstituted fluorene group.

The compound represented by Formula H-2 may be any one selected from Compound Group H. However, the compounds shown in Compound Group H are presented only as examples, and the compound represented by Formula H-2 is not limited to Compound Group H.

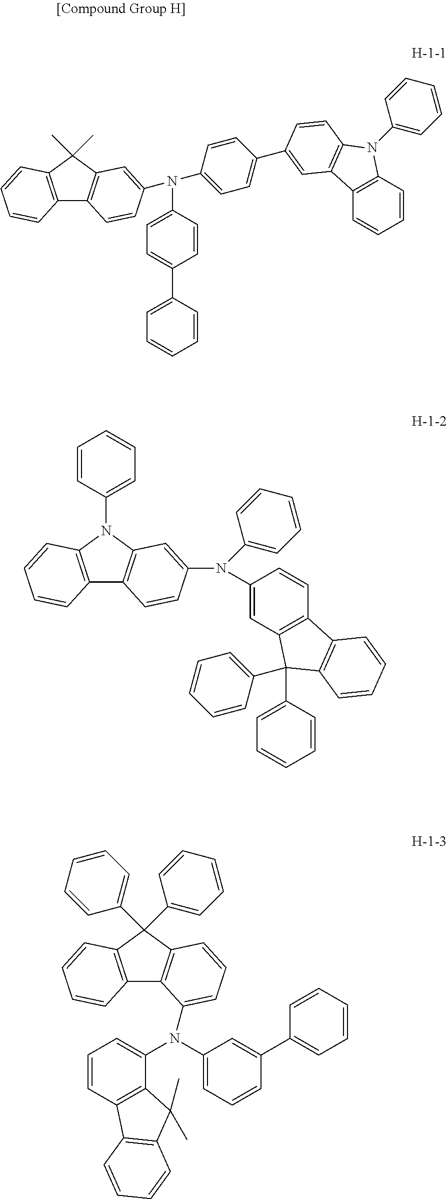 [SMILES: CC1(C)c2ccccc2-c2ccc(N(c3ccc(-c4ccccc4)cc3)c3ccc(-c4ccc5c(c4)c4ccccc4n5-c4ccccc4)cc3)cc21 ; c1ccc(N(c2ccc3c(c2)C(c2ccccc2)(c2ccccc2)c2ccccc2-3)c2ccc3c4ccccc4n(-c4ccccc4)c3c2)cc1 ; CC1(C)c2ccccc2-c2cccc(N(c3cccc(-c4ccccc4)c3)c3cccc4c3-c3ccccc3C4(c3ccccc3)c3ccccc3)c21]

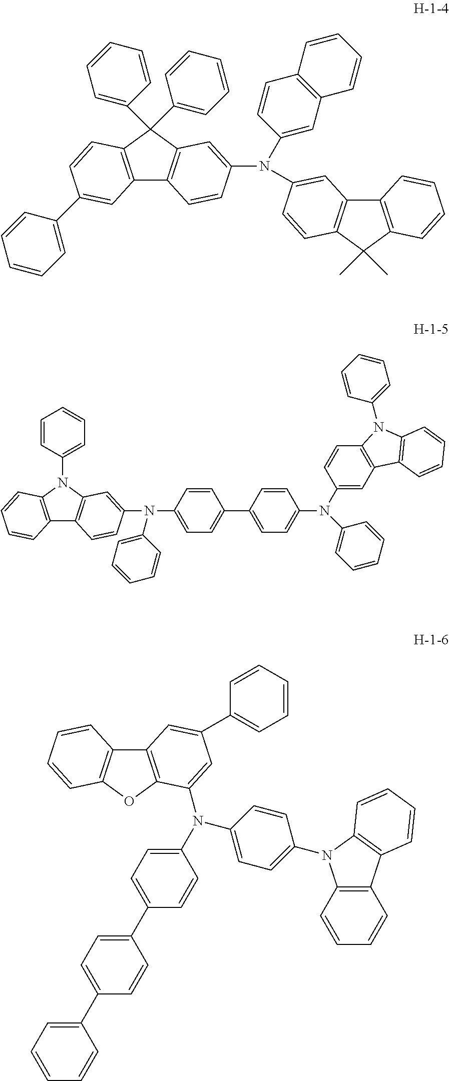 [SMILES: CC1(C)c2ccccc2-c2cc(N(c3ccc4c(c3)C(c3ccccc3)(c3ccccc3)c3ccc(-c5ccccc5)cc3-4)c3ccc4ccccc4c3)ccc21 ; c1ccc(N(c2ccc(-c3ccc(N(c4ccccc4)c4ccc5c6ccccc6n(-c6ccccc6)c5c4)cc3)cc2)c2ccc3c(c2)c2ccccc2n3-c2ccccc2)cc1 ; c1ccc(-c2ccc(-c3ccc(N(c4ccc(-n5c6ccccc6c6ccccc65)cc4)c4cc(-c5ccccc5)cc5c4oc4ccccc45)cc3)cc2)cc1]

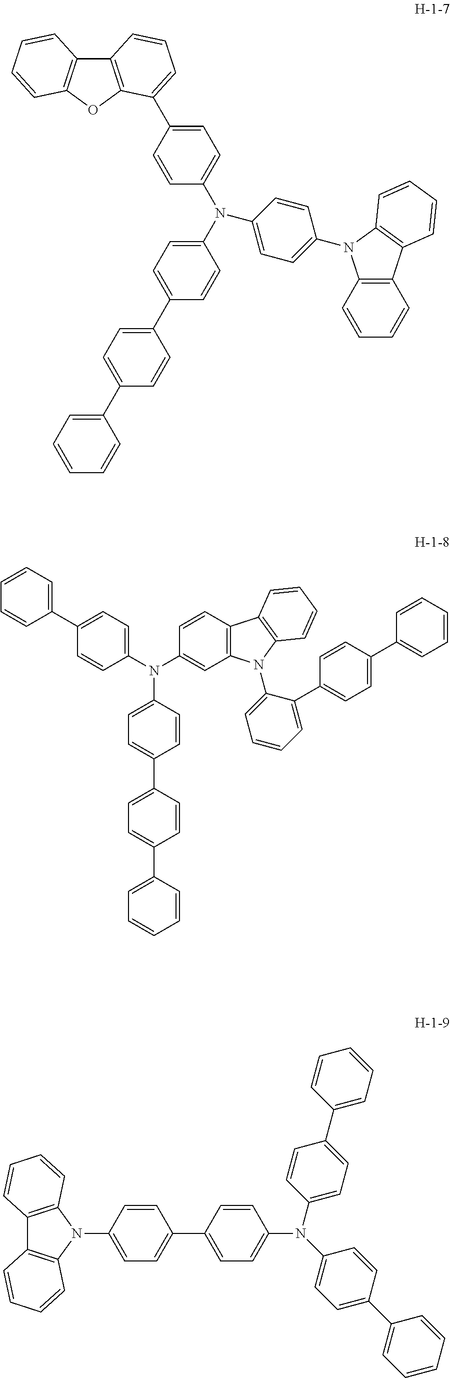 [SMILES: c1ccc(-c2ccc(-c3ccc(N(c4ccc(-c5cccc6c5oc5ccccc56)cc4)c4ccc(-n5c6ccccc6c6ccccc65)cc4)cc3)cc2)cc1 ; c1ccc(-c2ccc(-c3ccc(N(c4ccc(-c5ccccc5)cc4)c4ccc5c6ccccc6n(-c6ccccc6-c6ccc(-c7ccccc7)cc6)c5c4)cc3)cc2)cc1 ; c1ccc(-c2ccc(N(c3ccc(-c4ccccc4)cc3)c3ccc(-c4ccc(-n5c6ccccc6c6ccccc65)cc4)cc3)cc2)cc1]

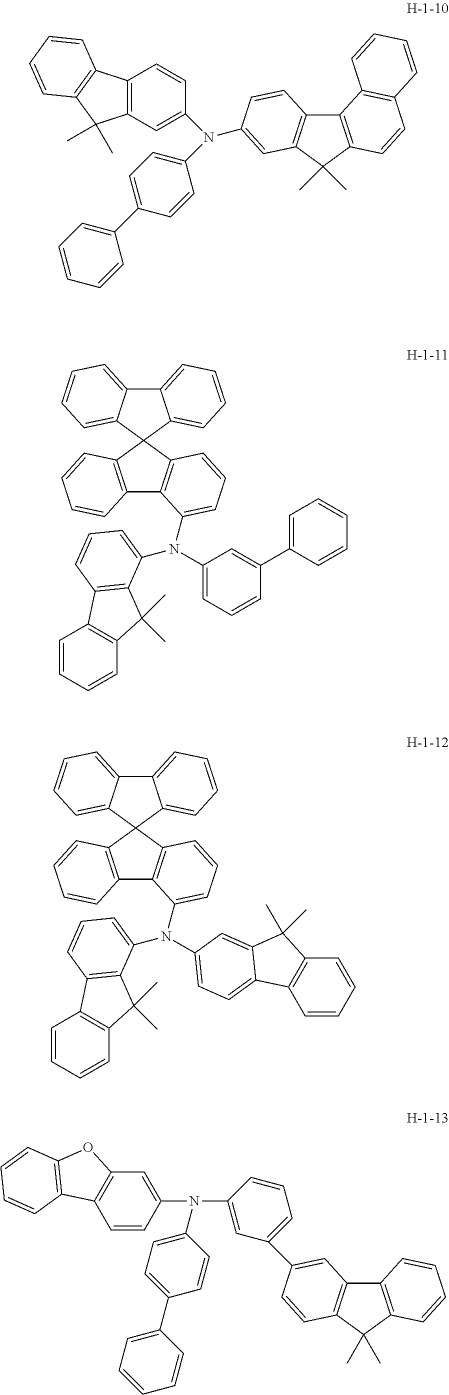 [SMILES: CC1(C)c2ccccc2-c2ccc(N(c3ccc(-c4ccccc4)cc3)c3ccc4c(c3)C(C)(C)c3ccc5ccccc5c3-4)cc21 ; CC1(C)c2ccccc2-c2cccc(N(c3cccc(-c4ccccc4)c3)c3cccc4c3-c3ccccc3C43c4ccccc4-c4ccccc43)c21 ; CC1(C)c2ccccc2-c2ccc(N(c3cccc4c3-c3ccccc3C43c4ccccc4-c4ccccc43)c3cccc4c3C(C)(C)c3ccccc3-4)cc21 ; CC1(C)c2ccccc2-c2cc(-c3cccc(N(c4ccc(-c5ccccc5)cc4)c4ccc5c(c4)oc4ccccc45)c3)ccc21]

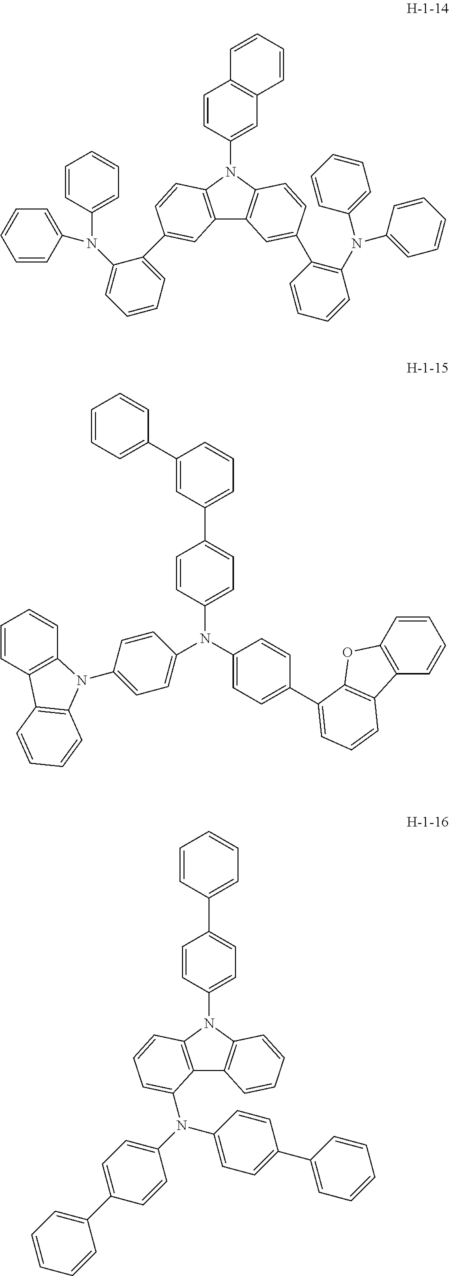 [SMILES: c1ccc(N(c2ccccc2)c2ccccc2-c2ccc3c(c2)c2cc(-c4ccccc4N(c4ccccc4)c4ccccc4)ccc2n3-c2ccc3ccccc3c2)cc1 ; c1ccc(-c2cccc(-c3ccc(N(c4ccc(-c5cccc6c5oc5ccccc56)cc4)c4ccc(-n5c6ccccc6c6ccccc65)cc4)cc3)c2)cc1 ; c1ccc(-c2ccc(N(c3ccc(-c4ccccc4)cc3)c3cccc4c3c3ccccc3n4-c3ccc(-c4ccccc4)cc3)cc2)cc1]

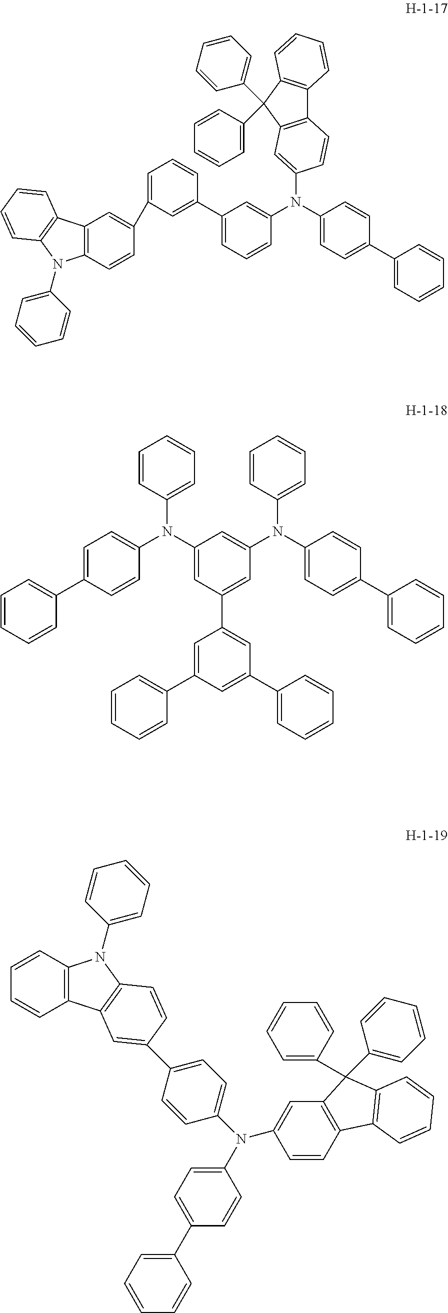 [SMILES: c1ccc(-c2ccc(N(c3cccc(-c4cccc(-c5ccc6c(c5)c5ccccc5n6-c5ccccc5)c4)c3)c3ccc4c(c3)C(c3ccccc3)(c3ccccc3)c3ccccc3-4)cc2)cc1 ; c1ccc(-c2ccc(N(c3ccccc3)c3cc(-c4cc(-c5ccccc5)cc(-c5ccccc5)c4)cc(N(c4ccccc4)c4ccc(-c5ccccc5)cc4)c3)cc2)cc1 ; c1ccc(-c2ccc(N(c3ccc(-c4ccc5c(c4)c4ccccc4n5-c4ccccc4)cc3)c3ccc4c(c3)C(c3ccccc3)(c3ccccc3)c3ccccc3-4)cc2)cc1]

The hole transport region HTR may include a phthalocyanine compound such as copper phthalocyanine, N¹,N¹′-([1,1′-biphenyl]-4,4′-diyl)bis(N¹-phenyl-N⁴,N⁴-di-m-tolylbenzene-1,4-diamine) (DNTPD), 4,4′,4′-[tris(3-methylphenyl)phenylamino] triphenylamine (m-MTDATA), 4,4′,4″-tris(N,N-diphenylamino)triphenylamine (TDATA), 4,4′,4″-tris[N(2-naphthyl)-N-phenylamino]-triphenylamine (2-TNATA), poly(3,4-ethylenedioxythiophene)/poly(4-styrenesulfonate) (PEDOT/PSS), polyaniline/dodecylbenzenesulfonic acid (PANI/DBSA), polyaniline/camphor sulfonic acid (PANI/CSA), polyaniline/poly(4-styrenesulfonate) (PANI/PSS), N,N′-di(1-naphthalene-1-yl)-N,N′-diphenyl-benzidine (NPB), triphenylamine-containing polyetherketone (TPAPEK), 4-isopropyl-4′-methyldiphenyliodonium [tetrakis(pentafluorophenyl)borate], and dipyrazino[2,3-f:2′,3′-h] quinoxaline-2,3,6,7,10,11-hexacarbonitrile (HATCN).

The hole transport region HTR may include carbazole derivatives such as N-phenyl carbazole and polyvinyl carbazole, fluorene-based derivatives, N,N′-bis(3-methylphenyl)-N,N′-diphenyl-[1,1′-biphenyl]-4,4′-diamine (TPD), triphenylamine-based derivatives such as 4,4′,4″-tris(N-carbazolyl)triphenylamine (TCTA), N,N′-di(1-naphthalene-1-yl)-N,N′-diphenyl-benzidine (NPB), 4,4′-cyclohexylidene bis[N,N-bis(4-methylphenyl)benzeneamine] (TAPC), 4,4′-bis[N,N′-(3-tolyl)amino]-3,3′-dimethylbiphenyl (HMTPD), 1,3-bis(N-carbazolyl)benzene (mCP), etc.

The hole transport region HTR may include 9-(4-tert-butylphenyl)-3,6-bis(triphenylsilyl)-9H-carbazole (CzSi), 9-phenyl-9H-3,9′-bicarbazole (CCP), 1,3-bis(1,8-dimethyl-9H-carbazol-9-yl)benzene (mDCP), etc.

The hole transport region HTR may include the compounds of the hole transport region in at least one of the hole injection layer HIL, the hole transport layer HTL, or the electron blocking layer EBL.

A thickness of the hole transport region HTR may be in a range of about 100 Å to about 10,000 Å. For example, the thickness of the hole transport region HTR may be in a range of about 100 Å to about 5,000 Å. In case that the hole transport region HTR includes a hole injection layer HIL, a thickness of the hole injection region HIL may be, for example, in a range of about 30 Å to about 1,000 Å. In case that the hole transport region HTR includes a hole transport layer HTL, a thickness of the hole transport layer HTL may be in a range of about 30 Å to about 1,000 Å. In case that the hole transport region HTR includes an electron blocking layer, a thickness of the electron blocking layer EBL may be in a range of about 10 Å to about 1,000 Å. If the thicknesses of the hole transport region HTR, the hole injection layer HIL, the hole transport layer HTL, and the electron blocking layer EBL satisfy the above-described ranges, satisfactory hole transport properties may be achieved without a substantial increase of driving voltage.

The hole transport region HTR may further include a charge generating material to increase conductivity, in addition to the above-described materials. The charge generating material may be dispersed uniformly or non-uniformly in the hole transport region HTR. The charge generating material may be, for example, a p-dopant. The p-dopant may include at least one of metal halide compounds, quinone derivatives, metal oxides, and cyano group-containing compounds, without limitation. For example, the p-dopant may include metal halide compounds such as CuI and RbI, quinone derivatives such as tetracyanoquinodimethane (TCNQ) and 2,3,5,6-tetrafluoro-7,7′,8,8-tetracyanoquinodimethane (F4-TCNQ), metal oxides such as tungsten oxide and molybdenum oxide, cyano group-containing compounds such as dipyrazino[2,3-f: 2′,3′-h] quinoxaline-2,3,6,7,10,11-hexacarbonitrile (HATCN) and 4-[[2,3-bis[cyano-(4-cyano-2,3,5,6-tetrafluorophenyl)methylidene]cyclopropylidene]-cyanomethyl]-2,3,5,6-tetrafluorobenzonitrile (NDP9), etc., without limitation.

As described above, the hole transport region HTR may further include at least one of a buffer layer (not shown) or an electron blocking layer EBL, in addition to the hole injection layer HIL and the hole transport layer HTL. The buffer layer (not shown) may compensate for a resonance distance according to a wavelength of light emitted from an emission layer EML and may increase emission efficiency. Materials which may be included in the hole transport region HTR may be used as materials included in the buffer layer (not shown). The electron blocking layer EBL may block the injection of electrons from the electron transport region ETR to the hole transport region HTR.

The emission layer EML is provided on the hole transport region HTR. The emission layer EML may have a thickness, for example, in a range of about 100 Å to about 1,000 Å. For example, the emission layer EML may have a thickness in a range of about 100 Å to about 300 Å. The emission layer EML may be a layer formed of a single material, a layer formed of different materials, or a structure having multiple layers formed of different materials.

In the light emitting element ED according to an embodiment, the emission layer EML may include the nitrogen-containing compound according to an embodiment. In an embodiment, the emission layer EML may include the nitrogen-containing compound according to an embodiment as a host. The nitrogen-containing compound according to an embodiment may be a host material of the emission layer EML. In the description, the nitrogen-containing compound according to an embodiment, which will be explained later, may be referred to as a first compound.

The nitrogen-containing compound according to an embodiment may include a first heterocycle including a nitrogen atom as a ring-forming atom. The first heterocycle may include a six-member heterocycle including two nitrogen atoms as ring-forming atoms. In an embodiment, the first heterocycle may be a substituted or unsubstituted pyrimidine group. The first heterocycle may be bonded to a second heterocycle or may be bonded to at least one first substituent. The second heterocycle may be a substituted or unsubstituted pyridine group, a substituted or unsubstituted pyrimidine group, or a substituted or unsubstituted 1,3,5-triazine group. In the description, the second heterocycle may be a heterocycle represented by Formula 3, which will be explained later. The first substituent may be a substituted or unsubstituted o-biphenyl group, or a six-member heterocycle including a nitrogen atom as a ring-forming atom.

In the nitrogen-containing compound according to an embodiment, if the first heterocycle is bonded to a second heterocycle, the first heterocycle may be bonded to the second heterocycle via a first connecting group. For example, the first heterocycle and the second heterocycle may be connected not via a direct linkage, but via the first connecting group. In an embodiment, the first connecting group may include a structure in which a substituted or unsubstituted aryl group of 6 to 30 ring-forming carbon atoms is bonded to a silicon atom. If the first heterocycle and the second heterocycle are connected via the first connecting group, the first heterocycle and the second heterocycle may respectively be bonded to the silicon atom or to an aryl group of the first connecting group. In the description, the first connecting group may be a substituent represented by any one of Formula 2-1 to Formula 2-3, which will be explained later.

In the nitrogen-containing compound according to an embodiment, if the first heterocycle includes at least one first substituent, the first heterocycle may be directly bonded to the first substituent. For example, the first heterocycle and the first substituent may not be connected via an additional connecting group therebetween, but may be directly bonded to each other without a separate connecting group therebetween. The nitrogen-containing compound according to an embodiment may include a first substituent bonded to the first heterocycle. However, embodiments are not limited thereto, and the nitrogen-containing compound according to an embodiment may include two or more first substituents.

The nitrogen-containing compound may be represented by Formula 1-1 or Formula 1-2.

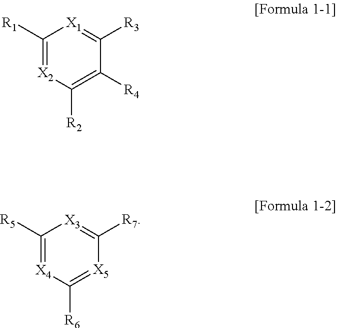

Formula 1-1 represents a case where a first heterocycle is bonded to a second heterocycle via a first connecting group. Formula 1-1 represents a case where a first heterocycle includes two nitrogen atoms, and any one of the carbon atoms composing the first heterocycle is bonded to the first connecting group. Formula 1-2 represents a case where a first substituent is bonded to the first heterocycle. Formula 1-2 represents a case where the first heterocycle and the first substituent are directly bonded. Formula 1-2 represents a case where a first heterocycle includes two nitrogen atoms as ring-forming atoms, and at least one of the carbon atoms composing the first heterocycle is bonded to a first substituent.

In Formula 1-1, X₁ and X₂ may each be N.

In Formula 1-2, X₃ to X₅ may each independently be N or C(R₈), and two of X₃ to X₅ may each be N. In Formula 1-2, two of X₃ to X₅ may each be N, and the remainder of X₃ to X₅ may be C(R₈).

In Formula 1-1 and Formula 1-2, R₁ to R₄ and R₈ may each independently be a hydrogen atom, a deuterium atom, a halogen atom, a substituted or unsubstituted alkyl group of 1 to 20 carbon atoms, a substituted or unsubstituted aryl group of 6 to 30 ring-forming carbon atoms, or a substituted or unsubstituted heteroaryl group of 2 to 30 ring-forming carbon atoms. In an embodiment, R₁ to R₄ and R₈ may each independently be a hydrogen atom, a substituted or unsubstituted aryl group of 6 to 30 ring-forming carbon atoms, or a substituted or unsubstituted heteroaryl group of 2 to 30 ring-forming carbon atoms. For example, R₁ to R₄ and R₈ may each independently be a hydrogen atom, a substituted or unsubstituted phenyl group, or a substituted or unsubstituted carbazole group.

In Formula 1-2, R₅ to R₇ may each independently be a substituted or unsubstituted o-biphenyl group, or a substituted or unsubstituted heteroaryl group of 2 to 30 ring-forming carbon atoms. In Formula 1-2, at least one of R₅ to R₇ may each independently be a substituted or unsubstituted o-biphenyl group, or a substituted or unsubstituted six-member heterocyclic group including a nitrogen atom as a ring-forming atom.

In an embodiment, the first connecting group may be represented by any one of Formula 2-1 to Formula 2-3. In Formula 1-1, at least one of R₁ to R₄ may each independently be a group represented by any one of Formula 2-1 to Formula 2-3.

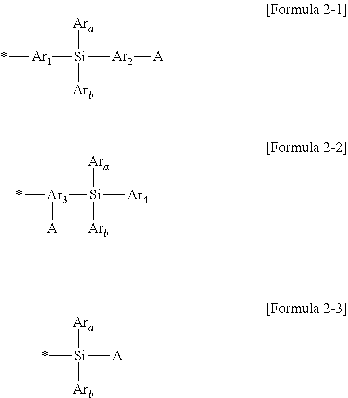

In Formula 2-1 to Formula 2-3, Arₐ, Ar_{b}, and Ar₄ may each independently be a substituted or unsubstituted aryl group of 6 to 30 ring-forming carbon atoms. For example, Arₐ, Ar_{b}, and Ar₄ may each independently be a substituted or unsubstituted phenyl group.

In Formula 2-1, Ar₁ and Ar₂ may each independently be a substituted or unsubstituted arylene group of 6 to 30 ring-forming carbon atoms. For example, Ar₁ and Ar₂ may be each independently a substituted or unsubstituted phenylene group.

In Formula 2-2, Ar₃ may be a substituted or unsubstituted trivalent aryl group of 6 to 30 ring-forming carbon atoms. For example, Ar₃ may be a substituted or unsubstituted trivalent phenyl group.

In Formula 2-1 to Formula 2-3, A may be a group represented by Formula 3.

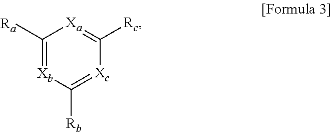

In Formula 3, Xₐ to X_{c} may each independently be N or C(R_{d}). In an embodiment, at least one of Xₐ to X_{c} may each independently be N. In an embodiment, two of Xₐ to X_{c} may each be N.

In Formula 3, Rₐ to R_{d} may each independently be a hydrogen atom, a deuterium atom, a halogen atom, a substituted or unsubstituted alkyl group of 1 to 20 carbon atoms, a substituted or unsubstituted aryl group of 6 to 30 ring-forming carbon atoms, or a substituted or unsubstituted heteroaryl group of 2 to 30 ring-forming carbon atoms; except that one of Rₐ to R_{d} may be a connecting part to the group represented by any one of Formula 2-1 to Formula 2-3. In an embodiment, Rₐ to R_{d} may each independently be a hydrogen atom, a substituted or unsubstituted aryl group of 6 to 30 ring-forming carbon atoms, or a substituted or unsubstituted heteroaryl group of 2 to 30 ring-forming carbon atoms. For example, Rₐ to R_{d} may each independently be a hydrogen atom, a substituted or unsubstituted phenyl group, or a substituted or unsubstituted carbazole group.

In an embodiment, the nitrogen-containing compound represented by Formula 1-1 may be represented by any one of Formula 4-1 to Formula 4-7.

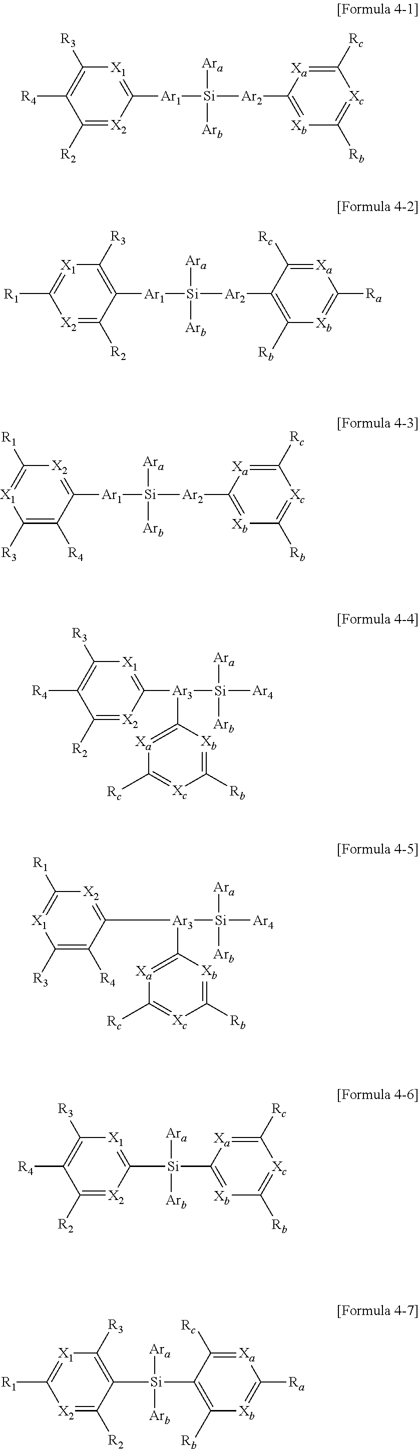

Formula 4-1 to Formula 4-7 each correspond to the nitrogen-containing compound having a structure represented by Formula 1-1, wherein the first connecting group bonded to the first heterocycle is specified in Formula 1-1, and the bonding positions of each of the first heterocycle and the second heterocycle to the first connecting group is specified. Formula 4-1 shows a structure where R₁ of Formula 1-1 and Rₐ of Formula 3 are bonded to the first connecting group represented by Formula 2-1. Formula 4-2 shows a structure where X_{c} of Formula 3 is represented by C(R_{d}), and R₄ of Formula 1-1 and R_{d} of Formula 3 are bonded to the first connecting group represented by Formula 2-1. Formula 4-3 shows a structure where R₂ of Formula 1-1 and Rₐ of Formula 3 are bonded to the first connecting group represented by Formula 2-1. Formula 4-4 shows a structure where R₁ of Formula 1-1 and Rₐ of Formula 3 are bonded to the first connecting group represented by Formula 2-2. Formula 4-5 shows a structure where R₄ of Formula 1-1 and Rₐ of Formula 3 are bonded to the first connecting group represented by Formula 2-2. Formula 4-6 shows a structure where R₁ of Formula 1-1 and Rₐ of Formula 3 are bonded to the first connecting group represented by Formula 2-3. Formula 4-7 shows a structure where X_{c} of Formula 3 is represented by C(R_{d}), and R₄ of Formula 1-1 and R_{d} of Formula 3 are bonded to the first connecting group represented by Formula 2-3.

In Formula 4-1 to Formula 4-7, X₁, X₂, R₁ to R₃, Xₐ to X_{c}, Rₐ to R_{c}, Arₐ, Ar_{b}, and Ar₁ to Ar₄ are each the same as defined in Formula 1-1, Formula 2-1 to Formula 2-3, and Formula 3.

In the nitrogen-containing compound represented by Formula 1-1, at least one of Rᵢ to R₄ may each independently be a group represented by any one of Formula 5-1 to Formula 5-3.

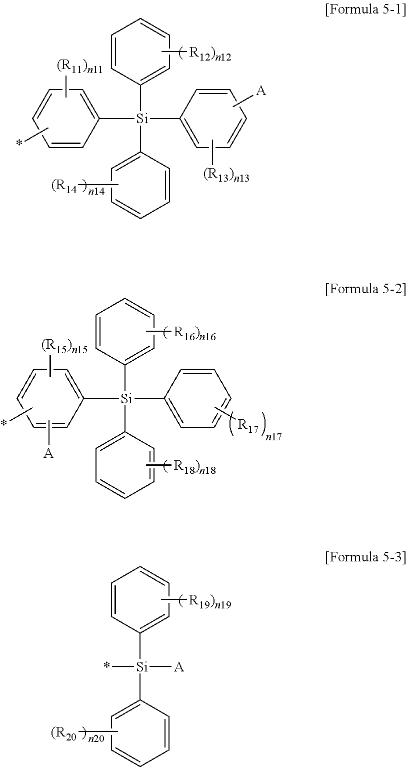

Formula 5-1 to Formula 5-3 are embodiments of a structure of the first connecting group. Formula 5-1 shows a case of Formula 2-1 where Arₐ and Ar_{b} are each independently a substituted or unsubstituted phenyl group, and Ar₁ and Ar₂ are each independently a substituted or unsubstituted phenylene group. Formula 5-2 shows a case of Formula 2-2 where Arₐ and Ar_{b} are each independently a substituted or unsubstituted phenyl group, Arₐ is a substituted or unsubstituted trivalent phenyl group, and Ar₄ is a substituted or unsubstituted phenyl group. Formula 5-3 shows a case of Formula 2-3 where Arₐ and Ar_{b} are each independently a substituted or unsubstituted phenyl group.

In Formula 5-1 to Formula 5-3, R₁₁ to R₂₀ may each independently be a hydrogen atom, a deuterium atom, a halogen atom, a substituted or unsubstituted alkyl group of 1 to 20 carbon atoms, a substituted or unsubstituted aryl group of 6 to 30 ring-forming carbon atoms, or a substituted or unsubstituted heteroaryl group of 2 to 30 ring-forming carbon atoms.

In Formula 5-1, n11 and n13 may each independently be an integer from 0 to 4. If n11 and n13 are each 0, the nitrogen-containing compound according to an embodiment may not be substituted with R₁₁ and R₁₃, respectively. A case where n11 and n13 are each 4, and R₁₁ and R₁₃ are all hydrogen atoms, may be the same as a case where n11 and n13 are each 0. If n11 and n13 are each 2 or more, multiple R₁₁ groups and multiple R₁₃ groups may be all the same, or at least one thereof may be different.

In Formula 5-1 to Formula 5-3, n12, n14, and n16 to n20 may each independently be an integer from 0 to 5. If n12, n14, and n16 to n20 are each 0, the nitrogen-containing compound according to an embodiment may not be substituted with R₁₂, R₁₄, and R₁₆ to R₂₀, respectively. A case where n12, n14, and n16 to n20 are each 5, and R₁₂, R₁₄, and R₁₆ to R₂₀ are all hydrogen atoms, may be the same as a case where n12, n14, and n16 to n20 are each 0. If n12, n14, and n16 to n20 are each 2 or more, multiple groups of each of R₁₂, R₁₄, and R₁₆ to R₂₀ may be all the same, or at least one thereof may be different.

In Formula 5-2, n15 may be an integer from 0 to 3. If n15 is 0, the nitrogen-containing compound according to an embodiment may not be substituted with R₁₅. In Formula 5-2, a case where n15 is 3, and R₁₅ are all hydrogen atoms, may be the same as a case in Formula 5-2 where n15 is 0. If n15 is 2 or more, multiple R₁₅ groups may be all the same, or at least one thereof may be different.

In Formula 5-1 to Formula 5-3, A is the same as defined in Formula 2-1 to Formula 2-3.

In an embodiment, at least one of R₁ to R₄ may each independently be a group represented by any one of Formula 6-1 to Formula 6-3.

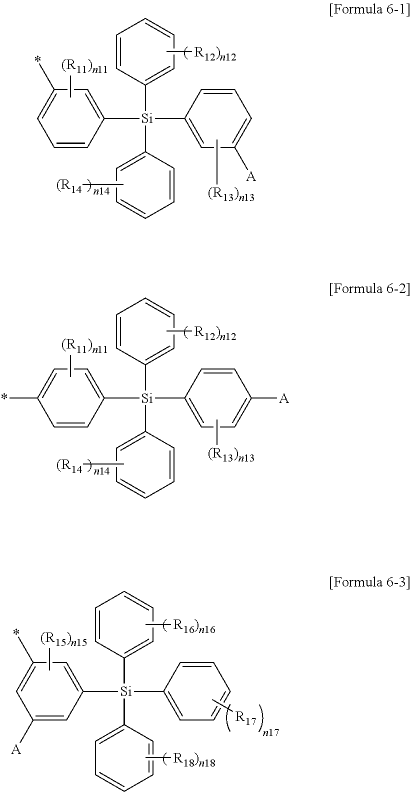

Formula 6-1 to Formula 6-3 each represent a case where the structure of the first connecting group is specified, and the bonding positions of the first heterocycle and the second heterocycle to the first connecting group are each specified.

In Formula 6-1 to Formula 6-3, R₁₁ to R₁₈, n11 to n18, and A are each the same as defined in Formula 2-1 to Formula 2-3 and Formula 5-1 to Formula 5-3.

In the nitrogen-containing compound represented by Formula 1-2, at least one of R₅ to R₇ may each independently be a group represented by any one of Formula 7-1 to Formula 7-5.

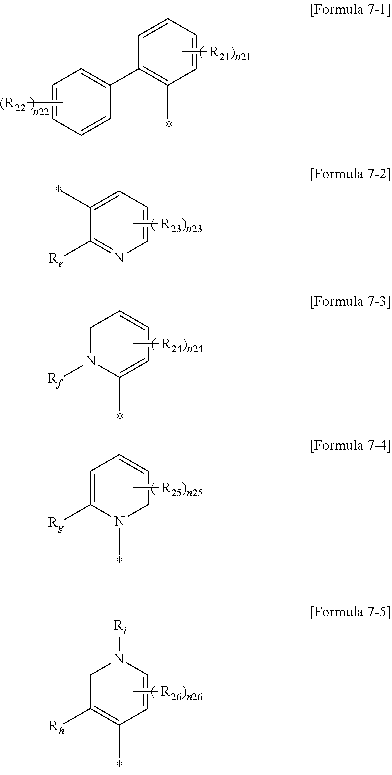

Formula 7-1 to Formula 7-5 are each an embodiment of a structure of the first substituent bonded to Formula 1-2. In an embodiment, the first substituent may be any substituent selected from Formula 7-1 to Formula 7-5.

In Formula 7-2 to Formula 7-5, Rₑ to Rₕ may each independently be a substituted or unsubstituted aryl group of 6 to 30 ring-forming carbon atoms. For example, Rₑ to Rₕ may each independently be a substituted or unsubstituted phenyl group.

In Formula 7-1 to Formula 7-5, Rᵢ and R₂₁ to R₂₆ may each independently be a hydrogen atom, a deuterium atom, a halogen atom, a substituted or unsubstituted alkyl group of 1 to 20 carbon atoms, a substituted or unsubstituted aryl group of 6 to 30 ring-forming carbon atoms, or a substituted or unsubstituted heteroaryl group of 2 to 30 ring-forming carbon atoms. For example, Rᵢ and R₂₁ to R₂₆ may each independently be a hydrogen atom, or a substituted or unsubstituted phenyl group.

In Formula 7-1 to Formula 7-5, n21 and n26 may each independently be an integer from 0 to 4; n22, n24, and n25 may each independently be an integer from 0 to 5; and n23 may be an integer from 0 to 3.

If n21 and n26 are each 0, the nitrogen-containing compound according to an embodiment may not be substituted with R₂₁ and R₂₆, respectively. A case where n21 and n26 are each 4, and R₂₁ and R₂₆ are all hydrogen atoms, may be the same as a case where n21 and n26 are each 0. If n21 and n26 are each 2 or more, multiple R₂₁ groups and multiple R₂₆ groups may be all the same, or at least one thereof may be different.

If n22, n24, and n25 are each 0, the nitrogen-containing compound according to an embodiment may not be substituted with R₂₂, R₂₄, and R₂₅, respectively. A case where n22, n24, and n25 are each 5, and R₂₂, R₂₄, and R₂₅ are all hydrogen atoms, may be the same as a case where n22, n24, and n25 are each 0. If n22, n24, and n25 are each 2 or more, multiple groups of each of R₂₂, R₂₄, and R₂₅ may be all the same, or at least one thereof may be different.

If n23 is 0, the nitrogen-containing compound according to an embodiment may not be substituted with R₂₃. A case where n23 is 3, and R₂₃ are all hydrogen atoms, may be the same as a case where n23 is 0. If n23 is 2 or more, multiple R₂₃ groups may be all the same, or at least one thereof may be different.

In an embodiment, at least one of R₅ to R₇ may each independently be a group represented by any one of Formula 8-1 to Formula 8-5.

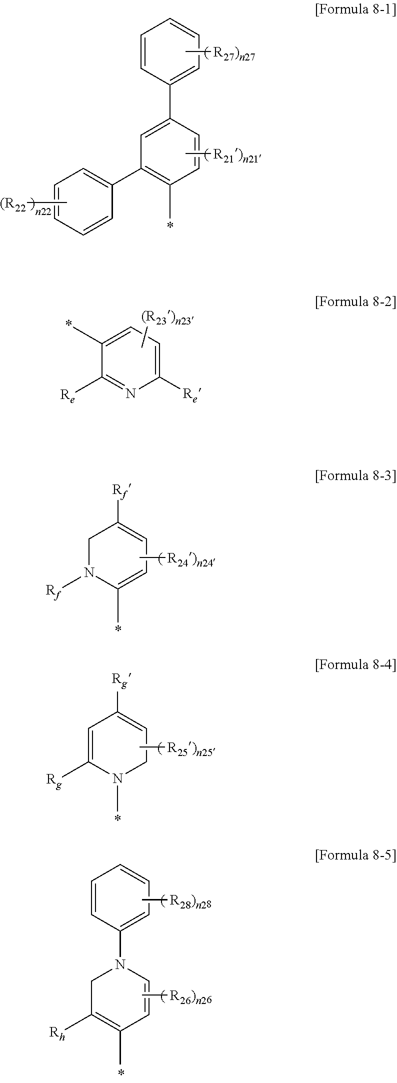

Formula 8-1 to Formula 8-5 are each an embodiment of a structure of the first substituent bonded to Formula 1-2. In an embodiment, the first substituent may be any substituent selected from Formula 7-1 to Formula 7-5.

In Formula 8-1 and Formula 8-5, R₂₇ and R₂₈ may each independently be a hydrogen atom, a deuterium atom, a halogen atom, a substituted or unsubstituted alkyl group of 1 to 20 carbon atoms, a substituted or unsubstituted aryl group of 6 to 30 ring-forming carbon atoms, or a substituted or unsubstituted heteroaryl group of 2 to 30 ring-forming carbon atoms. For example, R₂₇ and R₂₈ may each be a hydrogen atom.

In Formula 8-2 to Formula 8-4, Rₑ′ to R_{g}′ may each independently be a substituted or unsubstituted aryl group of 6 to 30 ring-forming carbon atoms. For example, Rₑ′ to R_{g}′ may each independently be a substituted or unsubstituted phenyl group. In an embodiment, Rₑ and Rₑ′ of Formula 8-2 may be the same, R_{f} and R_{f}′ of Formula 8-3 may be the same, and R_{g} and R_{g}′ of Formula 8-4 may be the same. However, embodiments are not limited thereto.

In Formula 8-1 to Formula 8-4, R₂₁′ and R₂₃′ to R₂₅′ may each independently be a hydrogen atom, a deuterium atom, a halogen atom, a substituted or unsubstituted alkyl group of 1 to 20 carbon atoms, a substituted or unsubstituted aryl group of 6 to 30 ring-forming carbon atoms, or a substituted or unsubstituted heteroaryl group of 2 to 30 ring-forming carbon atoms. For example, R₂₁′ and R₂₃′ to R₂₅′ may each independently be a hydrogen atom.

In Formula 8-1 to Formula 8-5, n21′ may be an integer from 0 to 3, n23′ may be an integer from 0 to 2, n24′ and n25′ may each independently be an integer from 0 to 4, and n27 and n28 may each independently be an integer from 0 to 5.

If n21′ is 0, the nitrogen-containing compound according to an embodiment may not be substituted with R₂₁′. A case where n21′ is 3, and R₂₁′ are all hydrogen atoms, may be the same as a case where n21′ is 0. If n21′ is 2 or more, multiple R₂₁′ groups may be all the same, or at least one thereof may be different.

If n23′ is 0, the nitrogen-containing compound according to an embodiment may not be substituted with R₂₃′. A case where n23′ is 2, and R₂₃′ are all hydrogen atoms, may be the same as a case where n23′ is 0. If n23′ is 2, multiple R₂₃′ groups may be all the same, or at least one thereof may be different.

If n24′ and n25′ are each 0, the nitrogen-containing compound according to an embodiment may not be substituted with R₂₄′ and R₂₅′, respectively. A case where n24′ and n25′ are each 4, and R₂₄′ and R₂₅′ are all hydrogen atoms, may be the same as a case where n24′ and n25′ are each 0. If n24′ and n25′ are each 2 or more, multiple R₂₄′ groups and multiple R₂₅ groups may be all the same, or at least one thereof may be different.

If n27 and n28 are each 0, the nitrogen-containing compound according to an embodiment may not be substituted with R₂₇ and R₂₈, respectively. A case where n27 and n28 are each 5, and R₂₇ and R₂₈ are all hydrogen atoms, may be the same as a case where n27 and n28 are each 0. If n27 and n28 are each 2 or more, multiple R₂₇ groups and multiple R₂₈ groups may be all the same, or at least one thereof may be different.

In Formula 8-1 to Formula 8-5, R₂₂, R₂₆, n22, n26, and Rₑ to Rₕ are each the same as described in Formula 7-1 to Formula 7-5.

In an embodiment, the nitrogen-containing compound represented by Formula 1-2 may be represented by any one of Formula 9-1 to Formula 9-5.

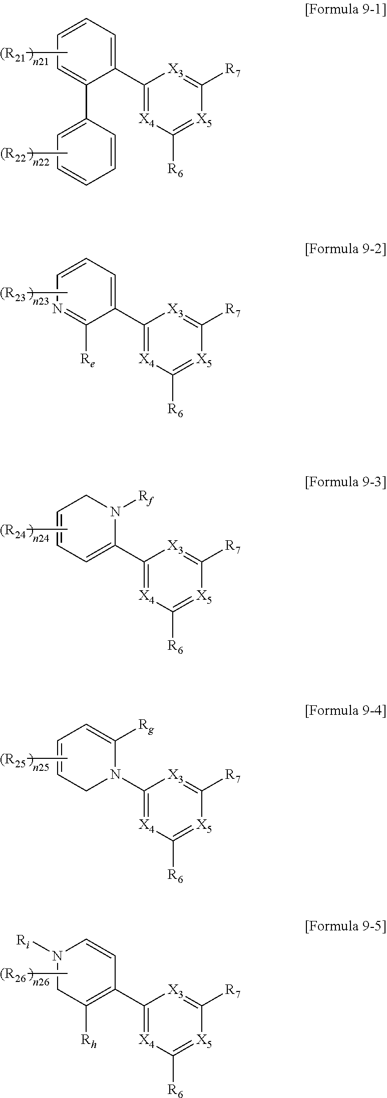

Formula 9-1 to Formula 9-5 each represent an embodiment where R₅ in Formula 1-2 is specified.

In Formula 9-1 to Formula 9-5, Rₑ to Rₕ may each independently be a substituted or unsubstituted aryl group of 6 to 30 ring-forming carbon atoms. For example, Rₑ to Rₕ may each independently be a substituted or unsubstituted phenyl group.

In Formula 9-1 to Formula 9-5, Rᵢ and R₂₁ to R₂₆ may each independently be a hydrogen atom, a deuterium atom, a halogen atom, a substituted or unsubstituted alkyl group of 1 to 20 carbon atoms, a substituted or unsubstituted aryl group of 6 to 30 ring-forming carbon atoms, or a substituted or unsubstituted heteroaryl group of 2 to 30 ring-forming carbon atoms.

In Formula 9-1 to Formula 9-5, n21 and n26 may each independently be an integer from 0 to 4; n22, n24, and n25 may each independently be an integer from 0 to 5; and n23 may be an integer from 0 to 3. In Formula 9-1 to Formula 9-5, n21 to n26 may each be the same as described in Formula 7-1 to Formula 7-5.

In Formula 9-1 to Formula 9-5, X₃ to X₅, R₆, and R₇ are each the same as defined in Formula 1-2.

In an embodiment, at least one R₆ and R₇ may each independently be a group represented by Formula 10.

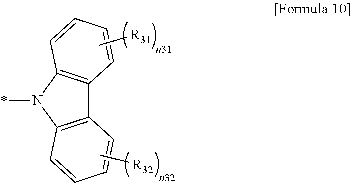

In Formula 10, R₃₁ and R₃₂ may each independently be a hydrogen atom, a deuterium atom, a halogen atom, a substituted or unsubstituted alkyl group of 1 to 20 carbon atoms, a substituted or unsubstituted aryl group of 6 to 30 ring-forming carbon atoms, or a substituted or unsubstituted heteroaryl group of 2 to 30 ring-forming carbon atoms. For example, R₃₁ and R₃₂ may each be a hydrogen atom.

In Formula 10, n31 and n32 may each independently be an integer from 0 to 4. A case where n31 and n32 are each 4, and R₃₁ and R₃₂ are all hydrogen atoms, may be the same as a case where n31 and n32 are each 0. If n31 and n32 are each 2 or more, multiple R₃₁ groups and multiple R₃₂ groups may be all the same, or at least one thereof may be different.

In an embodiment, the nitrogen-containing compound represented by Formula 1-2 may be represented by Formula 11-1 or Formula 11-2.

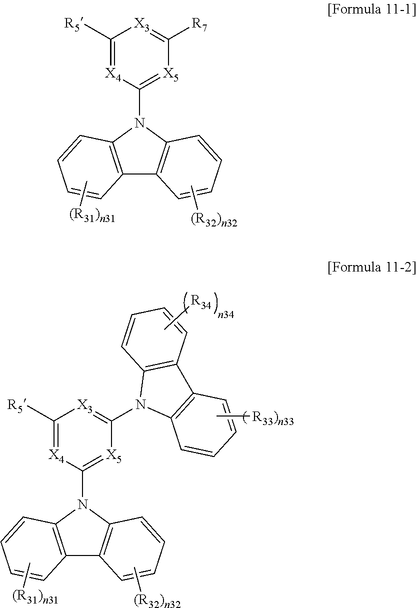

In Formula 11-1 and Formula 11-2, R₅′ may be a group represented by any one of Formula 7-1 to Formula 7-5.

In Formula 11-2, R₃₃ and R₃₄ may each independently be a hydrogen atom, a deuterium atom, a halogen atom, a substituted or unsubstituted alkyl group of 1 to 20 carbon atoms, a substituted or unsubstituted aryl group of 6 to 30 ring-forming carbon atoms, or a substituted or unsubstituted heteroaryl group of 2 to 30 ring-forming carbon atoms.

In Formula 11-2, n33 and n34 may each independently be an integer from 0 to 4. A case where n33 and n34 are each 4, and R₃₃ and R₃₄ are all hydrogen atoms, may be the same as a case where n33 and n34 are each 0. If n33 and n34 are each 2 or more, multiple R₃₃ groups and multiple R₃₄ groups may be all the same, or at least one thereof may be different.

In Formula 11-1 and Formula 11-2, R₇, R₃₁, R₃₂, n31, and n32 are each the same as defined in Formula 1-2 and Formula 10.

The nitrogen-containing compound according to an embodiment may be any compound selected from Compound Group 1. The light emitting device ED according to an embodiment may include at least one nitrogen-containing compound selected from Compound Group 1 in an emission layer EML.

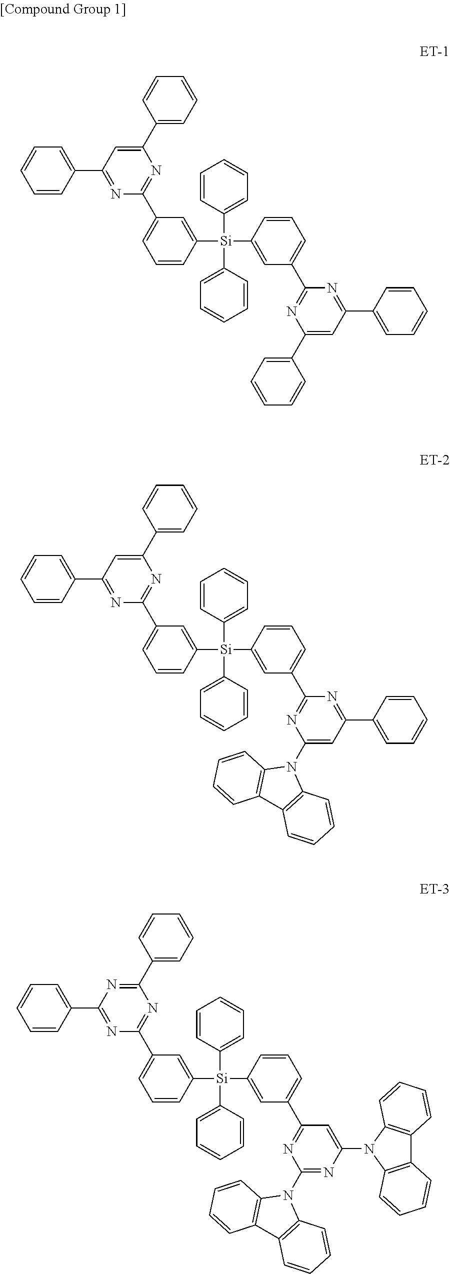 [SMILES: c1ccc(-c2cc(-c3ccccc3)nc(-c3cccc([Si](c4ccccc4)(c4ccccc4)c4cccc(-c5nc(-c6ccccc6)cc(-c6ccccc6)n5)c4)c3)n2)cc1 ; c1ccc(-c2cc(-c3ccccc3)nc(-c3cccc([Si](c4ccccc4)(c4ccccc4)c4cccc(-c5nc(-c6ccccc6)cc(-n6c7ccccc7c7ccccc76)n5)c4)c3)n2)cc1 ; c1ccc(-c2nc(-c3ccccc3)nc(-c3cccc([Si](c4ccccc4)(c4ccccc4)c4cccc(-c5cc(-n6c7ccccc7c7ccccc76)nc(-n6c7ccccc7c7ccccc76)n5)c4)c3)n2)cc1]

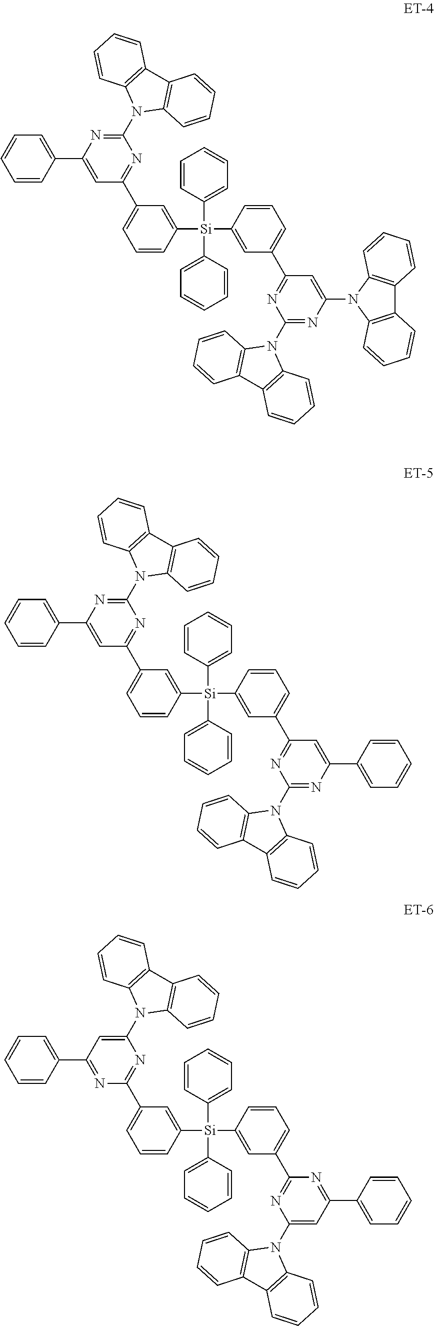 [SMILES: c1ccc(-c2cc(-c3cccc([Si](c4ccccc4)(c4ccccc4)c4cccc(-c5cc(-n6c7ccccc7c7ccccc76)nc(-n6c7ccccc7c7ccccc76)n5)c4)c3)nc(-n3c4ccccc4c4ccccc43)n2)cc1 ; c1ccc(-c2cc(-c3cccc([Si](c4ccccc4)(c4ccccc4)c4cccc(-c5cc(-c6ccccc6)nc(-n6c7ccccc7c7ccccc76)n5)c4)c3)nc(-n3c4ccccc4c4ccccc43)n2)cc1 ; c1ccc(-c2cc(-n3c4ccccc4c4ccccc43)nc(-c3cccc([Si](c4ccccc4)(c4ccccc4)c4cccc(-c5nc(-c6ccccc6)cc(-n6c7ccccc7c7ccccc76)n5)c4)c3)n2)cc1]

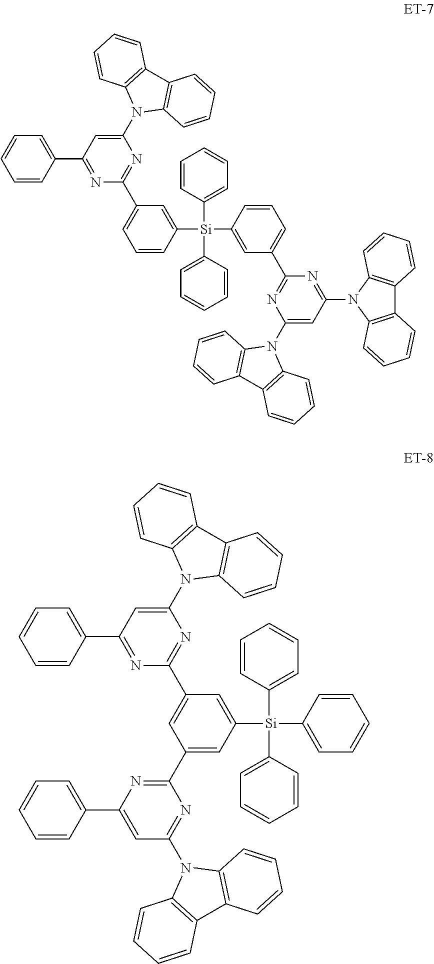 [SMILES: c1ccc(-c2cc(-n3c4ccccc4c4ccccc43)nc(-c3cccc([Si](c4ccccc4)(c4ccccc4)c4cccc(-c5nc(-n6c7ccccc7c7ccccc76)cc(-n6c7ccccc7c7ccccc76)n5)c4)c3)n2)cc1 ; c1ccc(-c2cc(-n3c4ccccc4c4ccccc43)nc(-c3cc(-c4nc(-c5ccccc5)cc(-n5c6ccccc6c6ccccc65)n4)cc([Si](c4ccccc4)(c4ccccc4)c4ccccc4)c3)n2)cc1]

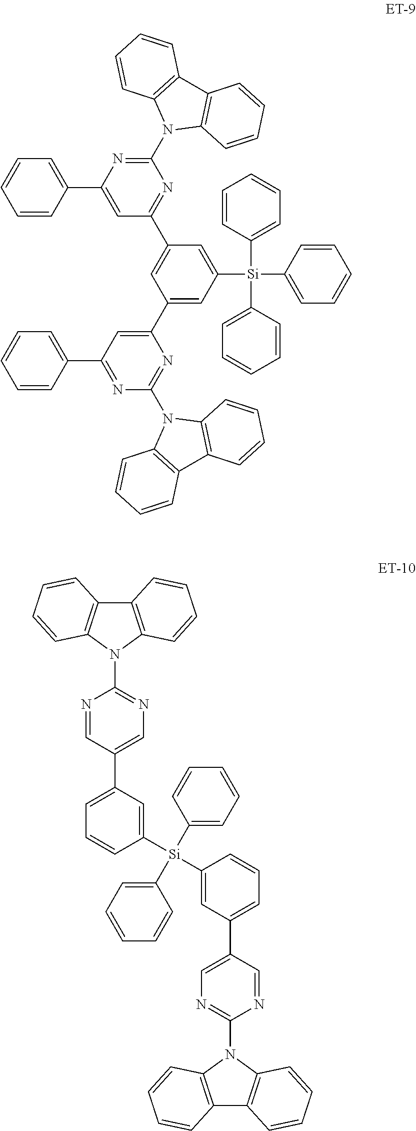 [SMILES: c1ccc(-c2cc(-c3cc(-c4cc(-c5ccccc5)nc(-n5c6ccccc6c6ccccc65)n4)cc([Si](c4ccccc4)(c4ccccc4)c4ccccc4)c3)nc(-n3c4ccccc4c4ccccc43)n2)cc1 ; c1ccc([Si](c2ccccc2)(c2cccc(-c3cnc(-n4c5ccccc5c5ccccc54)nc3)c2)c2cccc(-c3cnc(-n4c5ccccc5c5ccccc54)nc3)c2)cc1]

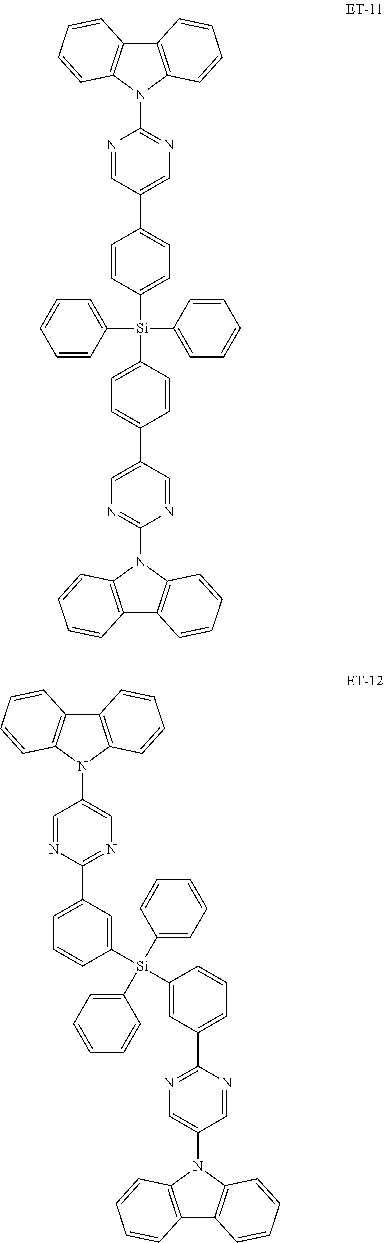 [SMILES: c1ccc([Si](c2ccccc2)(c2ccc(-c3cnc(-n4c5ccccc5c5ccccc54)nc3)cc2)c2ccc(-c3cnc(-n4c5ccccc5c5ccccc54)nc3)cc2)cc1 ; c1ccc([Si](c2ccccc2)(c2cccc(-c3ncc(-n4c5ccccc5c5ccccc54)cn3)c2)c2cccc(-c3ncc(-n4c5ccccc5c5ccccc54)cn3)c2)cc1]

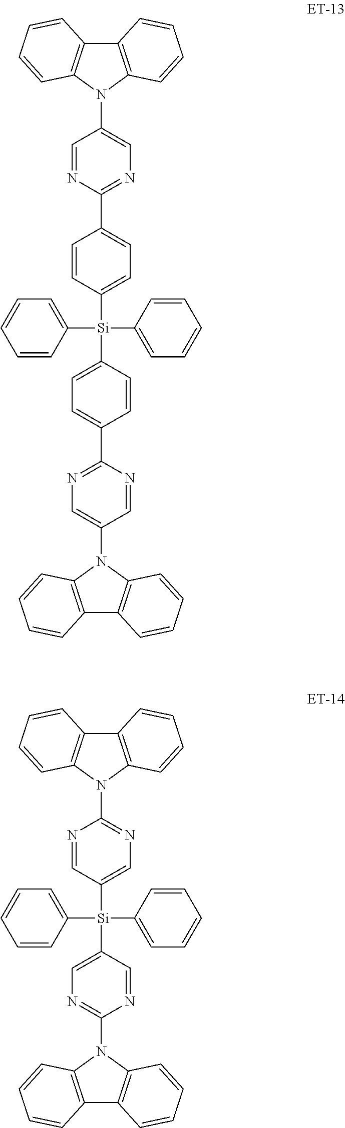 [SMILES: c1ccc([Si](c2ccccc2)(c2ccc(-c3ncc(-n4c5ccccc5c5ccccc54)cn3)cc2)c2ccc(-c3ncc(-n4c5ccccc5c5ccccc54)cn3)cc2)cc1 ; c1ccc([Si](c2ccccc2)(c2cnc(-n3c4ccccc4c4ccccc43)nc2)c2cnc(-n3c4ccccc4c4ccccc43)nc2)cc1]

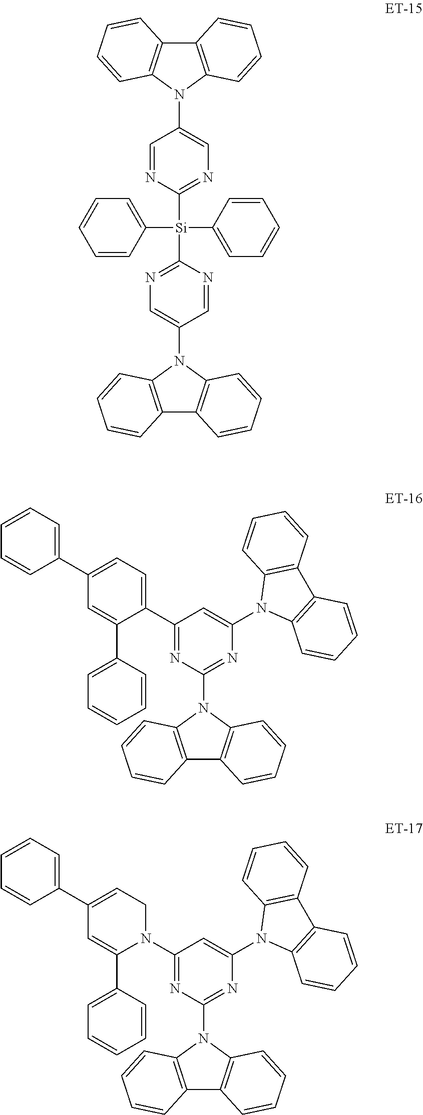 [SMILES: c1ccc([Si](c2ccccc2)(c2ncc(-n3c4ccccc4c4ccccc43)cn2)c2ncc(-n3c4ccccc4c4ccccc43)cn2)cc1 ; c1ccc(-c2ccc(-c3cc(-n4c5ccccc5c5ccccc54)nc(-n4c5ccccc5c5ccccc54)n3)c(-c3ccccc3)c2)cc1 ; C1=C(c2ccccc2)C=C(c2ccccc2)N(c2cc(-n3c4ccccc4c4ccccc43)nc(-n3c4ccccc4c4ccccc43)n2)C1]

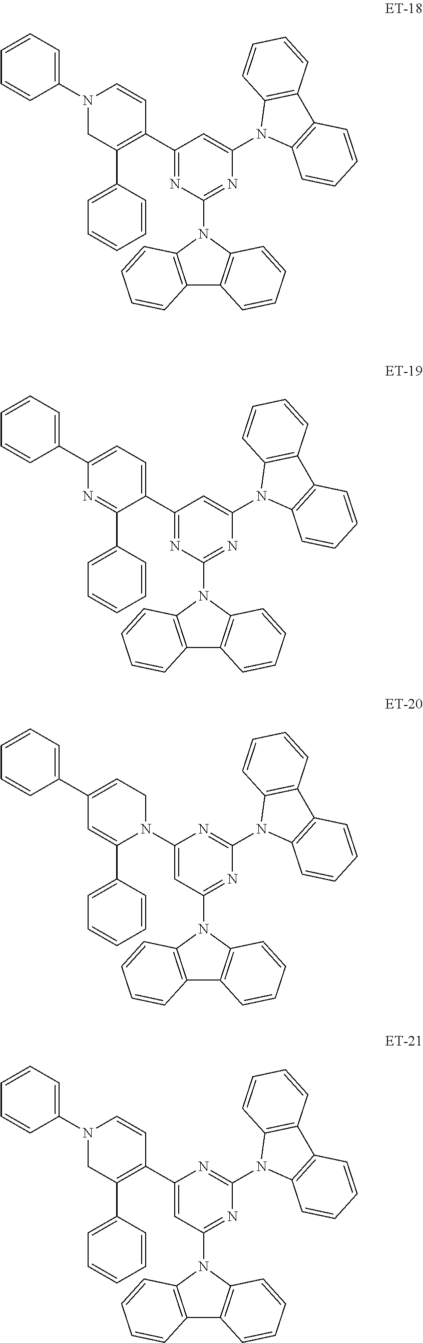 [SMILES: C1=CN(c2ccccc2)CC(c2ccccc2)=C1c1cc(-n2c3ccccc3c3ccccc32)nc(-n2c3ccccc3c3ccccc32)n1 ; c1ccc(-c2ccc(-c3cc(-n4c5ccccc5c5ccccc54)nc(-n4c5ccccc5c5ccccc54)n3)c(-c3ccccc3)n2)cc1 ; C1=C(c2ccccc2)C=C(c2ccccc2)N(c2cc(-n3c4ccccc4c4ccccc43)nc(-n3c4ccccc4c4ccccc43)n2)C1 ; C1=CN(c2ccccc2)CC(c2ccccc2)=C1c1cc(-n2c3ccccc3c3ccccc32)nc(-n2c3ccccc3c3ccccc32)n1]

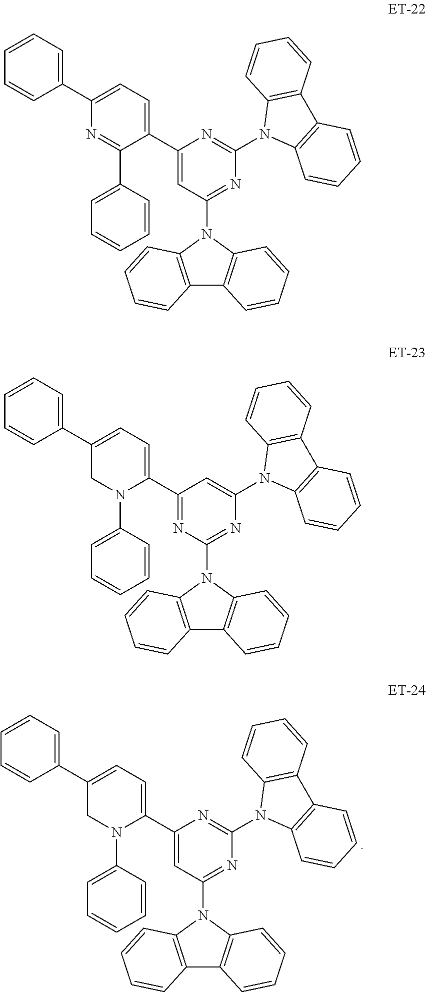 [SMILES: c1ccc(-c2ccc(-c3cc(-n4c5ccccc5c5ccccc54)nc(-n4c5ccccc5c5ccccc54)n3)c(-c3ccccc3)n2)cc1 ; C1=C(c2ccccc2)CN(c2ccccc2)C(c2cc(-n3c4ccccc4c4ccccc43)nc(-n3c4ccccc4c4ccccc43)n2)=C1 ; C1=C(c2ccccc2)CN(c2ccccc2)C(c2cc(-n3c4ccccc4c4ccccc43)nc(-n3c4ccccc4c4ccccc43)n2)=C1]

The nitrogen-containing compound according to an embodiment introduces a ring or a substituent bonded to a first heterocycle, so that electron control may readily occur, and light emission due to interaction with a dopant may be suppressed. Accordingly, if the nitrogen-containing compound according to an embodiment serves as a host of the emission layer EML of the light emitting device ED, high emission efficiency and blue light with high color purity may be achieved.

In light emitting devices of the related art, due to strong charge interaction between a host and a dopant in an emission layer, a first emission peak due to light emission from the dopant itself and a second emission peak due to the interaction between the host and the dopant are produced, and color deterioration and efficiency degradation occur. If a difference between a lowest unoccupied molecular orbital (LUMO) energy level of the host and the LUMO energy level of the dopant is large, an energy barrier may be formed between the host and the dopant. The formation of such an energy barrier may accelerate the deterioration of a material and may be a main factor towards the deterioration of the device life. In the description, the energy barrier between a host and a dopant may be a difference between the LUMO energy level of the host and the LUMO energy level of the dopant.

The nitrogen-containing compound according to an embodiment includes a first heterocycle containing two nitrogen atoms as ring-forming atoms. The first heterocycle may be bonded to a second heterocycle via a first connecting group, or may be directly bonded to a first substituent. Accordingly, the nitrogen-containing compound according to an embodiment reduces interaction with a dopant, thereby providing deep blue light with high color purity. The nitrogen-containing compound according to an embodiment may show a shallow LUMO energy level, and accordingly, may suppress the efficiency deterioration and lifetime reduction by the energy barrier with the dopant. In the description, a shallow energy level may be a reduction of an absolute value of the energy level from a vacuum level to the negative direction. In the description, a deep energy level may be an increase of an absolute value of the energy level from a vacuum level to the negative direction.

The nitrogen-containing compound represented by Formula 1-1 according to an embodiment may include a structure in which a first heterocycle and a second heterocycle are connected via a first connecting group. The second heterocycle includes a nitrogen atom as a ring-constituting atom, and the second heterocycle may be connected with the first heterocycle via a first connecting group containing a silicon atom. Through the connection to the first heterocycle via the first connecting group, the second heterocycle may contribute to the control of a LUMO energy level of the nitrogen-containing compound as a whole. For example, the nitrogen-containing compound according to an embodiment represented by Formula 1-1 may have a shallow LUMO energy level, and accordingly, if the nitrogen-containing compound represented by Formula 1-1 is used as a host material in the emission layer EML, the energy barrier between the host and the dopant in the emission layer EML may be reduced, and emission efficiency may increase. The nitrogen-containing compound represented by Formula 1-1 includes a structure in which a first heterocycle and a second heterocycle are connected via a first connecting group, and may have a molecular structure which may suppress charge interaction with a dopant in an emission layer, and accordingly, may suppress the light emission due to interaction with the dopant, thereby further improving emission efficiency and device lifetime.

The nitrogen-containing compound represented by Formula 1-2 may include a structure in which a first heterocycle is directly bonded to a first substituent. The first substituent may be bonded to the first heterocycle to control the LUMO energy level of the nitrogen-containing compound as a whole and induce a molecular arrangement which may suppress the charge interaction with a dopant molecule in an emission layer. Accordingly, interaction with the dopant may be suppressed, color purity and life characteristics may be improved, the LUMO energy level may become shallow, the energy transition with the dopant may increase, and emission efficiency may be improved.

In an embodiment, the emission layer EML may include a host and a dopant, and the emission layer EML may include the nitrogen-containing compound as the host. The nitrogen-containing compound represented by Formula 1-1 or Formula 1-2 may be the host material of the emission layer.

For example, in the light emitting device ED according to an embodiment, the emission layer EML may include a host for emitting phosphorescence and a dopant for emitting phosphorescence, and may include the nitrogen-containing compound according to an embodiment as the host for emitting phosphorescence. In another embodiment, the light emitting device ED, the emission layer EML may include a host for emitting fluorescence and a dopant for emitting fluorescence, and the host for emitting fluorescence may include the nitrogen-containing compound according to an embodiment.

In the light emitting device ED according to an embodiment, an emission layer EML may include a host for emitting delayed fluorescence and a dopant for emitting delayed fluorescence, and the host for emitting delayed fluorescence may include the nitrogen-containing compound according to an embodiment. In the light emitting device ED according to an embodiment, an emission layer EML may include a host for emitting blue thermally activated delayed fluorescence (TADF) and a dopant for emitting blue thermally activated delayed fluorescence, and the host for emitting blue thermally activated delayed fluorescence may include the nitrogen-containing compound according to an embodiment. The emission layer EML may include at least one nitrogen-containing compound selected from Compound Group 1 as the host material of the emission layer EML.

In the light emitting device ED according to an embodiment, a host may not emit light in the light emitting device ED but may transfer energy to a dopant. The emission layer EML may include one or more hosts. For example, the emission layer EML may include two different types of hosts. However, embodiments are not limited thereto, and the emission layer EML may include one type of a host, or a mixture of two or more different types of hosts.

In an embodiment, the emission layer EML may include two different hosts. The host may include a first compound represented by Formula 1-1 or Formula 1-2, and a second compound which is different from the first compound. In an embodiment, the host may include the first compound represented by Formula 1-1 or Formula 1-2, and a second compound represented by Formula H-1. The first compound may be an electron transport host, and the second compound may be a hole transport host. In an embodiment, the emission layer EML includes the first compound and the second compound, and the first compound and the second compound may form an exciplex.

In the light emitting device ED of an embodiment, an exciplex may be formed by a hole transport host and an electron transport host in an emission layer. A triplet energy of the exciplex formed by the hole transport host and the electron transport host may correspond to a difference between the lowest unoccupied molecular orbital (LUMO) energy level of the electron transport host and the highest occupied molecular orbital (HOMO) energy level of the hole transport host.

For example, an absolute value of the triplet energy level (T1) of the exciplex formed by the hole transport host and the electron transport host may be in a range of about 2.4 eV to about 3.0 eV. The triplet energy level of the exciplex may be a value that is smaller than an energy gap of each host material. The exciplex may have a triplet energy level equal to or less than about 3.0 eV, which may be smaller than an energy gap of each of the hole transport host and the electron transport host.

The emission layer EML according to an embodiment may include a second compound including a carbazole derivative moiety. The second compound may be represented by Formula H-1.

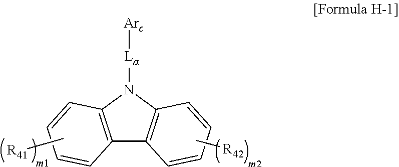

In Formula H-1, Lₐ may be a direct linkage, a substituted or unsubstituted arylene group of 6 to 30 ring-forming carbon atoms, or a substituted or unsubstituted heteroarylene group of 2 to 30 ring-forming carbon atoms. In Formula H-1, Ar_{c} may be a substituted or unsubstituted aryl group of 6 to 30 ring-forming carbon atoms, or a substituted or unsubstituted heteroaryl group of 2 to 30 ring-forming carbon atoms.

In Formula H-1, R₄₁ and R₄₂ may each independently be a hydrogen atom, a deuterium atom, a halogen atom, a substituted or unsubstituted alkyl group of 1 to 20 carbon atoms, a substituted or unsubstituted aryl group of 6 to 30 ring-forming carbon atoms, or a substituted or unsubstituted heteroaryl group of 2 to 30 ring-forming carbon atoms. For example, R₄₁ and R₄₂ may each independently be a hydrogen atom or a deuterium atom.

In Formula H-1, m1 and m2 may each independently be an integer from 0 to 4. If m1 and m2 are each 0, the second compound may not be substituted with R₄₁ and R₄₂, respectively. A case of Formula H-1 where m1 and m2 are each 4, and R₄₁ and R₄₂ are all hydrogen atoms, may be the same as a case of Formula H-1 where m1 and m2 are each 0. If m1 and m2 are each 2 or more, multiple R₄₁ groups and multiple R₄₂ groups may be all the same, or at least one thereof may be different. For example, in Formula H-1, m1 and m2 may each be 0, so that a carbazole group in Formula H-1 may be unsubstituted.

For example, in Formula H-1, Lₐ may be a direct linkage, a phenylene group, a divalent biphenyl group, a divalent carbazole group, or the like, but embodiments are not limited thereto. For example, in Formula H-1, Ar_{c} may be a substituted or unsubstituted carbazole group, a substituted or unsubstituted dibenzofuran group, a substituted or unsubstituted dibenzothiophene group, a substituted or unsubstituted biphenyl group, or the like, but embodiments are not limited thereto.

If the emission layer EML of the light emitting device ED according to an embodiment includes the first compound represented by Formula 1-1 or Formula 1-2, and includes the second compound represented by Formula H-1, excellent emission efficiency and long-life characteristics may be achieved.

In an embodiment, the second compound represented by Formula H-1 may be any compound selected from Compound Group 2. The emission layer EML may include at least one compound selected from Compound Group 2 as a host material.

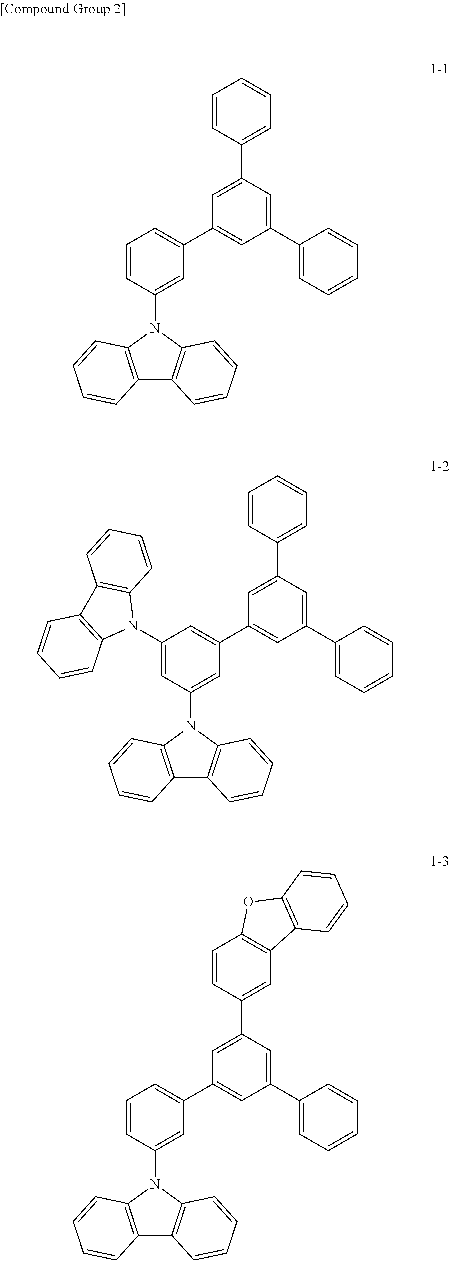 [SMILES: c1ccc(-c2cc(-c3ccccc3)cc(-c3cccc(-n4c5ccccc5c5ccccc54)c3)c2)cc1 ; c1ccc(-c2cc(-c3ccccc3)cc(-c3cc(-n4c5ccccc5c5ccccc54)cc(-n4c5ccccc5c5ccccc54)c3)c2)cc1 ; c1ccc(-c2cc(-c3cccc(-n4c5ccccc5c5ccccc54)c3)cc(-c3ccc4oc5ccccc5c4c3)c2)cc1]

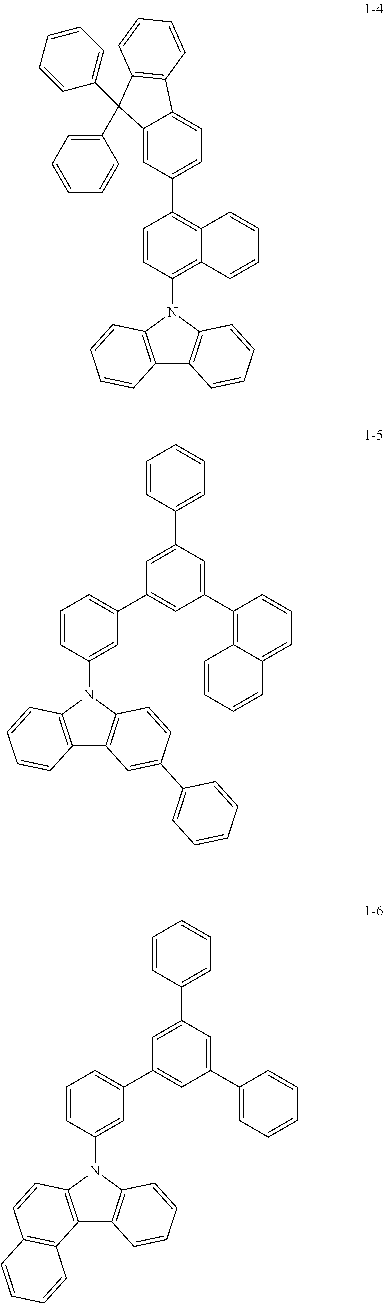 [SMILES: c1ccc(C2(c3ccccc3)c3ccccc3-c3ccc(-c4ccc(-n5c6ccccc6c6ccccc65)c5ccccc45)cc32)cc1 ; c1ccc(-c2cc(-c3cccc(-n4c5ccccc5c5cc(-c6ccccc6)ccc54)c3)cc(-c3cccc4ccccc34)c2)cc1 ; c1ccc(-c2cc(-c3ccccc3)cc(-c3cccc(-n4c5ccccc5c5c6ccccc6ccc54)c3)c2)cc1]

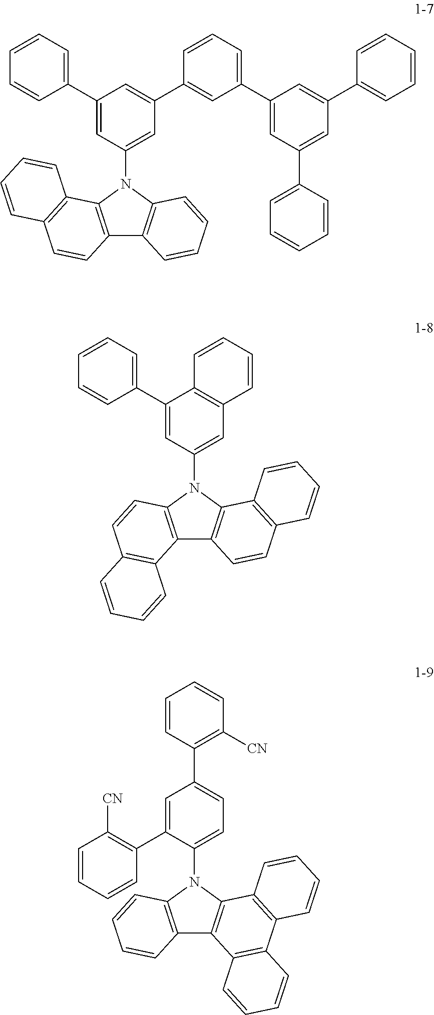 [SMILES: c1ccc(-c2cc(-c3ccccc3)cc(-c3cccc(-c4cc(-c5ccccc5)cc(-n5c6ccccc6c6ccc7ccccc7c65)c4)c3)c2)cc1 ; c1ccc(-c2cc(-n3c4ccc5ccccc5c4c4ccc5ccccc5c43)cc3ccccc23)cc1 ; N#Cc1ccccc1-c1ccc(-n2c3ccccc3c3c4ccccc4c4ccccc4c32)c(-c2ccccc2C#N)c1]

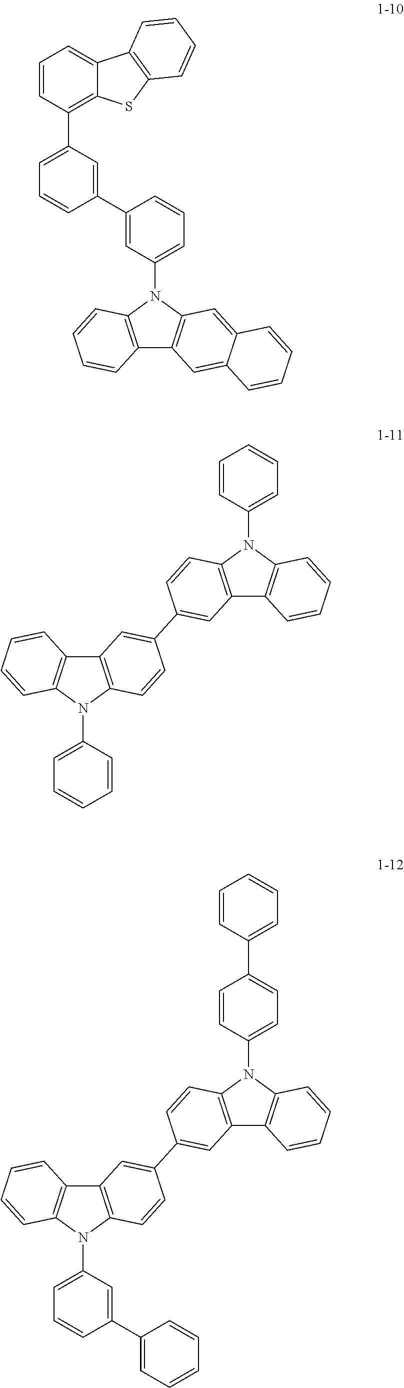 [SMILES: c1cc(-c2cccc(-n3c4ccccc4c4cc5ccccc5cc43)c2)cc(-c2cccc3c2sc2ccccc23)c1 ; c1ccc(-n2c3ccccc3c3cc(-c4ccc5c(c4)c4ccccc4n5-c4ccccc4)ccc32)cc1 ; c1ccc(-c2ccc(-n3c4ccccc4c4cc(-c5ccc6c(c5)c5ccccc5n6-c5cccc(-c6ccccc6)c5)ccc43)cc2)cc1]

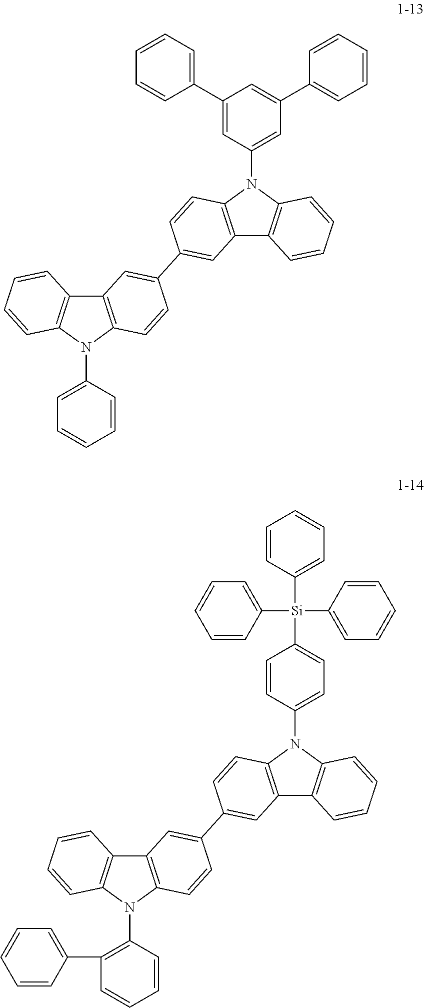 [SMILES: c1ccc(-c2cc(-c3ccccc3)cc(-n3c4ccccc4c4cc(-c5ccc6c(c5)c5ccccc5n6-c5ccccc5)ccc43)c2)cc1 ; c1ccc(-c2ccccc2-n2c3ccccc3c3cc(-c4ccc5c(c4)c4ccccc4n5-c4ccc([Si](c5ccccc5)(c5ccccc5)c5ccccc5)cc4)ccc32)cc1]

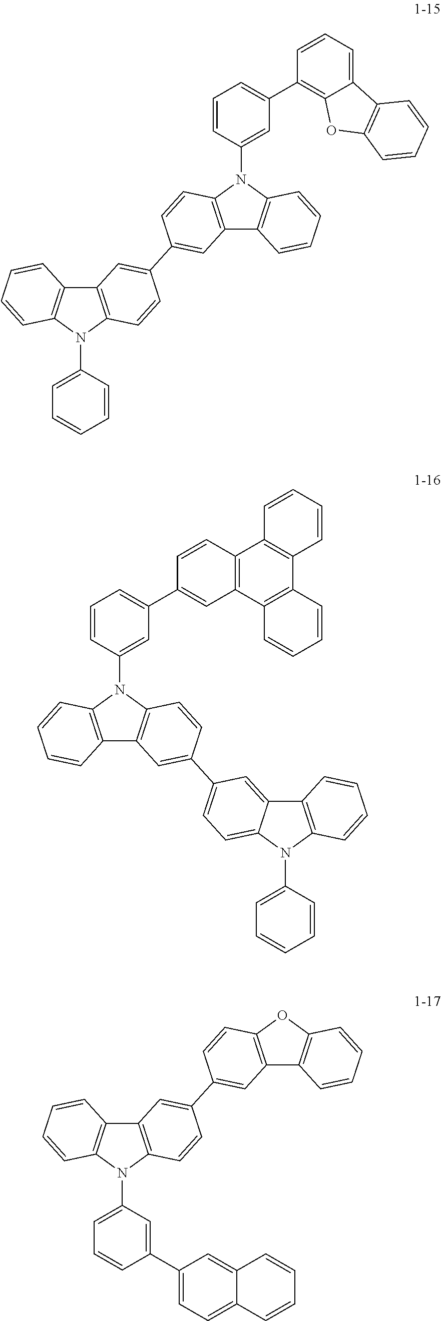 [SMILES: c1ccc(-n2c3ccccc3c3cc(-c4ccc5c(c4)c4ccccc4n5-c4cccc(-c5cccc6c5oc5ccccc56)c4)ccc32)cc1 ; c1ccc(-n2c3ccccc3c3cc(-c4ccc5c(c4)c4ccccc4n5-c4cccc(-c5ccc6c7ccccc7c7ccccc7c6c5)c4)ccc32)cc1 ; c1cc(-c2ccc3ccccc3c2)cc(-n2c3ccccc3c3cc(-c4ccc5oc6ccccc6c5c4)ccc32)c1]

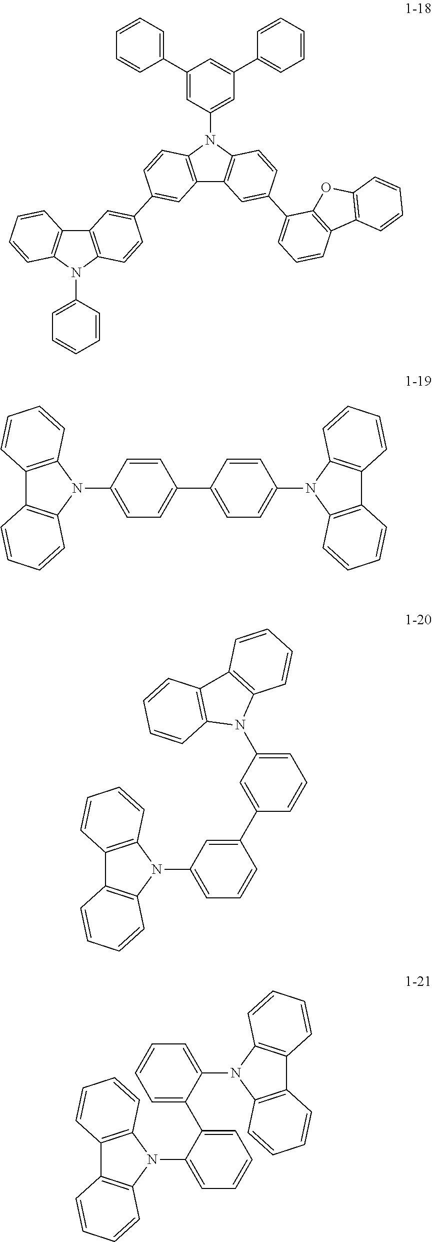 [SMILES: c1ccc(-c2cc(-c3ccccc3)cc(-n3c4ccc(-c5ccc6c(c5)c5ccccc5n6-c5ccccc5)cc4c4cc(-c5cccc6c5oc5ccccc56)ccc43)c2)cc1 ; c1ccc2c(c1)c1ccccc1n2-c1ccc(-c2ccc(-n3c4ccccc4c4ccccc43)cc2)cc1 ; c1cc(-c2cccc(-n3c4ccccc4c4ccccc43)c2)cc(-n2c3ccccc3c3ccccc32)c1 ; c1ccc(-n2c3ccccc3c3ccccc32)c(-c2ccccc2-n2c3ccccc3c3ccccc32)c1]

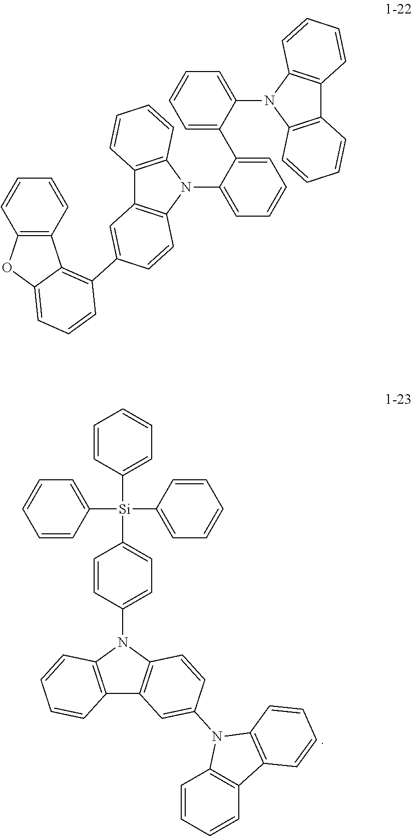 [SMILES: c1ccc(-n2c3ccccc3c3ccccc32)c(-c2ccccc2-n2c3ccccc3c3cc(-c4cccc5oc6ccccc6c45)ccc32)c1 ; c1ccc([Si](c2ccccc2)(c2ccccc2)c2ccc(-n3c4ccccc4c4cc(-n5c6ccccc6c6ccccc65)ccc43)cc2)cc1]

The light emitting device ED according to an embodiment may further include a third compound, in addition to the first compound represented by Formula 1-1 or Formula 1-2, in the emission layer EML. The emission layer EML may include an organometallic complex including platinum (Pt) as a central metal atom and ligands bonded to the central metal atom, as the third compound. In the light emitting device ED according to an embodiment, the emission layer EML may include a compound represented by Formula D-1 as the third compound.

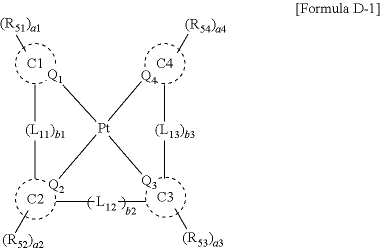

In Formula D-1, Q₁ to Q₄ may each independently be C or N.

In Formula D-1, C1 to C4 may each independently be a substituted or unsubstituted hydrocarbon ring of 5 to 30 ring-forming carbon atoms, or a substituted or unsubstituted heterocycle of 2 to 30 ring-forming carbon atoms.

In Formula D-1, L₁₁ to L₁₃ may each independently be a direct linkage, *—O—*, *—S—*,

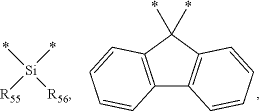

a substituted or unsubstituted divalent alkyl group of 1 to 20 carbon atoms, a substituted or unsubstituted arylene group of 6 to 30 ring-forming carbon atoms, or a substituted or unsubstituted heteroarylene group of 2 to 30 ring-forming carbon atoms. In L₁₁ to L₁₃, —* each represented a binding site to one of C1 to C4.

In Formula D-1, R₅₁ to R₅₆ may each independently be a hydrogen atom, a deuterium atom, a halogen atom, a cyano group, a substituted or unsubstituted amine group, a substituted or unsubstituted alkyl group of 1 to 20 carbon atoms, a substituted or unsubstituted aryl group of 6 to 30 ring-forming carbon atoms, or a substituted or unsubstituted heteroaryl group of 2 to 30 ring-forming carbon atoms, or may be combined with an adjacent group to form a ring. For example, R₅₁ to R₅₄ may each independently be a methyl group or a t-butyl group.

In Formula D-1, a1 to a4 may each independently be an integer from 0 to 4. If a1 to a4 are each 2 or more, multiple groups of each of R₅₁ to R₅₄ may be all the same, or at least one thereof may be different.

In Formula D-1, b1 to b3 may each independently be 0 or 1. If b1 is 0, C1 and C2 may not be connected to each other. If b2 is 0, C2 and C3 may not be connected to each other. If b3 is 0, C3 and C4 may not be connected to each other.

In an embodiment, in Formula D-1, C1 to C4 may each independently be a substituted or unsubstituted hydrocarbon ring or a substituted or unsubstituted heterocycle, represented by any one of C-1 to C-4

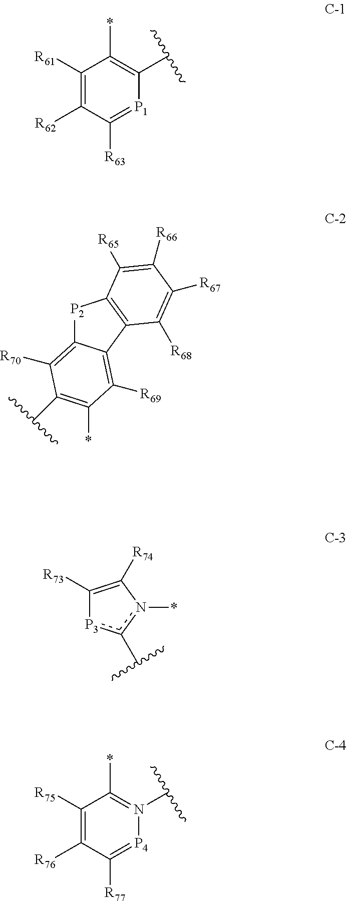

In C-1 to C-4, P₁ may be C—* or C(R₆₄), P₂ may be N—* or N(R₇₁), P₃ may be N—* or N(R₇₂), and P₄ may be C—* or C(R₇₈). R₆₁ to R₇₈ may each independently be a substituted or unsubstituted alkyl group of 1 to 20 carbon atoms, a substituted or unsubstituted aryl group of 6 to 30 ring-forming carbon atoms, or a substituted or unsubstituted heteroaryl group of 2 to 30 ring-forming carbon atoms, or may be combined with an adjacent group to form a ring.

In C-1 to C-4,

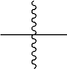

represents a binding site to a Pt central metal atom, and —* represents a binding site to one of C1 to C4 or to one of L₁₁ to L₁₃.

The third compound represented by Formula D-1 may be a phosphorescence dopant. In an embodiment, the third compound may be a light emitting dopant which emits blue light, and the emission layer EML may emit phosphorescence. For example, the emission layer EML may emit phosphorescence as blue light.

In an embodiment, the emission layer EML may include at least one compound selected from Compound Group 3 as the third compound. The emission layer EML may include at least one compound selected from Compound Group 3 as a phosphorescence dopant material.

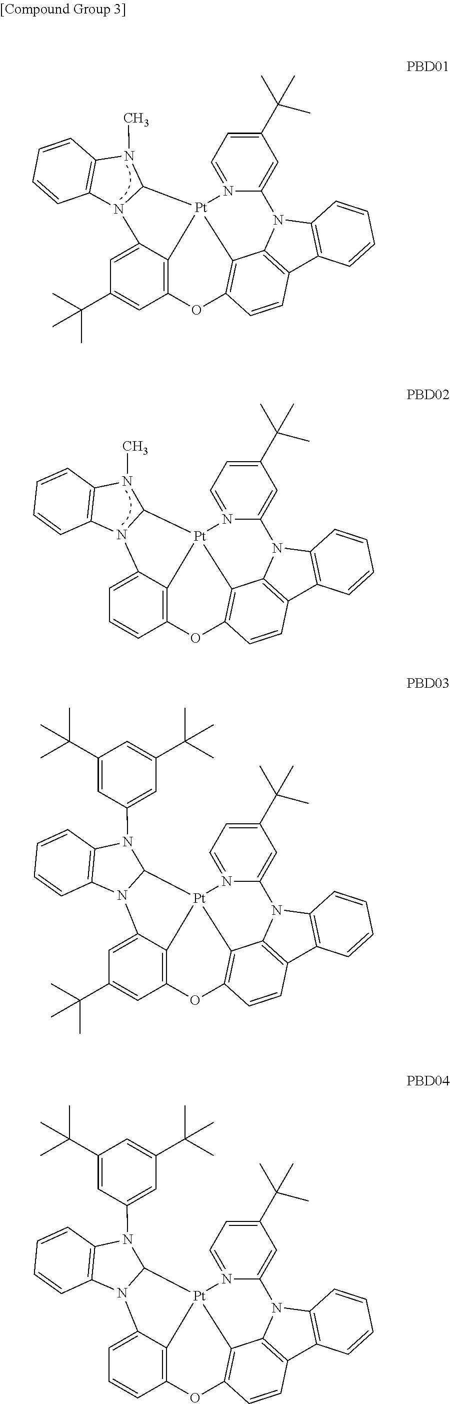

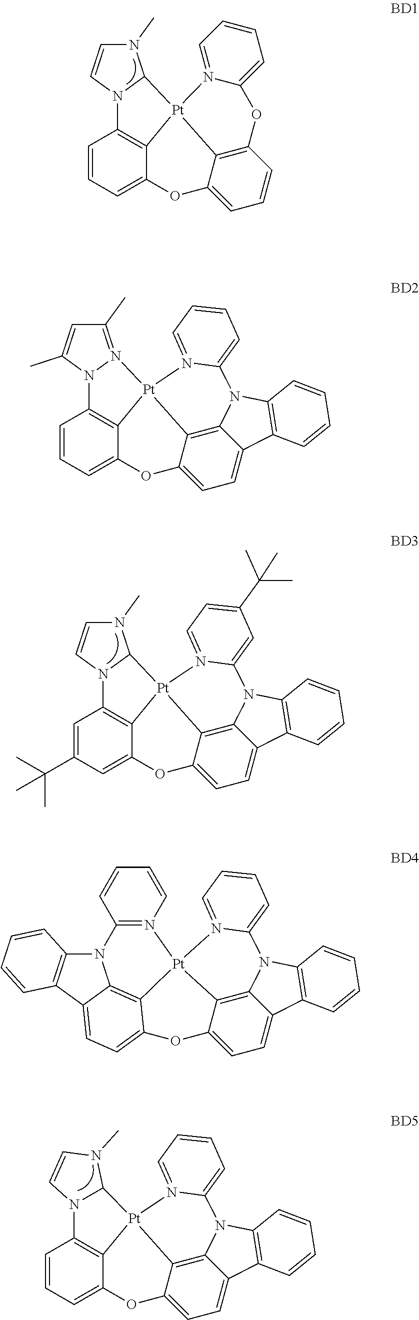

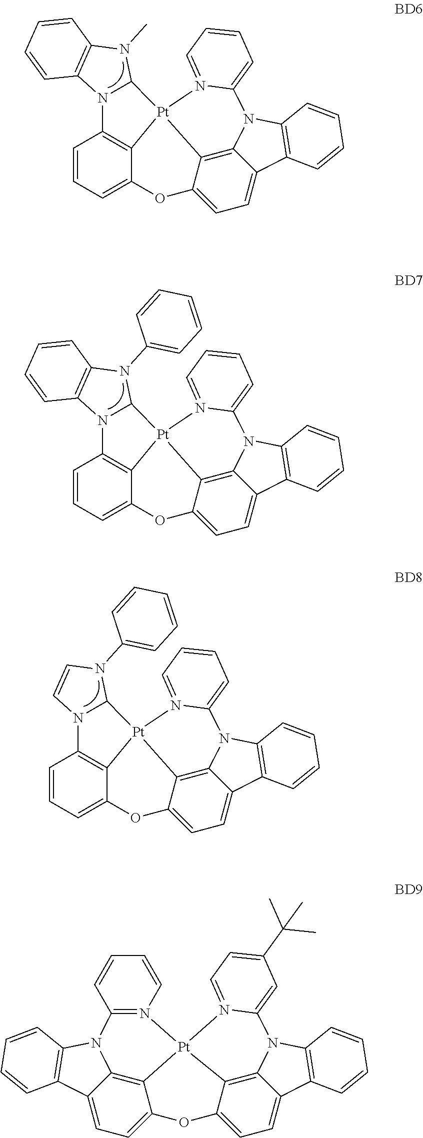

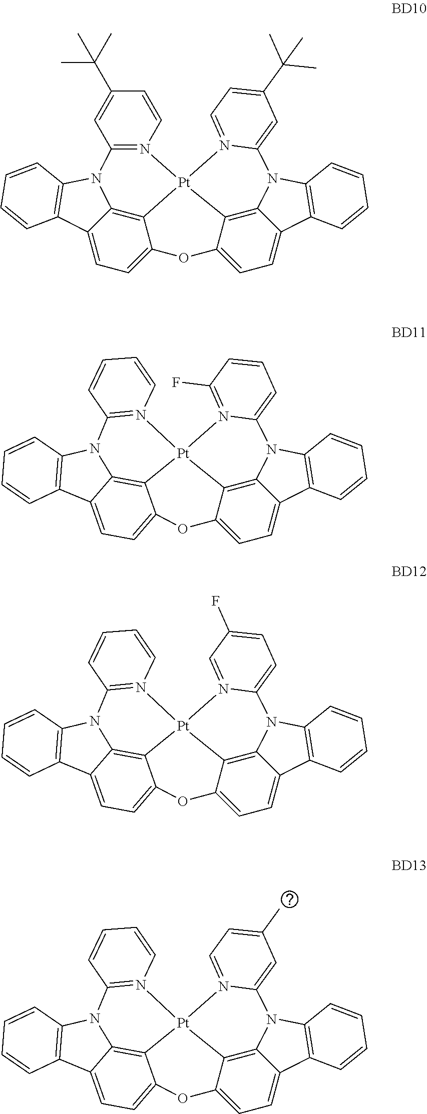

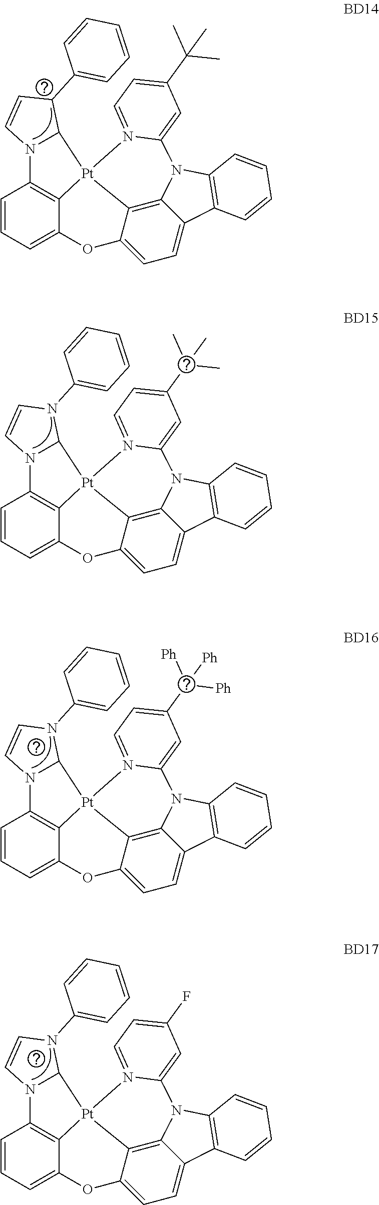

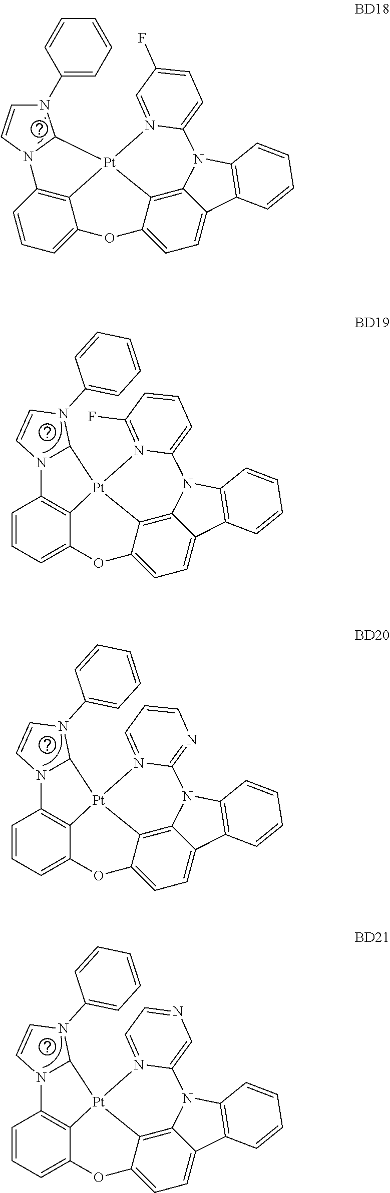

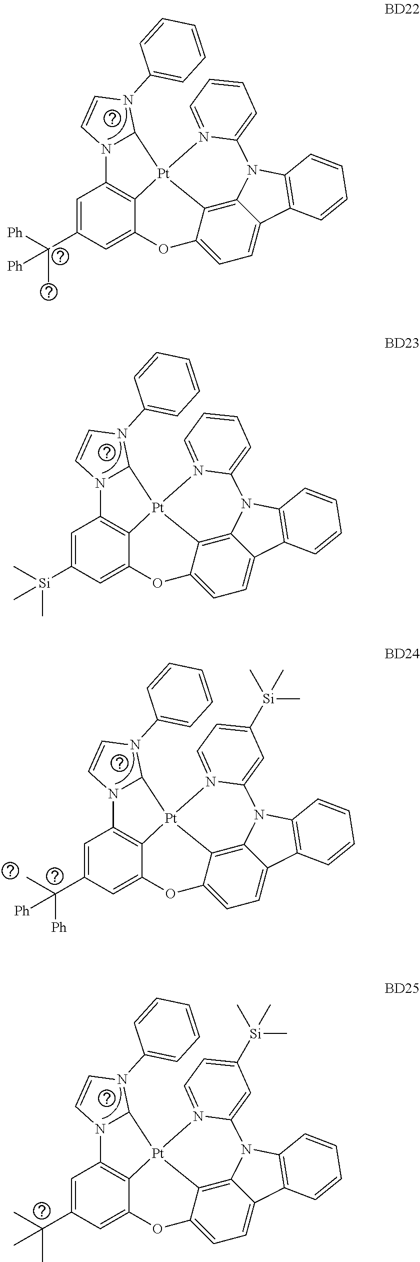

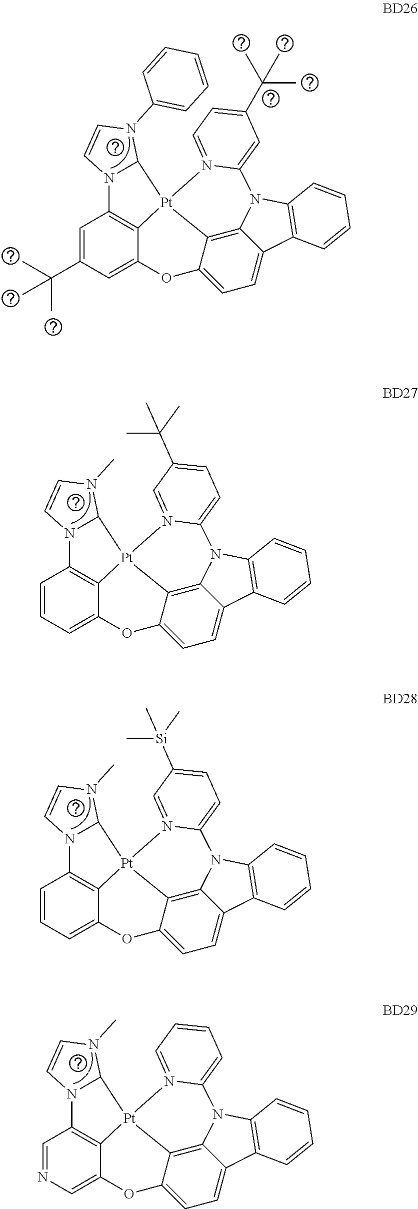

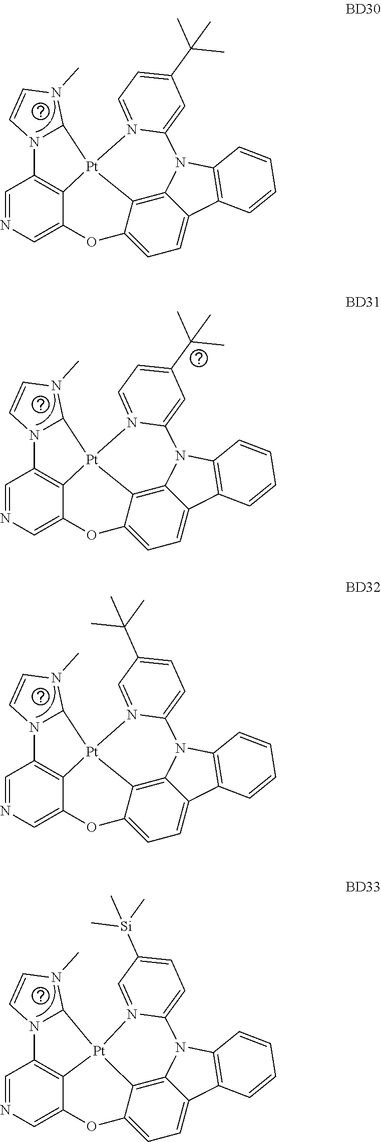

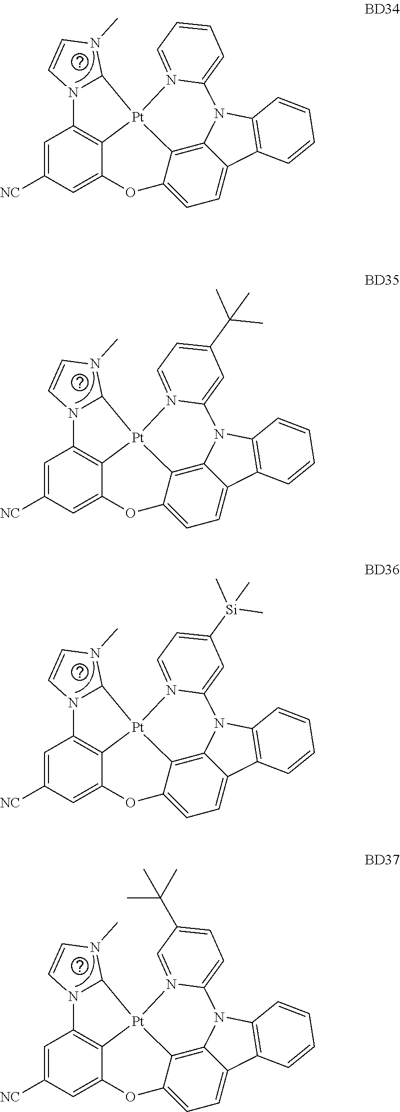

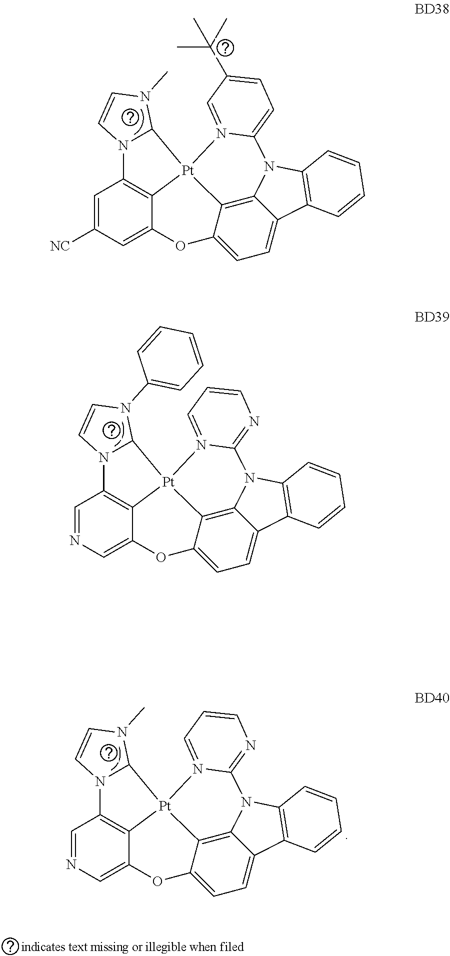

In the light emitting device ED according to an embodiment, if the emission layer EML includes the first compound, the second compound, and the third compound, an amount of the third compound in the emission layer EML may be in a range of about 10 wt % to about 20 wt %, based on a total weight of the first compound, the second compound, and the third compound. However, embodiments are not limited thereto. If the amount of the third compound satisfies the above-described range, energy transition from the first compound and the second compound to the third compound may increase, and accordingly, emission efficiency and device lifetime may be improved.

An amount of the first compound and the second compound in the emission layer EML may be a remainder excluding the above-described amount of the third compound. For example, a total amount of the first compound and the second compound in the emission layer EML may be in a range of about 80 wt % to about 90 wt %, based on a total weight of the first compound, the second compound, and the third compound.

Within a total amount of the first compound and the second compound in the emission layer EML, a weight ratio of the first compound to the second compound may be in a range of about 3:7 to about 7:3.

If the amount of the first compound and the second compound satisfies the above-described ratio, charge balance properties in the emission layer EML may be improved, and emission efficiency and device lifetime may be improved. If the amount of the first compound and the second compound deviates from the above-described range of ratios, charge balance in the emission layer EML may be poor, emission efficiency may be reduced, and the device may readily deteriorate.

If the first compound, the second compound, and the third compound, included in the emission layer EML satisfy the above-described amounts and ratios, excellent emission efficiency and long-life characteristics may be achieved.

In the light emitting element ED according to an embodiment, the emission layer EML may further include anthracene derivatives, pyrene derivatives, fluoranthene derivatives, chrysene derivatives, dihydrobenzanthracene derivatives, or triphenylene derivatives. For example, the emission layer EML may include anthracene derivatives or pyrene derivatives.

In the light emitting devices ED according to embodiments shown in FIG. 3 to FIG. 6, the emission layer EML may include a host of the related art and a dopant of the related art, in addition to the aforementioned host and dopant. For example, the emission layer EML may include a compound represented by Formula E-1. The compound represented by Formula E-1 may be used as a fluorescence host material.

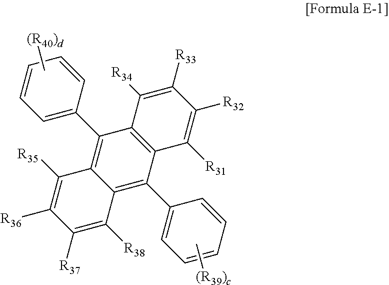

In Formula E-1, R₃₁ to R₄₀ may each independently be a hydrogen atom, a deuterium atom, a halogen atom, a substituted or unsubstituted silyl group, a substituted or unsubstituted thio group, a substituted or unsubstituted oxy group, a substituted or unsubstituted alkyl group of 1 to 10 carbon atoms, a substituted or unsubstituted alkenyl group of 2 to 10 carbon atoms, a substituted or unsubstituted aryl group of 6 to 30 ring-forming carbon atoms, or a substituted or unsubstituted heteroaryl group of 2 to 30 ring-forming carbon atoms, or may be combined with an adjacent group to form a ring. In Formula E-1, R₃₁ to R₄₀ may be combined with an adjacent group to form a saturated hydrocarbon ring, an unsaturated hydrocarbon ring, a saturated heterocycle, or an unsaturated heterocycle.

In Formula E-1, c and d may each independently be an integer from 0 to 5.

The compound represented by Formula E-1 may be any compound selected from Compound E1 to Compound E19.

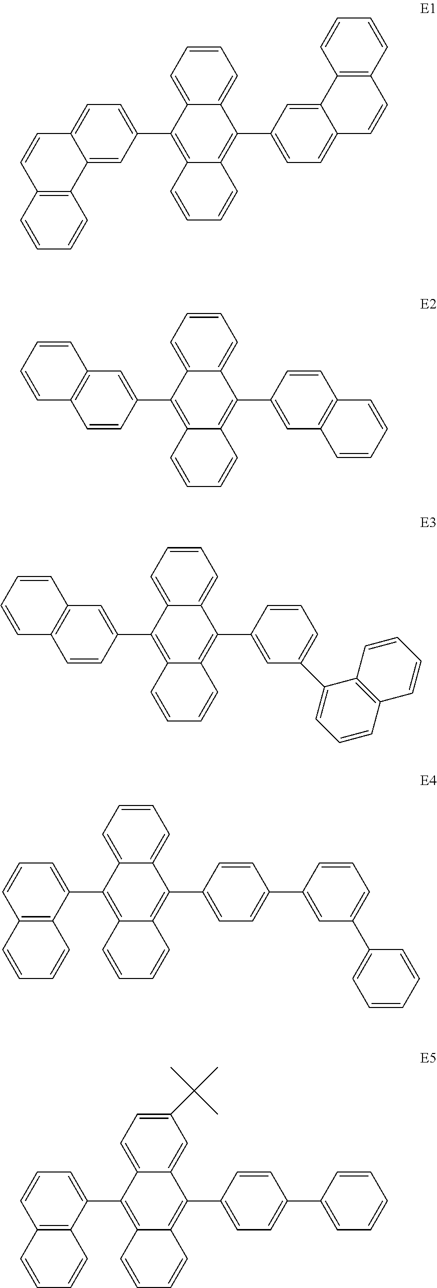

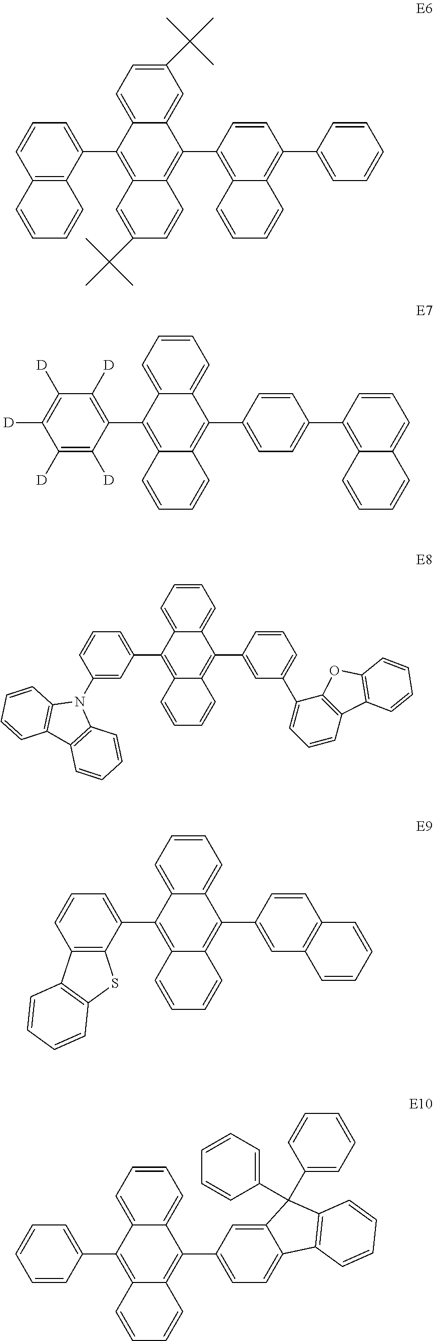

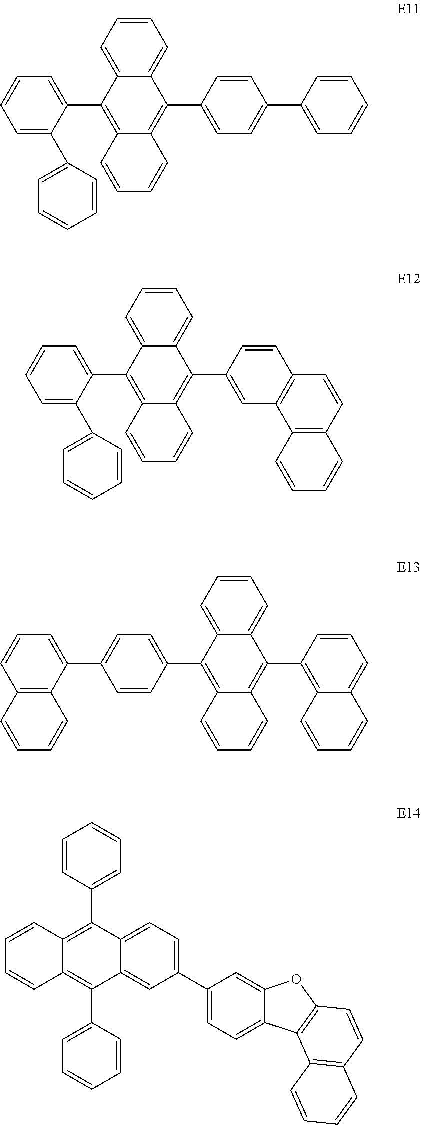

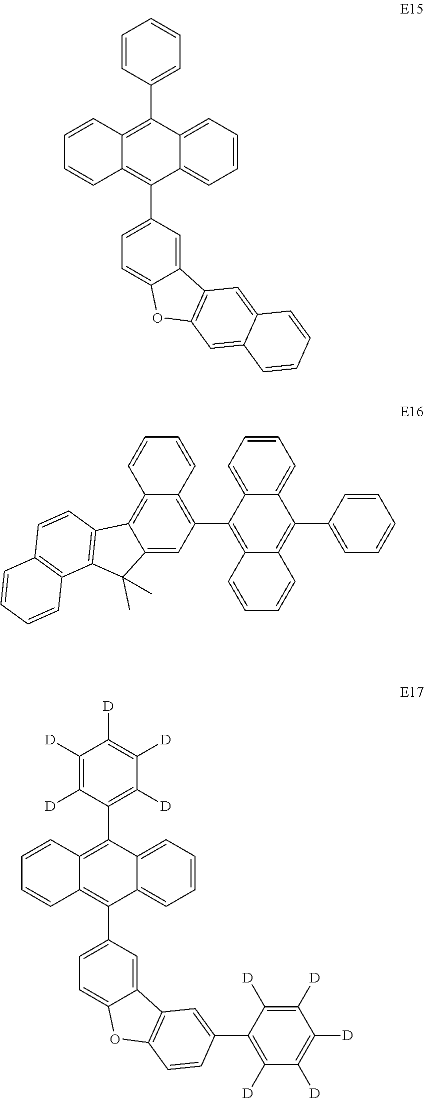

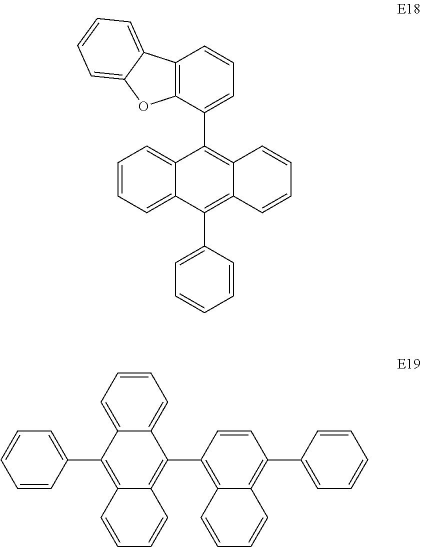

In an embodiment, the emission layer EML may include a compound represented by Formula E-2a or Formula E-2b. The compound represented by Formula E-2a or Formula E-2b may be used as a phosphorescence host material.

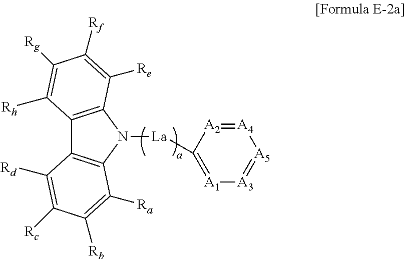

In Formula E-2a, a may be an integer from 0 to 10; and Lₐ may be a direct linkage, a substituted or unsubstituted arylene group of 6 to 30 ring-forming carbon atoms, or a substituted or unsubstituted heteroarylene group of 2 to 30 ring-forming carbon atoms. If a is 2 or more, multiple Lₐ groups may each independently be a substituted or unsubstituted arylene group of 6 to 30 ring-forming carbon atoms, or a substituted or unsubstituted heteroarylene group of 2 to 30 ring-forming carbon atoms.

In Formula E-2a, A₁ to A₅ may each independently be N or C(Rᵢ). In Formula E-2a, Rₐ to Rᵢ may each independently be a hydrogen atom, a deuterium atom, a substituted or unsubstituted amine group, a substituted or unsubstituted thio group, a substituted or unsubstituted oxy group, a substituted or unsubstituted alkyl group of 1 to 20 carbon atoms, a substituted or unsubstituted alkenyl group of 2 to 20 carbon atoms, a substituted or unsubstituted aryl group of 6 to 30 ring-forming carbon atoms, or a substituted or unsubstituted heteroaryl group of 2 to 30 ring-forming carbon atoms, or may be combined with an adjacent group to form a ring. For example, Rₐ to Rᵢ may be combined with an adjacent group to form a hydrocarbon ring or a heterocycle including N, O, S, etc. as a ring-forming atom.

In Formula E-2a, two or three of A₁ to A₅ may each be N, and the remainder of A₁ to A₅ may each independently be C(Rᵢ).

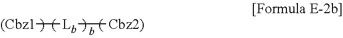

In Formula E-2b, Cbz1 and Cbz2 may each independently be an unsubstituted carbazole group, or a carbazole group substituted with an aryl group of 6 to 30 ring-forming carbon atoms. In Formula E-2b, L_{b} may be a direct linkage, a substituted or unsubstituted arylene group of 6 to 30 ring-forming carbon atoms, or a substituted or unsubstituted heteroarylene group of 2 to 30 ring-forming carbon atoms. In Formula E-2b, b may be an integer from 0 to 10, and if b is 2 or more, multiple L_{b} groups may each independently be a substituted or unsubstituted arylene group of 6 to 30 ring-forming carbon atoms, or a substituted or unsubstituted heteroarylene group of 2 to 30 ring-forming carbon atoms.

The compound represented by Formula E-2a or Formula E-2b may be any compound selected from Compound Group E-2. However, the compounds shown in Compound Group E-2 are only examples, and the compound represented by Formula E-2a or Formula E-2b is not limited to Compound Group E-2.

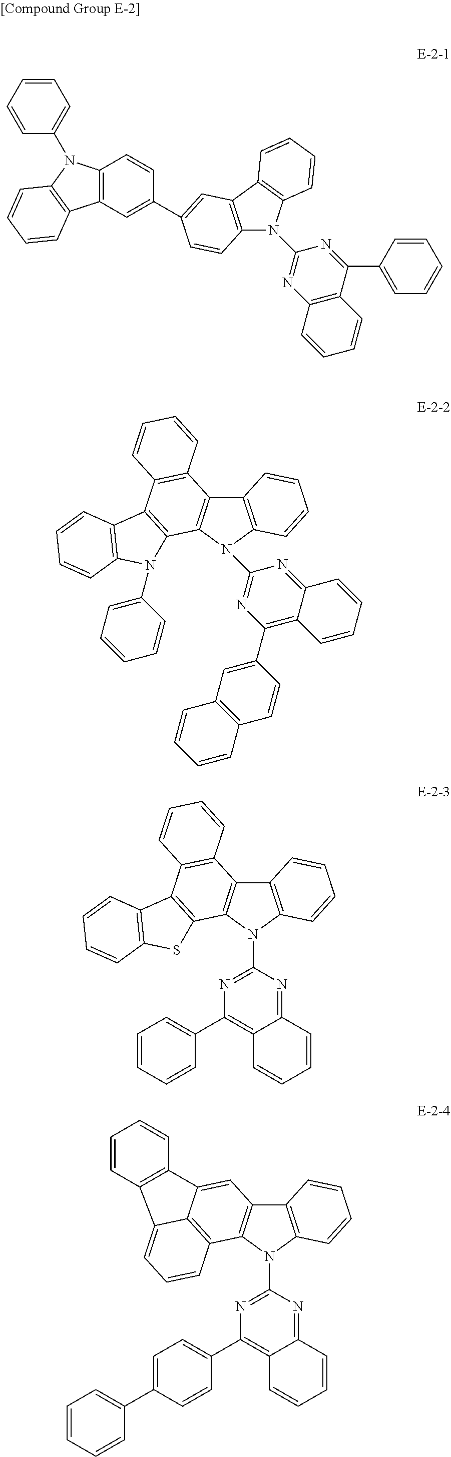

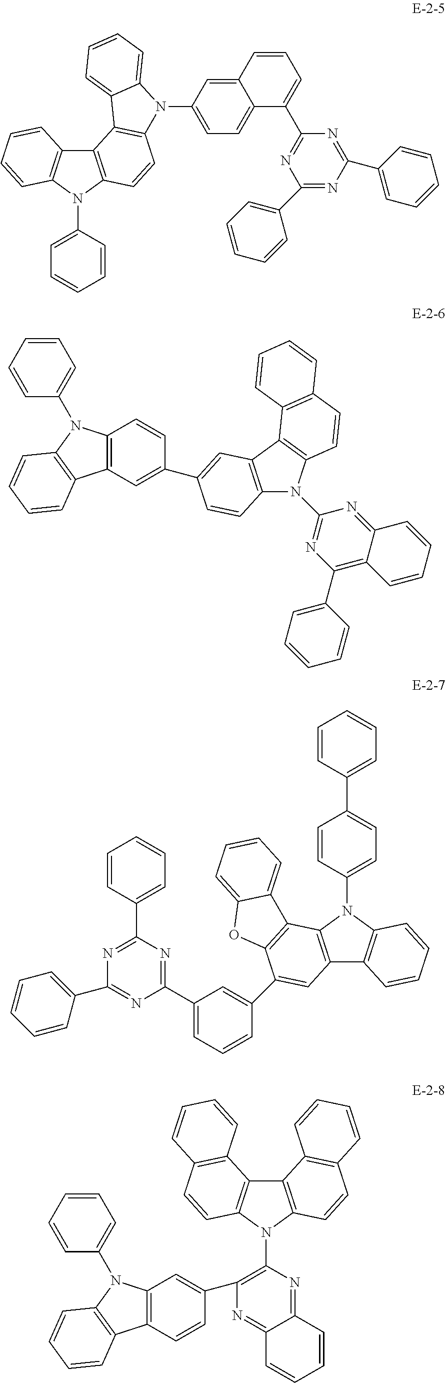 [SMILES: c1ccc(-c2nc(-c3ccccc3)nc(-c3cccc4cc(-n5c6ccccc6c6c7c8ccccc8n(-c8ccccc8)c7ccc65)ccc34)n2)cc1 ; c1ccc(-c2nc(-n3c4ccc(-c5ccc6c(c5)c5ccccc5n6-c5ccccc5)cc4c4c5ccccc5ccc43)nc3ccccc23)cc1 ; c1ccc(-c2ccc(-n3c4ccccc4c4cc(-c5cccc(-c6nc(-c7ccccc7)nc(-c7ccccc7)n6)c5)c5oc6ccccc6c5c43)cc2)cc1 ; c1ccc(-n2c3ccccc3c3ccc(-c4nc5ccccc5nc4-n4c5ccc6ccccc6c5c5c6ccccc6ccc54)cc32)cc1]

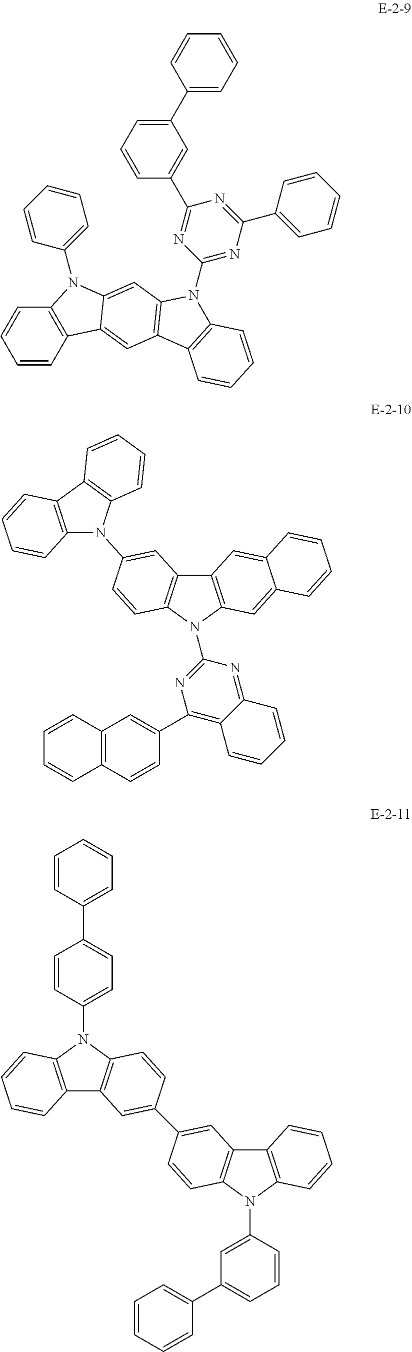 [SMILES: c1ccc(-c2cccc(-c3nc(-c4ccccc4)nc(-n4c5ccccc5c5cc6c7ccccc7n(-c7ccccc7)c6cc54)n3)c2)cc1 ; c1ccc2cc(-c3nc(-n4c5ccc(-n6c7ccccc7c7ccccc76)cc5c5cc6ccccc6cc54)nc4ccccc34)ccc2c1 ; c1ccc(-c2ccc(-n3c4ccccc4c4cc(-c5ccc6c(c5)c5ccccc5n6-c5cccc(-c6ccccc6)c5)ccc43)cc2)cc1]

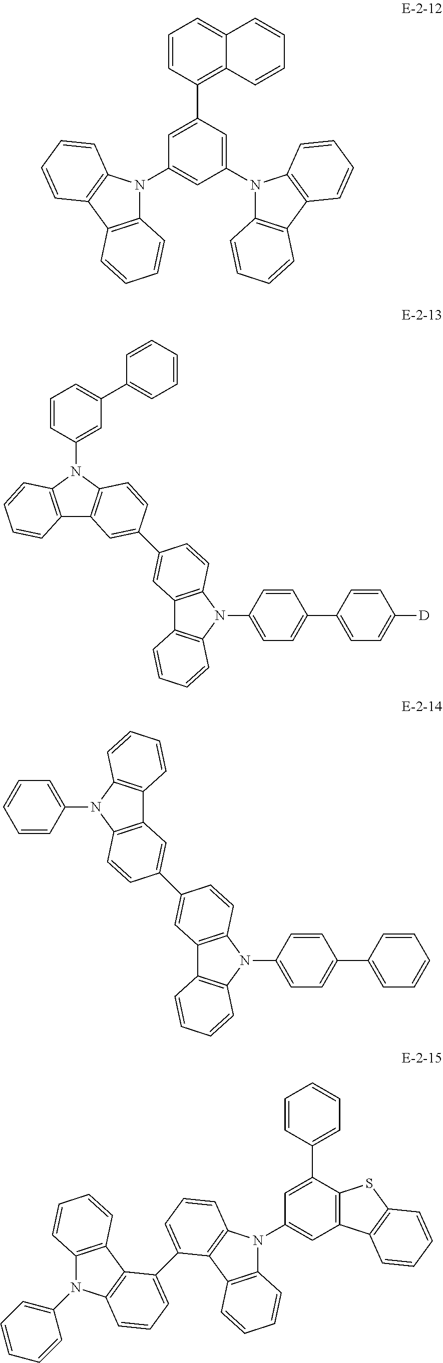 [SMILES: c1ccc2c(-c3cc(-n4c5ccccc5c5ccccc54)cc(-n4c5ccccc5c5ccccc54)c3)cccc2c1 ; [2H]c1ccc(-c2ccc(-n3c4ccccc4c4cc(-c5ccc6c(c5)c5ccccc5n6-c5cccc(-c6ccccc6)c5)ccc43)cc2)cc1 ; c1ccc(-c2ccc(-n3c4ccccc4c4cc(-c5ccc6c(c5)c5ccccc5n6-c5ccccc5)ccc43)cc2)cc1 ; c1ccc(-c2cc(-n3c4ccccc4c4c(-c5cccc6c5c5ccccc5n6-c5ccccc5)cccc43)cc3c2sc2ccccc23)cc1]

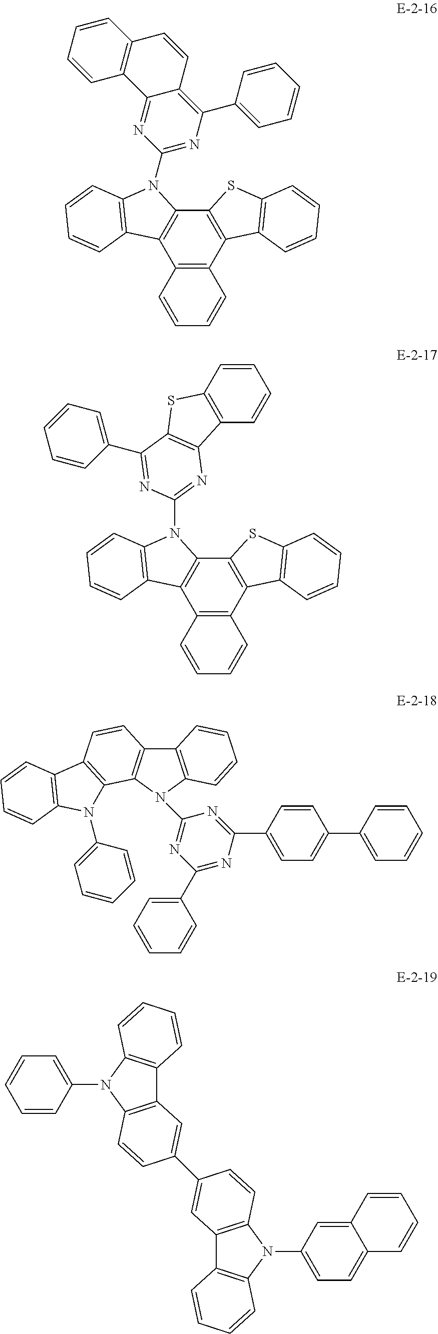

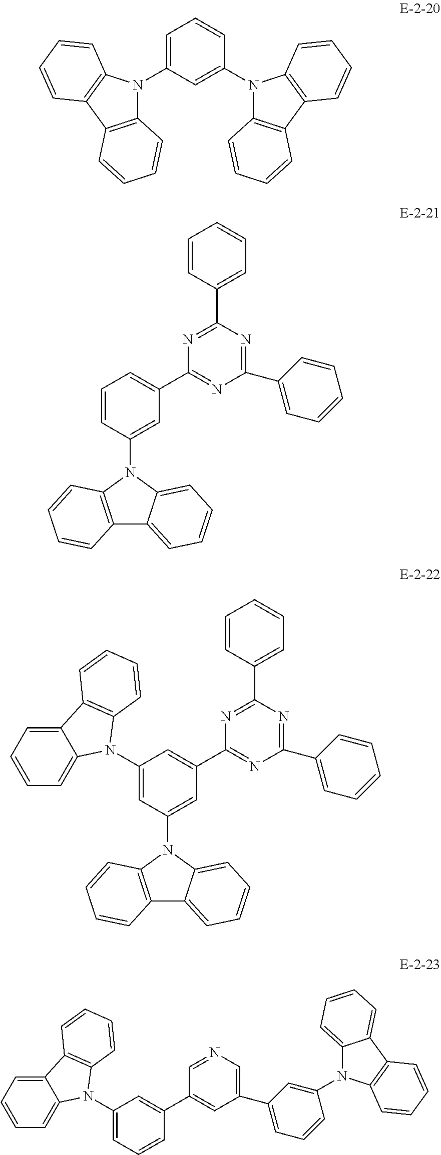 [SMILES: c1cc(-n2c3ccccc3c3ccccc32)cc(-n2c3ccccc3c3ccccc32)c1 ; c1ccc(-c2nc(-c3ccccc3)nc(-c3cccc(-n4c5ccccc5c5ccccc54)c3)n2)cc1 ; c1ccc(-c2nc(-c3ccccc3)nc(-c3cc(-n4c5ccccc5c5ccccc54)cc(-n4c5ccccc5c5ccccc54)c3)n2)cc1 ; c1cc(-c2cncc(-c3cccc(-n4c5ccccc5c5ccccc54)c3)c2)cc(-n2c3ccccc3c3ccccc32)c1]

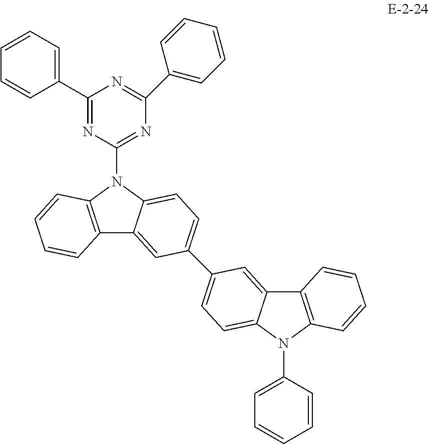 [SMILES: c1ccc(-c2nc(-c3ccccc3)nc(-n3c4ccccc4c4cc(-c5ccc6c(c5)c5ccccc5n6-c5ccccc5)ccc43)n2)cc1]

The emission layer EML may further include a material of the related art as a host material. For example, the emission layer EML may include as a host material, at least one of bis (4-(9H-carbazol-9-yl) phenyl) diphenylsilane (BCPDS), (4-(1-(4-(diphenylamino) phenyl) cyclohexyl) phenyl) diphenyl-phosphine oxide (POPCPA), bis[2-(diphenylphosphino)phenyl] ether oxide (DPEPO), 4,4′-bis(carbazol-9-yl)biphenyl (CBP), 1,3-bis(carbazol-9-yl)benzene (mCP), 2,8-bis(diphenylphosphoryl)dibenzo[b,d]furan (PPF), 4,4′,4″-tris(carbazol-9-yl)-triphenylamine (TCTA), or 1,3,5-tris(1-phenyl-1H-benzo[d]imidazole-2-yl)benzene (TPBi). However, embodiments are not limited thereto. For example, tris(8-hydroxyquinolino)aluminum (Alq₃), 9,10-di(naphthalene-2-yl)anthracene (ADN), 2-tert-butyl-9,10-di(naphth-2-yl)anthracene (TBADN), distyrylarylene (DSA), 4,4′-bis(9-carbazolyl)-2,2′-dimethyl-biphenyl (CDBP), 2-methyl-9,10-bis(naphthalen-2-yl)anthracene (MADN), hexaphenyl cyclotriphosphazene (CP1), 1,4-bis(triphenylsilyl)benzene (UGH2), hexaphenylcyclotrisiloxane (DPSiO₃), octaphenylcyclotetra siloxane (DPSiO₄), etc. may be used as the host material.

The emission layer EML may further include a compound represented by Formula M-a. The compound represented by Formula M-a may be used as a phosphorescence dopant material.

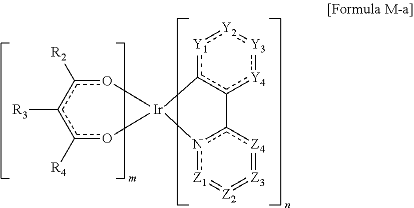

In Formula M-a, Y₁ to Y₄ and Z₁ to Z₄ may each independently be C(R₁) or N; and R₁ to R₄ may each independently be a hydrogen atom, a deuterium atom, a substituted or unsubstituted amine group, a substituted or unsubstituted thio group, a substituted or unsubstituted oxy group, a substituted or unsubstituted alkyl group of 1 to 20 carbon atoms, a substituted or unsubstituted alkenyl group of 2 to 20 carbon atoms, a substituted or unsubstituted aryl group of 6 to 30 ring-forming carbon atoms, or a substituted or unsubstituted heteroaryl group of 2 to 30 ring-forming carbon atoms, or may be combined with an adjacent group to form a ring. In Formula M-a, m may be 0 or 1, and n may be 2 or 3. In Formula M-a, if m is 0, then n may be 3, and if m is 1, then n may be 2.

The compound represented by Formula M-a may be used as a phosphorescence dopant.

The compound represented by Formula M-a may be any compound selected from Compounds M-a1 to M-a25. However, Compounds M-a1 to M-a25 are only examples, and the compound represented by Formula M-a is not limited to Compounds M-a1 to M-a25.

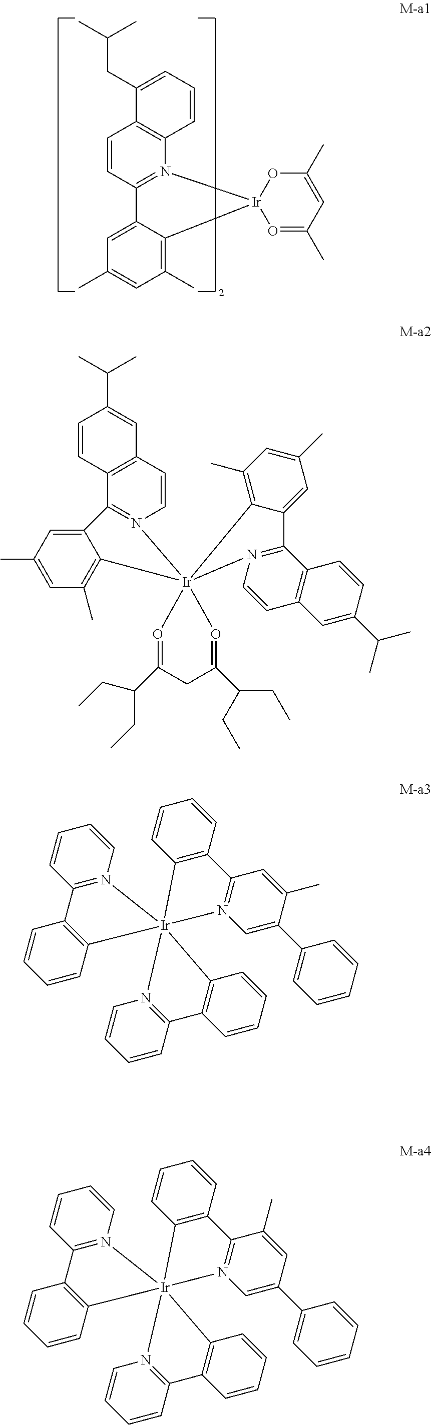

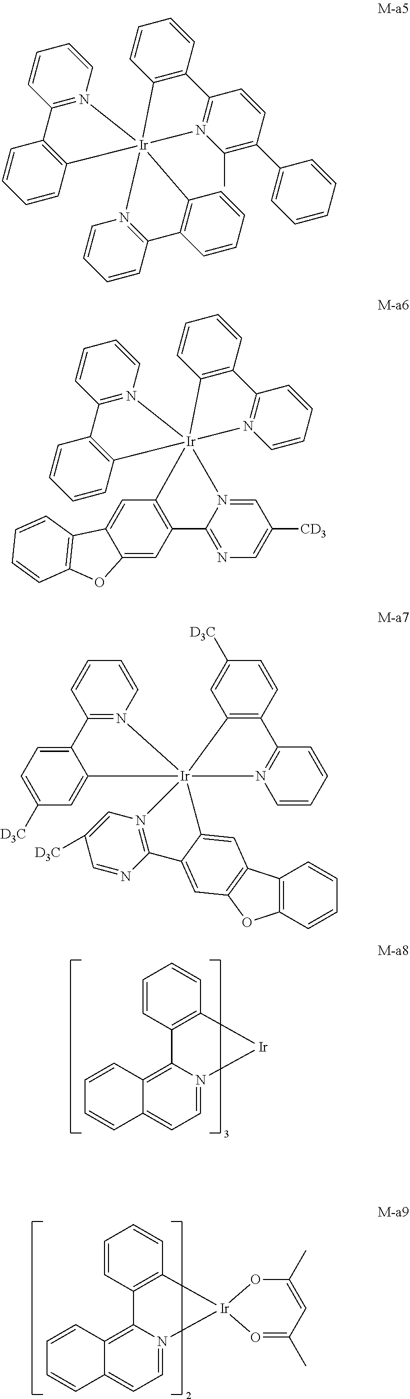

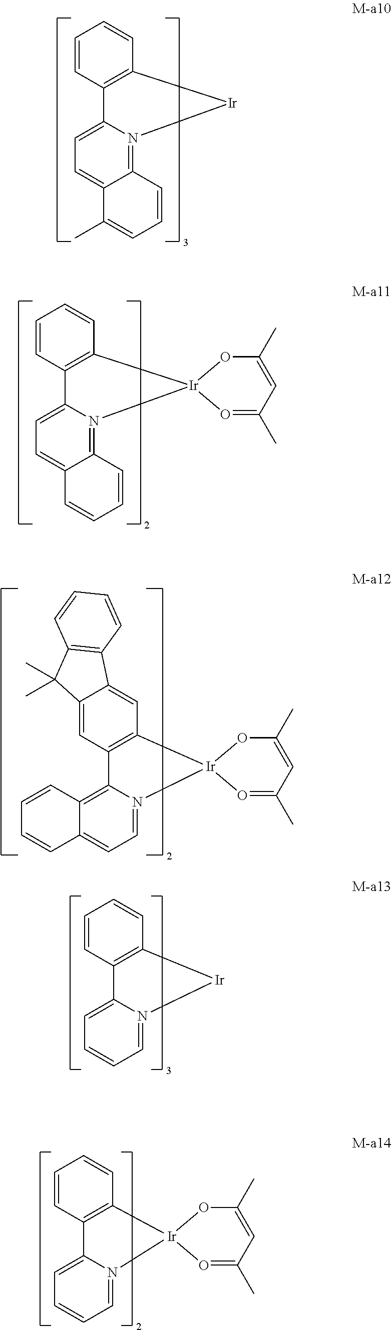

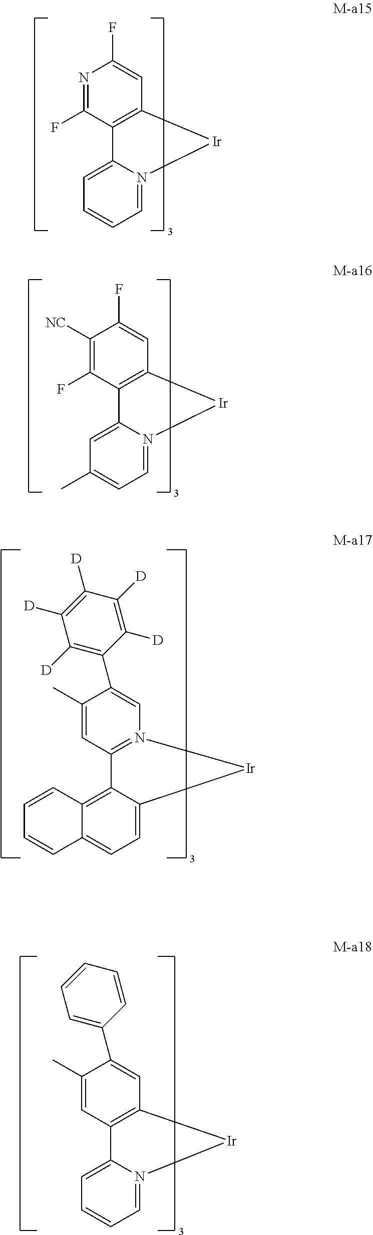

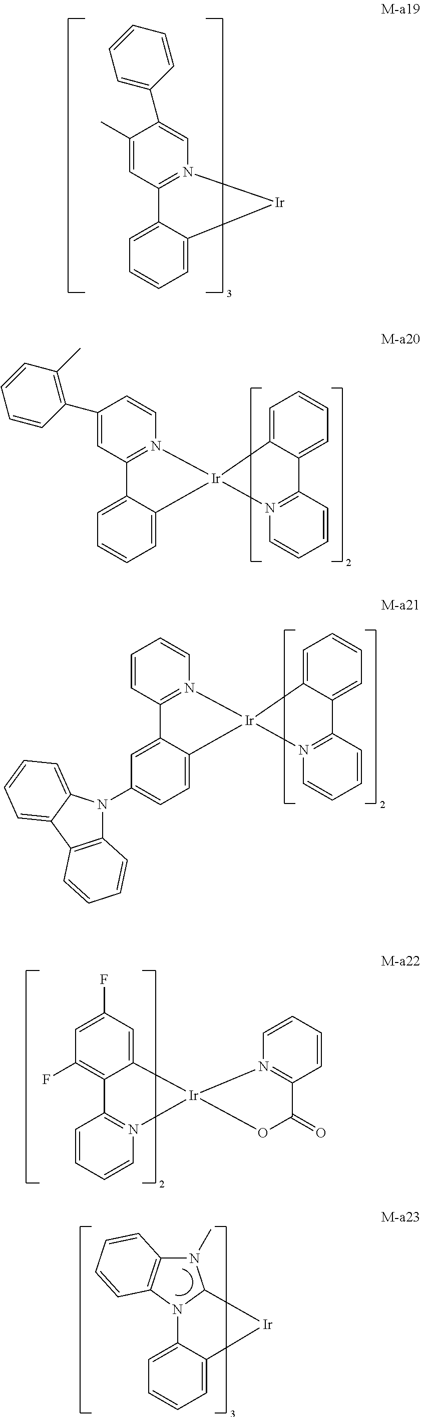

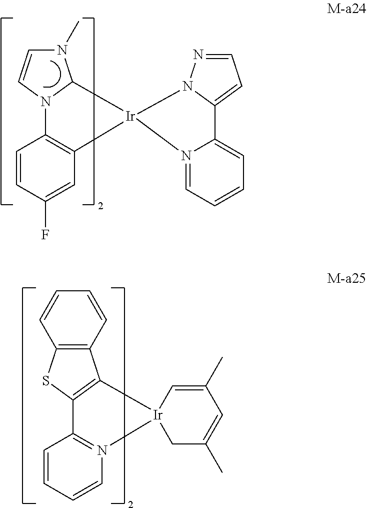

The emission layer EML may include a compound represented by any one of Formula F-a to Formula F-c. The compounds represented by Formula F-a to Formula F-c may be used as fluorescence dopant materials.

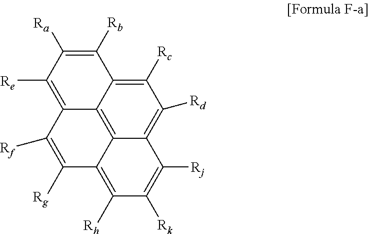

In Formula F-a, two of Rₐ to Rⱼ may each independently be substituted with a group represented by *—NAr₁Ar₂. In Formula F-a, the remainder of Rₐ to Rⱼ which are not substituted with the group represented by *—NAr₁Ar₂ may each independently be a hydrogen atom, a deuterium atom, a halogen atom, a cyano group, a substituted or unsubstituted amine group, a substituted or unsubstituted alkyl group of 1 to 20 carbon atoms, a substituted or unsubstituted aryl group of 6 to 30 ring-forming carbon atoms, or a substituted or unsubstituted heteroaryl group of 2 to 30 ring-forming carbon atoms.

In the group represented by *—NAr₁Ar₂, Ar₁ and Ar₂ may each independently be a substituted or unsubstituted aryl group of 6 to 30 ring-forming carbon atoms, or a substituted or unsubstituted heteroaryl group of 2 to 30 ring-forming carbon atoms. For example, at least one of Ar₁ and Ar₂ may be a heteroaryl group including O or S as a ring-forming atom.

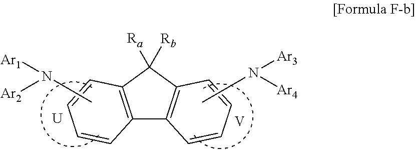

In Formula F-b, Rₐ and R_{b} may each independently be a hydrogen atom, a deuterium atom, a substituted or unsubstituted alkyl group of 1 to 20 carbon atoms, a substituted or unsubstituted alkenyl group of 2 to 20 carbon atoms, a substituted or unsubstituted aryl group of 6 to 30 ring-forming carbon atoms, or a substituted or unsubstituted heteroaryl group of 2 to 30 ring-forming carbon atoms, or may be combined with an adjacent group to form a ring.

In Formula F-b, Ar₁ to Ar₄ may each independently be an aryl group of 6 to 30 ring-forming carbon atoms, or a substituted or unsubstituted heteroaryl group of 2 to 30 ring-forming carbon atoms. For example, at least one of Ar₁ to Ar₄ may be a heteroaryl group including O or S as a ring-forming atom.

In Formula F-b, U and V may each independently be a substituted or unsubstituted hydrocarbon ring of 5 to 30 ring-forming carbon atoms, or a substituted or unsubstituted heterocycle of 2 to 30 ring-forming carbon atoms.

In Formula F-b, the number of rings represented by U and V may each independently be 0 or 1. For example, in Formula F-b, if the number of U or V is 1, a fused ring may be present at the part designated by U or V, and if the number of U or V is 0, a fused ring may not be present at the part designated by U or V. If the number of U is 0 and the number of V is 1, or if the number of U is 1 and the number of V is 0, a fused ring having a fluorene core of Formula F-b may be a cyclic compound with four rings. If the number of U and V is each 0, a fused ring having a fluorene core of Formula F-b may be a cyclic compound with three rings. If the number of U and V is each 1, a fused ring having a fluorene core of Formula F-b may be a cyclic compound with five rings.

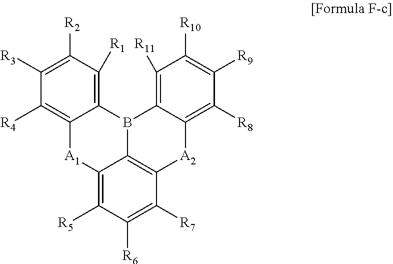

In Formula F-c, A₁ and A₂ may each independently be O, S, Se, or N(Rₘ); and Rₘ may be a hydrogen atom, a deuterium atom, a substituted or unsubstituted alkyl group of 1 to 20 carbon atoms, a substituted or unsubstituted aryl group of 6 to 30 ring-forming carbon atoms, or a substituted or unsubstituted heteroaryl group of 2 to 30 ring-forming carbon atoms. In Formula F-c, R₁ to R₁₁ may each independently be a hydrogen atom, a deuterium atom, a halogen atom, a cyano group, a substituted or unsubstituted amine group, a substituted or unsubstituted boryl group, a substituted or unsubstituted oxy group, a substituted or unsubstituted thio group, a substituted or unsubstituted alkyl group of 1 to 20 carbon atoms, a substituted or unsubstituted aryl group of 6 to 30 ring-forming carbon atoms, or a substituted or unsubstituted heteroaryl group of 2 to 30 ring-forming carbon atoms, or may be combined with an adjacent group to form a ring.

In Formula F-c, A₁ and A₂ may each independently be combined with the substituents of an adjacent ring to form a fused ring. For example, if A₁ and A₂ are each independently N(Rₘ), A₁ may be combined with R₄ or R₅ to form a ring. For example, A₂ may be combined with R₇ or R₈ to form a ring.

In an embodiment, the emission layer EML may include a dopant material of the related art, such as styryl derivatives (for example, 1,4-bis[2-(3-N-ethylcarbazoryl)vinyl]benzene (BCzVB), 4-(di-p-tolylamino)-4′-[(di-p-tolylamino)styryl]stilbene (DPAVB), N-(4-((E)-2-(6-((E)-4-(diphenylamino)styryl)naphthalen-2-yl)vinyl)phenyl)-N-phenylbenzenamine (N-BDAVBi), and 4,4′-bis[2-(4-(N,N-diphenylamino)phenyl)vinyl]biphenyl (DPAVBi)), perylene and the derivatives thereof (for example, 2,5,8,11-tetra-t-butylperylene (TBP)), pyrene and the derivatives thereof (for example, 1,1-dipyrene, 1,4-dipyrenylbenzene, and 1,4-bis(N,N-diphenylamino)pyrene), etc.

The emission layer EML may further include a phosphorescence dopant material of the related art. For example, the phosphorescence dopant may use a metal complex including iridium (Ir), platinum (Pt), osmium (Os), gold (Au), titanium (Ti), zirconium (Zr), hafnium (Hf), europium (Eu), terbium (Tb), or thulium (Tm). For example, iridium(III) bis(4,6-difluorophenylpyridinato-N,C2′)picolinate (FIrpic), bis(2,4-difluorophenylpyridinato)-tetrakis(1-pyrazolyl)borate iridium(III) (Fir6), or platinum octaethyl porphyrin (PtOEP) may be used as the phosphorescence dopant. However, embodiments are not limited thereto.

The emission layer EML may include a quantum dot. The quantum dot may be a Group II-VI compound, a Group III-VI compound, a Group compound, a Group III-V compound, a Group III-II-V compound, a Group IV-VI compound, a Group IV element, a Group IV compound, or any combination thereof.

The Group II-VI compound may include: a binary compound selected from the group consisting of CdSe, CdTe, CdS, ZnS, ZnSe, ZnTe, ZnO, HgS, HgSe, HgTe, MgSe, MgS, and mixtures thereof; a ternary compound selected from the group consisting of CdSeS, CdSeTe, CdSTe, ZnSeS, ZnSeTe, ZnSTe, HgSeS, HgSeTe, HgSTe, CdZnS, CdZnSe, CdZnTe, CdHgS, CdHgSe, CdHgTe, HgZnS, HgZnSe, HgZnTe, MgZnSe, MgZnS, and mixtures thereof; a quaternary compound selected from the group consisting of HgZnTeS, CdZnSeS, CdZnSeTe, CdZnSTe, CdHgSeS, CdHgSeTe, CdHgSTe, HgZnSeS, HgZnSeTe, HgZnSTe, and mixtures thereof; or any combination thereof.

The Group III-VI compound may include: a binary compound such as In₂S₃, and In₂Se₃; a ternary compound such as InGaS₃, and InGaSe₃; or any combination thereof.

The Group compound may include: a ternary compound selected from the group consisting of AgInS, AgInS₂, CuInS, CuInS₂, AgGaS₂, CuGaS₂, CuGaO₂, AgGaO₂, AgAlO₂ and mixtures thereof; a quaternary compound such as AgInGaS₂, and CuInGaS₂; or any combination thereof.

The Group III-V compound may include: a binary compound selected from the group consisting of GaN, GaP, GaAs, GaSb, AlN, AlP, AlAs, AlSb, InN, InP, InAs, InSb, and mixtures thereof; a ternary compound selected from the group consisting of GaNP, GaNAs, GaNSb, GaPAs, GaPSb, AlNP, AlNAs, AlNSb, AlPAs, AlPSb, InGaP, InAlP, InNP, InNAs, InNSb, InPAs, InPSb, and mixtures thereof; a quaternary compound selected from the group consisting of GaAlNP, GaAlNAs, GaAlNSb, GaAlPAs, GaAlPSb, GaInNP, GaInNAs, GaInNSb, GaInPAs, GaInPSb, InAlNP, InAlNAs, InAlNSb, InAlPAs, InAlPSb, and mixtures thereof; or any combination thereof. The Group III-V compound may further include a Group II metal. For example, InZnP, etc. may be selected as a Group III-II-V compound.

The Group IV-VI compound may include: a binary compound selected from the group consisting of SnS, SnSe, SnTe, PbS, PbSe, PbTe, and mixtures thereof; a ternary compound selected from the group consisting of SnSeS, SnSeTe, SnSTe, PbSeS, PbSeTe, PbSTe, SnPbS, SnPbSe, SnPbTe, and mixtures thereof; a quaternary compound selected from the group consisting of SnPbSSe, SnPbSeTe, SnPbSTe, and mixtures thereof; or any combination thereof. The Group IV element may be selected from the group consisting of Si, Ge, and a mixture thereof. The Group IV compound may be a binary compound selected from the group consisting of SiC, SiGe, and a mixture thereof.

A binary compound, a ternary compound, or a quaternary compound may be present in a particle at a uniform concentration or at a partially different concentration distribution state. In an embodiment, the quantum dot may have a core/shell structure in which a quantum dot surrounds another quantum dot. A quantum dot having a core/shell structure may have a concentration gradient at an interface between the core and the shell, in which the concentration of a material that is present in the shell decreases toward the core.

In embodiments, the quantum dot may have the above-described core-shell structure including a core including a nanocrystal and a shell surrounding the core. The shell of the quantum dot may serve as a protection layer that prevents the chemical deformation of the core to maintain semiconductor properties and/or may serve as a charging layer to impart the quantum dot with electrophoretic properties. The shell may be a single layer or a multilayer. Examples of the shell of the quantum dot may include a metal oxide, a non-metal oxide, a semiconductor compound, or any combination thereof.

For example, the metal oxide or the non-metal oxide may include: a binary compound such as SiO₂, Al₂O₃, TiO₂, ZnO, MnO, Mn₂O₃, Mn₃O₄, CuO, FeO, Fe₂O₃, Fe₃O₄, CoO, Co₃O₄ and NiO; a ternary compound such as MgAl₂O₄, CoFe₂O₄, NiFe₂O₄ and CoMn₂O₄; or any combination thereof, but embodiments are not limited thereto.

Examples of the semiconductor compound may include CdS, CdSe, CdTe, ZnS, ZnSe, ZnTe, ZnSeS, ZnTeS, GaAs, GaP, GaSb, HgS, HgSe, HgTe, InAs, InP, InGaP, InSb, AlAs, AlP, AlSb, etc., but embodiments are not limited thereto.

The quantum dot may have a full width at half maximum (FWHM) of an emission wavelength spectrum equal to or less than about 45 nm. For example, the quantum dot may have a FWHM of an emission wavelength spectrum equal to or less than about 40 nm. For example, the quantum dot may have a FWHM of an emission wavelength spectrum equal to or less than about 30 nm. Within these ranges, color purity or color reproducibility may be improved. Light emitted through a quantum dot may be emitted in all directions, so that viewing angle properties may be improved.

The shape of the quantum dot may be a shape that is generally used in the related art, without specific limitation. For example, the quantum dot may have a spherical shape, a pyramidal shape, a multi-arm shape, or a cubic shape, or the quantum dot may be in the form of a nanoparticle, a nanotube, a nanowire, a nanofiber, a nanoplate, etc.

The quantum dot may control the color of light emitted according to a particle size thereof, and accordingly, the quantum dot may have various emission colors such as blue, red, and green.

In the light emitting devices ED according to embodiments as shown in FIG. 3 to FIG. 6, the electron transport region ETR is provided on the emission layer EML. The electron transport region ETR may include at least one of a hole blocking layer HBL, an electron transport layer ETL, or an electron injection layer EIL. However, embodiments are not limited thereto.

The electron transport region ETR may be a layer formed of a single material, a layer formed of different materials, or a structure having multiple layers formed of different materials.

For example, the electron transport region ETR may have a single layer structure of an electron injection layer EIL or an electron transport layer ETL, or may have a single layer structure formed of an electron injection material and an electron transport material. In other embodiments, the electron transport region ETR may have a single layer structure formed of different materials, or may have a structure in which an electron transport layer ETL/electron injection layer EIL, or a hole blocking layer HBL/electron transport layer ETL/electron injection layer EIL, are stacked in its respective stated order from the emission layer EML, but embodiments are not limited thereto. A thickness of the electron transport region ETR may be, for example, in a range of about 1,000 Å to about 1,500 Å.

The electron transport region ETR may be formed using various methods such as a vacuum deposition method, a spin coating method, a cast method, a Langmuir-Blodgett (LB) method, an inkjet printing method, a laser printing method, and a laser induced thermal imaging (LITI) method.

The electron transport region ETR may include a compound represented by Formula ET-1.

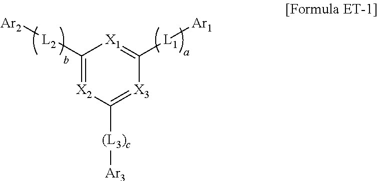

In Formula ET-1, at least one of X₁ to X₃ may be N, and the remainder of X₁ to X₃ may each independently be C(Rₐ). In Formula ET-1, Rₐ may be a hydrogen atom, a deuterium atom, a substituted or unsubstituted alkyl of 1 to 20 carbon atoms, a substituted or unsubstituted aryl group of 6 to 30 ring-forming carbon atoms, or a substituted or unsubstituted heteroaryl group of 2 to 30 ring-forming carbon atoms. In Formula ET-1, Ar₁ to Ar₃ may each independently be a hydrogen atom, a deuterium atom, a substituted or unsubstituted alkyl group of 1 to 20 carbon atoms, a substituted or unsubstituted aryl group of 6 to 30 ring-forming carbon atoms, or a substituted or unsubstituted heteroaryl group of 2 to 30 ring-forming carbon atoms.

In Formula ET-1, a to c may each independently be an integer from 0 to 10. In Formula ET-1, L₁ to L₃ may each independently be a direct linkage, a substituted or unsubstituted arylene group of 6 to 30 ring-forming carbon atoms, or a substituted or unsubstituted heteroarylene group of 2 to 30 ring-forming carbon atoms. If a to c are each 2 or more, multiple groups of each of L₁ to L₃ may each independently be a substituted or unsubstituted arylene group of 6 to 30 ring-forming carbon atoms, or a substituted or unsubstituted heteroarylene group of 2 to 30 ring-forming carbon atoms.

The electron transport region ETR may include an anthracene-based compound. However, embodiments are not limited thereto, and the electron transport region ETR may include, for example, tris(8-hydroxyquinolinato)aluminum (Alq₃), 1,3,5-tri[(3-pyridyl)-phen-3-yl]benzene, 2,4,6-tris(3′-(pyridin-3-yl)biphenyl-3-yl)-1,3,5-tri azine, 2-(4-(N-phenylbenzoimidazolyl-1-ylphenyl)-9,10-dinaphthylanthracene, 1,3,5-tri(1-phenyl-1H-benzo[d]imidazol-2-yl)benzene (TPBi), 2,9-dimethyl-4,7-diphenyl-1,10-phenanthroline (BCP), 4,7-diphenyl-1,10-phenanthroline (Bphen), 3-(4-biphenylyl)-4-phenyl-5-tert-butylphenyl-1,2,4-triazole (TAZ), 4-(naphthalen-1-yl)-3,5-diphenyl-4H-1,2,4-triazole (NTAZ), 2-(4-biphenylyl)-5-(4-tert-butylphenyl)-1,3,4-oxadiazole (tBu-PBD), bis(2-methyl-8-quinolinolato-N1,O8)-(1,1′-biphenyl-4-olato)aluminum (BAlq), berylliumbis(benzoquinolin-10-olate (Bebq₂), 9,10-di(naphthalene-2-yl)anthracene (ADN), 1,3-bis[3,5-di(pyridin-3-yl)phenyl]benzene (BmPyPhB), and mixtures thereof, without limitation.

The electron transport region ETR may include at least one of Compounds ET1 to ET36.

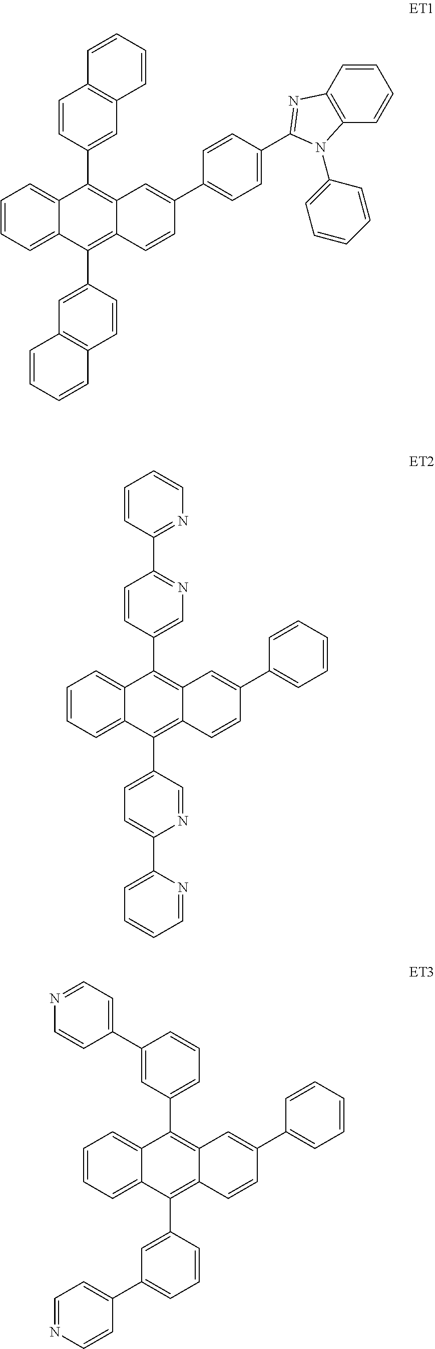 [SMILES: c1ccc(-n2c(-c3ccc(-c4ccc5c(-c6ccc7ccccc7c6)c6ccccc6c(-c6ccc7ccccc7c6)c5c4)cc3)nc3ccccc32)cc1 ; c1ccc(-c2ccc3c(-c4ccc(-c5ccccn5)nc4)c4ccccc4c(-c4ccc(-c5ccccn5)nc4)c3c2)cc1 ; c1ccc(-c2ccc3c(-c4cccc(-c5ccncc5)c4)c4ccccc4c(-c4cccc(-c5ccncc5)c4)c3c2)cc1]

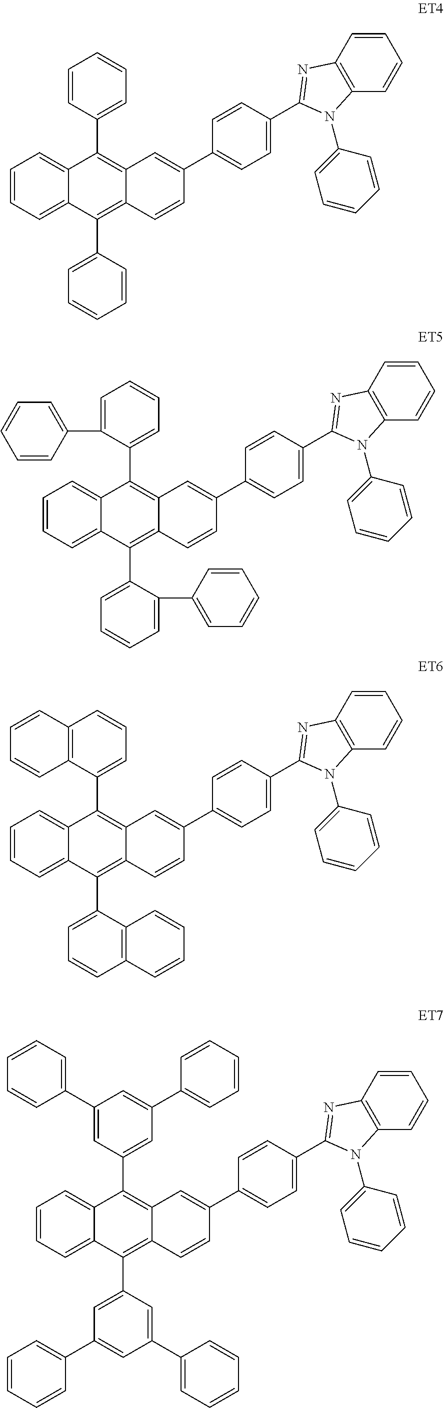 [SMILES: c1ccc(-c2c3ccccc3c(-c3ccccc3)c3cc(-c4ccc(-c5nc6ccccc6n5-c5ccccc5)cc4)ccc23)cc1 ; c1ccc(-c2ccccc2-c2c3ccccc3c(-c3ccccc3-c3ccccc3)c3cc(-c4ccc(-c5nc6ccccc6n5-c5ccccc5)cc4)ccc23)cc1 ; c1ccc(-n2c(-c3ccc(-c4ccc5c(-c6cccc7ccccc67)c6ccccc6c(-c6cccc7ccccc67)c5c4)cc3)nc3ccccc32)cc1 ; c1ccc(-c2cc(-c3ccccc3)cc(-c3c4ccccc4c(-c4cc(-c5ccccc5)cc(-c5ccccc5)c4)c4cc(-c5ccc(-c6nc7ccccc7n6-c6ccccc6)cc5)ccc34)c2)cc1]

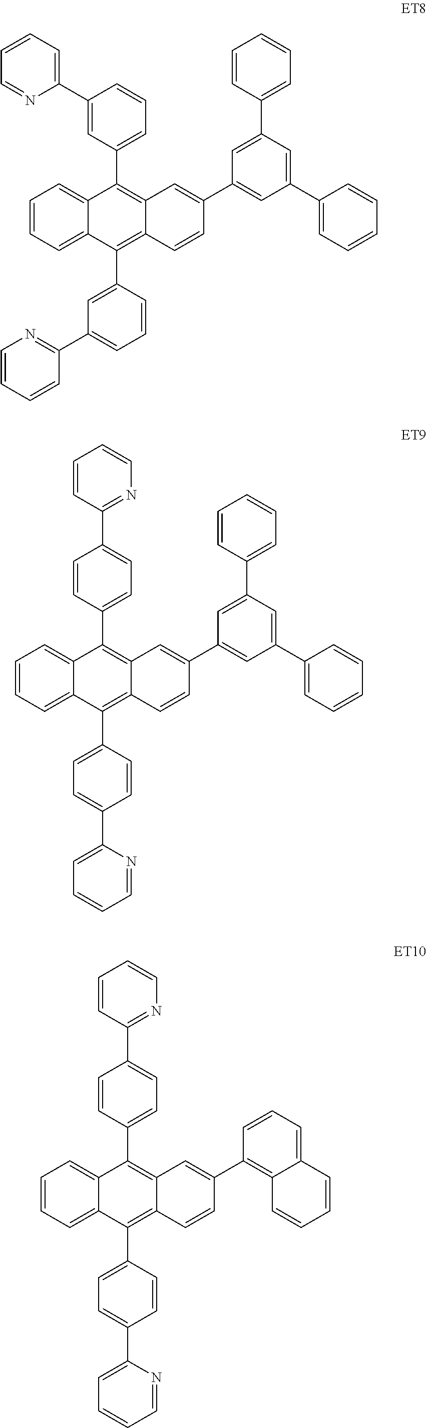 [SMILES: c1ccc(-c2cc(-c3ccccc3)cc(-c3ccc4c(-c5cccc(-c6ccccn6)c5)c5ccccc5c(-c5cccc(-c6ccccn6)c5)c4c3)c2)cc1 ; c1ccc(-c2cc(-c3ccccc3)cc(-c3ccc4c(-c5ccc(-c6ccccn6)cc5)c5ccccc5c(-c5ccc(-c6ccccn6)cc5)c4c3)c2)cc1 ; c1ccc(-c2ccc(-c3c4ccccc4c(-c4ccc(-c5ccccn5)cc4)c4cc(-c5cccc6ccccc56)ccc34)cc2)nc1]

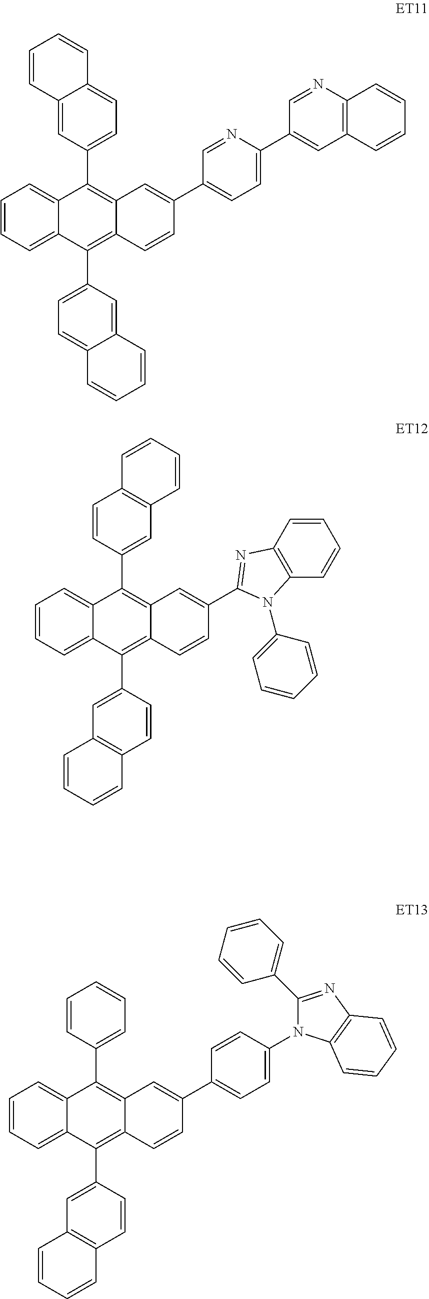 [SMILES: c1ccc2cc(-c3c4ccccc4c(-c4ccc5ccccc5c4)c4cc(-c5ccc(-c6cnc7ccccc7c6)nc5)ccc34)ccc2c1 ; c1ccc(-n2c(-c3ccc4c(-c5ccc6ccccc6c5)c5ccccc5c(-c5ccc6ccccc6c5)c4c3)nc3ccccc32)cc1 ; c1ccc(-c2c3ccccc3c(-c3ccc4ccccc4c3)c3ccc(-c4ccc(-n5c(-c6ccccc6)nc6ccccc65)cc4)cc23)cc1]

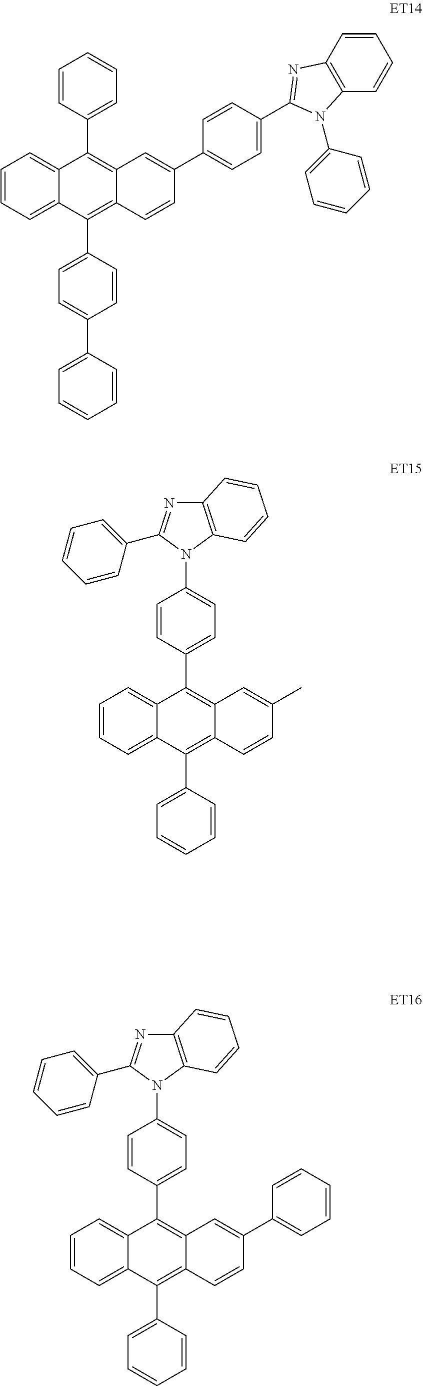 [SMILES: c1ccc(-c2ccc(-c3c4ccccc4c(-c4ccccc4)c4cc(-c5ccc(-c6nc7ccccc7n6-c6ccccc6)cc5)ccc34)cc2)cc1 ; Cc1ccc2c(-c3ccccc3)c3ccccc3c(-c3ccc(-n4c(-c5ccccc5)nc5ccccc54)cc3)c2c1 ; c1ccc(-c2ccc3c(-c4ccccc4)c4ccccc4c(-c4ccc(-n5c(-c6ccccc6)nc6ccccc65)cc4)c3c2)cc1]

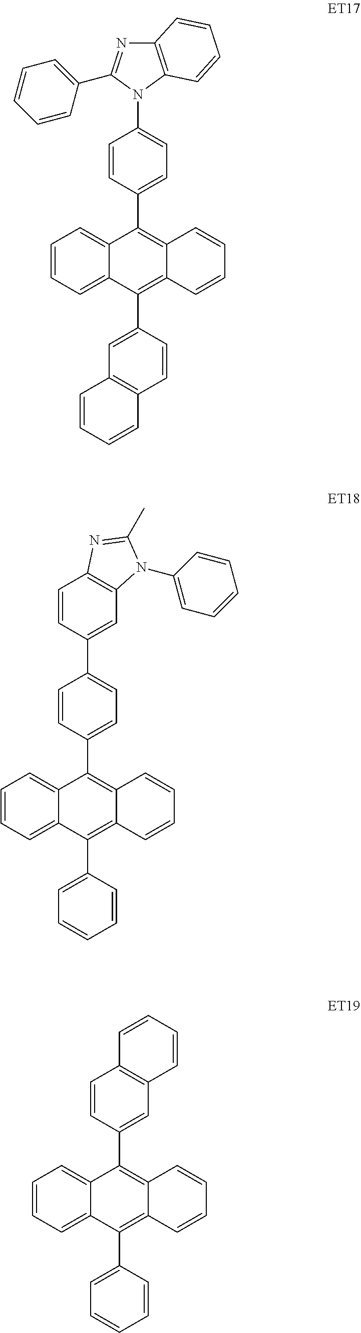

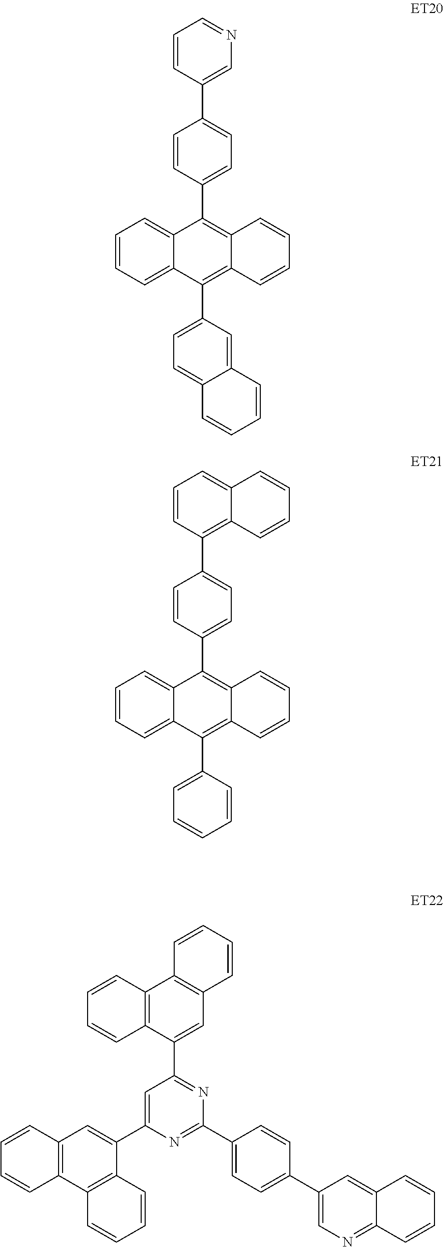

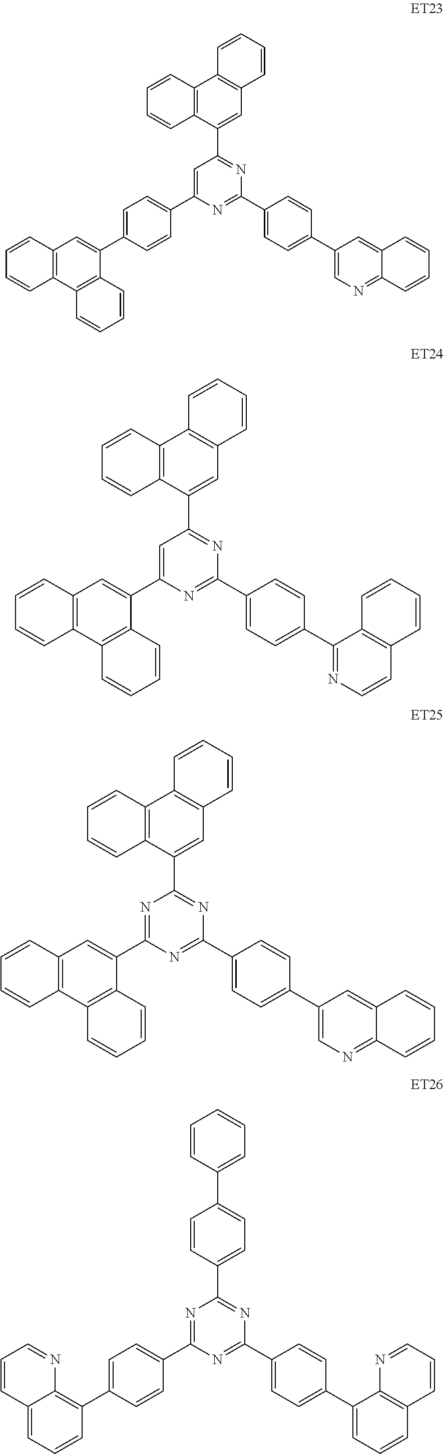 [SMILES: c1ccc2ncc(-c3ccc(-c4nc(-c5ccc(-c6cc7ccccc7c7ccccc67)cc5)cc(-c5cc6ccccc6c6ccccc56)n4)cc3)cc2c1 ; c1ccc2c(-c3ccc(-c4nc(-c5cc6ccccc6c6ccccc56)cc(-c5cc6ccccc6c6ccccc56)n4)cc3)nccc2c1 ; c1ccc2ncc(-c3ccc(-c4nc(-c5cc6ccccc6c6ccccc56)nc(-c5cc6ccccc6c6ccccc56)n4)cc3)cc2c1 ; c1ccc(-c2ccc(-c3nc(-c4ccc(-c5cccc6cccnc56)cc4)nc(-c4ccc(-c5cccc6cccnc56)cc4)n3)cc2)cc1]

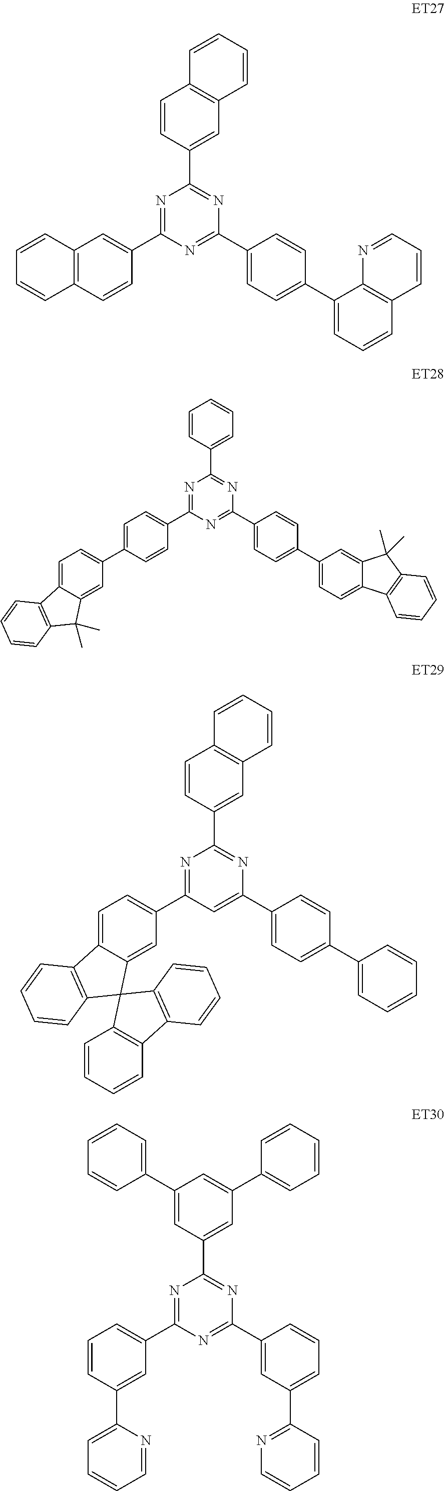

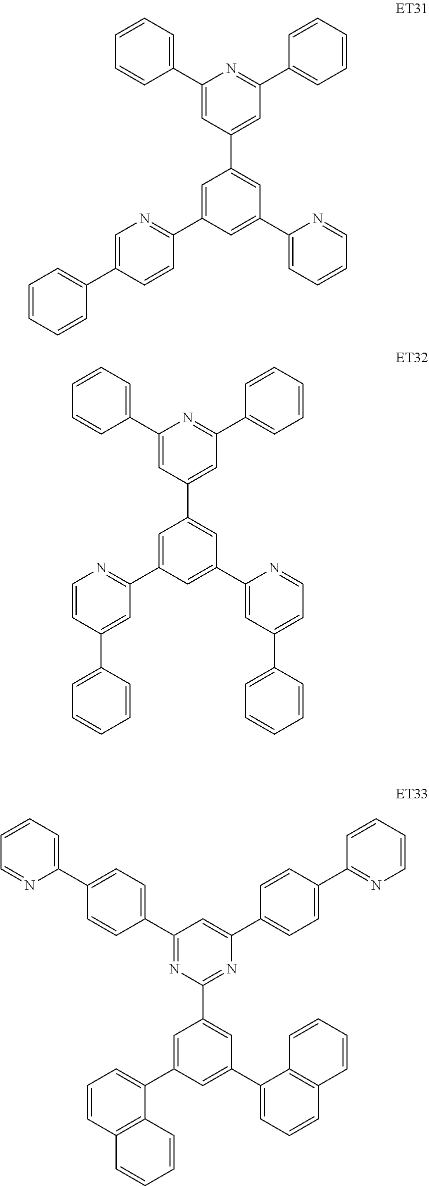 [SMILES: c1ccc(-c2ccc(-c3cc(-c4cc(-c5ccccc5)nc(-c5ccccc5)c4)cc(-c4ccccn4)c3)nc2)cc1 ; c1ccc(-c2ccnc(-c3cc(-c4cc(-c5ccccc5)nc(-c5ccccc5)c4)cc(-c4cc(-c5ccccc5)ccn4)c3)c2)cc1 ; c1ccc(-c2ccc(-c3cc(-c4ccc(-c5ccccn5)cc4)nc(-c4cc(-c5cccc6ccccc56)cc(-c5cccc6ccccc56)c4)n3)cc2)nc1]

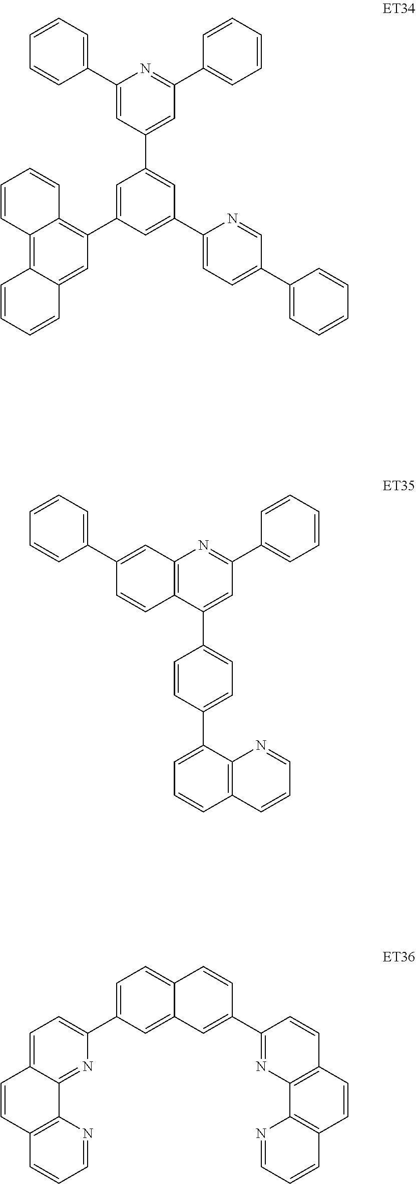 [SMILES: c1ccc(-c2ccc(-c3cc(-c4cc(-c5ccccc5)nc(-c5ccccc5)c4)cc(-c4cc5ccccc5c5ccccc45)c3)nc2)cc1 ; c1ccc(-c2ccc3c(-c4ccc(-c5cccc6cccnc56)cc4)cc(-c4ccccc4)nc3c2)cc1 ; c1cnc2c(c1)ccc1ccc(-c3ccc4ccc(-c5ccc6ccc7cccnc7c6n5)cc4c3)nc12]

The electron transport region ETR may include: a metal halide such as LiF, NaCl, CsF, RbCl, RbI, CuI, or KI; a lanthanide metal such as Yb; or a co-depositing material of the metal halide and the lanthanide metal. For example, the electron transport region ETR may include KI:Yb, RbI:Yb, LiF:Yb, etc., as the co-depositing material. The electron transport region ETR may include a metal oxide such as Li₂O and BaO, or 8-hydroxy-lithium quinolate (Liq). However, embodiments are not limited thereto. The electron transport region ETR may also be formed of a material that is a mixture of an electron transport material and an insulating organometallic salt. The organometallic salt may be a material having an energy band gap equal to or greater than about 4 eV. For example, the organometallic salt may include metal acetates, metal benzoates, metal acetoacetates, metal acetylacetonates, or metal stearates.

The electron transport region ETR may include at least one of 2,9-dimethyl-4,7-diphenyl-1,10-phenanthroline (BCP), diphenyl(4-(triphenylsilyl)phenyl)phosphine oxide (TSPO1) or 4,7-diphenyl-1,10-phenanthroline (Bphen) in addition to the aforementioned materials. However, embodiments are not limited thereto.

The electron transport region ETR may include the compounds of the electron transport region in at least one of an electron injection layer EIL, an electron transport layer ETL, or a hole blocking layer HBL.

If the electron transport region ETR includes an electron transport layer ETL, a thickness of the electron transport layer ETL may be in a range of about 100 Å to about 1,000 Å. For example, the thickness of the electron transport layer ETL may be in a range of about 150 Å to about 500 Å. If the thickness of the electron transport layer ETL satisfies any of the above-described ranges, satisfactory electron transport properties may be obtained without a substantial increase of driving voltage. If the electron transport region ETR includes an electron injection layer EIL, a thickness of the electron injection layer EIL may be in a range of about 1 Å to about 100 Å. For example, the thickness of the electron injection layer EIL may be in a range of about 3 Å to about 90 Å. If the thickness of the electron injection layer EIL satisfies any of the above described ranges, satisfactory electron injection properties may be obtained without inducing a substantial increase of driving voltage.

The second electrode EL2 is provided on the electron transport region ETR. The second electrode EL2 may be a common electrode. The second electrode EL2 may be a cathode or an anode, but embodiments are not limited thereto. For example, if the first electrode EL1 is an anode, the second cathode EL2 may be a cathode, and if the first electrode EL1 is a cathode, the second electrode EL2 may be an anode.

The second electrode EL2 may be a transmissive electrode, a transflective electrode, or a reflective electrode. If the second electrode EL2 is a transmissive electrode, the second electrode EL2 may include a transparent metal oxide, for example, ITO, IZO, ZnO, ITZO, etc.

If the second electrode EL2 is a transflective electrode or a reflective electrode, the second electrode EL2 may include Ag, Mg, Cu, Al, Pt, Pd, Au, Ni, Nd, Ir, Cr, Li, Ca, LiF/Ca, LiF/Al, Mo, Ti, Yb, W, a compound thereof, or a mixture thereof (for example, AgMg, AgYb, or MgAg). In another embodiment, the second electrode EL2 may have a multilayered structure including a reflective layer or a transflective layer formed of the above-described materials and a transparent conductive layer formed of ITO, IZO, ZnO, ITZO, etc. For example, the second electrode EL2 may include the aforementioned metal materials, combinations of two or more metal materials selected from the aforementioned metal materials, or oxides of the aforementioned metal materials.

Although not shown in the drawings, the second electrode EL2 may be electrically connected to an auxiliary electrode. If the second electrode EL2 is electrically connected to the auxiliary electrode, the resistance of the second electrode EL2 may decrease.

In an embodiment, the light emitting device ED may further include a capping layer CPL disposed on the second electrode EL2. The capping layer CPL may be a multilayer or a single layer.

In an embodiment, the capping layer CPL may include an organic layer or an inorganic layer. For example, if the capping layer CPL includes an inorganic material, the inorganic material may include an alkali metal compound such as LiF, an alkaline earth metal compound such as MgF₂, SiON, SiNx, SiOy, etc.

For example, if the capping layer CPL includes an organic material, the organic material may include 2,2′-dimethyl-N,N′-di-[(1-naphthyl)-N,N′-diphenyl]-1,1′-biphenyl-4,4′-diamine (α-NPD), NPB, TPD, m-MTDATA, Alq₃, CuPc, N4,N4,N4′,N4′-tetra(biphenyl-4-yl) biphenyl-4,4′-diamine (TPD15), 4,4′,4″-tris(carbazol-9-yl) triphenylamine (TCTA), etc., or may include an epoxy resin, or acrylate such as methacrylate. In an embodiment, a capping layer CPL may include at least one of Compounds P1 to P5, but embodiments are not limited thereto.

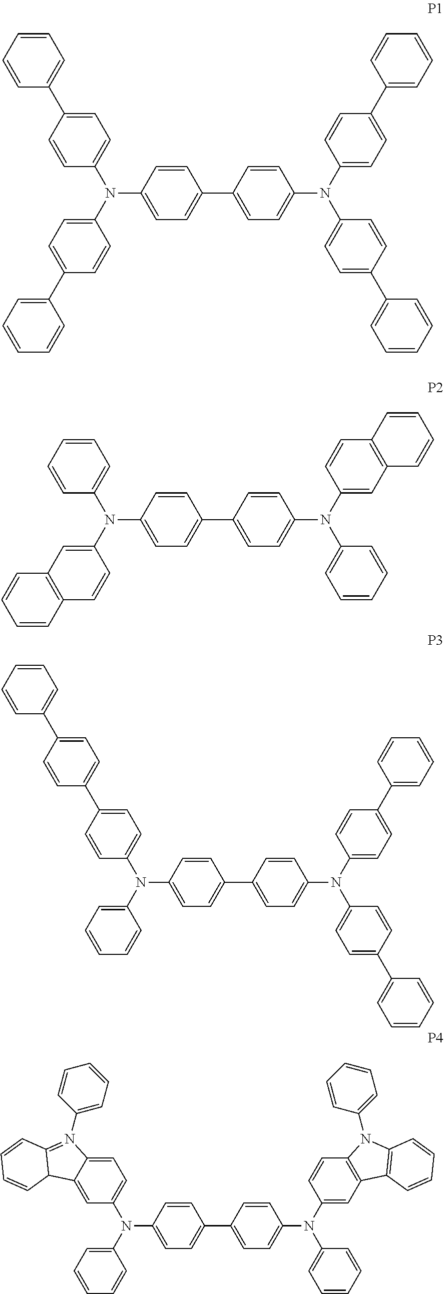

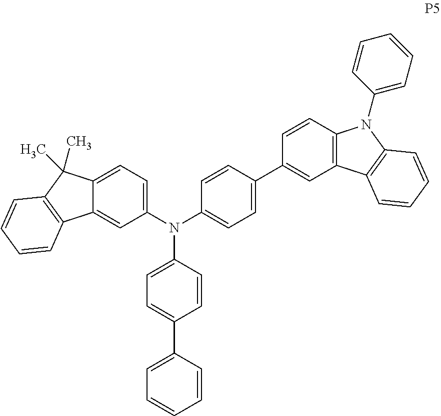 [SMILES: CC1(C)c2ccccc2-c2cc(N(c3ccc(-c4ccccc4)cc3)c3ccc(-c4ccc5c(c4)c4ccccc4n5-c4ccccc4)cc3)ccc21]

A refractive index of the capping layer CPL may be equal to or greater than about 1.6. For example, the refractive index of the capping layer CPL may be equal to or greater than about 1.6, with respect to light in a wavelength range of about 550 nm to about 660 nm.

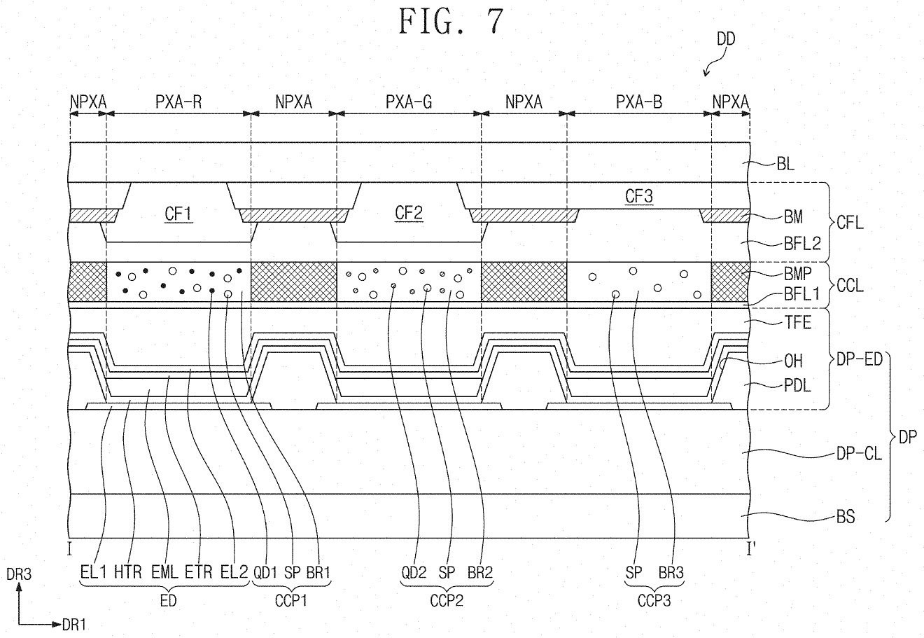

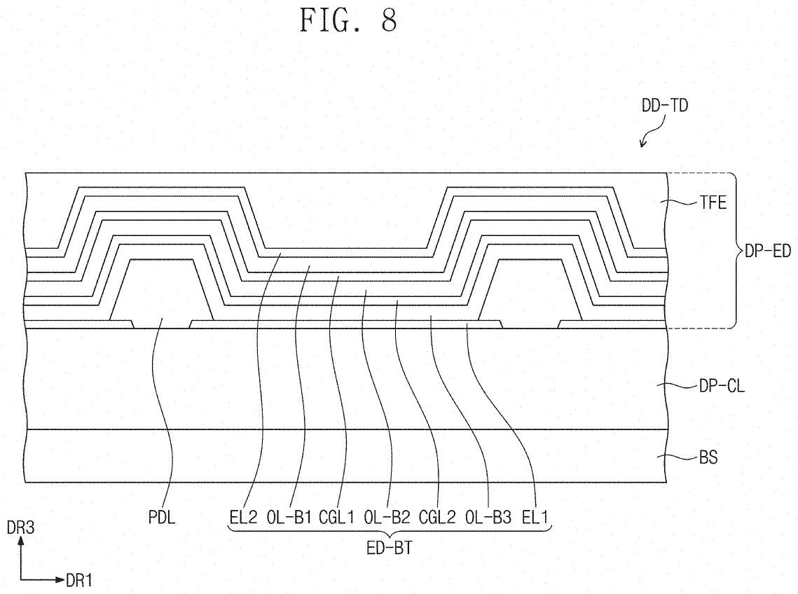

FIG. 7 and FIG. 8 are each a schematic cross-sectional view of a display apparatus according to embodiments. In the explanation on the display apparatuses according to embodiments with reference to FIG. 7 and FIG. 8, the features which have been described above with respect to FIG. 1 to FIG. 6 will not be explained again, and the differing features will be explained.

Referring to FIG. 7, a display apparatus DD-a according to an embodiment may include a display panel DP including a display device layer DP-ED, a light controlling layer CCL disposed on the display panel DP, and a color filter layer CFL.

In an embodiment shown in FIG. 7, the display panel DP includes a base layer BS, a circuit layer DP-CL provided on the base layer BS, and a display device layer DP-ED, and the display device layer DP-ED may include a light emitting device ED.

The light emitting device ED may include a first electrode ELL a hole transport region HTR disposed on the first electrode ELL an emission layer EML disposed on the hole transport region HTR, an electron transport region ETR disposed on the emission layer EML, and a second electrode EL2 disposed on the electron transport region ETR. A structure of the light emitting device ED shown in FIG. 7 may be the same as a structure of a light emitting device according to one of FIG. 3 to FIG. 6 as described herein.

The emission layer EML of a light emitting device ED included in a display apparatus DD-a according to an embodiment, may include the nitrogen-containing compound according to an embodiment.

Referring to FIG. 7, the emission layer EML may be disposed in an opening OH defined in a pixel definition layer PDL. For example, the emission layer EML, which is separated by the pixel definition layer PDL and correspondingly provided to each of luminous areas PXA-R, PXA-G, and PXA-B, may emit light in a same wavelength region. In the display apparatus DD-a according to an embodiment, the emission layer EML may emit blue light. Although not shown in the drawings, in an embodiment, the emission layer EML may be provided as a common layer for all of the luminous areas PXA-R, PXA-G, and PXA-B.

The light controlling layer CCL may be disposed on the display panel DP. The light controlling layer CCL may include a light converter. The light converter may be a quantum dot or a phosphor. The light converter may transform the wavelength of a provided light and may emit the resulting light. For example, the light controlling layer CCL may be a layer including a quantum dot or a layer including a phosphor.

The light controlling layer CCL may include light controlling parts CCP1, CCP2, and CCP3. The light controlling parts CCP1, CCP2, and CCP3 may be separated from one another.

Referring to FIG. 7, a partition pattern BMP may be disposed between the separated light controlling parts CCP1, CCP2, and CCP3, but embodiments are not limited thereto. In FIG. 7, the partition pattern BMP is shown so that it does not overlap the light controlling parts CCP1, CCP2, and CCP3, but at least a portion of the edges of the light controlling parts CCP1, CCP2, and CCP3 may overlap the partition pattern BMP.

The light controlling layer CCL may include a first light controlling part CCP1 including a first quantum dot QD1 converting first color light provided from the light emitting device ED into second color light, a second light controlling part CCP2 including a second quantum dot QD2 converting first color light into third color light, and a third light controlling part CCP3 transmitting first color light.

In an embodiment, the first light controlling part CCP1 may provide red light which is the second color light, and the second light controlling part CCP2 may provide green light which is the third color light. The third color controlling part CCP3 may transmit and provide blue light which is the first color light provided from the light emitting device ED. For example, the first quantum dot QD1 may be a red quantum dot, and the second quantum dot QD2 may be a green quantum dot. The quantum dots QD1 and QD2 may be a quantum dot as described herein.

The light controlling layer CCL may further include a scatterer SP. The first light controlling part CCP1 may include the first quantum dot QD1 and the scatterer SP, the second light controlling part CCP2 may include the second quantum dot QD2 and the scatterer SP, and the third light controlling part CCP3 may not include a quantum dot but may include the scatterer SP.

The scatterer SP may be an inorganic particle. For example, the scatterer SP may include at least one of TiO₂, ZnO, Al₂O₃, SiO₂, or hollow silica. The scatterer SP may include at least one of TiO₂, ZnO, Al₂O₃, SiO₂, and hollow silica, or the scatterer SP may be a mixture of two or more materials selected from TiO₂, ZnO, Al₂O₃, SiO₂, and hollow silica.

The first light controlling part CCP1, the second light controlling part CCP2, and the third light controlling part CCP3 may each include base resins BR1, BR2, and BR3 dispersing the quantum dots QD1 and QD2 and the scatterer SP. In an embodiment, the first light controlling part CCP1 may include the first quantum dot QD1 and the scatterer SP dispersed in the first base resin BR1, the second light controlling part CCP2 may include the second quantum dot QD2 and the scatterer SP dispersed in the second base resin BR2, and the third light controlling part CCP3 may include the scatterer SP dispersed in the third base resin BR3. The base resins BR1, BR2, and BR3 are mediums in which the quantum dots QD1 and QD2 and the scatterer SP are dispersed, and may be composed of various resin compositions which may be generally referred to as a binder. For example, the base resins BR1, BR2, and BR3 may be acrylic resins, urethane-based resins, silicone-based resins, epoxy-based resins, etc. The base resins BR1, BR2, and BR3 may be transparent resins. In an embodiment, the first base resin BR1, the second base resin BR2, and the third base resin BR3 may be the same as or different from each other.

The light controlling layer CCL may include a barrier layer BFL1. The barrier layer BFL1 may block the penetration of moisture and/or oxygen (hereinafter, will be referred to as “humidity/oxygen”). The barrier layer BFL1 may be disposed on the light controlling parts CCP1, CCP2, and CCP3 to block the exposure of the light controlling parts CCP1, CCP2, and CCP3 to humidity/oxygen. The barrier layer BFL1 may cover the light controlling parts CCP1, CCP2, and CCP3. A barrier layer BFL2 may be provided between the light controlling parts CCP1, CCP2, and CCP3 and a color filter layer CFL.

The barrier layers BFL1 and BFL2 may each independently include at least one inorganic layer. For example, the barrier layers BFL1 and BFL2 may each include an inorganic material. For example, the barrier layers BFL1 and BFL2 may include silicon nitride, aluminum nitride, zirconium nitride, titanium nitride, hafnium nitride, tantalum nitride, silicon oxide, aluminum oxide, titanium oxide, tin oxide, cerium oxide, silicon oxynitride, or a metal thin film securing light transmittance. The barrier layers BFL1 and BFL2 may each further include an organic layer. The barrier layers BFL1 and BFL2 may each independently be formed of a single layer or of multiple layers.

In the display apparatus DD-a according to an embodiment, the color filter layer CFL may be disposed on the light controlling layer CCL. In an embodiment, the color filter layer CFL may be disposed directly on the light controlling layer CCL. For example, the barrier layer BFL2 may be omitted.

The color filter layer CFL may include a light blocking part BM (not shown) and filters CF1, CF2, and CF3. The color filter layer CFL may include a first filter CF1 transmitting second color light, a second filter CF2 transmitting third color light, and a third filter CF3 transmitting first color light. For example, the first filter CF1 may be a red filter, the second filter CF2 may be a green filter, and the third filter CF3 may be a blue filter. Each of the filters CF1, CF2, and CF3 may each include a polymer photosensitive resin and a pigment or dye. The first filter CF1 may include a red pigment or dye, the second filter CF2 may include a green pigment or dye, and the third filter CF3 may include a blue pigment or dye. However, embodiments are not limited thereto, and the third filter CF3 may not include a pigment or dye. The third filter CF3 may include a polymer photosensitive resin and may not include a pigment or dye. The third filter CF3 may be transparent. The third filter CF3 may be formed of a transparent photosensitive resin.

In an embodiment, the first filter CF1 and the second filter CF2 may each be a yellow filter. The first filter CF1 and the second filter CF2 may be provided in one body without distinction.

The light blocking part BM (not shown) may be a black matrix. The light blocking part BM (not shown) may be formed of an organic light blocking material or an inorganic light blocking material including a black pigment or black dye. The light blocking part BM (not shown) may prevent light leakage and may separate the boundaries between adjacent filters CF1, CF2, and CF3. In an embodiment, the light blocking part BM (not shown) may be formed of a blue filter.

The first to third filters CF1, CF2, and CF3 may each be disposed corresponding to a red luminous area PXA-R, a green luminous area PXA-G, and a blue luminous area PXA-B, respectively.

A base substrate BL may be disposed on the color filter layer CFL. The base substrate BL may provide a base surface on which the color filter layer CFL, the light controlling layer CCL, etc. are disposed. The base substrate BL may be a glass substrate, a metal substrate, a plastic substrate, etc. However, embodiments are not limited thereto, and the base substrate BL may include an inorganic layer, an organic layer, or a composite material layer. Although not shown in the drawings, in an embodiment, the base substrate BL may be omitted.

FIG. 8 is a schematic cross-sectional view showing a portion of the display apparatus DD-a according to an embodiment. In FIG. 8, a schematic cross-sectional view of a portion corresponding to the display panel DP in FIG. 7 is shown. In a display apparatus DD-TD according to an embodiment, a light emitting device ED-BT may include light emitting structures OL-B1, OL-B2, and OL-B3. The light emitting device ED-BT may include a first electrode EL1 and a second electrode EL2 which face each other, and the light emitting structures OL-B1, OL-B2, and OL-B3 stacked in a thickness direction and provided between the first electrode EL1 and the second electrode EL2. The light emitting structures OL-B1, OL-B2, and OL-B3 may each include an emission layer EML (FIG. 7), and a hole transport region HTR and an electron transport region ETR, disposed with the emission layer EML (FIG. 7) therebetween.

For example, the light emitting device ED-BT included in the display apparatus DD-TD according to an embodiment may be a light emitting device having a tandem structure and including multiple emission layers.

In an embodiment shown in FIG. 8, light emitted from the light emitting structures OL-B1, OL-B2, and OL-B3 may all be blue light. However, embodiments are not limited thereto, and light emitted from the light emitting structures OL-B1, OL-B2 and OL-B3 may have different wavelength regions from each other. For example, the light emitting device ED-BT including the light emitting structures OL-B1, OL-B2, and OL-B3 which emit light in different wavelength regions may emit white light.

Charge generating layers CGL1 and CGL2 may be disposed between neighboring light emitting structures OL-B1, OL-B2, and OL-B3. Charge generating layers CGL1 and CGL2 may each independently include a p-type charge generating layer and/or an n-type charge generating layer.

At least one of the light emitting structures OL-B1, OL-B2, and OL-B3 included in the display apparatus DD-TD may include the nitrogen-containing compound according to an embodiment. For example, at least one of the emission layers included in the light emitting device ED-BT may include the nitrogen-containing compound according to an embodiment.

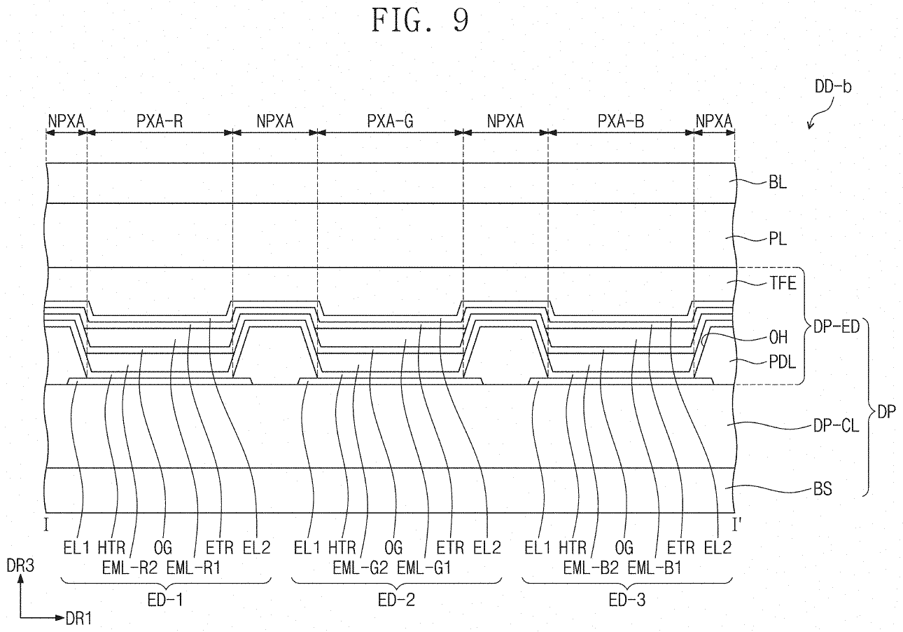

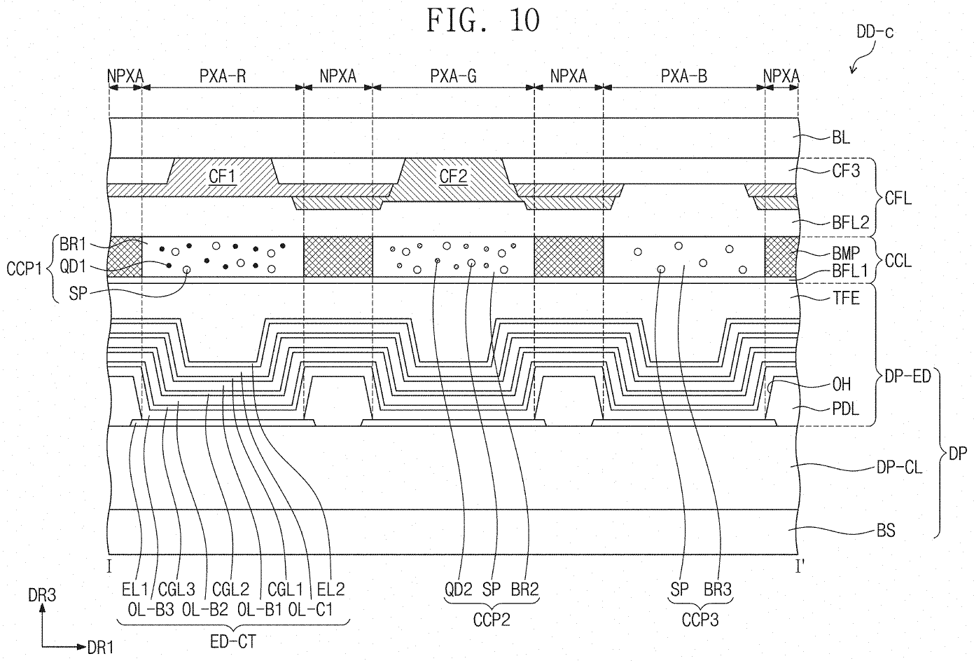

Referring to FIG. 9, a display apparatus DD-b according to an embodiment may include light emitting devices ED-1, ED-2, and ED-3, which may each include two emission layers that are stacked. In contrast to the display apparatus DD shown in FIG. 2, the embodiment shown in FIG. 9 is different at least in that the first to third light emitting devices ED-1, ED-2, and ED-3 each include two emission layers that are stacked in a thickness direction. In each of the first to third light emitting devices ED-1, ED-2, and ED-3, the two emission layers may emit light in a same wavelength region.

The first light emitting device ED-1 may include a first red emission layer EML-R1 and a second red emission layer EML-R2. The second light emitting device ED-2 may include a first green emission layer EML-G1 and a second green emission layer EML-G2. The third light emitting device ED-3 may include a first blue emission layer EML-B1 and a second blue emission layer EML-B2. An emission auxiliary part OG may be disposed between the first red emission layer EML-R1 and the second red emission layer EML-R2, between the first green emission layer EML-G1 and the second green emission layer EML-G2, and between the first blue emission layer EML-B1 and the second blue emission layer EML-B2.

The emission auxiliary part OG may be a single layer or a multilayer. The emission auxiliary part OG may include a charge generating layer. For example, the emission auxiliary part OG may include an electron transport region, a charge generating layer, and a hole transport region, which are stacked in that order. The emission auxiliary part OG may be provided as a common layer for all of the first to third light emitting devices ED-1, ED-2, and ED-3. However, embodiments are not limited thereto, and the emission auxiliary part OG may be provided by being patterned in the openings OH defined in a pixel definition layer PDL.

The first red emission layer EML-R1, the first green emission layer EML-G1, and the first blue emission layer EML-B1 may each be disposed between the electron transport region ETR and the emission auxiliary part OG. The second red emission layer EML-R2, the second green emission layer EML-G2, and the second blue emission layer EML-B2 may each be disposed between the emission auxiliary part OG and the hole transport region HTR.

For example, the first light emitting device ED-1 may include a first electrode EL1, a hole transport region HTR, a second red emission layer EML-R2, an emission auxiliary part OG, a first red emission layer EML-R1, an electron transport region ETR, and a second electrode EL2, stacked in that order. The second light emitting device ED-2 may include a first electrode EL1, a hole transport region HTR, a second green emission layer EML-G2, an emission auxiliary part OG, a first green emission layer EML-G1, an electron transport region ETR, and a second electrode EL2, stacked in that order. The third light emitting device ED-3 may include a first electrode EL1, a hole transport region HTR, a second blue emission layer EML-B2, an emission auxiliary part OG, a first blue emission layer EML-B1, an electron transport region ETR, and a second electrode EL2, stacked in that order.

An optical auxiliary layer PL may be disposed on a display element layer DP-ED. The optical auxiliary layer PL may include a polarization layer. The optical auxiliary layer PL may be disposed on a display panel DP and may control light that is reflected at the display panel DP from an external light. Although not shown in the drawings, in an embodiment, the optical auxiliary layer PL may be omitted from the display apparatus DD-b.

At least one emission layer included in the display apparatus DD-b according to an embodiment shown in FIG. 9 may include the nitrogen-containing compound according to an embodiment. For example, in an embodiment, at least one of the first blue emission layer EML-B1 or the second blue emission layer EML-B2 may include the nitrogen-containing compound according to an embodiment.

In contrast to FIG. 8 and FIG. 9, FIG. 10 shows a display apparatus DD-c that is different at least in that it includes four light emitting structures OL-B1, OL-B2, OL-B3, and OL-C1. A light emitting device ED-CT may include a first electrode EL1 and a second electrode EL2 which face each other, and first to fourth light emitting structures OL-B1, OL-B2, OL-B3, and OL-C1 that are stacked in a thickness direction between the first electrode EL1 and the second electrode EL2. Charge generating layers CGL1, CGL2, and CGL3 may be disposed between the first to fourth light emitting structures OL-B1, OL-B2, OL-B3, and OL-C1. Among the four light emitting structures, the first to third light emitting structures OL-B1, OL-B2, and OL-B3 may each emit blue light, and the fourth light emitting structure OL-C1 may emit green light. However, embodiments are not limited thereto, and the first to fourth light emitting structures OL-B1, OL-B2, OL-B3, and OL-C1 may each emit different wavelengths of light.

The charge generating layers CGL1, CGL2, and CGL3 disposed between the neighboring light emitting structures OL-B1, OL-B2, OL-B3, and OL-C1 may each independently include a p-type charge generating layer and/or an n-type charge generating layer.

In the display apparatus DD-c according to an embodiment, at least one of the light emitting structures OL-B1, OL-B2, OL-B3, and OL-C1 may include the nitrogen-containing compound according to an embodiment. For example, in an embodiment, at least one of the first to third light emitting structures OL-B1, OL-B2, and OL-B3 may include the nitrogen-containing compound according to an embodiment.

Hereinafter, the nitrogen-containing compound according to an embodiment and the light emitting device according to an embodiment will be explained in detail with reference to the Examples and the Comparative Examples. The Examples below are only provided as illustrations to assist in understanding the disclosure, and the scope thereof is not limited thereto.

### EXAMPLES

1. Synthesis of Nitrogen-Containing Compound

A synthesis method of the nitrogen-containing compound according to an embodiment will be explained in detail with reference to the synthesis methods of Compounds ET-2, ET-6, and ET-19. The synthesis methods of the nitrogen-containing compounds explained hereinafter are provided only as examples, and the synthesis methods of the nitrogen-containing compound according to embodiments are not limited to the Examples below.

(1) Synthesis of Compound ET-2

1) Synthesis of Intermediate 1

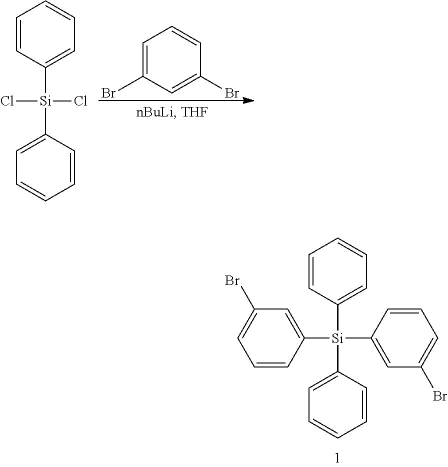

To a round-bottom flask, dichlorodiphenylsilane (1 eq) was put, and THF (150 mL) was put. At about −78° C., nBuLi (2 M in hexane, 2.2 eq) was slowly added dropwise, and after about 40 minutes, 1,3-dibromobenzene (2.5 eq) was slowly added dropwise. After slowly elevating the temperature to room temperature, the resultant was stirred overnight, quenched with water and a NH₄Cl solution, and washed with ethyl acetate/H₂O. The resultant was dried over MgSO₄, and column chromatography was performed using methylene chloride:ethyl acetate (2:1) to obtain Intermediate 1 (yield 77%).

C₂₄H₁₈Br₂Si [M]+: calculated: 491.9, measured: 491

2) Synthesis of Intermediate 2

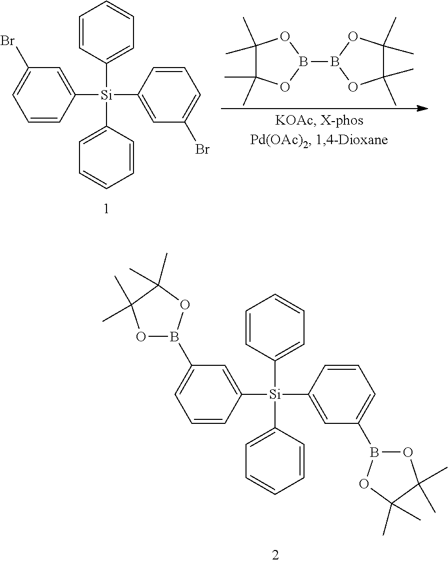

To a round-bottom flask, Intermediate 1 (1 eq), Pd(OAc)₂ (0.05 eq), X-Phos (0.1 eq), KOAc (2.1 eq), 1,4-dioxane (400 mL), and H₂O (100 mL) were put and stirred. After reducing the temperature to about 0° C., a 1 M solution of 4,4,4′,4′,5,5,5′,5′-octamethyl-2,2′-bi(1,3,2-dioxaborolane) (2.2 eq) dissolved in THF was added thereto dropwise. After slowly elevating the temperature to room temperature, the resultant was stirred overnight. After finishing the reaction, ethyl acetate/H₂O was added, stirring was performed for about 30 minutes, and only an organic layer was separated using a separatory funnel. The resultant was dried over MgSO₄, and filtration was performed using silica, followed by drying. The solid thus dried was dissolved in methylene chloride, and column chromatography was performed using methylene chloride:ethyl acetate (1:1) to obtain Intermediate 2 (yield 81%).

C₃₆H₄₂B₂O₄Si [M]+: calculated: 588.3, measured: 587

3) Synthesis of Compound ET-2

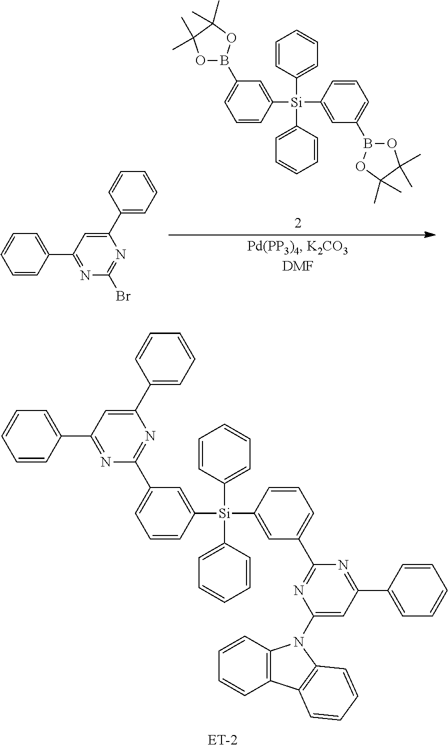

To a round-bottom flask, 2-bromo-4,6-diphenylpyrimidine (2.2 eq), Pd(PPh₃)₄ (0.05 eq), K₂CO₃ (2.4 eq), DMF (400 mL), and H₂O (100 mL) were put and stirred. After reducing the temperature to about 0° C., a 1 M solution of Intermediate 2 dissolved in THF was added thereto dropwise. After slowly elevating the temperature to room temperature, the resultant was stirred overnight. After finishing the reaction, ethyl acetate/H₂O was added, stirring was performed for about 30 minutes, and only an organic layer was separated using a separatory funnel. The resultant was dried over MgSO₄, methylene chloride filtration was performed, and the solid thus obtained was washed using MeOH and dried. The solid thus dried was put in toluene (100 mL) and dissolved by boiling, and solidified by adding dropwise methylene chloride:hexane=1:1 (100 mL). The solid was dissolved in methylene chloride again, and column chromatography was performed using methylene chloride:hexane (2:1) to obtain Compound ET-2 (yield 70%).

C₆₂H₄₃N₅Si [M]+: calculated: 885.3, measured: 884

Elemental Analysis for calculated: C, 84.04; H, 4.89; N, 7.90, Si, 3.17

(2) Synthesis of Compound ET-6

1) Synthesis of Intermediate 3

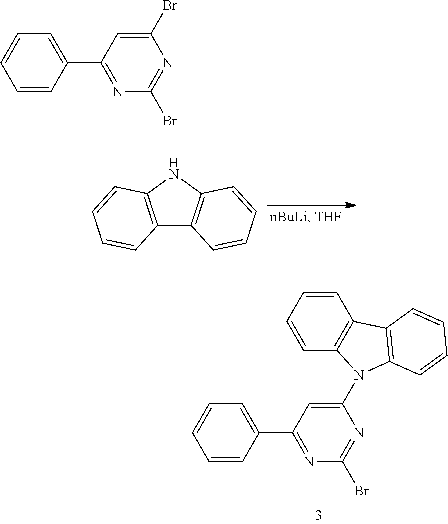 [SMILES: Brc1cc(-c2ccccc2)nc(Br)n1 ; c1ccc2c(c1)[nH]c1ccccc12 ; Brc1nc(-c2ccccc2)cc(-n2c3ccccc3c3ccccc32)n1]

To a round-bottom flask, 9H-carbazole (1.1 eq) was dissolved in THF (100 mL), and the temperature was reduced to about 0 degrees. nBuLi (1.2 eq, 2 M solution in hexane) was added thereto dropwise for about 30 minutes, and a solution of 2,4-dibromo-6-phenylpyrimidine (1.1 eq) dissolved in THF (2 M solution) was added thereto dropwise for about 10 minutes. After stirring for about 1 hour, H₂O was added, and only an organic layer was separated using a separatory funnel. Column chromatography was performed using methylene chloride:hexane (1:1) to obtain Intermediate 3 (yield 85%).

C₂₂H₁₄BrN₃ [M]+: calculated: 399.0, measured: 398

2) Synthesis of Compound ET-6

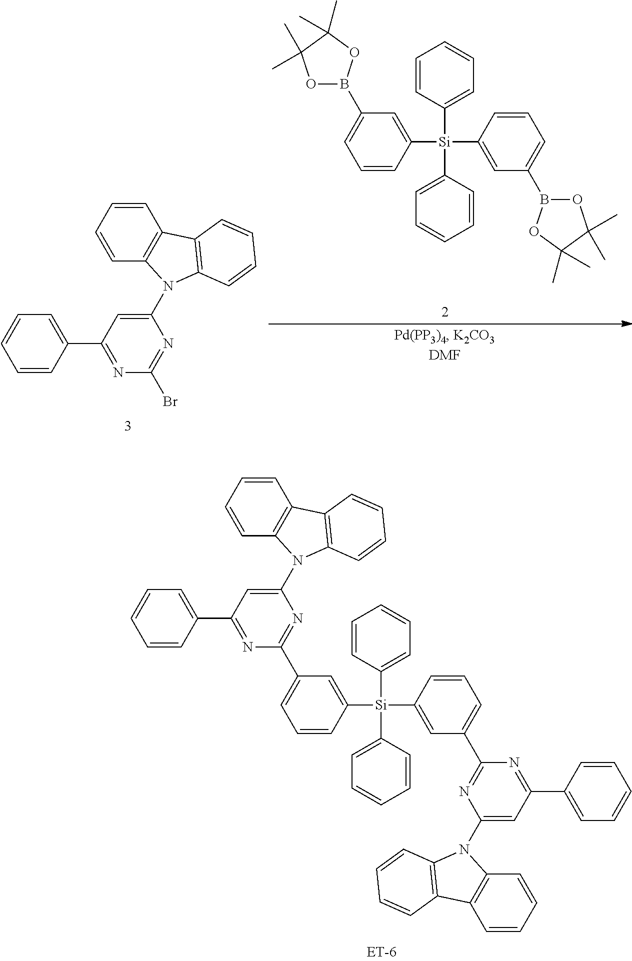

Compound ET-6 was synthesized (yield 68%) by the same method as the synthesis process of Compound ET-2 except for using Intermediate 3 instead of 2-bromo-4,6-diphenylpyrimidine.

C₆₈H₄₆N₆Si [M]+: calculated: 974.4, measured: 973

Elemental Analysis for calculated: C, 83.75; H, 4.75; N, 8.62, Si, 2.88

(3) Synthesis of Compound ET-19

1) Synthesis of Intermediate 4

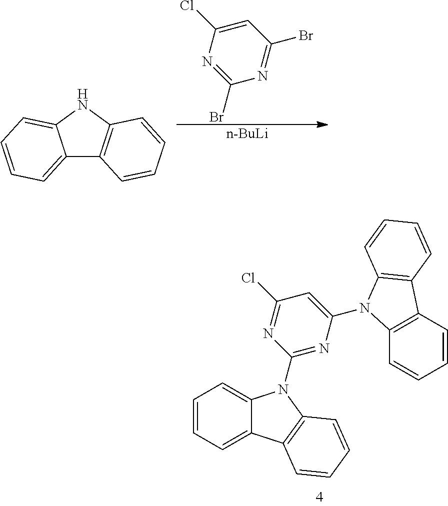

To a round-bottom flask, 9H-carbazole (2.1 eq) was dissolved in THF (100 mL), and the temperature was reduced to about −78 degrees. nBuLi (2.2 eq, 2 M solution in hexane) was added thereto dropwise for about 30 minutes, and the temperature was slowly elevated to about 0 degrees. A solution (1 M solution) of 2,4-dibromo-6-chloropyrimidine (1.1 eq) was slowly added thereto dropwise for about 30 minutes, followed by stirring for about 1 hour. After adding H₂O, only an organic layer was separated using a separatory funnel. Column chromatography was performed using methylene chloride:hexane (2:1) to obtain Intermediate 4 (yield 65%).

C₂₈H₁₇ClN₄ [M]+: calculated: 444.1, measured: 443

2) Synthesis of Intermediate 5

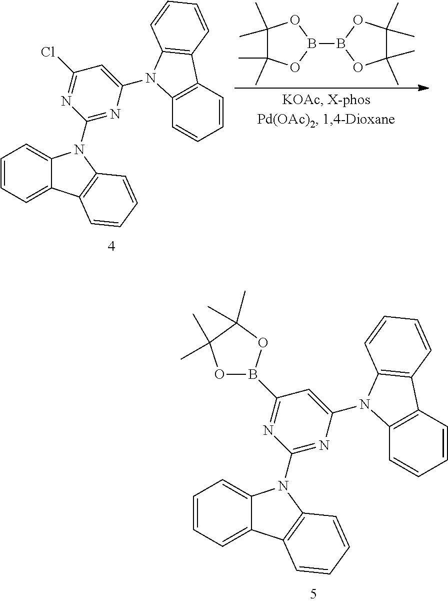

To a round-bottom flask, Intermediate 4 (1 eq), Pd(OAc)₂ (0.05 eq), X-Phos (0.1 eq), KOAc (2.1 eq), THF (400 mL), and H₂O (100 mL) were put and stirred. After reducing the temperature to about 0° C., a 1 M solution of 4,4,4′,4′,5,5,5′,5′-octamethyl-2,2′-bi(1,3,2-dioxaborolane) (1.2 eq) dissolved in THF was added thereto dropwise. After slowly elevating the temperature to room temperature, the resultant was stirred overnight. After finishing the reaction, ethyl acetate/H₂O was added, stirring was performed for about 30 minutes, and only an organic layer was separated using a separatory funnel. The resultant was dried over MgSO₄, and filtration was performed using silica, followed by drying. The solid thus dried was dissolved in methylene chloride, and column chromatography was performed using methylene chloride:ethyl acetate (1:1) to obtain Intermediate 5 (yield 87%).

C₃₆H₂₉BN₄O₂ [M]+: calculated: 536.2, measured: 535

3) Synthesis of Compound ET-19

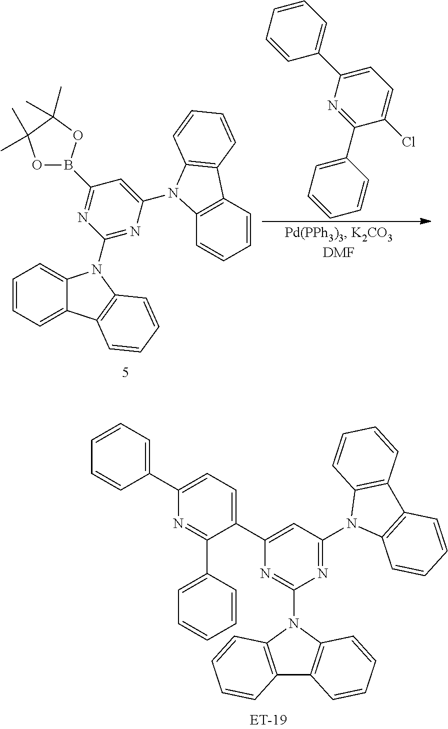 [SMILES: CC1(C)OB(c2cc(-n3c4ccccc4c4ccccc43)nc(-n3c4ccccc4c4ccccc43)n2)OC1(C)C ; Clc1ccc(-c2ccccc2)nc1-c1ccccc1 ; c1ccc(-c2ccc(-c3cc(-n4c5ccccc5c5ccccc54)nc(-n4c5ccccc5c5ccccc54)n3)c(-c3ccccc3)n2)cc1]

To a round-bottom flask, 3-chloro-2,6-diphenylpyridine (1.1 eq), Pd(PPh₃)₄ (0.05 eq), K₂CO₃ (1.2 eq), DMF (200 mL), and H₂O (50 mL) were put and stirred. After reducing the temperature to about 0° C., a 2 M solution of Intermediate 5 dissolved in THF was added thereto dropwise. After slowly elevating the temperature to room temperature, the resultant was stirred overnight. After finishing the reaction, ethyl acetate/H₂O was added, stirring was performed for about 30 minutes, and only an organic layer was separated using a separatory funnel. The resultant was dried over MgSO₄, and methylene chloride filtration was performed. The solid thus obtained was washed using MeOH and dried. The solid thus dried was dissolved in methylene chloride, and column chromatography was performed using methylene chloride:hexane (1:1) to obtain Compound ET-19 (yield 77%).

C₄₅H₂₉N₅ [M]+: calculated: 639.2, measured: 637

Elemental Analysis for calculated: C, 84.48; H, 4.57; N, 10.95.

2. Manufacture and Evaluation of Light Emitting Device Including Nitrogen-Containing Compound

A light emitting device according to an embodiment, including a nitrogen-containing compound according to an embodiment in an emission layer was manufactured by a method described below. Light emitting devices of Example 1 to Example 8 were manufactured using the Example Compounds of Compounds ET-2, ET-6, and ET-19 as the host materials of emission layers. Comparative Example 1 to Comparative Example 3 correspond to light emitting devices manufactured using Comparative Compound C1 to Comparative Compound C3 as the host materials of emission layers.

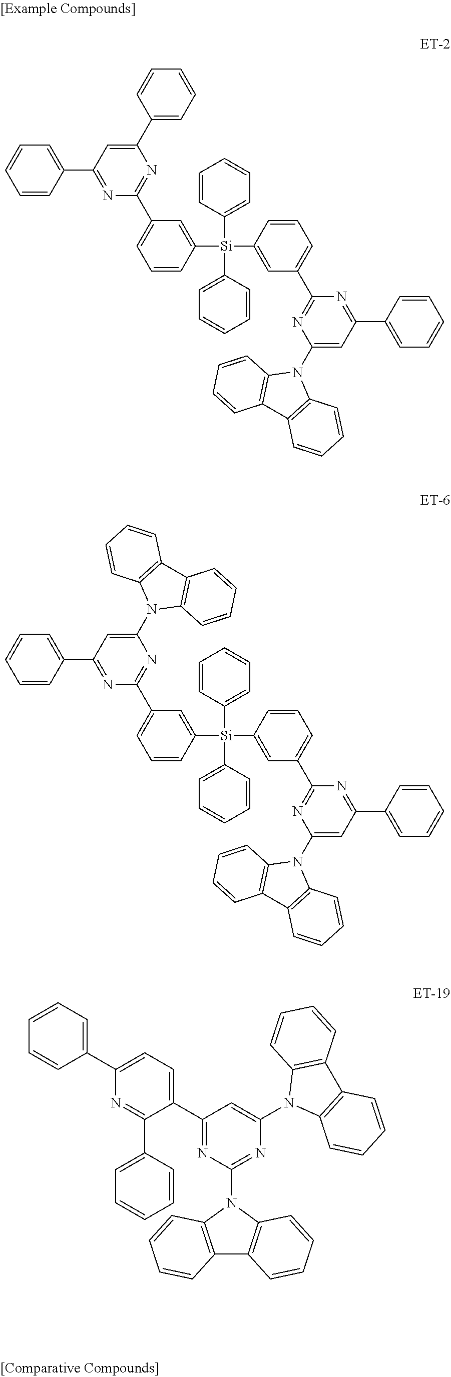 [SMILES: c1ccc(-c2cc(-c3ccccc3)nc(-c3cccc([Si](c4ccccc4)(c4ccccc4)c4cccc(-c5nc(-c6ccccc6)cc(-n6c7ccccc7c7ccccc76)n5)c4)c3)n2)cc1 ; c1ccc(-c2cc(-n3c4ccccc4c4ccccc43)nc(-c3cccc([Si](c4ccccc4)(c4ccccc4)c4cccc(-c5nc(-c6ccccc6)cc(-n6c7ccccc7c7ccccc76)n5)c4)c3)n2)cc1 ; c1ccc(-c2ccc(-c3cc(-n4c5ccccc5c5ccccc54)nc(-n4c5ccccc5c5ccccc54)n3)c(-c3ccccc3)n2)cc1]

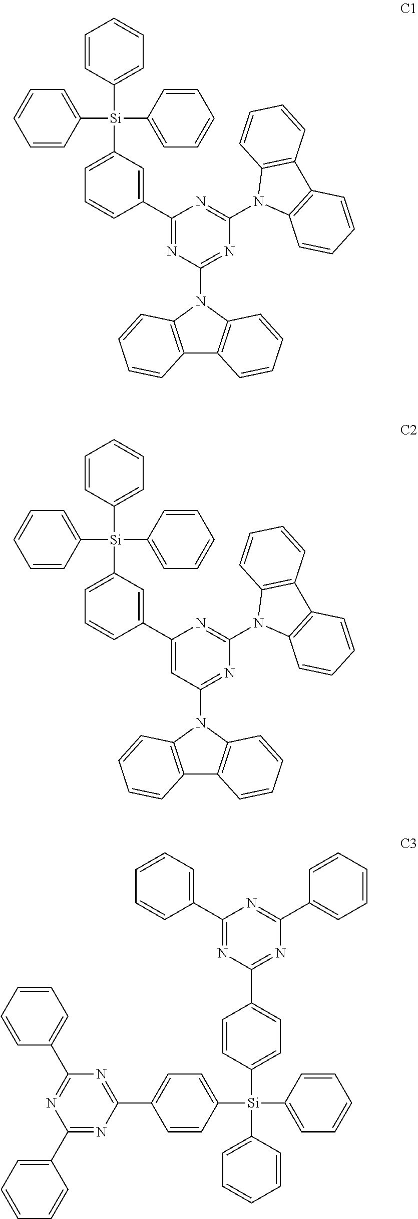 [SMILES: c1ccc([Si](c2ccccc2)(c2ccccc2)c2cccc(-c3nc(-n4c5ccccc5c5ccccc54)nc(-n4c5ccccc5c5ccccc54)n3)c2)cc1 ; c1ccc([Si](c2ccccc2)(c2ccccc2)c2cccc(-c3cc(-n4c5ccccc5c5ccccc54)nc(-n4c5ccccc5c5ccccc54)n3)c2)cc1 ; c1ccc(-c2nc(-c3ccccc3)nc(-c3ccc([Si](c4ccccc4)(c4ccccc4)c4ccc(-c5nc(-c6ccccc6)nc(-c6ccccc6)n5)cc4)cc3)n2)cc1]

(Manufacture of Light Emitting Devices)

### Example 1

An ITO glass substrate was cut into a size of about 50 mm×50 mm×0.5 mm, washed by ultrasonic waves using isopropyl alcohol and distilled water for about 10 minutes each, and cleaned by exposing to ultraviolet rays for about 10 minutes and exposing to ozone, and the glass substrate was installed in a vacuum deposition apparatus. A hole injection layer was formed of m-MTDATA to a thickness of about 40 Å, and a hole transport layer was formed of NPB to a thickness of about 10 Å. After that, an emission layer was formed of a host obtained by mixing Compound 1-23 and Example Compound ET-2 in a weight ratio of about 7:3, and doped with Compound PBD-01 in a 13% ratio, to a thickness of about 400 Å. An electron transport layer was formed of ET1 to a thickness of about 300 Å, and a second electrode was formed of Al to a thickness of about 1,200 Å. All layers were formed by a vacuum deposition method.

### Example 2

A light emitting device was manufactured by the same method as Example 1 except for using a host obtained by mixing Compound 1-23 and Example Compound ET-2 in a weight ratio of about 6:4 during forming the emission layer, in Example 1.

### Example 3

A light emitting device was manufactured by the same method as Example 1 except for using Example Compound ET-6 instead of Example Compound ET-2 and using PBD-03 instead of PBD-01 as the dopant compound during forming an emission layer, in Example 1.

### Example 4

A light emitting device was manufactured by the same method as Example 1 except for using Example Compound ET-6 instead of Example Compound ET-2, using a host obtained by mixing Compound 1-23 and Example Compound ET-6 in a weight ratio of about 6:4, and using PBD-03 instead of PBD-01 as the dopant compound during forming an emission layer, in Example 1.

### Example 5

A light emitting device was manufactured by the same method as Example 1 except for using PBD-03 instead of PBD-01 as the dopant compound during forming an emission layer, in Example 1.

### Example 6

A light emitting device was manufactured by the same method as Example 1 except for using a host obtained by mixing Compound 1-23 and Example Compound ET-2 in a weight ratio of about 6:4, and using PBD-03 instead of PBD-01 as the dopant compound during forming an emission layer, in Example 1.

### Example 7

A light emitting device was manufactured by the same method as Example 1 except for using Example Compound ET-19 instead of Example Compound ET-2 during forming an emission layer, in Example 1.

### Example 8

A light emitting device was manufactured by the same method as Example 1 except for using Example Compound ET-19 instead of Example Compound ET-2 and using PBD-03 instead of PBD-01 as the dopant compound during forming an emission layer, in Example 1.

### Comparative Example 1

A light emitting device was manufactured by the same method as Example 1 except for using Comparative Compound C1 instead of Example Compound ET-2 during forming an emission layer, in Example 1.

### Comparative Example 2

A light emitting device was manufactured by the same method as Example 1 except for using Comparative Compound C2 instead of Example Compound ET-2 during forming an emission layer, in Example 1.

### Comparative Example 3

A light emitting device was manufactured by the same method as Example 1 except for using Comparative Compound C3 instead of Example Compound ET-2 during forming an emission layer, in Example 1.

The compounds used for the manufacture of the light emitting devices of the Examples and Comparative Examples are shown below. The materials are materials of the related art, and commercial products were purified by sublimation and used for the manufacture of the devices.

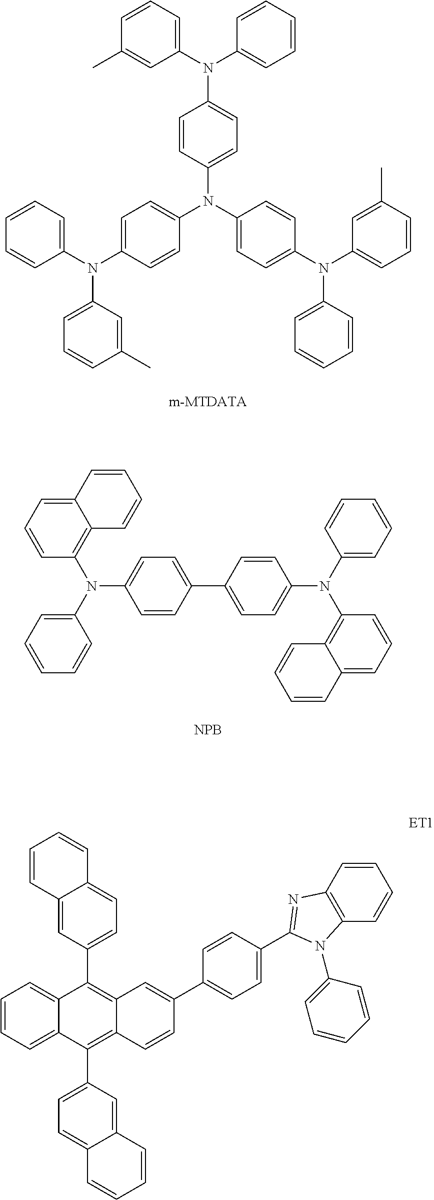 [SMILES: Cc1cccc(N(c2ccccc2)c2ccc(N(c3ccc(N(c4ccccc4)c4cccc(C)c4)cc3)c3ccc(N(c4ccccc4)c4cccc(C)c4)cc3)cc2)c1 ; c1ccc(N(c2ccc(-c3ccc(N(c4ccccc4)c4cccc5ccccc45)cc3)cc2)c2cccc3ccccc23)cc1 ; "NPB" ; c1ccc(-n2c(-c3ccc(-c4ccc5c(-c6ccc7ccccc7c6)c6ccccc6c(-c6ccc7ccccc7c6)c5c4)cc3)nc3ccccc32)cc1]

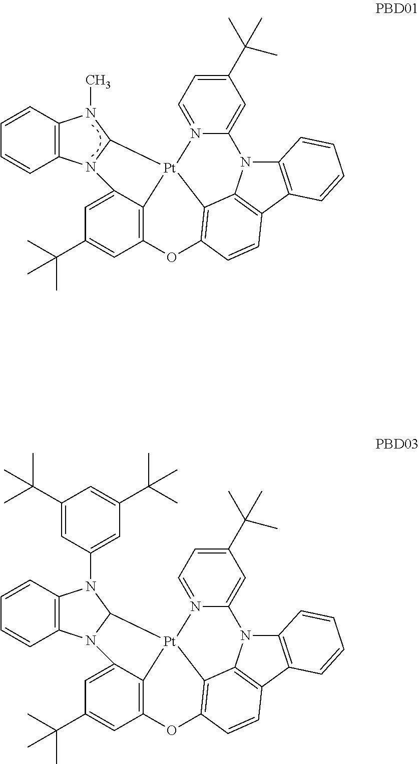

In Table 1, the simulation values of the lowest unoccupied molecular orbital (LUMO) energy level and the highest occupied molecular orbital (HOMO) energy level according to time-dependent density function theory (TD-DFT) of Example Compounds ET-2, ET-6, and ET-19, and Comparative Compounds C1 to C3 are shown. In Table 1, the measured values of the LUMO energy levels for Comparative Compounds C1 to C3 were measured by different pulse voltammetry (DPV).

TABLE 1

| | HOMO | LUMO |
|---|---|---|
| | (eV) | (eV) |
| Compound ET-1 | −6.36 | −1.91 |
| Compound ET-2 | −5.87 | −1.89 |
| Compound ET-6 | −5.82 | −1.92 |
| Compound ET-7 | −5.75 | −1.95 |
| Compound ET-8 | −5.75 | −1.91 |
| Compound ET-12 | −5.81 | −1.72 |
| Compound ET-19 | −5.90 | −1.70 |
| Comparative Compound C1 | −6.01 | −2.03 (measured value: −2.75) |
| Comparative Compound C2 | −5.80 | −1.93 (measured value: −2.65) |
| Comparative Compound C3 | −6.47 | −1.78 (measured value: −2.74) |

Referring to Table 1, it can be confirmed that the nitrogen-containing compounds of embodiments showed shallow LUMO energy levels when compared to the Comparative Compounds.

It can be confirmed that the measured values of the LUMO energy levels of Comparative Compounds C1 to C3 were −2.75 eV, −2.65 eV, and −2.74 eV, respectively, and showed deep LUMO energy levels when compared to the Example Compounds. Accordingly, if Comparative Compounds C1 to C3 are used as the host materials of emission layers, an energy barrier with a dopant may increase, and emission efficiency may be reduced.

(Evaluation of Properties of Light Emitting Device)

The device efficiency and device life of the light emitting devices manufactured using Example Compounds ET-2, ET-6, and ET-19, and Comparative Compound C1 to Comparative Compound C3 were evaluated. In Table 2, the evaluation results of the light emitting devices for Example 1 to Example 8, and Comparative Example 1 to Comparative Example 3 are shown. In the evaluation results on the properties for the Examples and Comparative Examples, shown in Table 2, the driving voltage and current density were measured using V7000 OLED IVL Test System, (Polaronix). The emission efficiency and device life are measured results at a current density of about 100 mA/cm². In Table 2, lifetime (T₉₀) is consumed time measured for the luminance reached 90% in contrast to an initial luminance.

TABLE 2

| | | | Weight | | | | | |
|---|---|---|---|---|---|---|---|---|
| | | | ratio of first | | | | | |
| | | | compound | | | Color | | |
| | First | Second | and second | Third | Driving | coordinate | Efficiency | Lifetime |
| | compound | compound | compound | compound | voltage | (CIE_y) | (cd/A) | (T₉₀) |
| Example 1 | Compound | HT-01 | 7:3 | PBD01 | 4.7 | 0.176 | 16.9 | 130 |
| | ET-2 | | | | | | | |
| Example 2 | Compound | HT-01 | 6:4 | PBD01 | 4.6 | 0.188 | 18.6 | 125 |
| | ET-2 | | | | | | | |
| Example 3 | Compound | HT-01 | 7:3 | PBD03 | 4.7 | 0.175 | 24.1 | 90 |
| | ET-6 | | | | | | | |
| Example 4 | Compound | HT-01 | 6:4 | PBD03 | 4.6 | 0.180 | 23.0 | 75 |
| | ET-6 | | | | | | | |
| Example 5 | Compound | HT-01 | 7:3 | PBD03 | 4.7 | 0.181 | 18.2 | 133 |
| | ET-2 | | | | | | | |
| Example 6 | Compound | HT-01 | 6:4 | PBD03 | 4.7 | 0.190 | 17.8 | 121 |
| | ET-2 | | | | | | | |
| Example 7 | Compound | HT-01 | 7:3 | PBD01 | 4.5 | 0.176 | 16.9 | 111 |
| | ET-19 | | | | | | | |
| Example 8 | Compound | HT-01 | 7:3 | PBD03 | 4.5 | 0.175 | 17.3 | 103 |
| | ET-19 | | | | | | | |
| Comparative | Comparative | HT-01 | 7:3 | PBD01 | 4.7 | 0.202 | 18.7 | 155 |
| Example 1 | Compound C1 | | | | | | | |
| Comparative | Comparative | HT-01 | 7:3 | PBD01 | 4.9 | 0.176 | 17.9 | 82 |
| Example 2 | Compound C2 | | | | | | | |
| Comparative | Comparative | HT-01 | 7:3 | PBD01 | 4.0 | 0.239 | 22.1 | 5 |
| Example 3 | Compound C3 | | | | | | | |

Referring to the results of Table 2, it can be confirmed that the Examples of the light emitting devices using the nitrogen-containing compounds according to embodiments as the host materials of emission layers, showed the same or lower driving voltage and similar or improved emission efficiency and device life when compared to the Comparative Examples. It can be confirmed that the Examples of the light emitting devices using the nitrogen-containing compounds according to embodiments as the host materials of emission layers all showed 2.0 or less of y color coordinate and excellent color purity. The nitrogen-containing compound according to an embodiment introduces a specific ring or a specific substituent bonded to a first heterocycle, and showed improved electron transport properties, suppressed light emission due to interaction with a dopant, and improved color purity.

The nitrogen-containing compound according to an embodiment includes a first heterocycle including two nitrogen atoms as ring-forming atoms. The first heterocycle may be connected with a second heterocycle via a first connecting group or may be directly bonded to a first substituent. Accordingly, the nitrogen-containing compound according to an embodiment may have a shallow LUMO energy level and reduced energy barrier with a dopant, thereby improving emission efficiency and device life characteristics.

Comparative Example 1 used Comparative Compound C1 including a triazine core, as a host material, and it can be confirmed that y color coordinate was increased when compared to the Examples using the Example Compounds including a pyrimidine core. Referring to Table 1 and Table 2, the LUMO value of Comparative Compound C1 was −2.03, and it can be confirmed that LUMO energy was deeper than the Example Compounds. Accordingly, in the case of Comparative Compound C1, a gap with LUMO energy with a dopant increased when compared to the Example Compounds, and material deterioration due to an energy barrier can occur. On the contrary, in the cases of the Example Compounds, in a structure essentially including a first heterocycle including a pyrimidine core, a second heterocycle connected with the first heterocycle via a first connecting group or a first substituent directly bonded to the first heterocycle may be included, and blue light having excellent color purity may be emitted, a shallow LUMO energy level may be shown, and material deterioration due to an energy barrier with a dopant may be prevented.

Comparative Compound C2 included in Comparative Example 2 includes a first heterocycle including a pyrimidine core but does not include a second heterocycle connected with the first heterocycle via a first connecting group, and it can be confirmed that both emission efficiency and device life were deteriorated when compared to the Examples. In the cases of the nitrogen-containing compounds according to embodiments, a second heterocycle connected with the first heterocycle via the first connecting group containing a silyl group, is included, and improved emission efficiency and life characteristics may be shown. In the case of Comparative Compound C2, a second heterocycle connected with the pyrimidine core via a first connecting group, was not included, and electron density was delocalized when compared to the Example Compounds, electron density was smaller when compared to Comparative Example 1 including a triazine core, a driving voltage was increased, and emission efficiency and device-life characteristics were degraded.

Comparative Compound C3 included in Comparative Example 3 has a structure in which two triazine groups are connected via a first connecting group, and, if applied to a light emitting device, it can be confirmed that device life was degraded when compared to the Examples. In the cases of the nitrogen-containing compounds according to embodiments, a first heterocycle including a pyrimidine core is essentially included, and a second heterocycle selected from pyridine, pyrimidine and triazine, connected with the first heterocycle via a first connecting group, is included, and improved life characteristics may be shown.

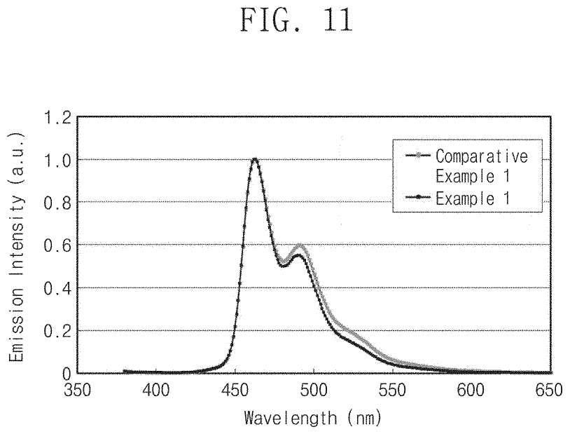

FIG. 11 is a graph showing emission spectra of light emitting devices of an Example and a Comparative Example. In FIG. 11, emission spectra of Example 1 and Comparative Example 1, manufactured in the above-described device manufacturing example are shown in accordance with wavelength.

Referring to FIG. 11, the spectrum of a light emitting device may include a first emission peak and a second emission peak. The first emission peak may be an emission peak emitted from a third compound. The first emission peak may be a phosphorescence emission peak emitted from a third compound itself. In an embodiment, a maximum emission wavelength of the first emission peak may be in a range of about 400 nm to about 470 nm. However, embodiments are not limited thereto. If the maximum emission wavelength of the first emission wavelength satisfies the above range, the light emitting device may emit blue light having excellent color purity. The second emission peak may be an emission peak derived from the charge interaction between the first compound and the third compound. The maximum emission wavelength of the second emission peak may be greater than the maximum emission wavelength of the first emission peak. The intensity of the second emission peak may be smaller than the intensity of the first emission peak.

When comparing the emission intensity of Example 1 and Comparative Example 1, it can be confirmed that the intensity of the first emission peak of Example 1 and the intensity of the first emission peak of Comparative Example 1 are similar, but the intensity of the second emission peak of Example 1 was reduced when compared to the intensity of the second emission peak of Comparative Example 1. Accordingly, in the case of the nitrogen-containing compound of the Example, emission efficiency emitted from the third compound which was used as a dopant was not reduced, while suppressing light emission due to the interaction with the dopant, and color purity can be improved.

The light emitting device of an embodiment may show improved device properties of high efficiency and long lifetime.

The nitrogen-containing compound of an embodiment is included in an emission layer of a light emitting device and may contribute to the increase of the efficiency and lifetime of the light emitting device.

Embodiments have been disclosed herein, and although terms are employed, they are used and are to be interpreted in a generic and descriptive sense only and not for purpose of limitation. In some instances, as would be apparent by one of ordinary skill in the art, features, characteristics, and/or elements described in connection with an embodiment may be used singly or in combination with features, characteristics, and/or elements described in connection with other embodiments unless otherwise specifically indicated. Accordingly, it will be understood by those of ordinary skill in the art that various changes in form and details may be made without departing from the spirit and scope of the disclosure as set forth in the claims.

What is claimed is:

1. A light emitting device, comprising:
a first electrode;
a second electrode facing the first electrode; and
an emission layer disposed between the first electrode and the second electrode, wherein
the emission layer comprises a first compound represented by Formula 1-1 or Formula 1-2: 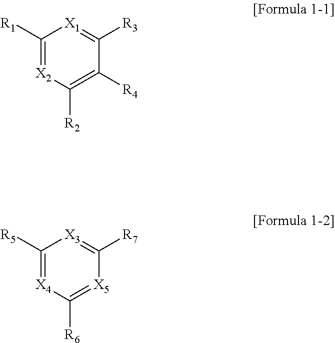
wherein in Formula 1-1 and Formula 1-2,
X₁ and X₂ are each N,
X₃ to X₅ are each independently N or C(R₈),
two of X₃ to X₅ are each N,
R₁ to R₄ and R₈ are each independently a hydrogen atom, a deuterium atom, a halogen atom, a substituted or unsubstituted alkyl group of 1 to 20 carbon atoms, a substituted or unsubstituted aryl group of 6 to 30 ring-forming carbon atoms, or a substituted or unsubstituted heteroaryl group of 2 to 30 ring-forming carbon atoms,
R₅ to R₇ are each independently a substituted or unsubstituted o-biphenyl group, or a substituted or unsubstituted heteroaryl group of 2 to 30 ring-forming carbon atoms,
at least one of R₅ to R₇ is each independently a substituted or unsubstituted o-biphenyl group, or a substituted or unsubstituted six-member heterocyclic group including a nitrogen atom as a ring-forming atom, and
at least one of R₁ to R₄ is each independently a group represented by one of Formula 2-1 to Formula 2-3: 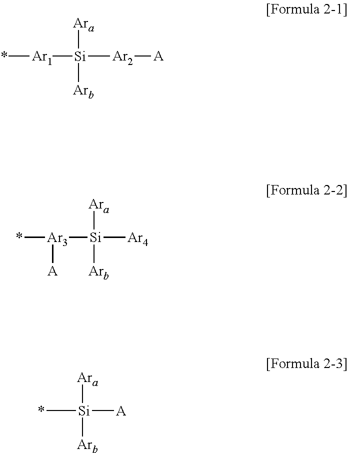
wherein in Formula 2-1 to Formula 2-3,
Arₐ, Ar_{b}, and Ar₄ are each independently a substituted or unsubstituted aryl group of 6 to 30 ring-forming carbon atoms,
Ar₁ and Ar₂ are each independently a substituted or unsubstituted arylene group of 6 to 30 ring-forming carbon atoms,
Ar₃ is a substituted or unsubstituted trivalent aryl group of 6 to 30 ring-forming carbon atoms, and
A is a group represented by Formula 3: 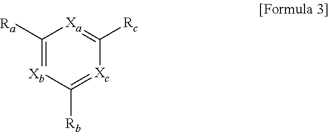
wherein in Formula 3,
Xₐ to X_{c} are each independently N or C(R_{d}), and
Rₐ to R_{d} are each independently a hydrogen atom, a deuterium atom, a halogen atom, a substituted or unsubstituted alkyl group of 1 to 20 carbon atoms, a substituted or unsubstituted aryl group of 6 to 30 ring-forming carbon atoms, or a substituted or unsubstituted heteroaryl group of 2 to 30 ring-forming carbon atoms,
except that one of Rₐ to R_{d} is a connecting part to the group represented by one of Formula 2-1 to Formula 2-3.

2. The light emitting device of claim 1, wherein the first compound represented by Formula 1-1 is represented by one of Formula 4-1 to Formula 4-7: 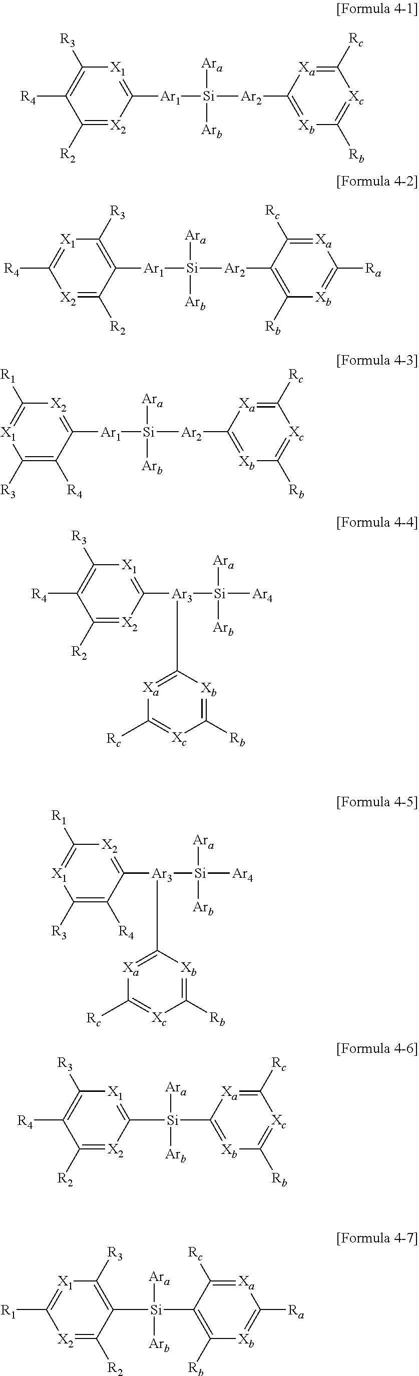
wherein in Formula 4-1 to Formula 4-7,
X₁, X₂, R₁ to R₃, Xₐ to X_{c}, Rₐ to R_{c}, Arₐ, Ar_{b}, and Ar₁ to Ar₄ are the each same as defined in Formula 1-1, Formula 2-1 to Formula 2-3, and Formula 3.

3. The light emitting device of claim 1, wherein at least one of R₁ to R₄ is each independently a group represented by one of Formula 5-1 to Formula 5-3: 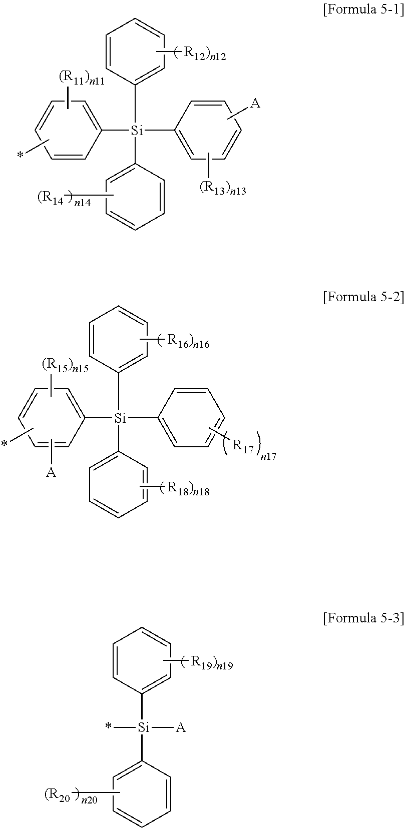
wherein in Formula 5-1 to Formula 5-3,
R₁₁ to R₂₀ are each independently a hydrogen atom, a deuterium atom, a halogen atom, a substituted or unsubstituted alkyl group of 1 to 20 carbon atoms, a substituted or unsubstituted aryl group of 6 to 30 ring-forming carbon atoms, or a substituted or unsubstituted heteroaryl group of 2 to 30 ring-forming carbon atoms,
n11 and n13 are each independently an integer from 0 to 4,
n12, n14, and n16 to n20 are each independently an integer from 0 to 5,
n15 is an integer from 0 to 3, and
A is the same as defined in Formula 2-1 to Formula 2-3.

4. The light emitting device of claim 3, wherein at least one of R₁ to R₄ is each independently a group represented by one of Formula 6-1 to Formula 6-3: 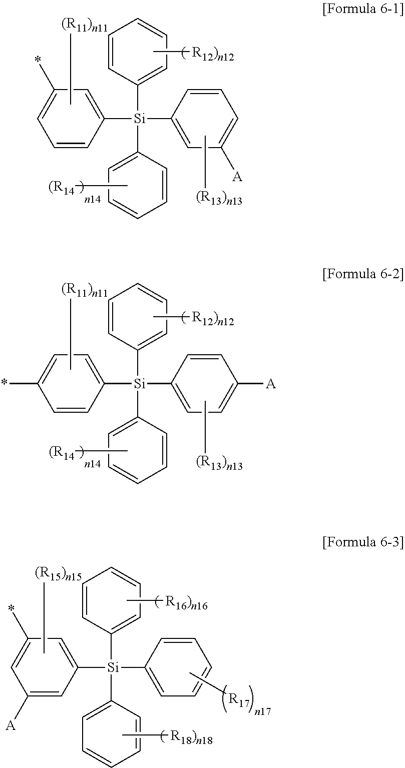
wherein in Formula 6-1 to Formula 6-3,
R₁₁ to R₁₈, n11 to n18, and A are each the same as defined in Formula 2-1 to Formula 2-3 and Formula 5-1 to Formula 5-3.

5. The light emitting device of claim 1, wherein at least one of R₅ to R₇ is each independently a group represented by one of Formula 7-1 to Formula 7-5: 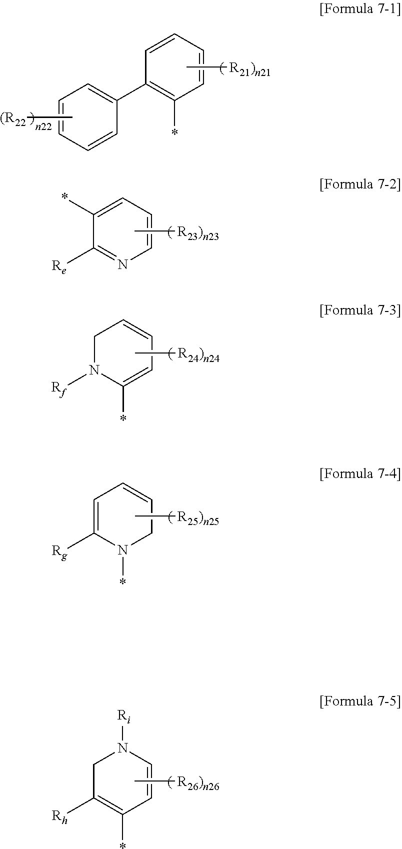
wherein in Formula 7-1 to Formula 7-5,
Rₑ to Rₕ are each independently a substituted or unsubstituted aryl group of 6 to 30 ring-forming carbon atoms,
Rᵢ and R₂₁ to R₂₆ are each independently a hydrogen atom, a deuterium atom, a halogen atom, a substituted or unsubstituted alkyl group of 1 to 20 carbon atoms, a substituted or unsubstituted aryl group of 6 to 30 ring-forming carbon atoms, or a substituted or unsubstituted heteroaryl group of 2 to 30 ring-forming carbon atoms,
n21 and n26 are each independently an integer from 0 to 4,
n22, n24, and n25 are each independently an integer from 0 to 5, and
n23 is an integer from 0 to 3.

6. The light emitting device of claim 5, wherein at least one of R₅ to R₇ is each independently a group represented by one of Formula 8-1 to Formula 8-5: 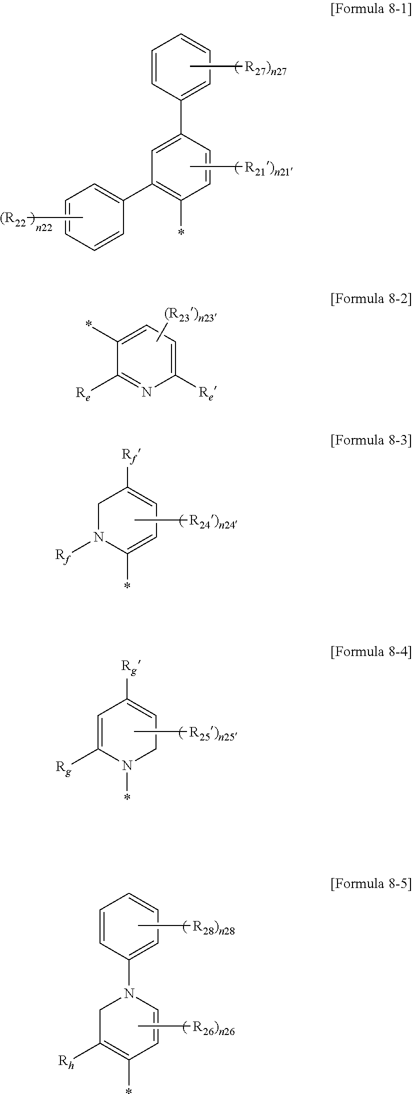
wherein in Formula 8-1 to Formula 8-5,
R₂₇ and R₂₈ are each independently a hydrogen atom, a deuterium atom, a halogen atom, a substituted or unsubstituted alkyl group of 1 to 20 carbon atoms, a substituted or unsubstituted aryl group of 6 to 30 ring-forming carbon atoms, or a substituted or unsubstituted heteroaryl group of 2 to 30 ring-forming carbon atoms,
Rₑ′ to R_{g}′ are each independently a substituted or unsubstituted aryl group of 6 to 30 ring-forming carbon atoms,
R₂₁′ and R₂₃′ to R₂₅′ are each independently a hydrogen atom, a deuterium atom, a halogen atom, a substituted or unsubstituted alkyl group of 1 to 20 carbon atoms, a substituted or unsubstituted aryl group of 6 to 30 ring-forming carbon atoms, or a substituted or unsubstituted heteroaryl group of 2 to 30 ring-forming carbon atoms,
n21′ is an integer from 0 to 3,
n23′ is an integer from 0 to 2,
n24′ and n25′ are each independently an integer from 0 to 4,
n27 and n28 are each independently an integer from 0 to 5, and
R₂₂, R₂₆, n22, n26, and Rₑ to Rₕ are each the same as defined in Formula 7-1 to Formula 7-5.

7. The light emitting device of claim 1, wherein the first compound represented by Formula 1-2 is represented by one of Formula 9-1 to Formula 9-5: 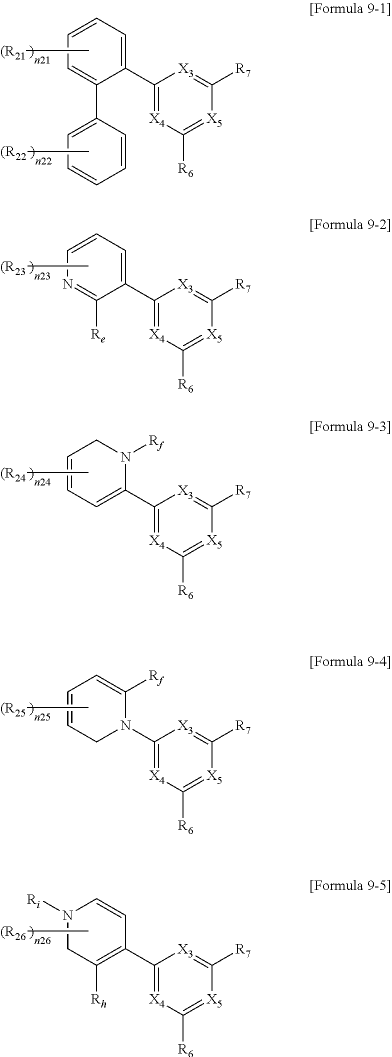
wherein in Formula 9-1 to Formula 9-5,
Rₑ to Rₕ are each independently a substituted or unsubstituted aryl group of 6 to 30 ring-forming carbon atoms,
Rᵢ and R₂₁ to R₂₆ are each independently a hydrogen atom, a deuterium atom, a halogen atom, a substituted or unsubstituted alkyl group of 1 to 20 carbon atoms, a substituted or unsubstituted aryl group of 6 to 30 ring-forming carbon atoms, or a substituted or unsubstituted heteroaryl group of 2 to 30 ring-forming carbon atoms,
n21 and n26 are each independently an integer from 0 to 4,
n22, n24, and n25 are each independently an integer from 0 to 5,
n23 is an integer from 0 to 3, and
X₃ to X₅, R₆, and R₇ are each the same as defined in Formula 1-2.

8. The light emitting device of claim 7, wherein at least one of R₆ and R₇ is each independently a group represented by Formula 10: 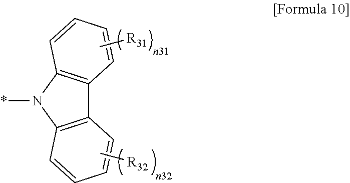
wherein in Formula 10,
R₃₁ and R₃₂ are each independently a hydrogen atom, a deuterium atom, a halogen atom, a substituted or unsubstituted alkyl group of 1 to 20 carbon atoms, a substituted or unsubstituted aryl group of 6 to 30 ring-forming carbon atoms, or a substituted or unsubstituted heteroaryl group of 2 to 30 ring-forming carbon atoms, and
n31 and n32 are each independently an integer from 0 to 4.

9. The light emitting device of claim 1, wherein the first compound comprises at least one compound selected from Compound Group 1:

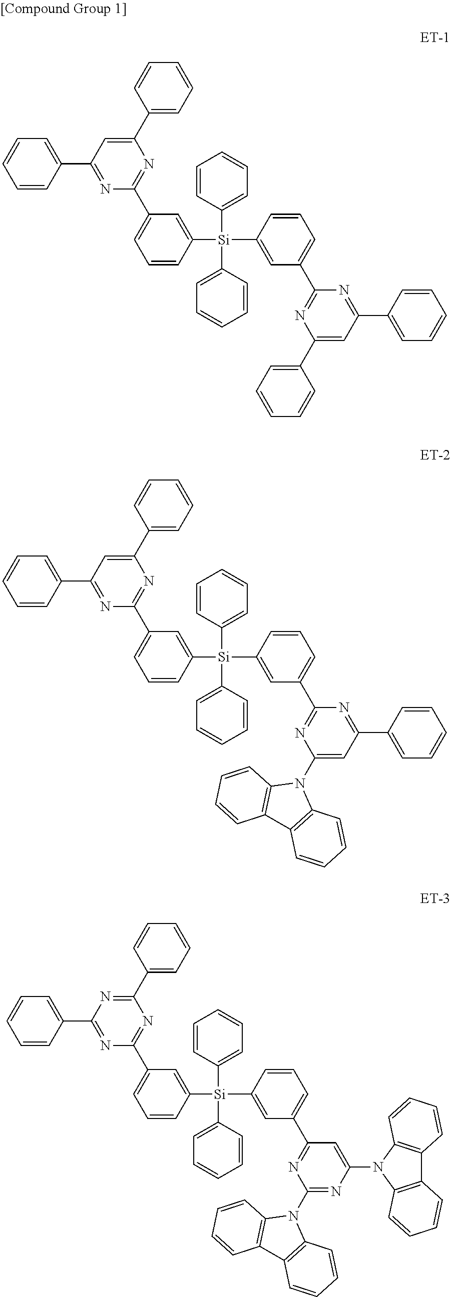 [SMILES: c1ccc(-c2cc(-c3ccccc3)nc(-c3cccc([Si](c4ccccc4)(c4ccccc4)c4cccc(-c5nc(-c6ccccc6)cc(-c6ccccc6)n5)c4)c3)n2)cc1 ; c1ccc(-c2cc(-c3ccccc3)nc(-c3cccc([Si](c4ccccc4)(c4ccccc4)c4cccc(-c5nc(-c6ccccc6)cc(-n6c7ccccc7c7ccccc76)n5)c4)c3)n2)cc1 ; c1ccc(-c2nc(-c3ccccc3)nc(-c3cccc([Si](c4ccccc4)(c4ccccc4)c4cccc(-c5cc(-n6c7ccccc7c7ccccc76)nc(-n6c7ccccc7c7ccccc76)n5)c4)c3)n2)cc1]

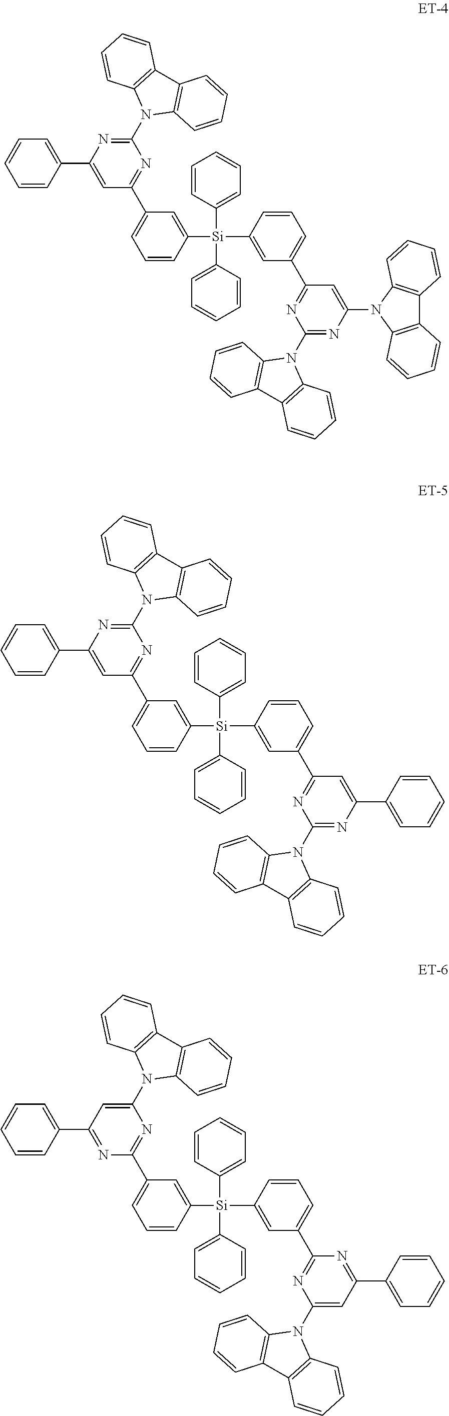 [SMILES: c1ccc(-c2cc(-c3cccc([Si](c4ccccc4)(c4ccccc4)c4cccc(-c5cc(-n6c7ccccc7c7ccccc76)nc(-n6c7ccccc7c7ccccc76)n5)c4)c3)nc(-n3c4ccccc4c4ccccc43)n2)cc1 ; c1ccc(-c2cc(-c3cccc([Si](c4ccccc4)(c4ccccc4)c4cccc(-c5cc(-c6ccccc6)nc(-n6c7ccccc7c7ccccc76)n5)c4)c3)nc(-n3c4ccccc4c4ccccc43)n2)cc1 ; c1ccc(-c2cc(-n3c4ccccc4c4ccccc43)nc(-c3cccc([Si](c4ccccc4)(c4ccccc4)c4cccc(-c5nc(-c6ccccc6)cc(-n6c7ccccc7c7ccccc76)n5)c4)c3)n2)cc1]

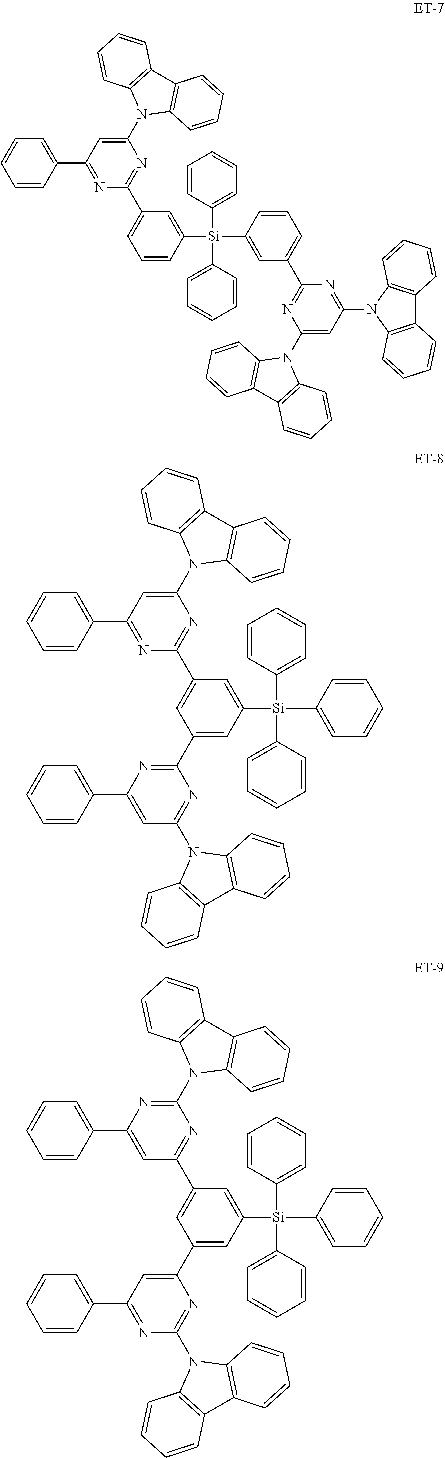

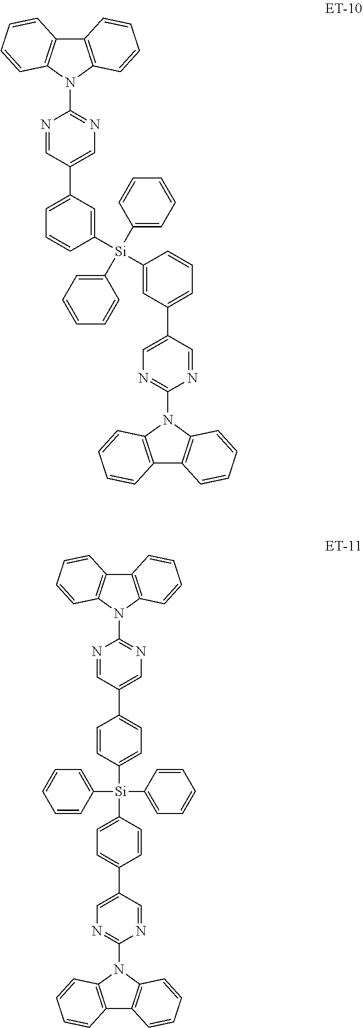 [SMILES: c1ccc([Si](c2ccccc2)(c2cccc(-c3cnc(-n4c5ccccc5c5ccccc54)nc3)c2)c2cccc(-c3cnc(-n4c5ccccc5c5ccccc54)nc3)c2)cc1 ; c1ccc([Si](c2ccccc2)(c2ccc(-c3cnc(-n4c5ccccc5c5ccccc54)nc3)cc2)c2ccc(-c3cnc(-n4c5ccccc5c5ccccc54)nc3)cc2)cc1]

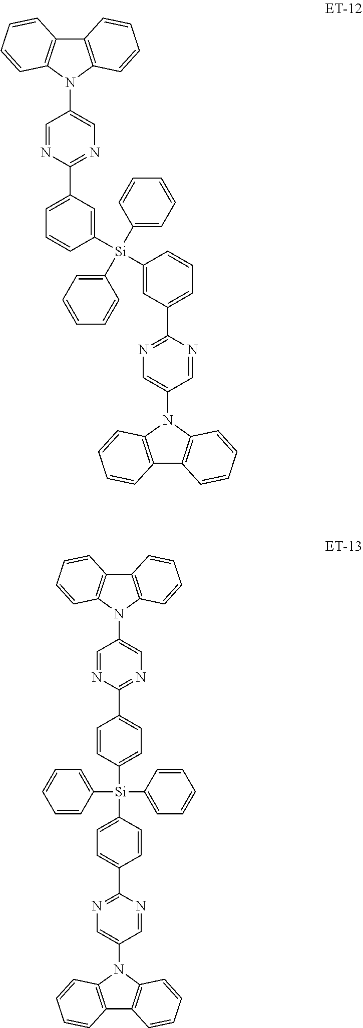 [SMILES: c1ccc([Si](c2ccccc2)(c2cccc(-c3ncc(-n4c5ccccc5c5ccccc54)cn3)c2)c2cccc(-c3ncc(-n4c5ccccc5c5ccccc54)cn3)c2)cc1 ; c1ccc([Si](c2ccccc2)(c2ccc(-c3ncc(-n4c5ccccc5c5ccccc54)cn3)cc2)c2ccc(-c3ncc(-n4c5ccccc5c5ccccc54)cn3)cc2)cc1]

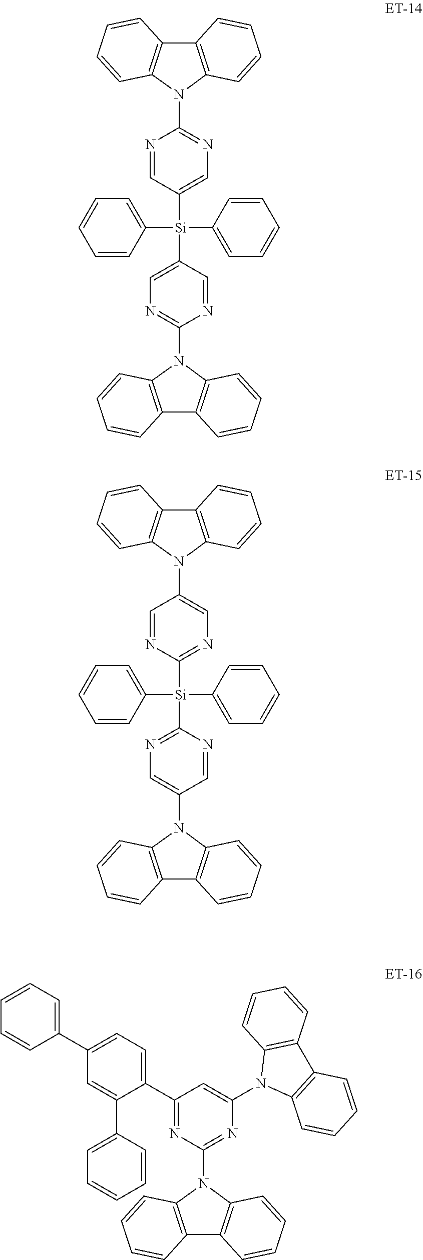 [SMILES: c1ccc([Si](c2ccccc2)(c2cnc(-n3c4ccccc4c4ccccc43)nc2)c2cnc(-n3c4ccccc4c4ccccc43)nc2)cc1 ; c1ccc([Si](c2ccccc2)(c2ncc(-n3c4ccccc4c4ccccc43)cn2)c2ncc(-n3c4ccccc4c4ccccc43)cn2)cc1 ; c1ccc(-c2ccc(-c3cc(-n4c5ccccc5c5ccccc54)nc(-n4c5ccccc5c5ccccc54)n3)c(-c3ccccc3)c2)cc1]

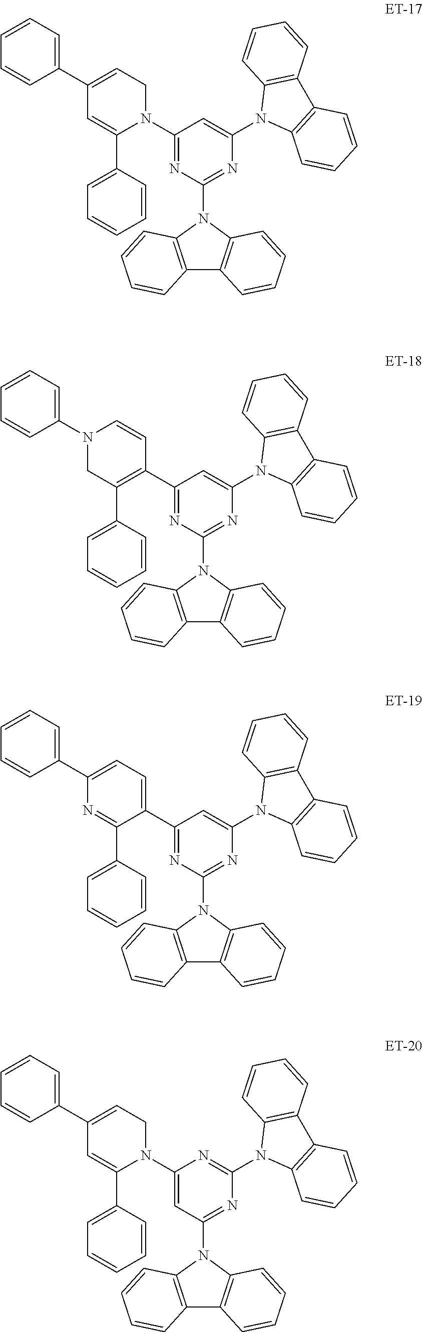 [SMILES: C1=C(c2ccccc2)C=C(c2ccccc2)N(c2cc(-n3c4ccccc4c4ccccc43)nc(-n3c4ccccc4c4ccccc43)n2)C1 ; C1=CN(c2ccccc2)CC(c2ccccc2)=C1c1cc(-n2c3ccccc3c3ccccc32)nc(-n2c3ccccc3c3ccccc32)n1 ; c1ccc(-c2ccc(-c3cc(-n4c5ccccc5c5ccccc54)nc(-n4c5ccccc5c5ccccc54)n3)c(-c3ccccc3)n2)cc1 ; C1=C(c2ccccc2)C=C(c2ccccc2)N(c2cc(-n3c4ccccc4c4ccccc43)nc(-n3c4ccccc4c4ccccc43)n2)C1]

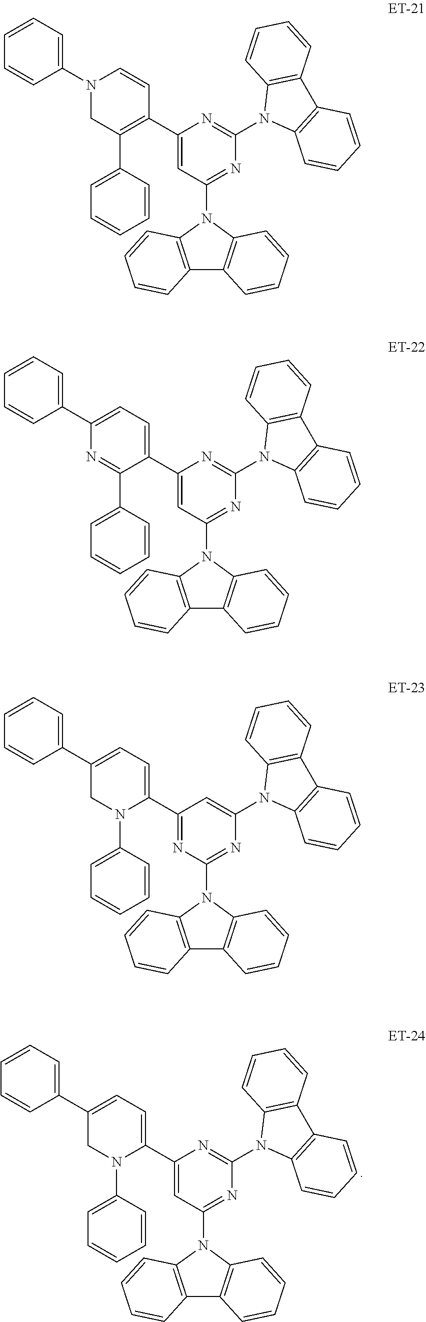 [SMILES: C1=CN(c2ccccc2)CC(c2ccccc2)=C1c1cc(-n2c3ccccc3c3ccccc32)nc(-n2c3ccccc3c3ccccc32)n1 ; c1ccc(-c2ccc(-c3cc(-n4c5ccccc5c5ccccc54)nc(-n4c5ccccc5c5ccccc54)n3)c(-c3ccccc3)n2)cc1 ; C1=C(c2ccccc2)CN(c2ccccc2)C(c2cc(-n3c4ccccc4c4ccccc43)nc(-n3c4ccccc4c4ccccc43)n2)=C1 ; C1=C(c2ccccc2)CN(c2ccccc2)C(c2cc(-n3c4ccccc4c4ccccc43)nc(-n3c4ccccc4c4ccccc43)n2)=C1]

10. The light emitting device of claim 1, wherein the emission layer further comprises a second compound represented by Formula H-1: 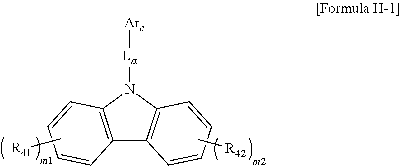
wherein in Formula H-1,
Lₐ is a direct linkage, a substituted or unsubstituted arylene group of 6 to 30 ring-forming carbon atoms, or a substituted or unsubstituted heteroarylene group of 2 to 30 ring-forming carbon atoms,
Ar_{c} is a substituted or unsubstituted aryl group of 6 to 30 ring-forming carbon atoms, or a substituted or unsubstituted heteroaryl group of 2 to 30 ring-forming carbon atoms,
R₄₁ and R₄₂ are each independently a hydrogen atom, a deuterium atom, a halogen atom, a substituted or unsubstituted alkyl group of 1 to 20 carbon atoms, a substituted or unsubstituted aryl group of 6 to 30 ring-forming carbon atoms, or a substituted or unsubstituted heteroaryl group of 2 to 30 ring-forming carbon atoms, and
m1 and m2 are each independently an integer from 0 to 4.

11. The light emitting device of claim 10, wherein the second compound comprises at least one compound selected from Compound Group 2:

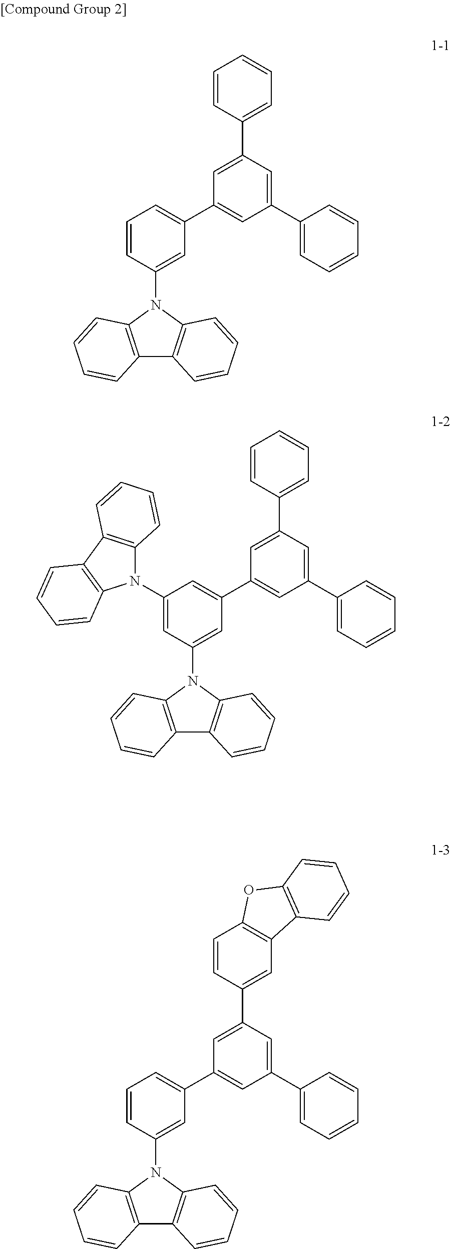 [SMILES: c1ccc(-c2cc(-c3ccccc3)cc(-c3cccc(-n4c5ccccc5c5ccccc54)c3)c2)cc1 ; c1ccc(-c2cc(-c3ccccc3)cc(-c3cc(-n4c5ccccc5c5ccccc54)cc(-n4c5ccccc5c5ccccc54)c3)c2)cc1 ; c1ccc(-c2cc(-c3cccc(-n4c5ccccc5c5ccccc54)c3)cc(-c3ccc4oc5ccccc5c4c3)c2)cc1]

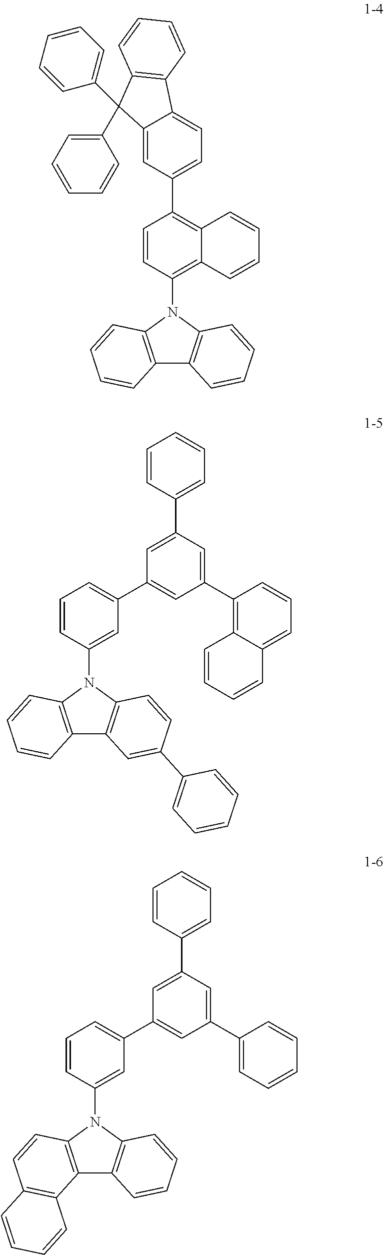

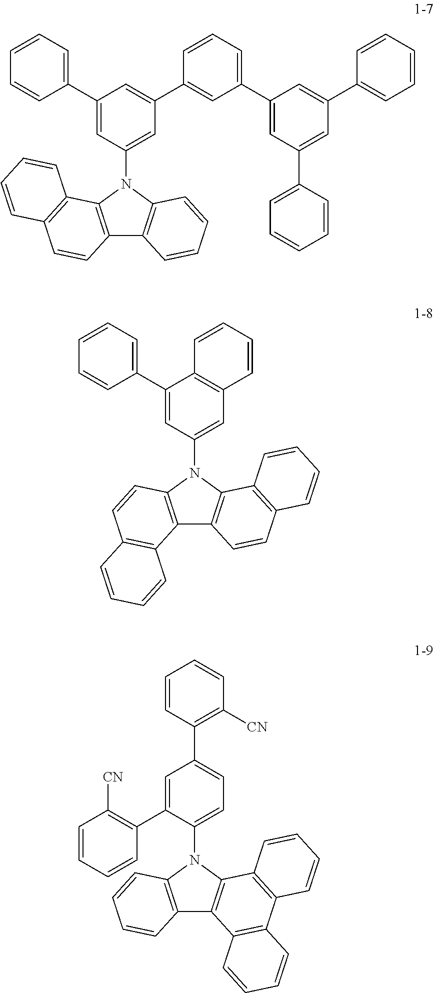 [SMILES: c1ccc(-c2cc(-c3ccccc3)cc(-c3cccc(-c4cc(-c5ccccc5)cc(-n5c6ccccc6c6ccc7ccccc7c65)c4)c3)c2)cc1 ; c1ccc(-c2cc(-n3c4ccc5ccccc5c4c4ccc5ccccc5c43)cc3ccccc23)cc1 ; N#Cc1ccccc1-c1ccc(-n2c3ccccc3c3c4ccccc4c4ccccc4c32)c(-c2ccccc2C#N)c1]

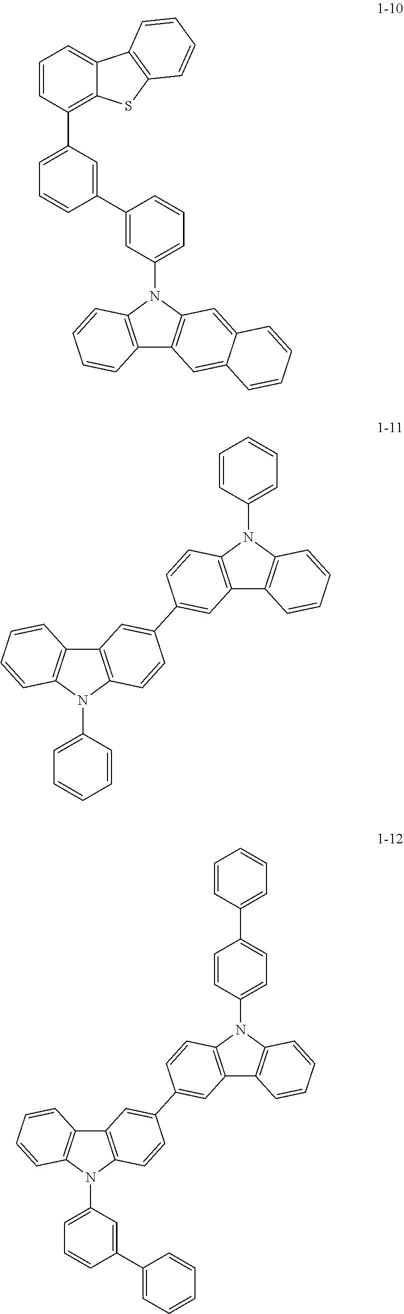 [SMILES: c1cc(-c2cccc(-n3c4ccccc4c4cc5ccccc5cc43)c2)cc(-c2cccc3c2sc2ccccc23)c1 ; c1ccc(-n2c3ccccc3c3cc(-c4ccc5c(c4)c4ccccc4n5-c4ccccc4)ccc32)cc1 ; c1ccc(-c2ccc(-n3c4ccccc4c4cc(-c5ccc6c(c5)c5ccccc5n6-c5cccc(-c6ccccc6)c5)ccc43)cc2)cc1]

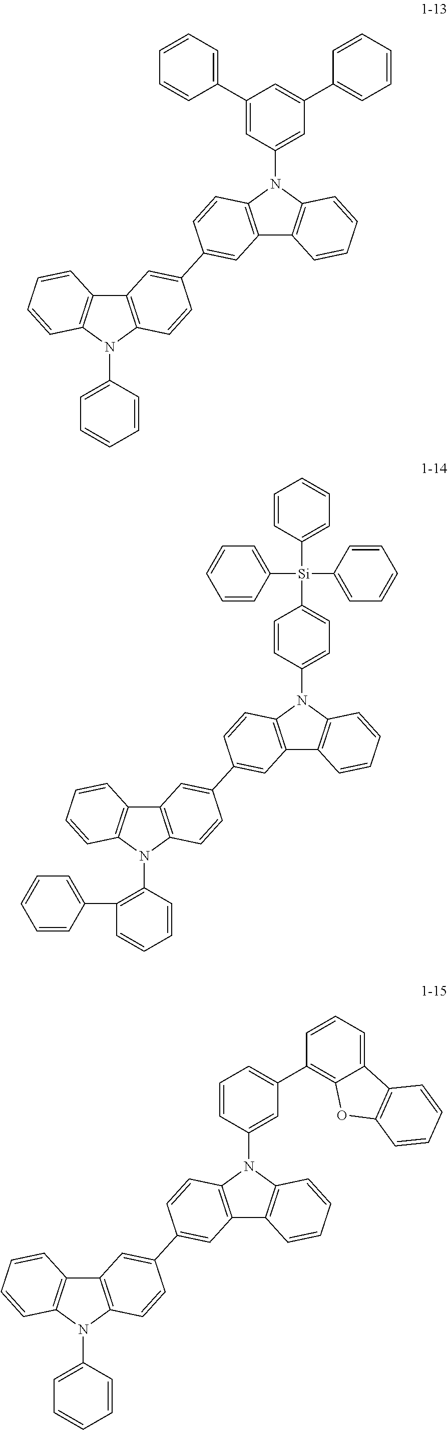 [SMILES: c1ccc(-c2cc(-c3ccccc3)cc(-n3c4ccccc4c4cc(-c5ccc6c(c5)c5ccccc5n6-c5ccccc5)ccc43)c2)cc1 ; c1ccc(-c2ccccc2-n2c3ccccc3c3cc(-c4ccc5c(c4)c4ccccc4n5-c4ccc([Si](c5ccccc5)(c5ccccc5)c5ccccc5)cc4)ccc32)cc1 ; c1ccc(-n2c3ccccc3c3cc(-c4ccc5c(c4)c4ccccc4n5-c4cccc(-c5cccc6c5oc5ccccc56)c4)ccc32)cc1]

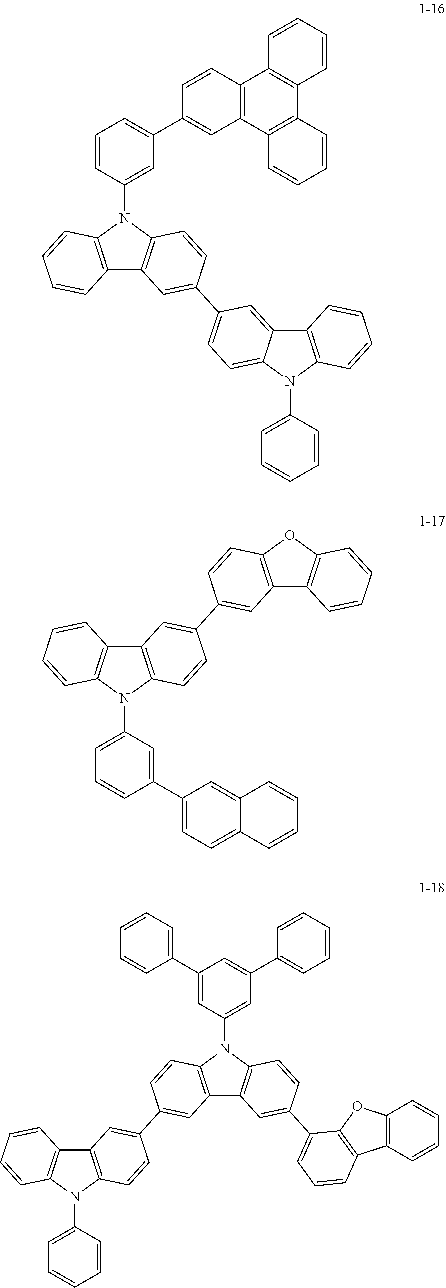 [SMILES: c1ccc(-n2c3ccccc3c3cc(-c4ccc5c(c4)c4ccccc4n5-c4cccc(-c5ccc6c7ccccc7c7ccccc7c6c5)c4)ccc32)cc1 ; c1cc(-c2ccc3ccccc3c2)cc(-n2c3ccccc3c3cc(-c4ccc5oc6ccccc6c5c4)ccc32)c1 ; c1ccc(-c2cc(-c3ccccc3)cc(-n3c4ccc(-c5ccc6c(c5)c5ccccc5n6-c5ccccc5)cc4c4cc(-c5cccc6c5oc5ccccc56)ccc43)c2)cc1]

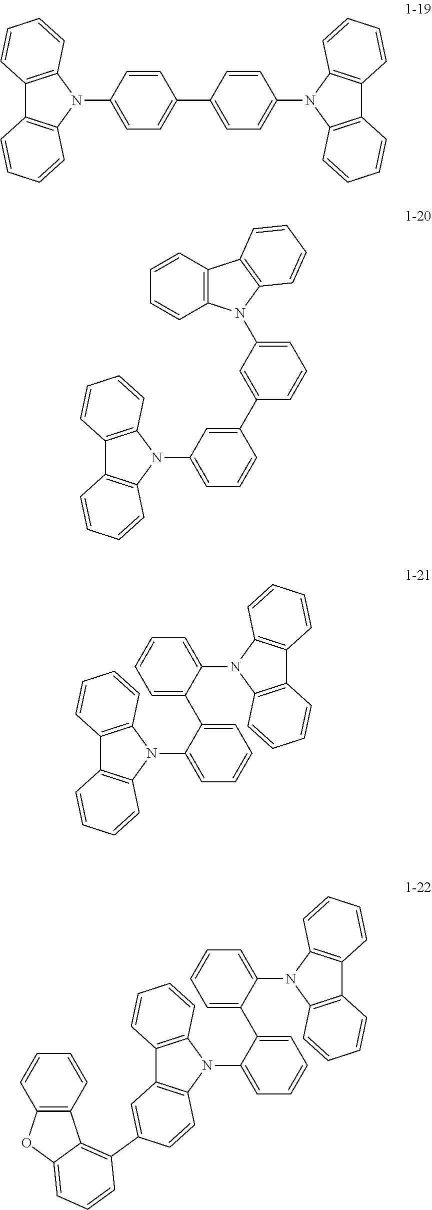 [SMILES: c1ccc2c(c1)c1ccccc1n2-c1ccc(-c2ccc(-n3c4ccccc4c4ccccc43)cc2)cc1 ; c1cc(-c2cccc(-n3c4ccccc4c4ccccc43)c2)cc(-n2c3ccccc3c3ccccc32)c1 ; c1ccc(-n2c3ccccc3c3ccccc32)c(-c2ccccc2-n2c3ccccc3c3ccccc32)c1 ; c1ccc(-n2c3ccccc3c3ccccc32)c(-c2ccccc2-n2c3ccccc3c3cc(-c4cccc5oc6ccccc6c45)ccc32)c1]

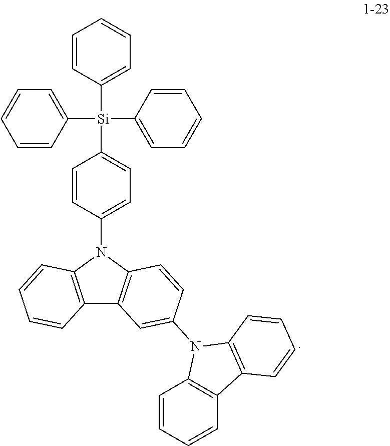 [SMILES: c1ccc([Si](c2ccccc2)(c2ccccc2)c2ccc(-n3c4ccccc4c4cc(-n5c6ccccc6c6ccccc65)ccc43)cc2)cc1]

12. The light emitting device of claim 1, wherein the emission layer further comprises a third compound represented by Formula D-1: 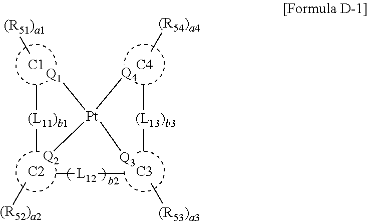
wherein in Formula D-1,
Q₁ to Q₄ are each independently C or N,
C1 to C4 are each independently a substituted or unsubstituted hydrocarbon ring of 5 to 30 ring-forming carbon atoms, or a substituted or unsubstituted heterocycle of 2 to 30 ring-forming carbon atoms,
L₁₁ to L₁₃ are each independently a direct linkage, *—O—*, *—S—*, 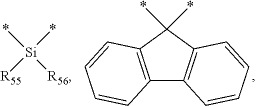
a substituted or unsubstituted divalent alkyl group of 1 to 20 carbon atoms, a substituted or unsubstituted arylene group of 6 to 30 ring-forming carbon atoms, or a substituted or unsubstituted heteroarylene group of 2 to 30 ring-forming carbon atoms,
R₅₁ to R₅₆ are each independently a hydrogen atom, a deuterium atom, a halogen atom, a cyano group, a substituted or unsubstituted amine group, a substituted or unsubstituted alkyl group of 1 to 20 carbon atoms, a substituted or unsubstituted aryl group of 6 to 30 ring-forming carbon atoms, or a substituted or unsubstituted heteroaryl group of 2 to 30 ring-forming carbon atoms, or are combined with an adjacent group to form a ring,
a1 to a4 are each independently an integer from 0 to 4, and
b1 to b3 are each independently 0 or 1.

13. The light emitting device of claim 12, wherein the third compound comprises at least one compound selected from Compound Group 3:

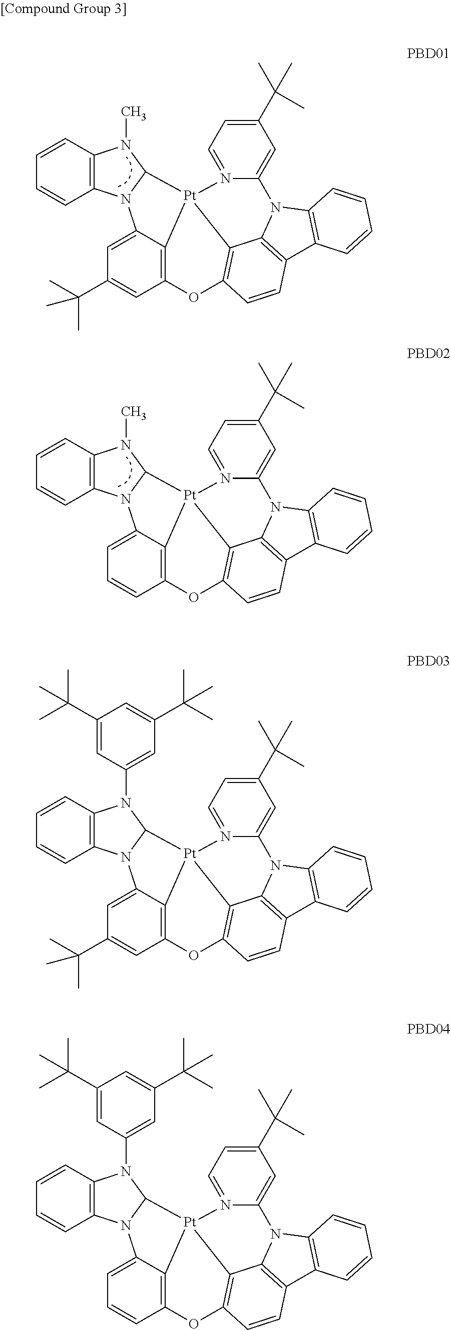

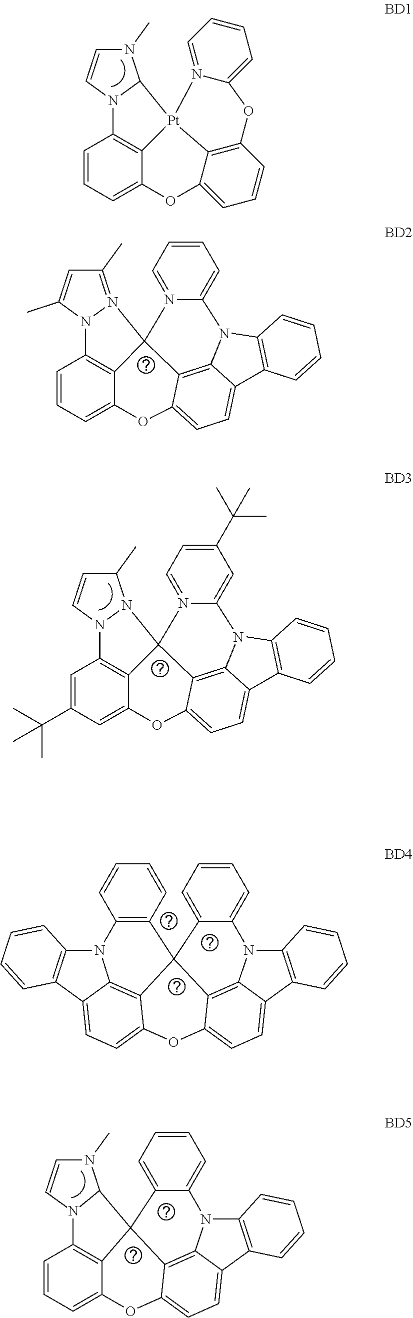

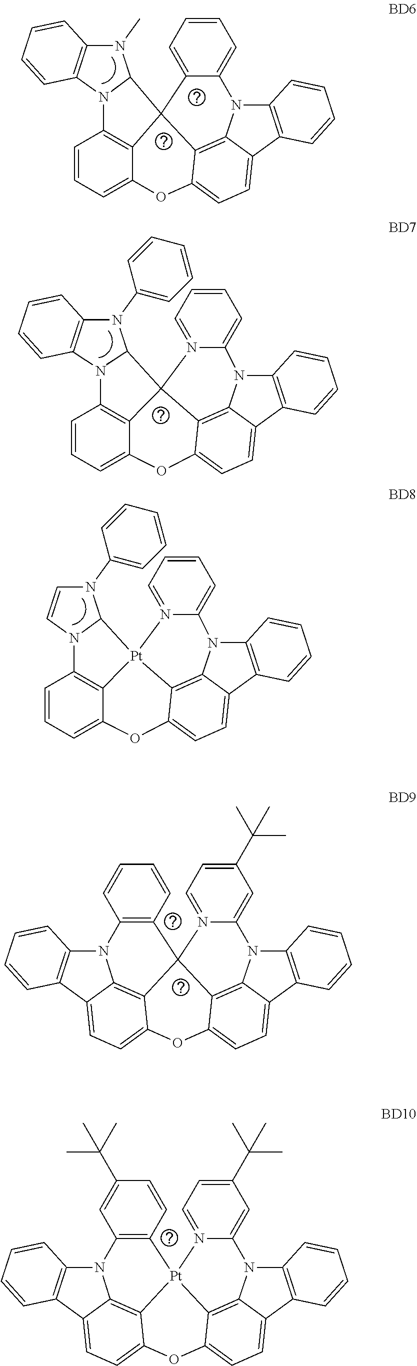

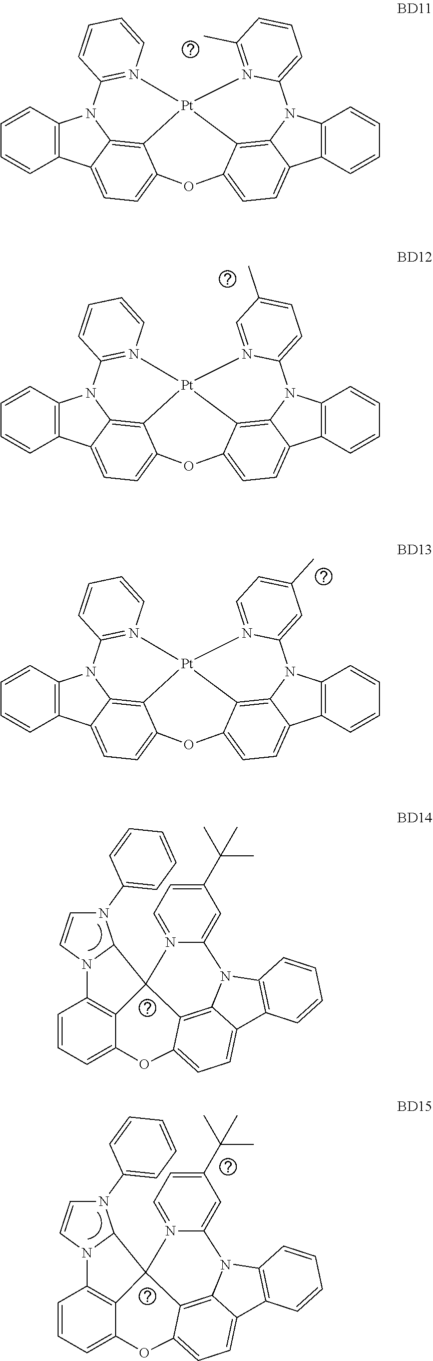

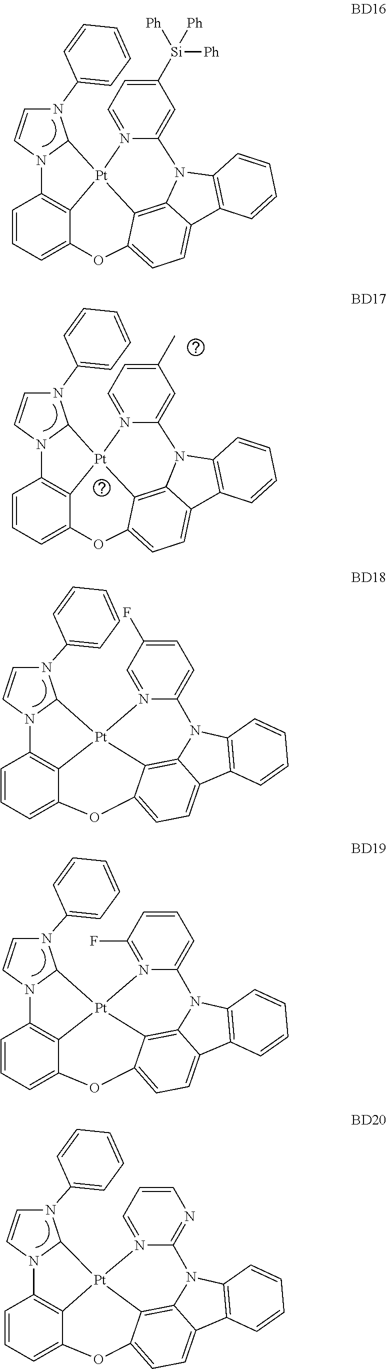

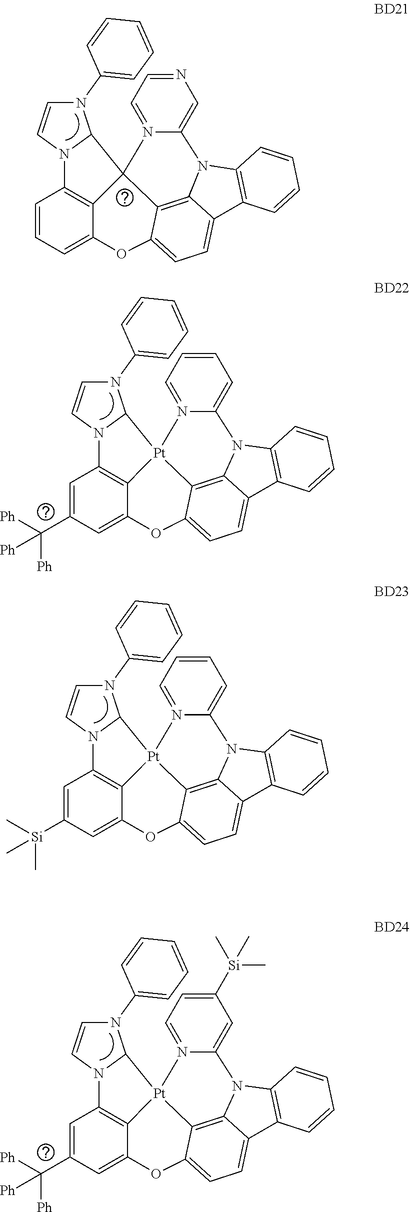

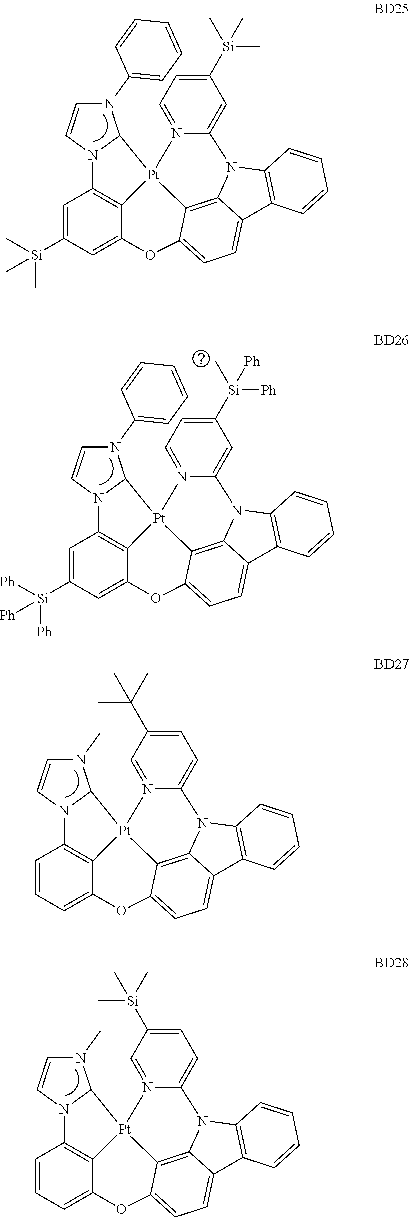

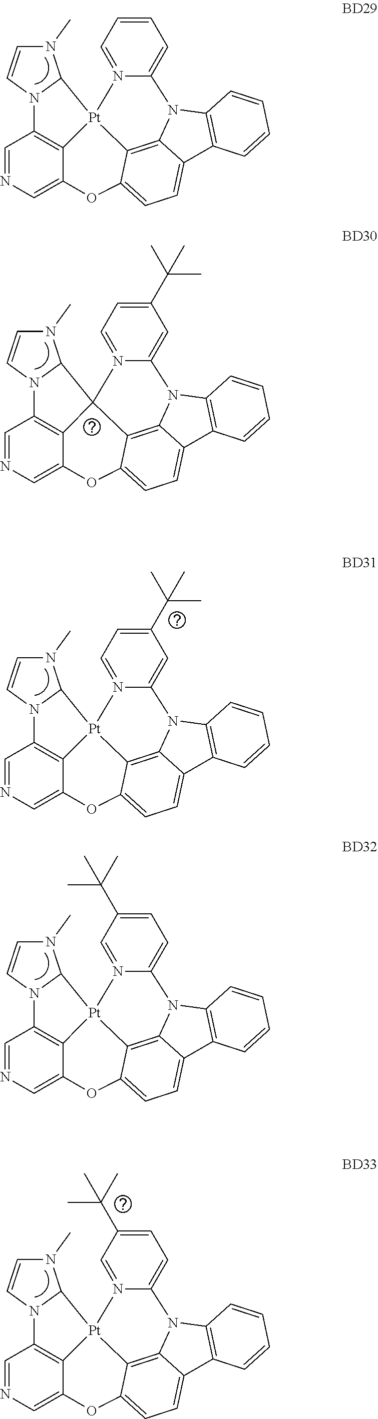

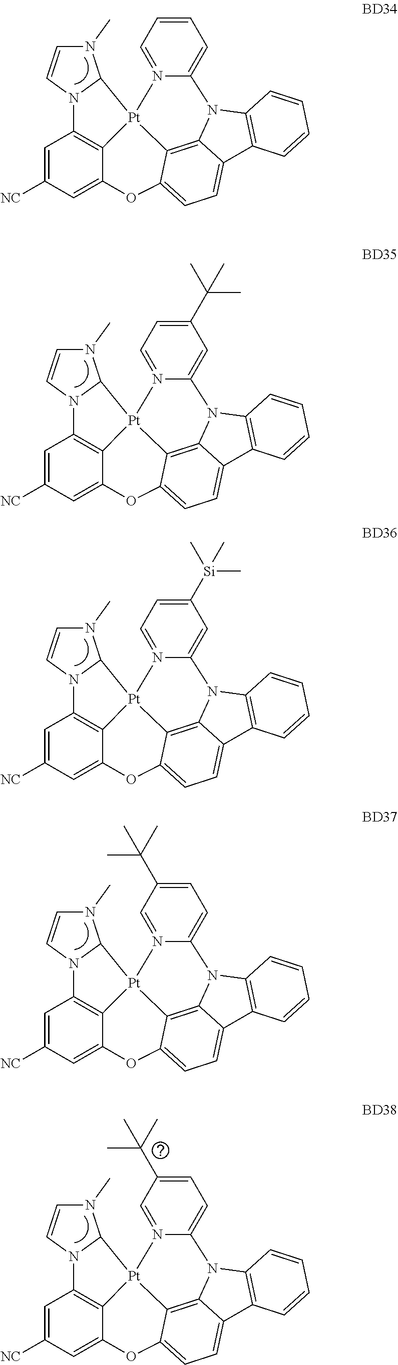

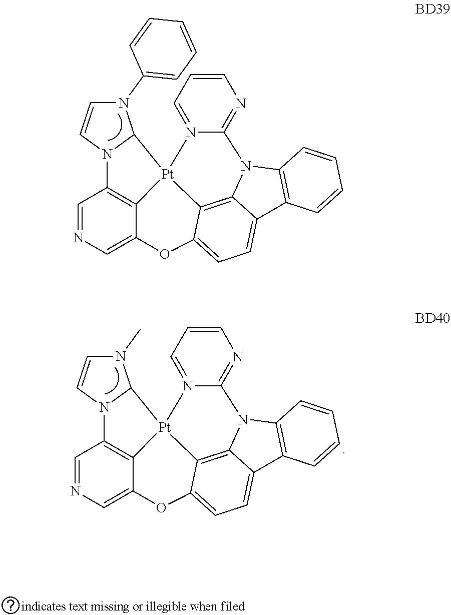

14. A nitrogen-containing compound represented by Formula 1-1 or Formula 1-2: 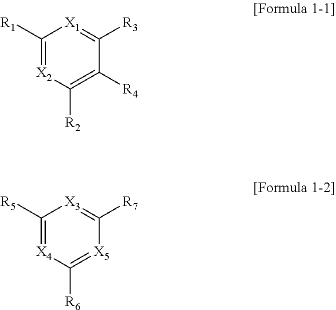
wherein in Formula 1-1 and Formula 1-2,
X₁ and X₂ are each N,
X₃ to X₅ are each independently N or C(R₈),
two of X₃ to X₅ are each N,
R₁ to R₄ and R₈ are each independently a hydrogen atom, a deuterium atom, a halogen atom, a substituted or unsubstituted alkyl group of 1 to 20 carbon atoms, a substituted or unsubstituted aryl group of 6 to 30 ring-forming carbon atoms, or a substituted or unsubstituted heteroaryl group of 2 to 30 ring-forming carbon atoms,
R₅ to R₇ are each independently a substituted or unsubstituted o-biphenyl group, or a substituted or unsubstituted heteroaryl group of 2 to 30 ring-forming carbon atoms, at least one of R₅ to R₇ is each independently a substituted or unsubstituted o-biphenyl group, or a substituted or unsubstituted six-member heterocyclic group including a nitrogen atom as a ring-forming atom, and
at least one of R₁ to R₄ is each independently a group represented by one of Formula 2-1 to Formula 2-3: 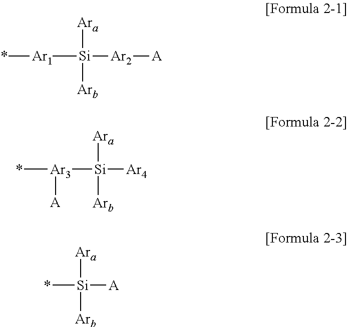
wherein in Formula 2-1 to Formula 2-3,
Arₐ, Ar_{b}, and Ar₄ are each independently a substituted or unsubstituted aryl group of 6 to 30 ring-forming carbon atoms,
Ar₁ and Ar₂ are each independently a substituted or unsubstituted arylene group of 6 to 30 ring-forming carbon atoms,
Ar₃ is a substituted or unsubstituted trivalent aryl group of 6 to 30 ring-forming carbon atoms, and
A is a group represented by Formula 3: 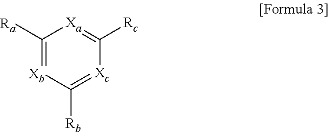
wherein in Formula 3,
Xₐ to X_{c} are each independently N or C(R_{d}), and
Rₐ to R_{d} are each independently a hydrogen atom, a deuterium atom, a halogen atom, a substituted or unsubstituted alkyl group of 1 to 20 carbon atoms, a substituted or unsubstituted aryl group of 6 to 30 ring-forming carbon atoms, or a substituted or unsubstituted heteroaryl group of 2 to 30 ring-forming carbon atoms,
except that one of Rₐ to R_{d} is a connecting part to the group represented by one of Formula 2-1 to Formula 2-3.

15. The nitrogen-containing compound of claim 14, wherein the nitrogen-containing compound represented by Formula 1-1 is represented by one of Formula 4-1 to Formula 4-7: 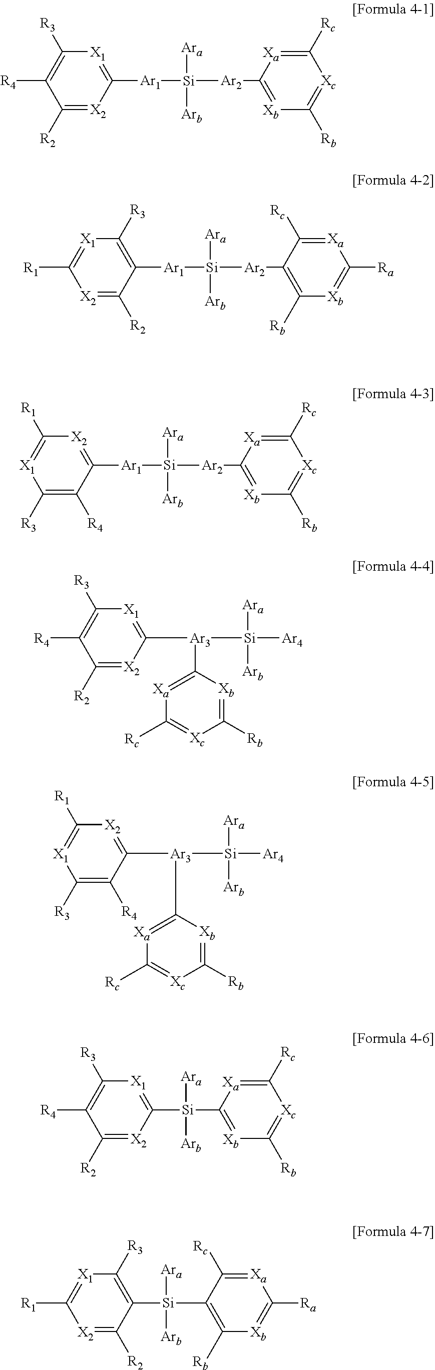
wherein in Formula 4-1 to Formula 4-7,
X₁, X₂, R₁ to R₃, Xₐ to X_{c}, Rₐ to R_{c}, Arₐ, Ar_{b}, and Ar₁ to Ar₄ are each the same as defined in Formula 1-1, Formula 2-1 to Formula 2-3, and Formula 3.

16. The nitrogen-containing compound of claim 14, wherein at least one of R₁ to R₄ is each independently a group represented by one of Formula 5-1 to Formula 5-3: 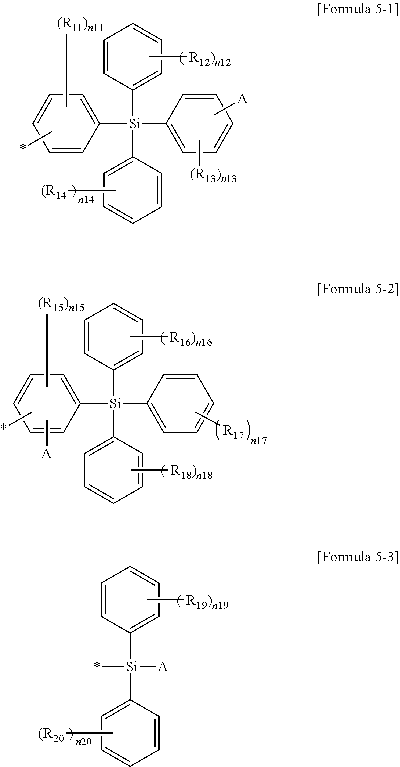
wherein in Formula 5-1 to Formula 5-3,
R₁₁ to R₂₀ are each independently a hydrogen atom, a deuterium atom, a halogen atom, a substituted or unsubstituted alkyl group of 1 to 20 carbon atoms, a substituted or unsubstituted aryl group of 6 to 30 ring-forming carbon atoms, or a substituted or unsubstituted heteroaryl group of 2 to 30 ring-forming carbon atoms,
n11 and n13 are each independently an integer from 0 to 4,
n12, n14, and n16 to n20 are each independently an integer from 0 to 5,
n15 is an integer from 0 to 3, and
A is the same as defined in Formula 2-1 to Formula 2-3.

17. The nitrogen-containing compound of claim 14, wherein at least one of R₅ to R₇ is each independently a group represented by one of Formula 7-1 to Formula 7-5: 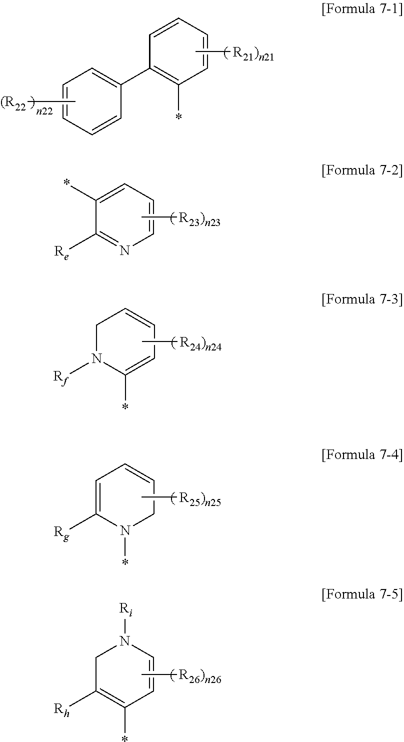
wherein in Formula 7-1 to Formula 7-5,
Rₑ to Rₕ are each independently a substituted or unsubstituted aryl group of 6 to 30 ring-forming carbon atoms,
Rᵢ and R₂₁ to R₂₆ are each independently a hydrogen atom, a deuterium atom, a halogen atom, a substituted or unsubstituted alkyl group of 1 to 20 carbon atoms, a substituted or unsubstituted aryl group of 6 to 30 ring-forming carbon atoms, or a substituted or unsubstituted heteroaryl group of 2 to 30 ring-forming carbon atoms,
n21 and n26 are each independently an integer from 0 to 4,
n22, n24, and n25 are each independently an integer from 0 to 5, and
n23 is an integer from 0 to 3.

18. The nitrogen-containing compound of claim 14, wherein the nitrogen-containing compound represented by Formula 1-2 is represented by one of Formula 9-1 to Formula 9-5: 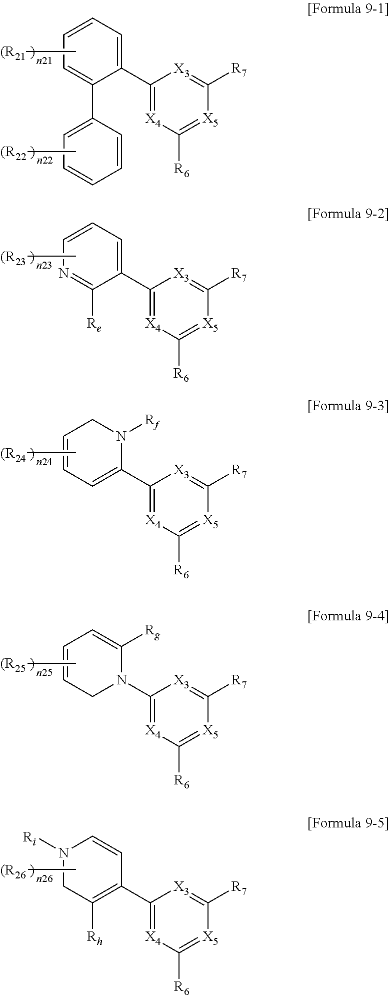
wherein in Formula 9-1 to Formula 9-5,
Rₑ to Rₕ are each independently a substituted or unsubstituted aryl group of 6 to 30 ring-forming carbon atoms,
Rᵢ and R₂₁ to R₂₆ are each independently a hydrogen atom, a deuterium atom, a halogen atom, a substituted or unsubstituted alkyl group of 1 to 20 carbon atoms, a substituted or unsubstituted aryl group of 6 to 30 ring-forming carbon atoms, or a substituted or unsubstituted heteroaryl group of 2 to 30 ring-forming carbon atoms,
n21 and n26 are each independently an integer from 0 to 4,
n22, n24, and n25 are each independently an integer from 0 to 5,
n23 is an integer from 0 to 3, and
X₃ to X₅, R₆, and R₇ are each the same as defined in Formula 1-2.

19. The nitrogen-containing compound of claim 18, wherein at least one of R₆ and R₇ is each independently a group represented by Formula 10: 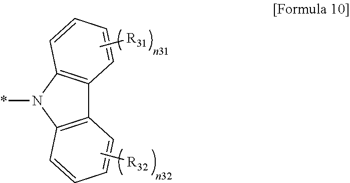
wherein in Formula 10,
R₃₁ and R₃₂ are each independently a hydrogen atom, a deuterium atom, a halogen atom, a substituted or unsubstituted alkyl group of 1 to 20 carbon atoms, a substituted or unsubstituted aryl group of 6 to 30 ring-forming carbon atoms, or a substituted or unsubstituted heteroaryl group of 2 to 30 ring-forming carbon atoms, and
n31 and n32 are each independently an integer from 0 to 4.

20. The nitrogen-containing compound of claim 14, wherein the nitrogen-containing compound is selected from Compound Group 1: 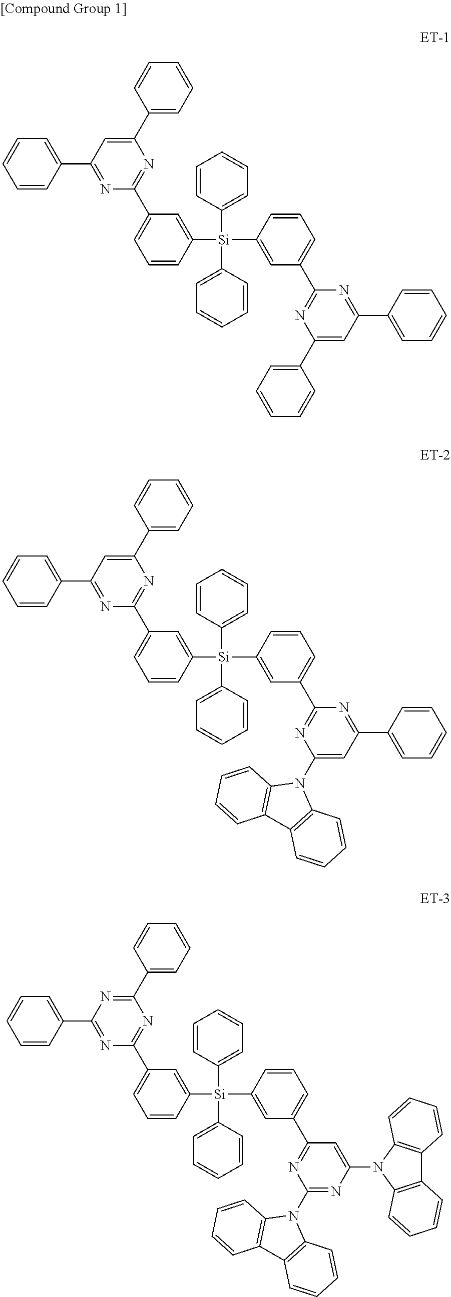 [SMILES: c1ccc(-c2cc(-c3ccccc3)nc(-c3cccc([Si](c4ccccc4)(c4ccccc4)c4cccc(-c5nc(-c6ccccc6)cc(-c6ccccc6)n5)c4)c3)n2)cc1 ; c1ccc(-c2cc(-c3ccccc3)nc(-c3cccc([Si](c4ccccc4)(c4ccccc4)c4cccc(-c5nc(-c6ccccc6)cc(-n6c7ccccc7c7ccccc76)n5)c4)c3)n2)cc1 ; c1ccc(-c2nc(-c3ccccc3)nc(-c3cccc([Si](c4ccccc4)(c4ccccc4)c4cccc(-c5cc(-n6c7ccccc7c7ccccc76)nc(-n6c7ccccc7c7ccccc76)n5)c4)c3)n2)cc1] 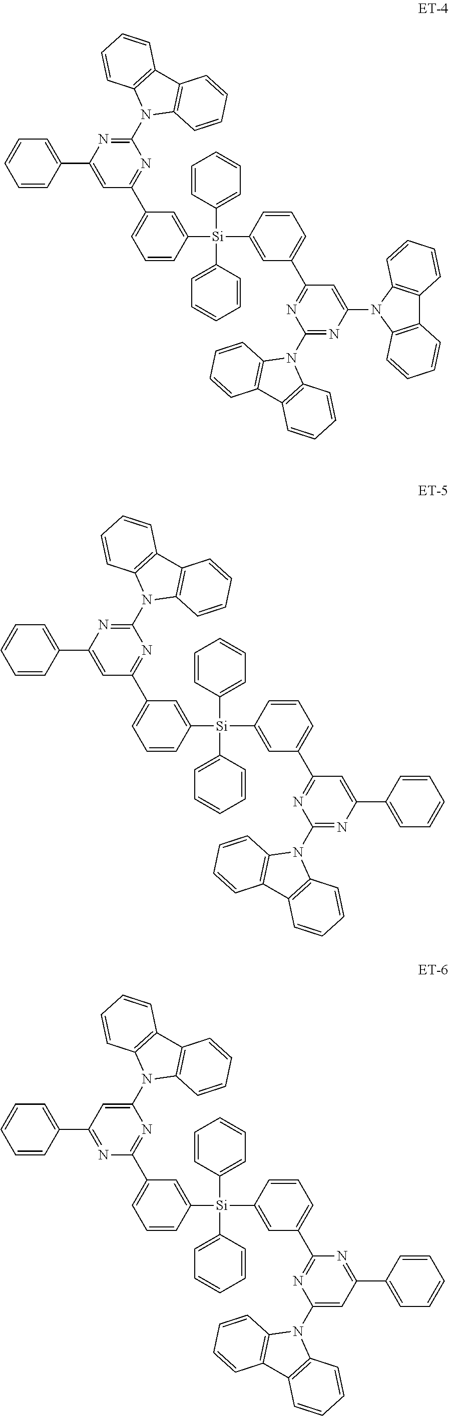 [SMILES: c1ccc(-c2cc(-c3cccc([Si](c4ccccc4)(c4ccccc4)c4cccc(-c5cc(-n6c7ccccc7c7ccccc76)nc(-n6c7ccccc7c7ccccc76)n5)c4)c3)nc(-n3c4ccccc4c4ccccc43)n2)cc1 ; c1ccc(-c2cc(-c3cccc([Si](c4ccccc4)(c4ccccc4)c4cccc(-c5cc(-c6ccccc6)nc(-n6c7ccccc7c7ccccc76)n5)c4)c3)nc(-n3c4ccccc4c4ccccc43)n2)cc1 ; c1ccc(-c2cc(-n3c4ccccc4c4ccccc43)nc(-c3cccc([Si](c4ccccc4)(c4ccccc4)c4cccc(-c5nc(-c6ccccc6)cc(-n6c7ccccc7c7ccccc76)n5)c4)c3)n2)cc1] 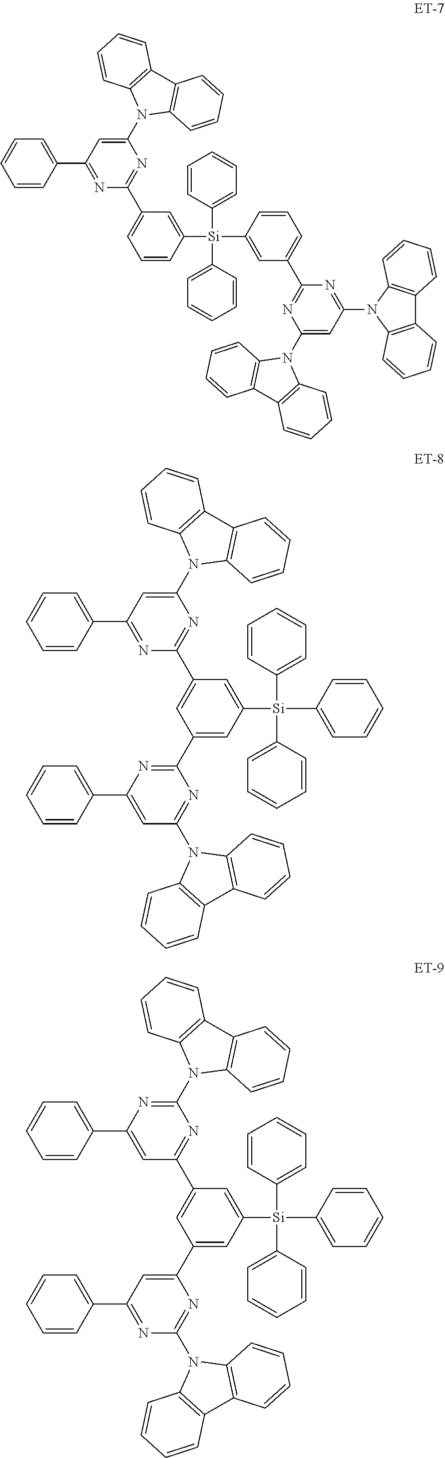 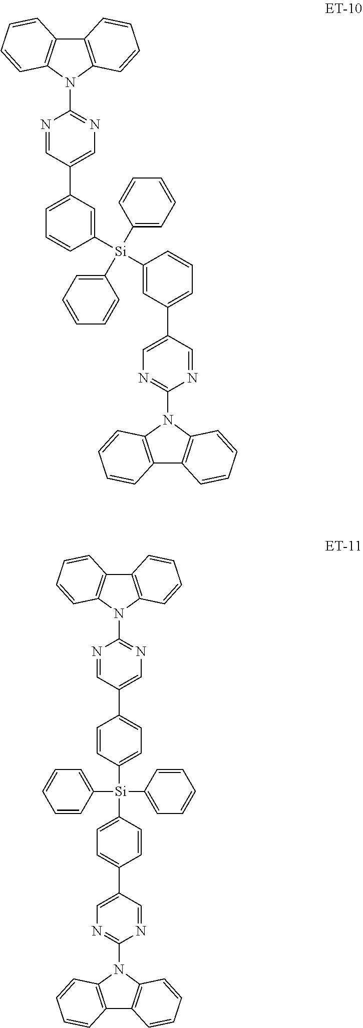 [SMILES: c1ccc([Si](c2ccccc2)(c2cccc(-c3cnc(-n4c5ccccc5c5ccccc54)nc3)c2)c2cccc(-c3cnc(-n4c5ccccc5c5ccccc54)nc3)c2)cc1 ; c1ccc([Si](c2ccccc2)(c2ccc(-c3cnc(-n4c5ccccc5c5ccccc54)nc3)cc2)c2ccc(-c3cnc(-n4c5ccccc5c5ccccc54)nc3)cc2)cc1] 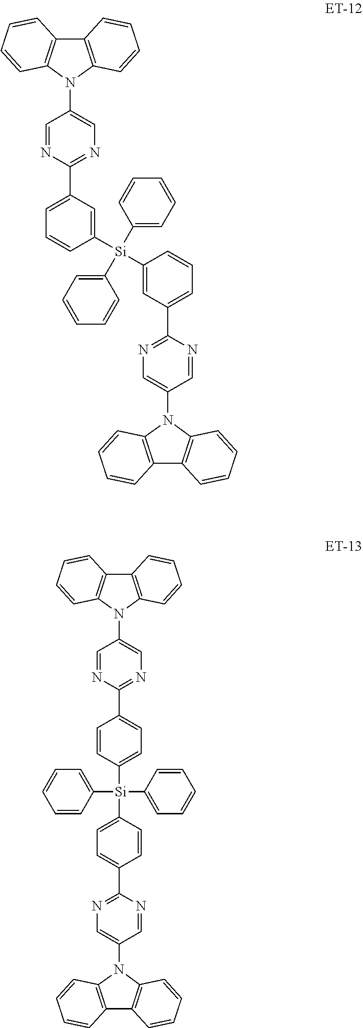 [SMILES: c1ccc([Si](c2ccccc2)(c2cccc(-c3ncc(-n4c5ccccc5c5ccccc54)cn3)c2)c2cccc(-c3ncc(-n4c5ccccc5c5ccccc54)cn3)c2)cc1 ; c1ccc([Si](c2ccccc2)(c2ccc(-c3ncc(-n4c5ccccc5c5ccccc54)cn3)cc2)c2ccc(-c3ncc(-n4c5ccccc5c5ccccc54)cn3)cc2)cc1] 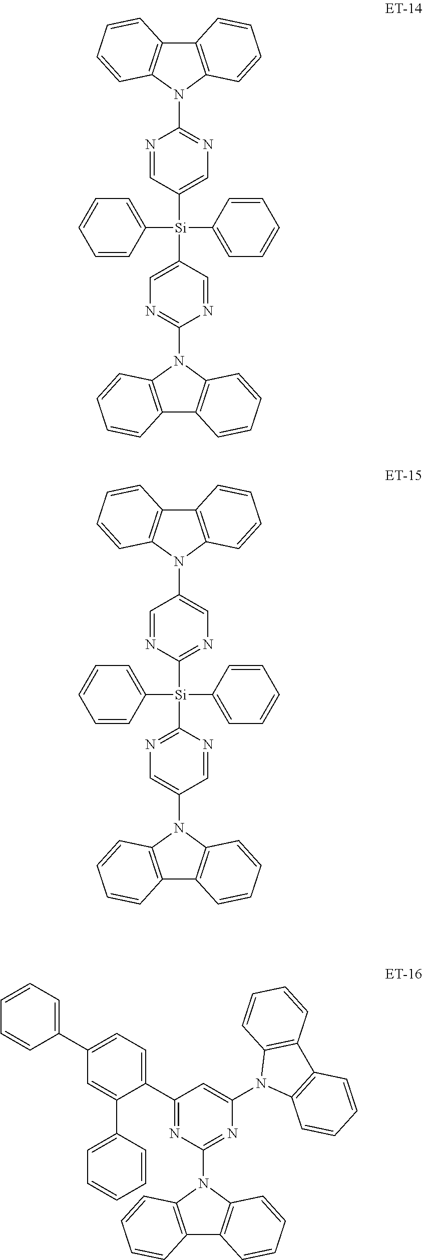 [SMILES: c1ccc([Si](c2ccccc2)(c2cnc(-n3c4ccccc4c4ccccc43)nc2)c2cnc(-n3c4ccccc4c4ccccc43)nc2)cc1 ; c1ccc([Si](c2ccccc2)(c2ncc(-n3c4ccccc4c4ccccc43)cn2)c2ncc(-n3c4ccccc4c4ccccc43)cn2)cc1 ; c1ccc(-c2ccc(-c3cc(-n4c5ccccc5c5ccccc54)nc(-n4c5ccccc5c5ccccc54)n3)c(-c3ccccc3)c2)cc1] 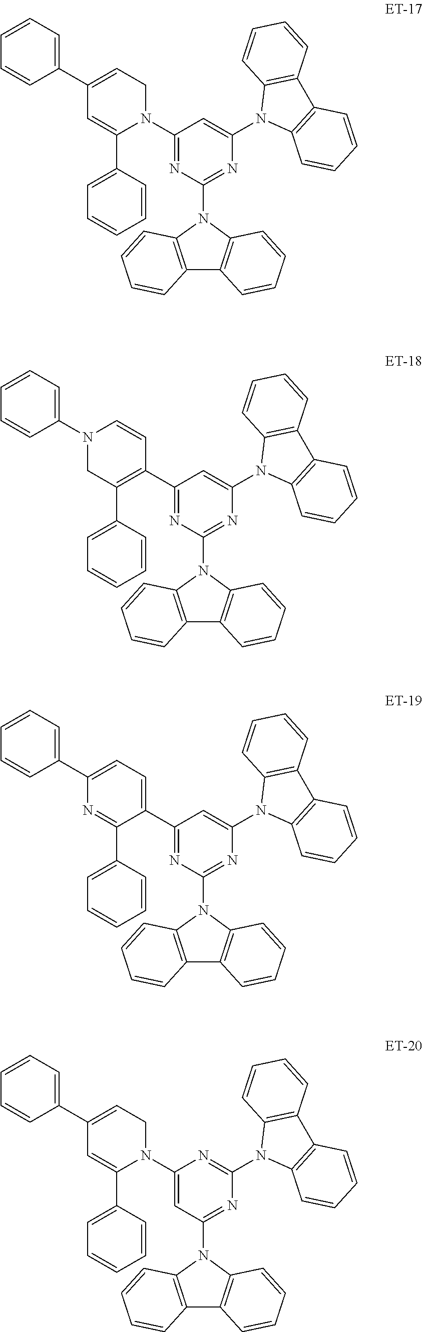 [SMILES: C1=C(c2ccccc2)C=C(c2ccccc2)N(c2cc(-n3c4ccccc4c4ccccc43)nc(-n3c4ccccc4c4ccccc43)n2)C1 ; C1=CN(c2ccccc2)CC(c2ccccc2)=C1c1cc(-n2c3ccccc3c3ccccc32)nc(-n2c3ccccc3c3ccccc32)n1 ; c1ccc(-c2ccc(-c3cc(-n4c5ccccc5c5ccccc54)nc(-n4c5ccccc5c5ccccc54)n3)c(-c3ccccc3)n2)cc1 ; C1=C(c2ccccc2)C=C(c2ccccc2)N(c2cc(-n3c4ccccc4c4ccccc43)nc(-n3c4ccccc4c4ccccc43)n2)C1] 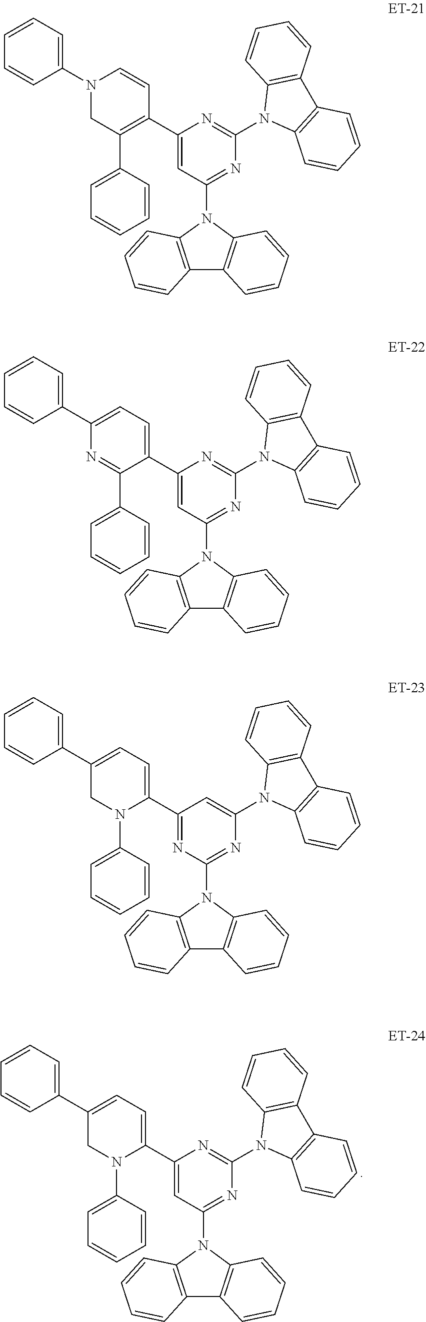 [SMILES: C1=CN(c2ccccc2)CC(c2ccccc2)=C1c1cc(-n2c3ccccc3c3ccccc32)nc(-n2c3ccccc3c3ccccc32)n1 ; c1ccc(-c2ccc(-c3cc(-n4c5ccccc5c5ccccc54)nc(-n4c5ccccc5c5ccccc54)n3)c(-c3ccccc3)n2)cc1 ; C1=C(c2ccccc2)CN(c2ccccc2)C(c2cc(-n3c4ccccc4c4ccccc43)nc(-n3c4ccccc4c4ccccc43)n2)=C1 ; C1=C(c2ccccc2)CN(c2ccccc2)C(c2cc(-n3c4ccccc4c4ccccc43)nc(-n3c4ccccc4c4ccccc43)n2)=C1]
